SEQUENCE LISTING (31 sequences)

SEQ ID NO: 1 - DNA, 897 bp, Human orthopneumovirus
  source 1..897: mol_type = genomic DNA
atgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtag

SEQ ID NO: 2 - DNA, 15104 bp, Human orthopneumovirus
  source 1..15104: mol_type = genomic DNA
ttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtgcgcgctggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggcgatcgacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgcgtcaacacatagcattagttaattaaaaattagggcccaacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaaccgcggagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccactcgagcttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 3 - DNA, 711 bp, Human metapneumovirus
  source 1..711: mol_type = genomic DNA
atggaggtgaaagtggagaacattcgaacaatagatatgctcaaagcaagagtaaaaaatcgtgtggcacgcagcaaatgctttaaaaatgcctctttggtcctcataggaataactacattgagtattgccctcaatatctatctgatcataaactataaaatgcaaaaaaacacatctgaatcagaacatcacaccagctcatcacccatggaatccagcagagaaactccaacggtccccacagacaactcagacaccaactcaagcccacagcatccaactcaacagtccacagaaggctccacactctactttgcagcctcagcaagctcaccagagacagaaccaacatcaacaccagatacaacaaaccgcccgcccttcgtcgacacacacacaacaccaccaagcgcaagcagaacaaagacaagtccggcagtccacacaaaaaacaacccaaggacaagctctagaacacattctccaccacgggcaacgacaaggacggcacgcagaaccaccactctccgcacaagcagcacaagaaagagaccgtccacagcatcagtccaacctgacatcagcgcaacaacccacaaaaacgaagaagcaagtccagcgagcccacaaacatctgcaagcacaacaagaatacaaaggaaaagcgtggaggccaacacatcaacaacatacaaccaaactagttaa

SEQ ID NO: 4 - DNA, 13350 bp, Human metapneumovirus
  source 1..13350: mol_type = genomic DNA
gtataaattagattccaaaaaaatatgggacaagtgaaaatgtctcttcaagggattcacctgagtgatttatcatacaagcatgctatattaaaagagtctcagtacacaataaaaagagatgtgggtacaacaactgcagtgacaccctcatcattgcaacaagaaataacactgttgtgtggagaaattctgtatgctaaacatgctgactacaaatatgctgcagaaataggaatacaatatattagcacagctttaggatcagagagagtgcagcagattctgaggaactcaggcagtgaagtccaagtggtcttaaccagaacgtactctctggggaaaattaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatattcactaaactagcatcaaccatagaagtgggactagagaccacagtcagaagggctaaccgtgtactaagtgatgcactcaagagataccctagaatggacataccaaagattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggggccggtcaaacaatgctaaggtggggggtcattgccaggtcatccaacaatataatgttaggacatgtatccgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgagggagagtaccaaacacagaattattttcagcagctgaaagttatgccaaaagtttgaaagaaagcaataaaataaatttctcttcattaggacttacagatgaagagaaagaggctgcagaacatttcttaaatgtgagtgacgacagtcaaaatgattatgagtaattaaaaaagtgggacaagtcaaaatgtcattccctgaaggaaaagatattcttttcatgggtaatgaagcagcaaaattagcagaagctttccagaaatcattaagaaaaccaggtcataaaagatctcaatctattataggagaaaaagtgaatactgtatcagaaacattggaattacctactatcagtagacctgcaaaaccaaccataccgtcagaaccaaagttagcatggacagataaaggtggggcaaccaaaactgaaataaagcaagcaatcaaagtcatggatcccattgaagaagaagagtctaccgagaagaaggtgctaccctccagtgatgggaaaacccctgcagaaaagaaactgaaaccatcaactaacaccaaaaagaaggtttcatttacaccaaatgaaccagggaaatatacaaagttggaaaaagatgctctagatttgctctcagataatgaagaagaagatgcagaatcttcaatcttaacctttgaagaaagagatacttcatcattaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagctaaagggaaagcagcagaaatgatggaagaggaaatgagtcaacgatcaaaaataggaaatggtagtgtaaaattaacagaaaaagcaaaagagctcaacaaaattgttgaagatgaaagcacaagtggagaatccgaagaagaagaagaaccaaaagacacacaagacaatagtcaagaagatgacatttaccagttaattatgtagtttaataaaaataaacaatgggacaagtaaaaatggagtcctacctagtagacacctatcaaggcattccttacacagcagctgttcaagttgatctaatagaaaaggacctgttacctgcaagcctaacaatatggttccctttgtttcaggccaacacaccaccagcagtgctgctcgatcagctaaaaaccctgacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaactggaatttgacaaactcacagtctgtgaagtaaaaacagtttacttaacaaccatgaaaccatacgggatggtatcaaaatttgtgagctcagccaaatcagttggcaaaaaaacacatgatctaatcgcactatgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattaattatgatcatgactatgaacaatcccaaaggcatattcaaaaagcttggagctgggactcaagtcatagtagaactaggagcatatgtccaggctgaaagcataagcaaaatatgcaagacttggagccatcaagggacaagatatgtcttgaagtccagataacaaccaagcaccttggccaagagctactaaccctatctcatagatcataaagtcaccattctagttatataaaaatcaagttagaacaagaattaaatcaatcaagaacgggacaaataaaaatgtcttggaaagtggtgatcattttttcattgttaataacacctcaacacggtcttaaagagagctacttagaagagtcatgtagcactataactgaaggatatctcagtgttctgaggacaggttggtacaccaatgtttttacactggaggtaggcgatgtagagaaccttacatgtgccgatggacccagcttaataaaaacagaattagacctgaccaaaagtgcactaagagagctcagaacagtttctgctgatcaactggcaagagaggagcaaattgaaaatcccagacaatctagattcgttctaggagcaatagcactcggtgttgcaactgcagctgcagttacagcaggtgttgcaattgccaaaaccatccggcttgaaagtgaagtaacagcaattaagaatgccctcaaaaagaccaatgaagcagtatctacattggggaatggagttcgtgtgttggcaactgcagtgagagagctgaaagattttgtgagcaagaatctaacacgtgcaatcaacaaaaacaagtgcgacattgctgacctgaaaatggccgttagcttcagtcaattcaacagaaggttcctaaatgttgtgcggcaattttcagacaacgctggaataacaccagcaatatctttggacttaatgacagatgctgaactagccagagctgtttccaacatgccaacatctgcaggacaaataaaactgatgttggagaaccgtgcaatggtaagaagaaaagggttcggattcctgataggagtttacggaagctccgtaatttacatggtgcaactgccaatctttggggttatagacacgccttgctggatagtaaaagcagccccttcttgttcaggaaaaaagggaaactatgcttgcctcttaagagaagaccaaggatggtattgtcaaaatgcagggtcaactgtttactacccaaatgaaaaagactgtgaaacaagaggagaccatgtcttttgcgacacagcagcaggaatcaatgttgctgagcagtcaaaggagtgcaacataaacatatctactactaattacccatgcaaagttagcacaggaagacatcctatcagtatggttgcactatctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgaaggcgaacagcatgttataaaaggaaggccagtgtcaagcagctttgacccagtcaagtttcctgaagatcaattcaatgttgcacttgaccaagttttcgagagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaaataaattaaaattaaaaataaaaatttgggacaaatcataatgtctcgcaaggctccgtgcaaatatgaagtgcggggcaaatgcaatagaggaagtgagtgcaagtttaaccacaattactggagttggccagatagatacttattaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaactatctgacagcaaacatgtagcacttcacaacttagtcctatcttatatggagatgagcaaaactcctgcatctttaatcaacaatctcaagagactgccgagagagaaactgaaaaaattagcaaagctcataattgacttatcagcaggtgctgaaaatgactcttcatatgccttgcaagacagtgaaagcactaatcaagtgcagtgagcatggtccagttttcattactatagaggttgatgacatgatatggactcacaaggacttaaaagaagctttatctgatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaaataattaaagaccactctggtaaagtgcttattgtacttaagttaatattagctttactaacatttctcacagtaacaatcaccatcaattatataaaagtggaaaacaatctgcaaatatgccagtcaaaaactgaatcagacaaaaaggactcatcatcaaataccacatcagtcacaaccaagactactctaaatcatgatatcacacagtattttaaaagtttgattcaaaggtatacaaactctgcaataaacagtgacacatgctggaaaataaacagaaatcaatgcacaaatataacaacatacaaatttttatgttttaaatctgaagacacaaaaaccaacaattgtgataaactgacagatttatgcagaaacaaaccaaaaccagcagttggagtgtatcacatagtagaatgccattgtatatacacagttaaatggaagtgctatcattacccaaccgatgaaacccaatcctaaatgttaacaccagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaaaaagtaaaaaacaataacatgggacaagtagttatggaggtgaaagtggagaacattcgaacaatagatatgctcaaagcaagagtaaaaaatcgtgtggcacgcagcaaatgctttaaaaatgcctctttggtcctcataggaataactacattgagtattgccctcaatatctatctgatcataaactataaaatgcaaaaaaacacatctgaatcagaacatcacaccagctcatcacccatggaatccagcagagaaactccaacggtccccacagacaactcagacaccaactcaagcccacagcatccaactcaacagtccacagaaggctccacactctactttgcagcctcagcaagctcaccagagacagaaccaacatcaacaccagatacaacaaaccgcccgcccttcgtcgacacacacacaacaccaccaagcgcaagcagaacaaagacaagtccggcagtccacacaaaaaacaacccaaggacaagctctagaacacattctccaccacgggcaacgacaaggacggcacgcagaaccaccactctccgcacaagcagcacaagaaagagaccgtccacagcatcagtccaacctgacatcagcgcaacaacccacaaaaacgaagaagcaagtccagcgagcccacaaacatctgcaagcacaacaagaatacaaaggaaaagcgtggaggccaacacatcaacaacatacaaccaaactagttaacaaaaaatacaaaataactctaagataaaccatgcagacaccaacaatggagaagccaaaagacaattcacaatctccccaaaaaggcaacaacaccatattagctctgcccaaatctccctggaaaaaacactcgcccatataccaaaaataccacaaccaccccaagaaaaaaactgggcaaaacaacacccaagagacaaataacaatggatcctctcaatgaatccactgttaatgtctatcttcctgactcatatcttaaaggagtgatttcctttagtgagactaatgcaattggttcatgtctcttaaaaagaccttacctaaaaaatgacaacactgcaaaagttgccatagagaatcctgttatcgagcatgttagactcaaaaatgcagtcaattctaagatgaaaatatcagattacaagatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcctaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatatggatgtatagtcaagagcaacaaaagcaaaagagtgagcttcttcacatacaatcaactgttaacatggaaagatgtgatgttaagtagattcaatgcaaatttttgtatatgggtaagcaacagtctgaatgaaaatcaagaagggctagggttgagaagtaatctgcaaggcatattaactaataagctatatgaaactgtagattatatgcttagtttatgttgcaatgaaggtttctcacttgtgaaagagttcgaaggctttattatgagtgaaattcttaggattactgaacatgctcaattcagtactagatttagaaatactttattaaatggattaactgatcaattaacaaaattaaaaaataaaaacagactcagagttcatggtaccgtgttagaaaataatgattatccaatgtacgaagttgtacttaagttattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatccttaggtgggagagcttgacagaactaagaggggcattcatattaaggattatcaaaggatttgtagacaacaacaaaagatggcccaaaattaaaaacttaaaagtgcttagtaagagatggactatgtacttcaaagcaaaaagttaccccagtcaacttgaattaagcgaacaagattttttagagcttgctgcaatacagtttgaacaagagttttctgtccctgaaaaaaccaaccttgagatggtattaaatgataaagctatatcacctcctaaaagattaatatggtctgtgtatccaaaaaattacttacctgagaaaataaaaaatcgatatctagaagagactttcaatgcaagtgatagtctcaaaacaagaagagtactagagtactatttgaaagataataaattcgaccaaaaagaacttaaaagttatgttgttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccttttttcccagaaaccttaacaaagtatggtgatctagatcttcagagaataatggaaatcaaatcggaactttcttctattaaaactagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgtccctatgacaacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctctattagatgttgtatctgtaaaaacacgatgtcaaatgacatctttattaaacggtgacaaccaatcaatagatgtaagtaaaccagttaagttatctgagggtttagatgaagtgaaagcagattatagcttggctgtaaaaatgttaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaataaaaaaggaaaatgaagtagtagatctatggatgaacataccaatgcagtttggaggaggagatccagtagtcttctatagatctttctatagaaggacccctgattttttaactgaagcaatcagtcatgtggatattctgttaagaatatcagccaacataagaaatgaagcgaaaataagtttcttcaaagccttactgtcaatagaaaaaaatgaacgtgctacactgacaacactaatgagagatcctcaagctgttggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtgaatatgatatcaggaacgaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtagggtcgagcactcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgagagatttgggaatgaagatattaatttggtcttccaaaatgcaatcagctgtggaattagcataatgagtgtagtagaacaattaactggtaggagtccaaaacagttagttttaatacctcaattagaagaaatagacattatgccaccaccagtgtttcaagggaaattcaattataagctagtagataagataacttctgatcaacatatcttcagtccagacaaaatagatatgttaacactggggaaaatgctcatgcccactataaaaggtcagaaaacagatcagttcctgaacaagagagagaattatttccatgggaataatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttgggggtcccaagggaaaaacattaaagatgaagatatagtagatgaatcaatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcaaagtttatttttaagaataactgttttgaactatacagatatggcacatgctctcacaagattaatcagaaagaagttgatgtgtgataatgcactattaactccgattccatccccaatggttaatttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagttataggagaattaagcaggataatagatagcggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacttatgggacagacctctatttattcgcaaagtatcatgctaaagactgcaatgtaaaattacctttttttgtgagatcagtagccacctttattatgcaaggtagtaaactgtcaggctcagaatgctacatactcttaacactaggccaccacaacaatttaccctgccatggagaaatacaaaattctaagatgaaaatagcagtgtgtaatgatttttatgctgcaaaaaaacttgacaataaatctattgaagccaactgtaaatcacttttatcagggctaagaataccgataaataagaaagaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagataaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttagaaactaacaactgtcattaagtttattaaaaataagaaattataattggatgtatacg

SEQ ID NO: 5 - DNA, 18 bp, Human orthopneumovirus
  source 1..18: mol_type = genomic DNA
tcaagaccaaaaacacaa

SEQ ID NO: 6 - DNA, 18 bp, synthetic construct
  misc_feature 1..18: note = Description of Artificial Sequence: Synthetic oligonucleotide
  source 1..18: mol_type = other DNA
tcaagacgaaaaatacaa

SEQ ID NO: 7 - DNA, 31 bp, Human orthopneumovirus
  source 1..31: mol_type = genomic DNA
accaaacaaaaaaccaggaaagaaaaccact

SEQ ID NO: 8 - DNA, 31 bp, synthetic construct
  misc_feature 1..31: note = Description of Artificial Sequence: Synthetic oligonucleotide
  source 1..31: mol_type = other DNA
accaaataaaaagccaggaaagaaaacgact

SEQ ID NO: 9 - DNA, 15 bp, Human orthopneumovirus
  source 1..15: mol_type = genomic DNA
ccaaaacaaacatca

SEQ ID NO: 10 - DNA, 15 bp, synthetic construct
  misc_feature 1..15: note = Description of Artificial Sequence: Synthetic oligonucleotide
  source 1..15: mol_type = other DNA
ccaaaacgaatatca

SEQ ID NO: 11 - DNA, 711 bp, Human metapneumovirus; identical to SEQ ID NO: 3
  source 1..711: mol_type = genomic DNA

SEQ ID NO: 12 - DNA, 712 bp, synthetic construct
  misc_feature 1..712: note = Description of Artificial Sequence: Synthetic polynucleotide
  source 1..712: mol_type = other DNA
ttaactagtttggttgtatgttgttgatgtgttggcctccacgcttttcctttgtattcttgttgtgcttgcagatgtttgtgggctcgctggacttgcttcttcgtttttgtgggttgttgcgctgatgtcaggttggactgatgctgtggacggtctctttcttgtgctgcttgtgcggagagtggtggttctgcgtgccgtccttgtcgttgcccgtggtggagaatgtgttctagagcttgtccttgggttgtttttttgtgtggactgccggacttgtctttgttctgcttgcgcttggtggtgttgtgtgtgtgtcgacgaagggcgggcggtttgttgtatctggtgttgatgttggttctgtctctggtgagcttgctgaggctgcaaagtagagtgtggagccttctgtggactgttgagttggatgctgtgggcttgagttggtgtctgagttgtctgtggggaccgttggagtttctctgctggattccatgggtgatgagctggtgtgatgttctgattcagatgtgtttttttgcattttatagtttatgatcagatagatattgagggcaatactcaatgtagttattcctatgaggaccaaagaggcatttttaaagcatttgctgcgtgccacacgattttttactcttgctttgagcatatctattgttcgaatgttctccactttcacctccat

SEQ ID NO: 13 - DNA, 11 bp, Human orthopneumovirus
  source 1..11: mol_type = genomic DNA
ctcaaacacaa

SEQ ID NO: 14 - DNA, 26 bp, Human orthopneumovirus
  source 1..26: mol_type = genomic DNA
acaaaacaacgccaaaacaaaccacc

SEQ ID NO: 15 - DNA, 11 bp, synthetic construct
  misc_feature 1..11: note = Description of Artificial Sequence: Synthetic oligonucleotide
  source 1..11: mol_type = other DNA
ctcaaacgcaa

SEQ ID NO: 16 - DNA, 26 bp, synthetic construct
  misc_feature 1..26: note = Description of Artificial Sequence: Synthetic oligonucleotide
  source 1..26: mol_type = other DNA
acaaagcaacgccaaaataagccacc

SEQ ID NO: 17 - DNA, 24 bp, Human orthopneumovirus
  source 1..24: mol_type = genomic DNA
aaaaaaccaaccctcaagacaacc

SEQ ID NO: 18 - DNA, 18 bp, Human orthopneumovirus
  source 1..18: mol_type = genomic DNA
cccaaacctcaaaccact

SEQ ID NO: 19 - DNA, 24 bp, synthetic construct
  misc_feature 1..24: note = Description of Artificial Sequence: Synthetic oligonucleotide
  source 1..24: mol_type = other DNA
aaaaagccaaccctcaagacgacc

SEQ ID NO: 20 - DNA, 18 bp, synthetic construct
  misc_feature 1..18: note = Description of Artificial Sequence: Synthetic oligonucleotide
  source 1..18: mol_type = other DNA
cccaagcctcaaacgact

SEQ ID NO: 21 - DNA, 30 bp, Human orthopneumovirus
  source 1..30: mol_type = genomic DNA
ccagaactcacaagtcaaatggaaaccttc

SEQ ID NO: 22 - DNA, 30 bp, synthetic construct
  misc_feature 1..30: note = Description of Artificial Sequence: Synthetic oligonucleotide
  source 1..30: mol_type = other DNA
ccagagctcacaagtcaaatggaaacgttc

SEQ ID NO: 23 - DNA, 23 bp, synthetic construct
  misc_feature 1..23: note = Description of Artificial Sequence: Synthetic oligonucleotide
  source 1..23: mol_type = other DNA
ttttttttttttttttttttttt

SEQ ID NO: 24 - DNA, 21 bp, synthetic construct
  misc_feature 1..21: note = Description of Artificial Sequence: Synthetic oligonucleotide
  source 1..21: mol_type = other DNA
ccgcgtctagttgttcatgaa

SEQ ID NO: 25 - DNA, 21 bp, synthetic construct
  misc_feature 1..21: note = Description of Artificial Sequence: Synthetic oligonucleotide
  source 1..21: mol_type = other DNA
aaggacgttcccaatagccaa

SEQ ID NO: 26 - DNA, 21 bp, synthetic construct
  misc_feature 1..21: note = Description of Artificial Sequence: Synthetic oligonucleotide
  source 1..21: mol_type = other DNA
atggattaaatcagtatctaa

SEQ ID NO: 27 - DNA, 21 bp, synthetic construct
  misc_feature 1..21: note = Description of Artificial Sequence: Synthetic oligonucleotide
  source 1..21: mol_type = other DNA
ctgcaagtatgttcactatga

SEQ ID NO: 28 - DNA, 21 bp, synthetic construct
  misc_feature 1..21: note = Description of Artificial Sequence: Synthetic oligonucleotide
  source 1..21: mol_type = other DNA
aaggatgagttaatagctaaa

SEQ ID NO: 29 - DNA, 21 bp, synthetic construct
  misc_feature 1..21: note = Description of Artificial Sequence: Synthetic oligonucleotide
  source 1..21: mol_type = other DNA
aaacaagtacttcttcggcga

SEQ ID NO: 30 - DNA, 21 bp, synthetic construct
  misc_feature 1..21: note = Description of Artificial Sequence: Synthetic oligonucleotide
  source 1..21: mol_type = other DNA
aaatagccgctgcttgtgaga

SEQ ID NO: 31 - DNA, 19 bp, synthetic construct
  misc_feature 1..19: note = Description of Artificial Sequence: Synthetic oligonucleotide
  source 1..19: mol_type = other DNA
acgtgacacgttcggagaa

(19) United States
(12) Patent Application Publication
(10) Pub. No.: US 2025/0250548 A1
(43) Pub. Date: Aug. 7, 2025

(54) METHODS AND COMPOSITIONS FOR TREATING NEGATIVE-SENSE SINGLE-STRANDED RNA VIRUS

(71) Applicants: The University of Chicago, Chicago, IL (US); Nationwide Children's Hospital, Columbus, OH (US); The Ohio State University, Columbus, OH (US)

(72) Inventors: Jianrong LI, Dublin, OH (US); Mark E. PEEPLES, Bexley, OH (US); Chuan HE, Chicago, IL (US); Stefan NIEWIESK, Dublin, OH (US); Mijia LU, Columbus, OH (US); Miaoge XUE, Columbus, OH (US); Zijie ZHANG, Chicago, IL (US); Boxuan ZHAO, Palo Alto, CA (US)

(73) Assignees: The University of Chicago, Chicago, IL (US); Nationwide Children's Hospital, Columbus, OH (US); The Ohio State University, Columbus, OH (US)

(21) Appl. No.: 19/013,940

(22) Filed: Jan. 8, 2025

Related U.S. Application Data
(62) Division of application No. 17/309,038, filed on Apr. 16, 2021, now Pat. No. 12,227,769.
(60) Provisional application No. 62/748,175, filed on Oct. 19, 2018.

(51) Int. Cl. C12N 7/00; A61K 35/76

(52) U.S. Cl. CPC C12N 7/00; A61K 35/76; C12N 2760/18522

(57) ABSTRACT

The current disclosure relates to methods, compositions and kits for detecting modified adenosine in a target RNA molecule. Aspects relate to a method for detecting modified adenosine in a target ribonucleic acid (RNA) comprising contacting the target RNA with an adenosine deaminase enzyme (adenosine deaminase, RNA-specific) to generate a target RNA with deaminated adenosines and sequencing the target RNA with deaminated adenosines; wherein the modified adenosine is detected when the nucleotide sequence includes adenosine within a m6A motif.

### CROSS-REFERENCE TO RELATED APPLICATIONS

This application is a divisional of U.S. patent application Ser. No. 17/309,038, filed Apr. 16, 2021, which is a national phase application under 35 U.S.C. § 371 of International Application No. PCT/US2019/056942, filed Oct. 18, 2019, which claims the benefit of priority of U.S. Provisional Patent Application No. 62/748,175 filed Oct. 19, 2018, all of which are hereby incorporated by reference in their entirety.

### STATEMENT OF GOVERNMENT SUPPORT

This invention was made with government support under AI090060 and AI112524 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Jan. 8, 2025, is named ARCDP0667USD1_SL.xml and is 67,453 bytes in size.

### BACKGROUND OF THE INVENTION

### I. Field of the Invention

The present invention relates generally to the field of medicine. More particularly, it concerns methods and compositions preventing respiratory syncytial virus infection and the disease it causes.

### II. Background

Human respiratory syncytial virus (RSV), a member of the Pneumoviridae [42] and a non-segmented negative-sense (NNS) RNA virus, is the most important cause of upper and lower respiratory tract infection of infants, young children, and immunocompromised individuals and second only to influenza virus for the elderly [43]. Worldwide it is estimated that RSV causes 3.4 million hospitalizations and between 66,000 and 199,000 deaths in children less than 5 years of age [44]. Despite major efforts, no vaccine or antiviral drug is yet available for RSV [43]. The Pneumoviridae family also includes human metapneumovirus (hMPV) which is responsible for 5 to 15% of all respiratory tract infections in infants and young children, a proportion second only to that of RSV [45, 46]. Other important pneumoviruses include avian metapneumovirus (aMPV), pneumonia virus of mice (PVM), and bovine RSV, which cause respiratory tract infections in animals [42]. Together, pneumoviruses are the major causative agents of respiratory tract infection in humans and animals. There is a need in the art for therapeutics that can effectively prevent or treat infections caused by RSV or other viruses in the family Pneumoviridae.

### SUMMARY OF THE INVENTION

The inventors found that the genome (negative-sense), antigenome (positive-sense replication intermediate), and mRNAs (transcription products) of negative-sense single-stranded RNA virus of the family Pneumoviridae, such as respiratory syncytial virus (RSV) or metapneumovirus (MPV) are m6A methylated by host cell methyl transferases, which positively regulates viral replication, gene expression, and virus production in human cells. Viral mutants lacking some or all N⁶-methyladenosine (m6A) modifications provide for attenuated virus with retained immunogenicity. Thus, the current disclosure fulfills a need in the art by providing methods and viral compositions that can be used to treat and/or prevent viral infections, including those caused specifically by RSV and MPV.

In some embodiments, there is an attenuated negative-sense single-stranded RNA virus of the family Pneumoviridae that is attenuated because the virus has reduced modification of its genome, antigenome, and/or mRNA with methylation, particularly m6A modification, as compared to a non-attenuated or wild-type virus. Additional embodiments concern nucleic acid molecules comprising a nucleic acid sequence having and/or encoding one or more altered m6A consensus sequence sites, host cells containing such nucleic acids, host cells with the ability to yield increased or decreased m6A modifications, including being capable of producing higher yields of viral vaccines that are attenuated by other methods or producing higher or lower yields of one or more proteins or viruses that result from increased methylation, methods of producing attenuated virus, and methods of inducing an immune response using such attenuated RSV.

Embodiments of the disclosure relate to a negative-sense single-stranded RNA virus of the family Pneumoviridae such as syncytial virus (RSV) or metapneumovirus (MPV). Thus, the embodiments described herein are wherein the virus comprises RSV. In other embodiments, the description relates to embodiments in which the virus comprises MPV. The virus may be one that is isolated or replicated in a mammal, such as a human, mouse, rabbit, or rat. In particular embodiments, the virus in one that is isolated from a human and/or is capable of infecting human cells.

Embodiments involve sequence alterations in the viral genome or antigenome encoding N⁶-methyladenosine (m6A) consensus sites in viral mRNA, antigenome, or genome that disrupt the consensus sequence sites for m6A modification such that these sites in the mRNA, antigenome, and/or genome are no longer modified by m6A. As the genome, antigenome, and mRNA all have m6A modifications, whether the consensus site alteration affects the genome or the antigenome/mRNA depends on whether the alteration is made in a way to result to a change in the consensus sequence in the genome or the antigenome/mRNA. A person of ordinary skill in the art understands the complementary nature of these sequences and can identify in which sequence a change needs to be made in order to effect a change in the genome, antigenome, or mRNA that destroys the consensus sequence, which is understood to be from 5′ to 3′: Pu [G>A]m⁶AC[A/C/U] motif (Pu represents purine). The complement from 5′ to 3′ would be understood to be [U/G/A]GU Py (Py represents pyrimidine). In some embodiments, alterations of a m6A consensus sequence may be 1, 2, 3, or 4 of the following changes: the initial Pu such that it no longer is a purine and is instead a pyrimidine; a U, C, or G substituted for the A that is 3′ to the m⁶-modified Pu; a G, U, A substituted for the C on the 3′ side of the A that is 3′ to the m⁶-modified Pu; or, a G substituted for [A/C/U]. Embodiments concern at least 1, 2, 3, or 4 substitutions of nucleic acid residues in a single consensus site.

While some embodiments concern substitution(s) of nucleic acid residue(s), an alteration may include the addition or deletion of nucleotides to alter the sequence such that it no longer functions as an m6A consensus sequence.

In specific embodiments, the sequence alterations change at least two nucleotides of at least one m6A consensus site. In other embodiments, there is at least one sequence alteration that comprises a resulting change of at least an adenine (A) in an m6A consensus site in viral mRNA, genome, and/or the antigenome. In additional embodiments, there is at least one sequence alteration that comprises a resulting change of at least a cytosine in an m6A consensus site in viral mRNA, genome, and/or antigenome. In particular embodiments, there are at least two sequence alterations and the two sequence alterations comprise a change of an adenine and cytosine in the same m6A consensus site in viral mRNA, genome, and/or antigenome. In other embodiments, the sequence alterations changing an m6A consensus site does not alter the amino acid sequence of an encoded polypeptide. In other embodiments, exactly, at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more modified m6A consensus sequence sites (or any range derivable therein) do not result in an altered amino acid at a series of m6A sites in one or multiple genes. In particular embodiments, none of the modified m6A consensus sequence sites results in an altered amino acid being encoded.

In some embodiments, multiple m6A consensus sequence sites may be modified. It is contemplated that exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more m6A consensus sequence sites (or any range derivable therein) in a viral genome, antigenome, and/or mRNA may be altered.

It is contemplated that in certain embodiments the sequence alterations result in reduction of m6A modifications of viral mRNA, genome, and/or antigenome. In some embodiments, there is about, at least about, or at most about a reduction of m6A modification in a virus genome, antigenome, or mRNA (total mRNA or a specific mRNA or a specific subset of mRNA) of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any range derivable therein) compared to the virus lacking the sequence alterations.

In some embodiments, the sequence alterations cause replication of the attenuated virus to be reduced by about, at least about or at most about 5, 10, 15, 20, 25, 30, 35, 40, 45, 50, 60, 70, 80, 90, 100, 110, 120, 130, 140, 150, 160, 170, 180, 190, 200% or more (or any range derivable therein) or by a fold decrease of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein). The reduction can be measured with respect to any measurement of viral replication, including, but not limited to any measurement set forth in the Examples. In certain embodiments, replication is at least 3-fold, 5-fold, 10-fold, or 20-fold reduced (or any range derivable therein) compared to the virus without mutations in the viral genome encoding one or more N⁶-methyladenosine (m6A) consensus sites in viral mRNA, genome, or the antigenome.

In some embodiments, the sequence alterations lead to a change in one or more m6A consensus sites in RSV mRNA, genome, and/or RSV antigenome corresponding to the G gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in regions 392-467 nt, 567-660 nt, and/or 716-795 nt of the G gene. In particular embodiments, there is 1, 2, 3, 4, 5, 6, 7, 8, 9, 10, 11, 12, 13, 14, 15 or more (or any range derivable therein) altered consensus sites in region 392-467 nt of the G gene. In further embodiments, there is 1, 2, 3, 4, 5, 6, 7, 8, 9, 10, 11, 12, 13, 14, 15 or more (or any range derivable therein) altered consensus sites in region 567-660 nt of the G gene. In other embodiments, there is 1, 2, 3, 4, 5, 6, 7, 8, 9, 10, 11, 12, 13, 14, 15 or more (or any range derivable therein) altered consensus sites in region 716-795 nt of the G gene. In specific embodiments, there is 1, 2, 3, 4, 5, 6, 7, 8, 9, 10, 11, 12, 13, 14, 15, 16, 17, 18, 19, 20 or more (or any range derivable therein) consensus sites in regions 392-467 nt and 567-660 nt of the G gene. In specific embodiments, there is 1, 2, 3, 4, 5, 6, 7, 8, 9, 10, 11, 12, 13, 14, 15, 16, 17, 18, 19, 20 or more (or any range derivable therein) in regions 392-467 nt and 716-795 nt of the G gene. In specific embodiments, there is 1, 2, 3, 4, 5, 6, 7, 8, 9, 10, 11, 12, 13, 14, 15, 16, 17, 18, 19, 20 or more (or any range derivable therein) in regions 567-660 nt and 716-795 nt of the G gene. In specific embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more (or any range derivable therein) in regions 392-467 nt, 567-660 nt, and 716-795 nt of the G gene.

In some embodiments, the sequence alterations lead to a change in one or more m6A consensus sites in MPV mRNA, genome, and/or MPV antigenome corresponding to the G gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein). In some embodiments, the consensus sequence sites comprise one or more of the sites corresponding to sites 1-14 of FIG. 50 of the MPV antigenome or one or more of the following m6A sites in the antigenome: site 1, 171-AAm⁶AC»TA-175; site 2, 187-GAm⁶A»GCA-191; site 3, 227-AAm⁶ACT»G-231; site 4, 246-AGm⁶AC»TA-250; site 5, 255-AGm⁶AC»TA-259; site 6, 341-AGm⁶ACA»G-345; site 7, 346-GAm⁶A»GCC-351; site 8, 422-GAm⁶ACA»G-426; site 9, 428-AGm⁶ACA»G-432; site 10, 453-AAm⁶AC»TA-457; site 11, 464-GGm⁶ACA»G-468; site 12, 476-GAm⁶ACA»G-480; site 13, 518-GAm⁶ACC»G-522; and site 14, 553-AGm⁶A»GCC-557. Thus, in particular embodiments, at least 1 of sites 1-14 is mutated. In some embodiments, at least or at most 2 sites of sites 1-14 is mutated. In some embodiments, at least or at most 3 sites of sites 1-14 is mutated. In some embodiments, at least or at most 4 sites of sites 1-14 is mutated. In some embodiments, at least or at most 5 sites of sites 1-14 is mutated. In some embodiments, at least or at most 6 sites of sites 1-14 is mutated. In some embodiments, at least or at most 7 sites of sites 1-14 is mutated. In some embodiments, at least or at most 8 sites of sites 1-14 is mutated. In some embodiments, at least or at most 9 sites of sites 1-14 is mutated. In some embodiments, at least or at most 10 sites of sites 1-14 is mutated. In some embodiments, at least or at most 11 sites of sites 1-14 is mutated. In some embodiments, at least or at most 12 sites of sites 1-14 is mutated. In some embodiments, at least or at most 13 sites of sites 1-14 is mutated. In some embodiments, at least 14 sites of sites 1-14 is mutated. In some embodiments, at least site 1 is mutated. In some embodiments, at least site 2 is mutated. In some embodiments, at least site 3 is mutated. In some embodiments, at least site 4 is mutated. In some embodiments, at least site 5 is mutated. In some embodiments, at least site 6 is mutated. In some embodiments, at least site 7 is mutated. In some embodiments, at least site 8 is mutated. In some embodiments, at least site 9 is mutated. In some embodiments, at least site 10 is mutated. In some embodiments, at least site 11 is mutated. In some embodiments, at least site 12 is mutated. In some embodiments, at least site 13 is mutated. In some embodiments, at least site 14 is mutated. In some embodiments, the genome is mutated. In some embodiments, mutated m6A consensus sites comprise one or more consensus sites corresponding to sites 1-6 of FIG. 51 in the MPV genome or one or more of the following m6A sites in the genome: site 1, 237-G»CGm⁶TC»GC-241; site 2, 290-AG»Am⁶TCC»A-294; site 3, 433-AGm⁶T»C CC-437; site 4, 441-A»C Gm⁶TC»GC-445; site 5, 570-AGm⁶T»C CC-574; and site 6, 616-AG»Am⁶TCC»G-620. Thus, in particular embodiments, at least 1 of sites 1-6 is mutated. In some embodiments, at least or at most 2 sites of sites 1-6 is mutated. In some embodiments, at least or at most 3 sites of sites 1-6 is mutated. In some embodiments, at least or at most 4 sites of sites 1-6 is mutated. In some embodiments, at least or at most 5 sites of sites 1-14 is mutated. In some embodiments, at least 6 sites of sites 1-6 is mutated. In some embodiments, at least site 1 is mutated. In some embodiments, at least site 2 is mutated. In some embodiments, at least site 3 is mutated. In some embodiments, at least site 4 is mutated. In some embodiments, at least site 5 is mutated. In some embodiments, at least site 6 is mutated.

In additional embodiments, there may be sequence alterations affecting an m6A consensus sequence in the genome, antigenome, or mRNA corresponding to the N, P, M, NS1, NS2, F, SH, M2-1, M2-2, and/or L genes. These may be instead of or in addition to sequence alterations affecting the G gene. In some embodiments, the sequence alterations lead to a change in one or more m6A consensus sites in viral mRNA, genome, and antigenome and/or the genome corresponding to the N gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the N gene. In some embodiments, the sequence alterations lead to a change in one or more m6A consensus sites in RSV mRNA and antigenome and/or the genome corresponding to the P gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the P gene. In some embodiments, the sequence alterations lead to a change in one or more m6A consensus sites in viral mRNA and antigenome and/or the genome corresponding to the M gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the M gene. In some embodiments, the sequence alterations lead to a change in one or more m6A consensus sites in mRNA and antigenome and/or the genome corresponding to the L gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the L gene. In some embodiments, the sequence alterations lead to a change in one or more m6A consensus sites in mRNA and antigenome and/or the genome corresponding to the NS1 gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the NS1 gene. In some embodiments, the sequence alterations lead to a change in one or more m6A consensus sites in mRNA and antigenome and/or the genome corresponding to the NS2 gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the NS2 gene. In some embodiments, the sequence alterations lead to a change in one or more m6A consensus sites in mRNA and antigenome and/or the genome corresponding to the F gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the F gene. In some embodiments, the sequence alterations lead to a change in one or more m6A consensus sites in mRNA and antigenome and/or the genome corresponding to the SH gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the SH gene. In some embodiments, the sequence alterations lead to a change in one or more m6A consensus sites in mRNA and antigenome and/or the genome corresponding to the M2-1 gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the M2-1 gene. In some embodiments, the sequence alterations lead to a change in one or more m6A consensus sites in mRNA and antigenome and/or the genome corresponding to the M2-2 gene. In certain embodiments, there is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more sequence alterations (or any range derivable therein) in 1, 2, 3, 4, 5, 6, 7, 8, 9, 10, 11, 12, 13, 14, 15, 16, 17, 18, 19, 20, 21, 22, 23, 24, 25 m6A consensus sequence sites (or any range derivable therein) in the genome, antigenome, or mRNA corresponding to the M2-2 gene. Moreover, it is specifically contemplated that A or B strains can be used, as well as any of genotypes GA1, GA2, GA3, GA4, GA5, GA6, GA7, SAA1, NA1, NA2, GB1, GB2, GB3, GB4, SAB1, SAB2, SAB3, BA1, BA2, BA3, BA4, BA5, or BA6.

The genome of human RSV was fully sequenced in 1997. Strain include, but not limited to, the following: A/A2, A/1998/12-21 A/RSV-12. A/Riyadh/2009 B/9320, B/NH1276, B/TX11-56, A/GN435/11, A/ON1. It is contemplated that sequence alterations described herein will affect m6A modifications in multiple strains.

In some embodiments, the MPV strain includes. NL/1/00. In some embodiments, the MPV strain includes HMPV subtype A and B lineages (A1, A2, B1, or B2), HMPV NL/1/00, HMPV CA/83/97, HMPV JP/240/03, HMPV CN/gz01/08, HMPV NL/1/99, HMPV CA/75/98.

In some embodiments, nucleic acid molecules having sequence alterations that lead to altered m6A modification in the viral genome or antigenome and mRNA are specifically contemplated. The nucleic acid molecules may be DNA or RNA. In some embodiments, there is an infectious cDNA viral clone containing sequence alterations; an infectious cDNA clone may contain a full-length antigenome of the virus in some embodiments. In other embodiments, there may be one or more genes or other genomic regions with altered m6A consensus sites in a DNA or RNA. In some embodiments, there is also a host cell that is cultured under conditions that accommodate virus replication.

Some embodiments concern methods for inhibiting a negative-sense single-stranded RNA virus of the family Pneumoviridae in a patient, for vaccinating a patient against a negative-sense single-stranded RNA virus of the family Pneumoviridae, for increasing immunity against a negative-sense single-stranded RNA virus of the family Pneumoviridae in a patient, for providing protective immunity against a negative-sense single-stranded RNA virus of the family Pneumoviridae in a patient, for inducing antibodies directed against a negative-sense single-stranded RNA virus of the family Pneumoviridae in a patient, for reducing the severity of an infection from a negative-sense single-stranded RNA virus of the family Pneumoviridae in a patient, for reducing mortality from infection by respiratory syncytial virus in a patient, as well as methods for producing an attenuated respiratory syncytial virus, for producing a vaccine against respiratory syncytial virus, and for producing a respiratory syncytial virus with reduced amount of m6A modification. It is specifically contemplated that any embodiment discussed in the context of a virus of the family Pneumoviridae can be specifically applied or implemented with respect to RSV, MSV, or both. Similarly, any embodiment discussed in the context of RSV can be applied to MSV or another virus of the family Pneumoviridae. It is also specifically contemplated that a specific virus virus of the family Pneumoviridae may be excluded in an embodiment.

In some embodiments methods comprise administering to the patient a composition comprising attenuated virus, such as attenuated negative-sense single-stranded RNA virus of the family Pneumoviridae, including attenuated RSV or MPV discussed in the above paragraphs and in other parts of this disclosure. In specific embodiments, methods comprise administering an effective amount of a composition comprising attenuated RSV. It is contemplated that a patient is administered 1, 2, 3, 4, 5 or more compositions comprising attenuated virus, which may be given at different intervals, with weeks, months, and/or years between an administration. It is contemplated that a patient may receive one or more boosters following an initial vaccination or set of vaccinations. It is contemplated that the amount of viral particles in a composition is 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰ viral particles (vp) or plaque forming units (pfu or any range derivable therein). In certain embodiments, there are 10⁴-10⁸ viral particles or pfu, in the composition.

In some embodiments, the methods, compositions or viruses of the disclosure are ones that are capable of inducing a higher expression of type I interferon in vivo. In some embodiments, the the methods, compositions or viruses of the disclosure are ones that are capable of attenuation in the respiratory tract, such as the lower respiratory track while retaining high immunogenicity. In some embodiments, the methods, compositions or viruses of the disclosure are ones that are capable of

While any human patient may be administered attenuated virus, in some embodiments the patient is a pediatric patient, meaning the patient is under the age of 18 years old. A patient under the age of 18 may be termed a pediatric patient. In additional embodiments, the patient is an infant, meaning less than 1 year old at the time of being administered a first and/or a last administration of a composition comprising attenuated virus. In other embodiments, the patient is 5 or younger, is 3 or younger, or is 2 or younger. In some embodiments, the patient is a premature infant. In other embodiments, the patient is a geriatric patient, such 50 or older, 55 or older, 60 or older, 65 or older, or 70 or older. In particular embodiments, the patient is at risk for a viral infection, such as an RSV or MPV infection, which includes but is not limited to medical clinicians, healthcare providers, teachers, hospital workers, or others in areas of higher than average infection rates. It is contemplated that the patient is a subject who is otherwise healthy and/or does not exhibit symptoms of a viral infection, such as an RSV or MPV infection. In specific embodiments, the patient does not exhibit one or more of nasal congestion, runny nose, mild cough, low-grade or high fever, barking cough, difficulty breathing, wheezing, difficulty drinking, lethargy, irritability, bluish color around mouth, lips and/or fingernails, or sleep apnea. In other embodiments, the patient is immunocompromised.

In some embodiments, there are methods for creating an attenuated virus comprising transfecting a cell line with a nucleic acid encoding an attenuated virus; culturing the cell line under conditions to promote viral replication; and collecting viral particles. In certain embodiments, the cell line used to grow the attenuated virus is VERO, MRC-5, HEp-2, A549, or HeLa. In particular embodiments, the cell line is cultured under serum-free conditions. In other embodiments, there are methods for producing an attenuated virus comprising infecting a cell line with an attenuated RSV; culturing the cell line under conditions to promote virus replication; and collecting viral particles. Other steps such as isolating the virus, purifying the virus, freezing the virus, testing the virus, and/or quantitating the virus are included in some embodiments.

In some embodiments, the cell line comprises cells that are reduced in endogenous expression of one or more m6A writer proteins. For example, the cells may comprise an inhibitor of a writer protein or mRNA or may comprise a genetic alteration of the endogenous writer gene. In some embodiments, the writer gene has been disrupted to that no function writer protein is produced in the viral particle or in the host cell. For example, the disruption may be through genetic alteration of the genomic or antigenomic DNA or through inhibition by, for example, siRNA, shRNA, morpholino, antisense nucleic acids, and other ways of inhibiting the production of a protein. In some embodiments, the gene encoding for the writer protein has been mutated by gene editing. In some embodiments, the writer protein comprises one or both of METTL3 and METTL14.

Other methods concern inhibiting a negative-sense single-stranded RNA virus of the family Pneumoviridae in a patient comprising administering to the patient an effective amount of a composition comprising an inhibitor of N⁶-methyladenosine (m6A) methylation. In some embodiments, the inhibitor is a S-adenosylhomocysteine (SAH) hydrolase inhibitor such as sinefungin. In particular embodiments, the SAH hydrolase inhibitor is 3-deazaadenosine (DAA) or carbocyclic 3-deazaadenosine. In particular embodiments, the patient may be a pediatric patient, meaning the patient is under the age of 18 years old. In additional embodiments, the patient is an infant, meaning less than 1 year old at the time of being administered a first or a last administration of a composition comprising an m6A inhibitor. In other embodiments, the patient is 5 or younger, is 3 or younger, or is 2 or younger. In some embodiments, the patient is a premature infant. In other embodiments, the patient is a geriatric patient, such 50 or older, 55 or older, 60 or older, 65 or older, or 70 or older. In particular embodiments, the patient is at risk for a viral infection, such as an RSV or MPV infection, which includes but is not limited to medical clinicians, healthcare providers, teachers, hospital workers, or others in areas of higher than average infection rates. It is contemplated that the patient is a subject who is otherwise healthy and/or does not exhibit symptoms of a viral infection, such as an RSV or MPV infection. In specific embodiments, the patient does not exhibit one or more of nasal congestion, runny nose, mild cough, low-grade or high fever, barking cough, difficulty breathing, wheezing, difficulty drinking, lethargy, irritability, bluish color around mouth, lips and/or fingernails, or sleep apnea. In other embodiments, the patient is immunocompromised. It is specifically contemplated that any embodiment may be implemented with respect to a pediatric patient, including or excluding a patient who is an infant.

Embodiments also concern a host cell comprising a heterologous nucleic acid encoding exactly or at least or at most 1, 2, 3, 4, or 5 N⁶-methyladenosine (m6A) reader, eraser, or writer proteins. The host cell may include the writer proteins METTL3 and/or METTL14. In other embodiments, the host cell may include the reader protein YTHDF1, YTHDF2, YTHDF3 and/or YTHDC1. In other embodiments, the cells may include one or more eraser proteins. In some embodiments, the eraser proteins comprise one or both of FTO and ALKBH5. In some embodiments, the cells comprise an inhibitor of an eraser proteins or the cells may be ones that are reduced in their expression of eraser proteins. For example, the cells may comprise a nucleic acid inhibitor of one or more eraser proteins, such as an siRNA, shRNA, antisense, or morpholino, or the cells may have a disruption of one or more eraser genes such that they gene does not produce a functional protein. In some embodiments, the reader, eraser, and/or writer proteins may be overexpressed relative to endogenous levels of expression or at least 1, 2, 3, 4, 5, 6, 7, 8, 9, 10, 20, 40, 50, or 100 times the expression levels of endogenous expression levels (or any range derivable therein). Such a host cell can be used to produce an attenuated virus whose replication is positively affected by m6A modification but that has not been attenuated through loss of m6A consensus sequence sites. Such viruses include RSV, poliovirus, measles virus, mumps virus, rubella virus, yellow fever virus, influenza virus, parainfluenza viruses, metapneumoviruses, Zika virus, dengue viruses, or rhinoviruses. Any embodiment may be implement with or specifically without any of these viruses. It is contemplated that any nonsegmented negative sense RNA virus may be produced in such a cell line, including but not limited to those non-segmented negative-sense (NNS) RNA viruses encompassing a wide range of significant human, animal, and plant pathogens in five families: Paramyxoviridae, Pneumoviridae, Rhabdoviridae, Filoviridae, and Bornaviridae. RSV belongs to the family Pneumoviridae. Other viruses in family Pneumoviridae. also include human metapneumovirus (hMPV). Methods of producing any of these viruses comprise in some embodiments culturing the above-described host cell that also contains an infectious virus or infectious virus clone under conditions to promote viral replication. Other steps may include collecting the replicated virus particles, isolating and/or purifying the virus.

In further embodiments, the cell line can be employed to enhance production of vaccine vectors that deliver various other virus antigens. Such vectors include, but are not limited to, adenovirus, Sendai virus, vesicular stomatitis virus, parainfluenza viruses, measles virus and Newcastle disease virus n some embodiments, the host cell further comprises a heterologous nucleic acid encoding the attenuated virus or a gene or genes whose expression is enhanced by m6A methylation.

The use of the word “a” or “an” when used in conjunction with the term “comprising” in the claims and/or the specification may mean “one,” but it is also consistent with the meaning of “one or more,” “at least one,” and “one or more than one.” Any embodiment discussed in the context of comprising may be substituted with the phrase consisting of or consisting essentially of.

It is contemplated that any embodiment discussed herein can be implemented with respect to any method or composition of the invention, and vice versa. Furthermore, compositions can be used to achieve any method embodiments. Any embodiment discussed in the Examples, Figures, or Description of the Drawings can be implemented in the context of any embodiment discussed elsewhere in this disclosure.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, non-recited elements or method steps. The term consisting essentially of, when referring to a therapeutic composition is intended to include all the recited active ingredients and excludes non-recited active ingredients, but also includes any other ingredients, such as excipients, that are not therapeutically active.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. The RSV genome and antigenome/mRNAs are m⁶A methylated. (A) Distribution of m⁶A peaks in the RSV antigenome and genome of virions grown in HeLa cells. Confluent HeLa cells were infected by rgRSV at an MOI of 1.0, supernatant was harvested at 36 h post-infection. RSV virions were purified by sucrose gradient ultracentrifugation. Total RNAs were extracted from purified virions and were subjected to m⁶A-specific antibody immunoprecipitation followed by high throughput sequencing (m⁶A-seq). A schematic diagram of the RSV antigenome encoding 10 genes is shown. The normalized coverage from m⁶A-seq of RSV RNA showing the distribution of m⁶A-immunoprecipitated (IP) reads mapped to the RSV antigenome and genome. The baseline distributions for antigenome and genome from input sample are shown as a blue and pink line respectively. Data presented are the averages from two independent virion samples (n=2). (B) Distribution of m⁶A peaks in the RSV mRNAs from RSV-infected HeLa cells. Confluent HeLa cells were infected by rgRSV at an MOI of 1.0, cell lysates were harvested at 36 h post-infection. Total RNAs were extracted from cell lysates, and were enriched for mRNA by binding to oligo dT, and subjected to m⁶A-seq. The distribution of m⁶A-immunoprecipitated (IP) reads were mapped to the RSV mRNAs. The baseline distributions for mRNAs from input sample are shown as a pink line. Data presented are the averages from two independent virus-infected HeLa cell samples (n=2). (C) Distribution of m⁶A peaks in the RSV antigenome and genome of virions grown in A549 cells. Data presented are the averages from two independent virion samples (n=2). (D) Distribution of m⁶A peaks in the RSV mRNAs from RSV-infected A549 cells. Data presented are the averages from two independent virus-infected A549 cell samples (n=2).

FIGS. 2A-K. YTHDF1, 2, 3 (reader) proteins promote RSV replication, gene expression, and progeny virus production. (A) Detection of YTHDF1, 2 3 in HeLa cells stably overexpressing YTHDF1-3. Western blot confirmed the overexpression of YTHDF1-3 proteins in HeLa cells using anti-Flag antibody. (B) YTHDF1, 2, 3 enhance GFP expression in rgRSV-infected cells. HeLa cells stably overexpressing these YTHDF proteins were infected with rgRSV at an MOI of 0.1, and GFP expression was monitored at the indicated times by fluorescence microscopy. (C) YTHDF1, 2, 3 increase the number of GFP-positive cells quantified by flow cytometry. (D) YTHDF1, 2, 3 enhance RSV protein expression. Total cell extracts were harvested from rgRSV-infected HeLa cells at the indicated times and subjected to Western blot using antibody against RSV N, F, or G protein. Western blots shown are the representatives of three independent experiments. RSV F (F0+F1) (E), G (F), and N (G) proteins were quantified by Image J Software. Data are expressed as mean of three independent experiments±standard deviation. (H) YTHDF1, 2, 3 increases RSV progeny virus production. The release of infectious RSV particles was monitored by a single-step growth curve. Virus titer was measured by TCID50. (I) YTHDF1, 2, 3 enhances RSV genomic RNA replication. Total RNA was purified from rgRSV-infected cells using TRizol, and genomic RNA was quantified by real-time RT-PCR using specific primers annealing to the RSV leader sequence and GFP gene. (J) YTHDF1, 2, 3 enhance mRNA transcription. Viral mRNA was separated from total RNA using the Dynabeads mRNA isolation kit and quantified by real-time PCR using primers annealing to the NS1 gene. (K) Ratio between mRNA and genomic RNA. The ratio between NS1 mRNA and genomic RNA was calculated for each cell line. All results are from three independent experiments. Flow cytometry data are expressed as mean±standard deviation. RNA copy and viral titer are the geometric mean titer (GMT) of three independent experiments±standard deviation.

FIGS. 3A-D. Knockdown of endogenous YTHDF1, 2, 3 (reader) proteins diminishes RSV gene expression. HeLa cells were transfected with 150 pmole of siRNA targeting YTHDF1, 2 3 or control siRNA. At 36 h post-transfection, cells were infected with rgRSV at an MOI of 0.5. (A) Immunoblot analysis of YTHDF1, 2, 3 in HeLa cells transfected with siRNA. (B) Immunoblot analysis of RSV G and F proteins. (C) Dynamics of GFP expression in YTHDF1, 2, 3 protein-depleted HeLa cells. (D) Quantification of GFP-positive cells by flow cytometry at 18 h post-inoculation. Fold of GFP signal compared to the control is shown. Western blots and GFP images shown are the representatives of three independent experiments. Flow cytometry data are expressed as mean±standard deviation. The P value (Student's t-test) for YTHDF1, 2, and 3 is 1.098×10⁻⁶, 0.00170, and 0.000972, respectively.

FIGS. 4A-F. Effects of m⁶A writer proteins on RSV gene expression. (A) Overexpression of m⁶A writer proteins enhances RSV gene expression. HeLa cells were transfected with plasmids encoding METTLE3 and/or METTL14. At 36 h post-transfection, cells were infected with rgRSV at an MOI of 0.5. At 18 h post-infection, cell lysates were harvested for Western blot analysis. (B) Overexpression of m⁶A writer proteins enhances RSV expression of GFP. The GFP expression in m⁶A writer protein-overexpressed HeLa cells following rgRSV infection was monitored by fluorescence microscopy. Representative images at 18 h post-infection were shown. (C) Quantification of GFP-positive cells by flow cytometry at 18 h post-infection. Fold of GFP signal compared to the control is shown. The P value (Student's t-test) for METTL3, METTL14, and METTL3 &METTL14 is 0.000276, 0.000873, and 0.00228, respectively. (D) Knockdown of m⁶A writer proteins diminishes RSV gene expression. HeLa cells were transfected with siRNA targeting METTL3 and/or METTL14. At 36 h post-transfection, cells were infected with rgRSV at an MOI of 0.5. At 18 h post-infection, cell lysates were harvested for Western blot analysis. (D) Knockdown of m⁶A writer proteins diminishes GFP expression. The GFP expression in m⁶A writer protein-depleted cells following rgRSV infection was monitored by fluorescence microscopy. (E) Quantification of GFP-positive cells by flow cytometry. Western blots and GFP images shown are the representatives of three independent experiments. Fold of GFP signal compared to the control is shown. Flow cytometry data are expressed as mean±standard deviation. The P value (Student's t-test) for METTL3, METTL14, and METTL3 &METTL14 is 0.00441, 0.00458, and 0.000134, respectively.

FIGS. 5A-F. Effects of m⁶A eraser proteins on RSV gene expression. (A) Overexpression of m⁶A eraser proteins diminishes RSV gene expression. HeLa cells were transfected with plasmids encoding ALKHB5 and/or FTO. At 36 h post-transfection, cells were infected with rgRSV at an MOI of 0.5. At 18 h post-infection, cell lysates were harvested for Western blot analysis. (B) Overexpression of m⁶A eraser proteins reduces GFP expression. The GFP expression in m⁶A eraser protein-overexpressed HeLa cells following rgRSV infection was monitored by fluorescence microscopy. Representative images at 18 h post-infection are shown. (C) Quantification of GFP-positive cells by flow cytometry at 18 h post-infection. The P value for ALKBH5, FTO, and ALKBH5&FTO is 1.913×10⁻⁶, 1.338×10⁻⁵, and 3.613×10⁻⁶, respectively. (D) Knockdown of m⁶A eraser proteins enhances RSV gene expression. HeLa cells were transfected with siRNA targeting ALKHB5 and/or FTO. At 36 h post-transfection, cells were infected with rgRSV at an MOI of 0.5. At 18 h post-infection, cell lysates were harvested for Western blot analysis. (D) Knockdown of m⁶A eraser proteins enhances GFP expression. The GFP expression in m⁶A eraser protein-depleted cells following rgRSV infection was monitored by fluorescence microscopy. (E) Quantification of GFP-positive cells by flow cytometry. Fold of GFP signal compared to the control is shown. Western blots and GFP images shown are representatives of three independent experiments. Flow cytometry data are expressed as mean±standard deviation. The P value for ALKBH5, FTO, and ALKBH5&FTO is 2.056×10⁻⁴, 3.382×10⁻⁵, and 6.499×10⁻⁶, respectively.

FIGS. 6A-E. RSV infection does not alter the m⁶A reader, writer, or eraser protein distribution in cells. HeLa cells were infected by rgRSV at an MOI of 10.0. At 24 h post-infection, mock- or rgRSV-infected cells were stained with anti-reader, writer, or eraser protein antibody and anti-RSV N protein antibody, and were analyzed by confocal microscope. Nuclei were labeled with DAPI. (A) m⁶A reader protein YTHDF1; (B) m⁶A writer protein METTL3; and (C) m⁶A eraser protein FTO. (D) Detection of m⁶A reader, writer, and eraser proteins by Western blot. Nuclear and cytoplasmic fractions were separated from mock- or rgRSV-infected HeLa cells, and were subjected to Western blot. Nuclear and cytoplasmic markers were indicated by Lamin A and α-Tubulin, respectively. Representative results from three independent experiments are shown.

FIGS. 7A-G. m⁶A-abrogating RSV mutants have defects in replication in immortalized cells. (A) Immunoblot analysis of RSV proteins. Confluent A549 cells were infected with each rgRSV at an MOI of 0.1, cell lysates were harvested at 18, 24, and 48 h post-infection, and RSV proteins were detected by specific antibodies against F and G protein. (B) GFP expression of m⁶A-deficient rgRSV mutants. (C) Quantification of GFP-positive cells by flow cytometry. The P value for rgRSV-G1, G2, G3, G12, and G123 at 18 h post-inoculation is 5.868×10⁻⁶, 7.130×10⁻⁵, 1.646×10⁻⁵, 6.489×10⁻⁵, and 6.983×10⁻⁶, respectively; at 24 h post-inoculation is 0.00261, 0.0418, 0.00766, 0.0138, and 0.0230 respectively; at 48 h post-inoculation is 3.545×10⁻⁶, 1.822×10⁻⁴, 9.828×10⁻⁶, 7.782×10⁻⁵, and 2.475×10⁻⁵, respectively. (D) Single step growth curve of m⁶A-deficient rgRSV mutants in HeLa cells. Confluent HeLa cells were infected with each rgRSV at an MOI of 1.0, supernatants were harvested at the indicated time, and viral titer was determined by TCID₅₀ assay. (E) RSV genomic RNA replication. Total RNA was purified from rgRSV-infected cells using TRizol, and genomic RNA was quantified by real-time RT-PCR using specific primers annealing to the RSV leader sequence and GFP gene. The P value for rgRSV-G1 at 18, 24, and 48 h is 0.000653, 8.658×10⁻⁵, and 1.330×10⁻⁵ respectively. The P value for rgRSV-G12 at 48 h is 0.000141. (F) RSV NS1 mRNA transcription. Viral mRNA was separated from total RNA using the Dynabeads mRNA isolation kit and quantified by real-time PCR using primers annealing to the NS1. The P value for rgRSV-G1 at 18, 24, and 48 h is 1.797×10⁻⁵, 1.112×10⁻⁴, and 0.00119, respectively. (G) RSV G mRNA transcription. The P value for rgRSV-G1 at 18, 24, and 48 h is 7.128×10⁻⁵, 5.019×10⁻⁶, and 1.222×10⁻⁶, respectively. The P value for rgRSV-G12 at 18, 24, and 48 h is 0.00942, 0.000199, and 1.347×10⁻⁵, respectively. Results are from three independent experiments. Flow cytometry data are expressed as mean±standard deviation. RNA copy and viral titer are the geometric mean titer (GMT) of three independent experiments±standard deviation. Western blots shown are the representatives of three independent experiments.

FIGS. 8A-C. m⁶A-abrogating RSV mutants have defects in replication in HAE culture. (A) Spreading of m⁶A deficient rgRSVs in HAE culture. HAE cultures were infected by 800 TCID₅₀ of each rgRSV. At the indicated time, virus spreading was monitored by fluorescence microscopy. Representative images at each time point were shown. (B) Quantification of GFP signal in HAE culture. GFP signal was quantified by Image J software, and data are expressed as mean±standard deviation. (C) Virus release from m⁶A deficient rgRSV-infected HAE culture. HAE cultures were infected by 800 TCID₅₀ of each rgRSV. After virus inoculation, supernatants were collected every 2 days until day 14 post-inoculation. Infectious virus in supernatants was determined by TCID₅₀ assay. Viral titers are the geometric mean titer (GMT) of three independent experiments±standard deviation.

FIGS. 9A-E. Pathogenicity and immunogenicity of m⁶A-deficient rgRSVs in cotton rats. (A) RSV titer in lungs. Four-week-old SPF cotton rats were inoculated intranasally with 2.0×10⁵ TCID₅₀ of each rgRSV. At day 4 post-infection, the cotton rats were sacrificed, and lungs and nasal turbinates were collected for virus titration by TCID₅₀ assay. Viral titers are the geometric mean titer (GMT) of 5 animals±standard deviation. Detection limit is 2.0 log TCID₅₀/g tissue. (B) RSV titer in nasal turbinates. (C) m⁶A deficient rgRSVs had less lung histopathological changes compared to rgRSV. Representative pathological changes from each group are shown. Right lung lobe of each cotton rat was fixed in 4% neutral buffered formaldehyde, embedded in paraffin, sectioned at 5 μm, and stained with hematoxylin-eosin (HE) for the examination of histological changes by light microscopy. Micrographs with 20× magnification are shown. (D) m⁶A deficient rgRSV provides complete protection against RSV challenge. Four-week-old SPF cotton rats were inoculated intranasally with 2.0×10⁵ TCID50 of each rgRSV. At week 4 post-immunization, cotton rats were challenged with 2.0×10⁵ TCID₅₀ rgRSV. At day 4 post-challenge, the cotton rats were sacrificed, and lungs and nasal turbinates were collected for virus titration by TCID50 assay. Viral titers are the geometric mean titer (GMT) of 5 animals±standard deviation. The detection limit is 2.0 log TCID50/g tissue. (E) rgRSV induced a high level of neutralizing antibody. Blood samples were collected from each rat weekly by retro-orbital bleeding. The RSV-neutralizing antibody titer was determined using a plaque reduction neutralization assay, as described in Materials and Methods. The P value for rgRSV-G1 at week 3 and 4 is 0.0166 and 0.0490, respectively.

FIGS. 10A-C. The attenuated phenotype of m⁶A deficient rgRSVs is m⁶A-related. (A) rgRSV-G1 and -G12 were less dependent on the m⁶A eraser protein. A549 cells were transfected with a plasmid encoding ALKHB5. At 36 h post-transfection, cells were infected with each rgRSV at an MOI of 0.5. At 18 h post-infection, cell lysates were harvested for Western blot analysis. (B) rgRSV-G1, 2, 3 expression was less dependent on m⁶A writer protein. A549 cells were transfected with control siRNA or siRNA targeting METTL4 and METTL13. At 36 h post-transfection, cells were infected with each rgRSV at an MOI of 0.5. At 18 h post-infection, cell lysates were harvested for Western blot analysis. The density of Western blot was quantified by Image J software, and the ratio of the protein bands was calculated. (C) Distribution of m⁶A peaks on the RSV mRNAs from A549 cells infected by rgRSV and rgRSV-G12. Confluent A549 cells were infected by each m⁶A-deficient rgRSV at an MOI of 1.0, cell lysates were harvested at 36 h post-infection. Total RNAs were extracted from cell lysates, and were enriched for mRNA by binding to oligo dT, and subjected to m⁶A-seq. The distribution of m⁶A-immunoprecipitated (IP) reads were mapped to the RSV mRNAs (pink block). The baseline distributions for mRNAs from input sample are shown as a pink line. Data presented are the mean coverage from two independent virus-infected A549 cell samples (n=2). Red arrow indicates the m⁶A enrichment in G mRNA.

FIGS. 11A-B. A methyltransferase inhibitor Cc3Ado inhibits RSV replication in HEp-2 cells and HAE cultures. (A) HEp-2 cells were infected with 1,000 TCID₅₀ of rgRSV or rgRSV-G1857A-G1853A in the presence or absence of 15 μg/ml of Cc3Ado, and GFP expression was photographed at day 3 post-infection. (B) HAE cultures were infected with 1,000 TCID₅₀ of rgRSV or rgRSV-G1857A-G1853A in the presence or absence of 50 μg/ml of Cc3Ado, and GFP expression recorded at day 3 post-infection. Data are representative of three independent experiments.

FIGS. 12A-E. RSV infection alters the methylome of host transcripts in HeLa cells. Total RNAs were isolated from mock-infected and rgRSV-infected HeLa cells. Poly(A) enriched mRNAs were purified and subjected to m⁶A-seq. (A) Metagene analysis of m^{6A} peaks distribution along the human mRNA in control and infected HeLa cells. (B) Metagene analysis of m⁶A peak distribution on lncRNA. (C and D) Distribution of m⁶A peaks in the 5′ UTR, CDS, and 3′ UTR of host cell mRNA transcripts. Charts show the proportion of m⁶A peaks in the indicated regions in uninfected (C) and rgRSV-infected HeLa cells (D). (E) GO graphs showing pathway clusters from differential expressed genes in rgRSV-infected HeLa cells. Data presented are the averages from duplicate samples (n=2).

FIGS. 13A-E. RSV infection alters the methylome of host transcripts in A549 cells. Total RNAs were isolated from mock-infected and rgRSV-infected A549 cells. Poly(A) enriched mRNAs were purified and subjected to m⁶A-seq. (A) Metagene analysis of m⁶A peak distribution along the human mRNA in control and infected A549 cells. (B) Metagene analysis of m⁶A peak distribution in lncRNA. (C and D) Distribution of m⁶A peaks in the 5′ UTR, CDS, and 3′ UTR of host cell RNA transcripts. Charts show the proportion of m⁶A peaks in the indicated regions in uninfected (C) and rgRSV-infected A549 cells (D). (E) GO graphs showing pathway clusters from differential expressed genes in rgRSV-infected A549 cells. Data presented are the average results from duplicate samples (n=2).

FIGS. 14A-C. Transient expression of YTHDF1, 2, 3 proteins enhances RSV gene expression in HeLa cells. HeLa cells were transfected with 1 μg of plasmids encoding YTHDF1, 2, 3 or pCAGGS. At 36 h post-transfection, cells were infected with rgRSV at an MOI of 0.5. (A) Immunoblot analysis of YTHDF1, 2, 3 protein expression. (B) Immunoblot analysis of RSV G and F protein expression. (C) GFP expression. Data are from three independent experiments. Western blots shown are representative of three independent experiments. Flow cytometry data are expressed as mean±standard deviation.

FIGS. 15A-C. Transient expression of YTHDF1, 2, or 3 proteins enhances RSV gene expression in A549 and Vero cells. A549 or Vero cells were transfected with 1 μg of plasmid. At 36 h post-transfection, cells were infected with rgRSV at an MOI of 0.5. (A) Immunoblot analysis of RSV F, G, N, and HA-tagged reader proteins in A549 cells. (B) Dynamics of GFP expression in YTHDF1, 2 or 3 transfected A549 cells at 24 h post-infection. (C) Immunoblot analysis of RSV G and F proteins in Vero cells. Data are from three independent experiments. Western blots shown are the representatives of three independent experiments.

FIGS. 16A-D. HeLa cells stably expressing m⁶A-related proteins did not significantly affect cell growth or metabolism. (A) The effect of overexpression of m⁶A-related protein on cell growth. A549 cells were transfected with 1 μg of plasmids encoding m⁶A-related genes. At 24 and 48 h post-transfection, cells were trypsinized and counted by flow cytometry. Flow cytometry data are plotted as mean of 3 independent experiments±standard deviation. (B) Raw flow cytometry plot at 48 h. (C) The effect of knockdown of m⁶A-related protein on cell growth. A549 cells were transfected with control siRNA or siRNA targeting m⁶A-related genes. Cell count from flow cytometry at 24 and 48 h. (D) Raw flow cytometry cell counts at 48 h post transfection.

FIGS. 17A-B. Distribution of m⁶A reader proteins YTHDF2 and 3 in mock and RSV-infected HeLa cells. HeLa cells were infected with rgRSV at an MOI of 10. At 24 h post-infection, mock- or rgRSV-infected cells were stained with anti-reader antibody and anti-RSV N protein antibody, and analyzed by confocal microscopy. Nuclei were labeled with DAPI. (A) Reader protein YTHDF2; and (B) Reader protein YTHDF3.

FIG. 18. Distribution of m⁶A writer protein METTL14 in mock and RSV-infected HeLa cells. HeLa cells were infected with rgRSV at an MOI of 10. At 24 h post-infection, mock- or rgRSV-infected cells were stained with anti-writer antibody and anti-RSV N protein antibody and analyzed by confocal microscopy. Nuclei were labeled with DAPI.

FIG. 19. Distribution of m⁶A eraser protein ALKBH5 in mock and RSV-infected HeLa cells. HeLa cells were infected with rgRSV at an MOI of 10. At 24 h post-infection, mock- or rgRSV-infected cells were stained with anti-eraser antibody and anti-RSV N protein antibody and analyzed by confocal microscopy. Nuclei were labeled with DAPI.

FIGS. 20A-D. m⁶A reader protein binds to RSV genomic RNA and mRNA. HeLa cells stably expressing YTHDF2 and vector control HeLa cells were infected with rgRSV at an MOI of 1.0. At 24 h post-infection, cells were lysed and cytoplasmic extracts were immunoprecipitated with an antibody against YTHDF2 (A) or an equivalent amount of HA-tag (non-specific IgG control) (C). The amount of vgRNA and mRNA captured by the YTHDF2 antibody (B) or the HA-tag antibody (D) was quantified by real-time RT-PCR, as was the input RNA, and graphed as the percentage of input. Data are representative of two experiments.

FIG. 21. Mutagenesis strategy in putative m⁶A site in the RSV G mRNA. Schematic diagram of the RSV genome with the mutations for altering the critical A or C residues in the m⁶A motifs to produce rgRSV lacking that putative m⁶A modification site in the G gene. Three m⁶A peaks, G1, G2, and G3, are shown; each containing 6, 7, and 4 m⁶A sites, respectively. Consensus m⁶A motifs and inactivating mutations are shown. Dashes represent nucleotides not shown. G gene sequence of RSV A2 strain (accession number M74568) is shown. FIG. 21 discloses SEQ ID NOS 5, 13-14, 6, 15-16, 7, 17-18, 8, 19-20, 9, 21, 10, and 22, respectively, in order of appearance.

FIGS. 22A-B. Replication of m⁶A deficient rgRSVs in A549 cells. (A) GFP expression of m⁶A deficient rgRSVs. Confluent A549 cells were infected with each m⁶A-deficient rgRSV mutant at an MOI of 1.0, GFP images were photographed at 18, 24, and 48 h post-infection. (B) Quantification of GFP-positive cells by flow cytometry. GFP images shown are representative of three independent experiments. Flow cytometry data are expressed as mean of three independent experiments±standard deviation.

FIG. 23. Immunization with m⁶A deficient rgRSVs protects cotton rats from lung damage after RSV challenge. Four-week-old SPF cotton rats were inoculated intranasally with 2.0×10⁵ TCID₅₀ of each rgRSV. At week 4 post-immunization, cotton rats were challenged with 2.0×10⁵ TCID₅₀ rgRSV. At day 4 post-challenge, the cotton rats were sacrificed, and right lung lobe of each cotton rat was fixed in 4% neutral buffered formaldehyde, embedded in paraffin, sectioned at 5 μm, and stained with hematoxylin-eosin (HE) for the examination of histological changes by light microscopy. Representative pathological changes from each group are shown.

FIGS. 24A-D. Distribution of m⁶A peaks on the RSV mRNAs from A549 cells infected by m⁶A-deficient rgRSVs. Confluent A549 cells were infected by each m⁶A-deficient rgRSV at an MOI of 1.0, cell lysates were harvested at 36 h post-infection. Total RNAs were extracted from cell lysates, and were enriched for mRNA by binding to oligo dT, and subjected to m⁶A-seq. The distribution of m⁶A-immunoprecipitated (IP) reads were mapped to the RSV mRNAs. The baseline distributions for mRNAs from input sample are shown as a line. Data presented are the mean coverage from two independent virus-infected A549 cell samples (n=2). Arrow indicates the m⁶A enrichment in G mRNA.

FIG. 25. Conserved m⁶A sites in different RSV strains. Based on the m⁶A-seq data from rgRSV-infected A549 cells, the G mRNA has a total of 25 putative m⁶A sites. 100 RSV strains with full-length G mRNA available in GeneBank were selected for sequence alignment. Conserved m⁶A sites in these 100 RSV strains were identified. X axis indicates the 25 putative m⁶A sites. Y axis indicates the numbers of RSV strains containing this specific m⁶A site in X axis.

FIG. 26A-C. The hMPV genome and antigenome are m⁶A methylated. A schematic diagram of the hMPV antigenome encoding 8 genes is shown. Total RNAs were extracted from sucrose gradient purified rhMPV virions grown in A549 cells and were subjected to m⁶A-specific antibody immunoprecipitation followed by high throughput sequencing (m⁶A-seq). (A) Distribution of m⁶A peaks in the hMPV antigenome and genome. Top panel: The m⁶A-seq of hMPV RNA showing the distribution of m⁶A reads mapped to the hMPV antigenome. The baseline signal from input samples is shown as a blue line. Lower panel: The distribution of m⁶A reads from m⁶A-seq was mapped to the hMPV genome. The baseline signal from input samples is shown as a light grey line. The arrow indicates the m⁶A peak. (B) Distribution of m⁶A peaks in the hMPV mRNAs. Data presented are the average results from duplicate samples (n=2). The arrow indicates the m⁶A peak. (C) List of m⁶A peaks in hMPV genome, antigenome, and mRNAs. a. Nucleotide sequence is referred to subtype A strain NL/1/00 (GenBank accession number AF371337). Nucleotide ranges are indicated. b. The hMPV genes are covered by m⁶A peaks. These regions may contain m⁶A sites. c. log 2 enrichment of the m⁶A peaks identified in hMPV antigenome, genome, and mRNA. The P value for each peak is indicated.

FIG. 27A-D. hMPV infection alters the transcriptome of host transcripts. Total RNAs were isolated from mock-infected and hMPV-infected A549 cells. Poly(A) enriched mRNAs were purified and subjected to m⁶A-seq. (A) Motif analysis to identify consensus sequences for m⁶A methylation sites in uninfected and hMPV-infected A549 cells. Frequency of nucleotides at the three positions flanking the central m⁶A sites is shown. (B) Metagene analysis of normalized m⁶A peak distribution along the human reference mRNA in control and infected cells. (C and D) GO graphs showing functional clusters from upregulated genes (C) or downregulated genes (D) identified in hMPV-infected cells.

FIG. 28A-G. m⁶A reader proteins promote hMPV replication, gene expression, and progeny virus production in A549 cells. (A) Overexpression of m⁶A reader proteins. A549 cells were transfected with plasmids encoding YTHDF1, 2, 3, or YTHDC1. At 24 h post-transfection, cells were lysed and subjected to Western blot. YTHDF1-3 proteins were detected by anti-HA tag antibody and YTHDC1 was detected by anti-YTHDC1 antibody. (B) m⁶A reader proteins enhance hMPV protein expression in A549 cells. A549 cells were transfected with plasmids encoding YTHDF1, 2, 3, or YTHDC1. At 24 h post-transfection, cells were infected with rhMPV at an MOI of 5.0. At 12, 18, 24, and 48 h post-infection, total cell extracts were harvested and subjected to Western blot using antibody against hMPV N or G protein. (C) m⁶A increases hMPV progeny virus production. The release of infectious hMPV particles was monitored by a single-step growth curve. Virus titer was measured by an immunostaining plaque assay. *P<0.05, **P<0.01, ***P<0.001, and ****P<0.0001. Exact P values of YTHDF1, 2, 3, or YTHDC1 compared to pCAGGS are as follows: 12 h, P=0.00124, P=0.00025, P=0.00305, P=0.00003; 18 h, P=0.00871, P=0.00277, P=0.00264, P=0.00207; 24 h, P=0.00115, P=0.00182, P=0.00305, P=0.00993; 48 h, P=0.00170, P=0.00379, P=0.00055, P=0.00165. (D) hMPV genome replication. Total RNA was purified from rhMPV-infected cells using TRizol, and genomic RNA was quantified by real-time RT-PCR using specific primers annealing to the hMPV leader sequence and N gene. (E) hMPV antigenome replication. hMPV antigenome was quantified with specific primers annealing to the hMPV trailer sequence and L gene. Exact P value: P=0.04937, P=0.04752, P=0.00925. (F) G mRNA transcription. N- and G-mRNA and GAPDH mRNA copies were quantified from cDNA pool generated from total RNA and Oligo (dT)₃ (SEQ ID NO: 23). RNA and mRNA copies were normalized by GAPDH. Exact P value: 12 h, P=0.00191, 18 h, P=0.00878, P=0.01498, P=0.01973; 24 h, P=0.00263, P=0.02688, P=0.01444; 48 h, P=0.03959, P=0.01511, P=0.03071. (G) N mRNA transcription. P=0.00364, P=0.00350. All results are from three independent experiments. Western blots shown are the representatives of three independent experiments. RNA copy and viral titer are the geometric mean titer (GMT) of three independent experiments±standard deviation.

FIG. 29A-J. YTHDF1, 2, 3 (reader) proteins promote hMPV replication, gene expression, and progeny virus production in HeLa cells. (A) Detection of YTHDF1, 2 3 in HeLa cells stably overexpressing YTHDF1-3. Western blot confirmed the overexpression of YTHDF1-3 proteins in HeLa cells using anti-Flag antibody. (B)YTHDF1, 2, 3 enhance hMPV protein expression in HeLa cells. HeLa cells stably overexpressing these YTHDF proteins were infected with rhMPV-GFP at an MOI of 0.5. Total cell extracts were harvested from hMPV-infected HeLa cells at the indicated times and subjected to Western blot using antibody against hMPV N, F, or G protein. Western blots shown are the representatives of three independent experiments. (C) YTHDF1, 2, 3 enhance GFP expression in hMPV-GFP-infected cells. HeLa cells stably overexpressing these YTHDF proteins were infected with rhMPV at an MOI of 1.0, and GFP expression was monitored at the indicated times by fluorescence microscopy. (D) YTHDF1, 2, 3 increase the number of GFP-positive cells quantified by flow cytometry. Flow cytometry data are expressed as mean±standard deviation. P values of YTHDF1, 2, and 3 compared to vector control are as follows: 12 h, P=0.00004, P=0.00219, P=0.01188; 18 h, P=0.00005, P=0.00714, P=0.00607; 24 h, P=0.00018, P=0.00507, P=0.00414; 48 h, P=0.0000004, P=0.000003, P=0.000002. (E) YTHDF1, 2, 3 enhance GFP intensity. P values of YTHDF1, 2, 3 compared to vector are as follows: 12 h, P=0.003802, P=0.004390, P=0.003389; 18 h, P=0.000003, P=0.000214, P=0.000004; 24 h, P=0.000003, P=0.000208, P=0.000023; 48 h, P=0.001032, P=0.000045, P=0.000033. (F) YTHDF1, 2, 3 increases hMPV progeny virus production. The release of infectious hMPV particles was monitored by a single-step growth curve. Virus titer was measured by an immunostaining plaque assay. Viral titers are the geometric mean titer (GMT) of three independent experiments±standard deviation. P values of YTHDF1, 2, 3 compared to vector are as follows: 12 h, P=0.02307, P=0.01020, P=0.00953; 18 h, P=0.00151, P=0.01140, P=0.00013; 24 h, P=0.00017, P=0.00178, P=0.00003; 48 h, P=0.00023, P=0.00018, P=0.00205. (G) hMPV genome RNA replication. Exact P values of YTHDF1, 2, 3 compared to vector are as follows: 12 h, P=0.05484, P=0.02933, P=0.02828; 18 h, P=0.00288, P=0.00122, P=0.00204; 24 h, P=0.02298, P=0.02186, P=0.00931; 48 h, P=0.26595, P=0.03483, P=0.03191. (H) hMPV antigenome RNA replication. Exact P values of YTHDF1, 2, 3 compared to vector are as follows: 12 h, P=0.10020, P=0.05994, P=0.02614; 18 h, P=0.00562, P=0.00251, P=0.00429; 24 h, P=0.09292, P=0.03506, P=0.00948; 48 h, P=0.01837, P=0.00887, P=0.01310. (I) G mRNA synthesis. Exact P values of YTHDF1, 2, 3 compared to vector are as follows: 12 h, P=0.00019, P=0.00031, P=0.00171; 18 h, P=0.00204, P=0.00081, P=0.00024; 24 h, P=0.00157, P=0.00037, P=0.00057; 48 h, P=0.01650, P=0.00304, P=0.00075. (J) N mRNA synthesis. Exact P values of YTHDF1, 2, 3 compared to vector are as follows: 12 h, P=0.01482, P=0.00724, P=0.00762; 18 h, P=0.03820, P=0.01051, P=0.01266; 24 h, P=0.13296, P=0.03210, P=0.01762; 48 h, P=0.24640, P=0.02671, P=0.01364. All RNA data were quantified by real-time RT-PCR.

FIG. 30A-G. Effects of m⁶A writer proteins on hMPV gene expression. (A) Overexpression of m⁶A writer proteins enhances hMPV gene expression. A549 cells were transfected with plasmids encoding HA-tagged METTLE3 and/or METTL14. At 24 h post-transfection, cells were infected with rhMPV at an MOI of 5.0. At 12, 18, and 24 h post-infection, cell lysates were harvested for Western blot analysis using antibody against hMPV G and N proteins. Overexpression of METTLE3 and METTL14 was confirmed by Western blot using anti-HA tag antibody. (B) m⁶A writer proteins increase hMPV progeny virus production. The release of infectious hMPV particles was monitored by a single-step growth curve. Virus titer was measured by an immunostaining plaque assay. P values of METTL3, METTL14 or METTL3 plus METTL14 compared to vector are as follows: 12 h, P=0.04474, P=0.00451, P=0.00192; 18 h, P=0.00386, P=0.00036, P=0.00130; 24 h, P=0.01348, P=0.00213, P=0.00842; 48 h, P=0.01133, P=0.00196, P=0.00355. (C) hMPV genome RNA replication. Exact P values of METTL3, METTL14, METTL3+METTL14 compared to pCAGGS are as follows: 12 h, P=0.00196, P=0.68663, P=0.01834; 18 h, P=0.09680, P=0.41626, P=0.02352; 24 h, P=0.36402, P=0.04403, P=0.00728; 48 h, P=0.01571, P=0.00400, P=0.15722. (D) hMPV antigenome RNA replication. Exact P values of METTL3, METTL14, METTL3+METTL14 compared to pCAGGS are as follows: 12 h, P=0.01062, P=0.00013, P=0.00011; 18 h, P=0.00061, P=0.00105, P=0.00004; 24 h, P=0.07619, P=0.01308, P=0.00171; 48 h, P=0.00036, P=0.00026, P=0.00092. (E) G mRNA synthesis. Exact P values of METTL3, METTL14, METTL3+METTL14 compared to pCAGGS are as follows: 12 h, P=0.83915, P=0.01768, P=0.01202; 18 h, P=0.00893, P=0.00688, P=0.00095; 24 h, P=0.42109, P=0.02079, P=0.00506; 48 h, P=0.07359, P=0.16698, P=0.01625. (F) N mRNA synthesis. All RNA data were quantified by real-time RT-PCR. Exact P values of METTL3, METTL14, METTL3+METTL14 compared to pCAGGS are as follows: 12 h, P=0.04018, P=0.17880, P=0.74770; 18 h, P=0.34251, P=0.14461, P=0.01772; 24 h, P=0.92864, P=0.60017, P=0.67147; 48 h, P=0.36368, P=0.86170, P=0.80096. (G) Co-localization of hMPV N and METTL14. A549 cells were infected by rhMPV at an MOI of 5.0. At 24 h post-infection, rhMPV-infected cells were stained with anti-METTL14 antibody and anti-hMPV N protein antibody, and were analyzed by confocal microscope. Nuclei were labeled with DAPI. Mock-infected controls were shown in FIG. 47A. Representative results from three independent experiments are shown.

FIG. 31A-H. Knockdown of m⁶A eraser proteins enhances hMPV gene expression. (A) Western blot of hMPV proteins. A549 cells were transfected with siRNA targeting FTO and/or ALKBH5. At 24 h post-transfection, cells were infected with rhMPV at an MOI of 0.5. At 12, 18, and 24 h post-infection, cell lysates were harvested for Western blot analysis using hMPV antibody. Knockdown was confirmed by Western blot using anti-FTO and anti-ALKBH5 antibody. (B) Quantification of hMPV G protein. The density of G protein in Western blot was quantified by Image J. Data are average of three independent experiments. Exact P values of ALKBH5, FTO, ALKBH5+FTO compared to SiRNA control are as follows: 12 h, P=0.37569, P=0.37406, P=0.30388; 18 h, P=2.2879E-6, P=1.2823E-5, P=0.00897; 24 h, P=0.00801, P=0.00099, P=0.12411. (C) Quantification of hMPV N protein. The density of N protein in Western blot was quantified by Image J. Data are average of three independent experiments. Exact P values of ALKBH5, FTO, ALKBH5+FTO compared to SiRNA control are as follows: 12 h, P=0.37201, P=0.51066, P=0.88151; 18 h, P=0.00576, P=0.05129, P=0.00048; 24 h, P=0.86571, P=6.2279E-6, P=0.57172. (D) Knockdown m⁶A eraser proteins increases hMPV progeny virus production. Exact P values of ALKBH5, FTO, ALKBH5+FTO compared to siRNA control are as follows: 12 h, P=0.1814, P=0.0660, P=0.0401; 18 h, P=0.0413, P=0.0362, P=0.0083; 24 h, P=0.0232, P=0.0338, P=0.0104; 48 h, P=0.0281, P=0.1210, P=0.0885. (E) hMPV genome RNA replication. Exact P values of ALKBH5, FTO, ALKBH5+FTO compared to SiRNA control are as follows: 12 h, P=0.02901, P=0.41757, P=0.00972; 18 h, P=0.00456, P=0.03716, P=0.00268; 24 h, P=0.02335, P=0.00699, P=0.00416; 48 h, P=0.04565, P=0.74169, P=0.18750. (F) hMPV antigenome RNA replication. Exact P values of ALKBH5, FTO, ALKBH5+FTO compared to SiRNA control are as follows: 12 h, P=0.00028, P=0.00097, P=0.00092; 18 h, P=0.00110, P=0.00059, P=0.00021; 24 h, P=0.00124, P=0.00120, P=0.00043; 48 h, P=0.00875, P=0.00382, P=0.00621. (G) G mRNA synthesis. Exact P values of ALKBH5, FTO, ALKBH5+FTO compared to SiRNA control are as follows: 12 h, P=0.00222, P=0.00131, P=0.00109; 18 h, P=0.00390, P=0.00174, P=0.00024; 24 h, P=0.00346, P=0.00052, P=0.00043; 48 h, P=0.00734, P=0.00129, P=0.00554. (H) N mRNA synthesis. Exact P values of ALKBH5, FTO, ALKBH5+FTO compared to SiRNA control are as follows: 12 h, P=0.00053, P=0.00020, P=0.00020; 18 h, P=0.01025, P=0.00365, P=0.00337; 24 h, P=0.03322, P=0.02088, P=0.01801; 48 h, P=0.05411, P=0.00441, P=0.01979. All RNA data were quantified by real-time RT-PCR.

FIG. 32A-G. m⁶A-abrogating hMPV mutants are attenuated in replication in cell culture and are defective in m⁶A methylation. (A) Immunostaining spots formed by recombinant hMPVs. Vero E6 cells were infected with recombinant hMPV mutants and incubated at 37° C. for 5 days. The cells were stained with an anti-hMPV N protein monoclonal antibody. The left 6 viruses were done as the same time. The right 2 viruses were done at the same time. (B) CPE caused by m⁶A-deficient rhMPV mutants in A549 cells. Confluent A549 cells were infected with each rhMPV at an MOI of 1.0, CPE was imaged at days 2 and 3. All m⁶A-deficient rhMPV mutants had an earlier cytopathic effect compared to rhMPV. (C) Growth curve of m⁶A-deficient rhMPV mutants in A549 cells. Confluent A549 cells were infected with each rhMPV at an MOI of 1.0, total virus in supernatant and cell lysate was harvested at the indicated time, and viral titer was determined by an immunostaining plaque assay. P values of rhMPV-G1-2, G1-7, G1-14, G8-9, or G8-14 compared to rhMPV are as follows: D1, P=0.000053, P=0.000022, P=0.000049, P=0.000097, P=0.000013; D2, P=0.000028, P=0.000081, P=0.000066, P=0.000023, P=0.000342; D3, P=0.000020, P=0.000021, P=0.000203, P=0.000004, P=0.000315; D4, P=0.000079, P=0.001343, P=0.000322, P=0.000122, P=0.005649. (D) Immunoblot analysis of hMPV proteins. Confluent A549 cells were infected with each rhMPV at an MOI of 1.0, cell lysates were harvested at 24, 48, and 72 h post-infection, and hMPV proteins were detected by specific antibodies against N and G protein by Western blot. (E) m⁶A-mutated hMPV RNA is defective in binding to m⁶A antibody by MeRIP assay. An MeRIP assay was carried out to determine the binding of RNA to m⁶A antibody using Magna MeRIP™ m⁶A kit. Anti-m⁶A antibody was first conjugated to magnetic beads. Total RNA (15 μg) was extracted from rhMPV or m⁶A deficient rhMPV-infected A549 cells, and incubated with m⁶A antibody-associated beads at 4° C. for 2 h with rotation. The RNA-associated magnetic beads were then washed for 3 times. Total RNA was extracted from beads by TRizol reagent and was quantified by real-time RT-PCR using primers annealing to hMPV antigenome, genome, and G mRNA. Exact P values of rhMPV-G1-2, G8-9, G1-7, G8-14 and G1-14 compared to rhMPV are as follows: Genome, P=0.06806, P=0.07424, P=0.18391, P=0.51012, P=0.22587; Antigenome, P=0.39634, P=0.69168, P=0.03009, P=0.01541, P=0.01527; G-mRNA, P=0.16337, P=0.05549, P=0.01933, P=0.01138, P=0.00785. (F) Quantification of m⁶A content by m⁶A RNA Methylation Assay Kit. Virion RNA was extracted from highly purified wild type and mutant rhMPVs. Total m⁶A content of each virion RNA was quantified by m⁶A RNA Methylation Assay Kit (Abcam, ab185912) as described in Materials and Methods. Exact P values of rhMPV-G1-2, G8-9, G8-14, G1-14, G(−)1-6 and rhMPV-WT(ALKBH5) compared to rhMPV-WT are as follows: P=0.00900, P=0.00149, P=1.7761E-5, P=2.6994E-7, P=3.6151E-9, P=1.425E-6. (G) m⁶A-deficient rhMPV RNA has reduced binding efficiency to reader proteins. A549 cells in T25 flasks were transfected with plasmid pYTHDF1-HA or pYTHDF2-HA, and were lysed in 650 μL 1× lysis buffer (abeam, ab152163) at 24 h post transfection. Cell lysate was divided into 3 tube (200 L/tube) and incubated with 2×10⁸ copies of virion RNA (rhMPV-G1-14, G8-14, G1-2, G8-9, or rhMPV) and then incubated with 50 μL Pierce anti-HA Magnetic beads at room temperature for 30 min. The amount of virion RNA captured by the YTHDF1 or YTHDF2 was quantified by real-time RT-PCR. Percent of bound RNA of hMPV mutants relative to rhMPV was calculated. Results are from three independent experiments. Exact P values of rhMPV-G8-14, G1-2 and G8-9 compared to rhMPV-G1-14 are as follows: YTHDF1, P=0.47021, P=0.00138, P=0.00004; YTHDF2, P=0.48721, P=0.02531, P=0.00407.

FIG. 33A-G. m⁶A deficient rhMPV trigger a higher type I IFN secretion. Dynamics of IFN-α (A) and IFN-β (B) secretion in A549 cells infected by hMPV at MOI of 4.0. A549 cells were infected with rhMPV or each rhMPV mutant at an MOI of 4.0, cell culture supernatants were harvested at 16, 24, and 40 h post-inoculation, and IFN-α and IFN-β in cell supernatants were measured by ELISA. A standard curve was generated using human IFN-α or IFN-β. Data shown are average of three independent experiments. IFN-α: P values of rhMPV-G1-2, G1-7, G1-14, G8-9, or G8-14 compared to rhMPV are as follows: 16 h, P=0.000002, P=0.000008, P=0.0000002, P=0.000001, P=0.000037; 24 h, P=0.000233, P=0.000651, P=0.000024, P=0.000473, P=0.000004; 40 h, P=0.000363, P=0.001191, P=0.000643, P=0.000098, P=0.000315. IFN-β, 16 h, P=0.000001, P=0.0000001, P=0.000001, P=0.000002, P=0.000001; 24 h, P=0.000109, P=0.000003, P=0.000089, P=0.000276, P=0.000027; 40 h, P=0.001712, P=0.002491, P=0.380890, P=0.007107, P=0.003963. (C) Dynamic of IFN-β secretion in A549 cells at MOI of 1.0. A549 cells were infected by rhMPV, rhMPV-G8-9, G1-2, or G1-14 at an MOI of 1.0, IFN-β in cell culture supernatants at 16, 24, and 40 h post-infection was measured by ELISA. Exact P values of rhMPV-G1-2, G8-9 and G1-14 compared to rhMPV are as follows: 16 h, P=8.209E-5, P=0.00096, P=3.341E-6; 24 h, P=0.00035, P=0.01008, P=0.00081; 40 h, P=0.52283, P=0.04606, P=0.49405. (D) Dynamics of IFN-β secretion in TPH-1 cells infected by hMPV at MOI of 4.0. TPH-1 cells were infected each hMPV at an MOI of 4.0, and IFN-β in cell culture supernatants was detected by ELISA kit. Exact P values of rhMPV-G1-2, G8-9 and G1-14 compared to rhMPV are as follows: 16 h, P=9.9466E-6, P=6.8232E-6, P=2.0562E-5; 24 h, P=5.2285E-6, P=1.8678E-6, P=1.9641E-6; 40 h, P=2.6328E-6, P=2.3568E-6, P=1.7651E-6; 48 h, P=1.3019E-6, P=2.4507E-6, P=9.6617E-7. (E) Dynamics of IFN-β secretion in TPH-1 cells infected by hMPV at MOI of 1.0. Exact P values of rhMPV-G1-2, G8-9 and G1-14 compared to rhMPV are as follows: 16 h, P=4.895E-6, P=2.3084E-6, P=9.1479E-7; 24 h, P=2.6423E-6, P=1.89E-6, P=4.3914E-6; 40 h, P=1.823E-6, P=2.9168E-6, P=2.005E-6; 48 h, P=1.9192E-6, P=1.0143E-6, P=6.8216E-6. (F and G) Dynamics of IFN-β secretion at MOI of 4.0. A549 cells (F) or TPH-1 cells (G) were infected with rhMPV-G1-14, G(−)1-6, or rhMPV at an MOI of 4.0, and IFN-β in cell culture supernatants was detected by ELISA kit. Panel F: Exact P values of rhMPV-G1-14 and G(−)1-6 compared to rhMPV are as follows: 16 h, P=1.4327E-5, P=7.3582E-5; 24 h, P=1.1248E-5, P=0.00011; 40 h, P=0.07540, P=0.01606. Panel G: Exact P values of rhMPV-G1-14 and G(−)1-6 compared to rhMPV are as follows: 16 h, P=2.0562E-5, P=1.1296E-6; 24 h, P=1.9641E-6, P=6.1132E-6; 40 h, P=1.7651E-6, P=2.1782E-6; 48 h, P=9.6617E-7, P=2.6455E-6.

FIG. 34A-I. Virion RNA of m⁶A deficient rhMPVs trigger a higher type I IFN secretion. (A) IFN-β response in A549 cells transfected with total RNA. Total RNA was extracted from rhMPV, rhMPV-G8-14, or rhMPV-G1-14-infected A549 cells at 24 h post-inoculation, and the antigenome was quantified by real-time RT-PCR. A549 cells in 24-well plates were transfected with 10⁸ antigenome RNA copies of total RNA with or without treatment of calf intestinal phosphatase (CIP). At 24 and 44 h post-transfection, IFN-β in culture medium was measured by ELISA. P values of rhMPV-G8-14 or G1-14 compared to rhMPV are as follows: 24 h, P=0.0000002, P=0.000039; 44 h, P=0.003365, P=0.000165. (B) IFN-β response in A549 cells transfected with virion RNA. Virion RNA was extracted from purified hMPV virions, the level of antigenome was quantified by real-time RT-PCR. A549 cells in 24-well plates were transfected with 2×10⁷ antigenome copies of virion RNA either with or without CIP treatment. At 24 and 44 h post-transfection, IFN-β in culture medium was measured by ELISA. P values of rhMPV-G8-14 or G1-14 compared to rhMPV are as follows: 24 h, P=0.000004, P=0.000004; 44 h, P=0.022736, P=0.012281. (C) IFN-β response in A549 cells transfected with viral G mRNA. Poly(A)-containing viral mRNA was isolated from total RNA purified from virus-infected cells using a Dynabeads mRNA isolation kit (Life Technologies). The hMPV G mRNA was further isolated by Dynabeads MyOne™ Streptavidin C1 conjugated with poly T-tailed G gene specific primer. The G mRNA copies were quantified by real-time RT-PCR. A549 cells were transfected with 10⁹ RNA copies of G mRNA either with or without CIP treatment. At 24 and 44 h post-transfection, IFN-β in culture medium was measured by ELISA. P values of rhMPV-G8-14 or G1-14 compared to rhMPV are as follows: 24 h, P=0.000371, P=0.002255; 44 h, P=0.000099, P=0.000001. (D, E and F) Comparison of IFN response of virion RNA of hMPV mutants. A549 cells were transfected with 10⁷ (D), 10⁶ (E) and 10⁵ (F) RNA copies of virion RNA of rhMPV-G1-14, G1-2, G8-9, and rhMPV, and dynamics of IFN response was detected by ELISA kit. (G, H, and I) Natural m⁶A-deficient virion RNA induces IFN response. A549 cells were transfected with 10⁷ (G), 10⁶ (H) and 10⁵ (I) RNA copies of virion RNA of rhMPV-G1-14, G(−)1-6, ALKBH5, and rhMPV, and dynamics of IFN response was detected by ELISA kit. P values for panels D-I are: (D) Exact P values of rhMPV-G1-2, G8-9 and G1-14 compared to rhMPV are as follows: 16 h, P=2.6989E-6, P=9.4265E-5, P=6.1728E-5; 24 h, P=3.6322E-5, P=0.00028 P=2.4336E-6; 40 h, P=0.25414, P=0.00044, P=0.00003. (E) Exact P values of rhMPV-G1-2, G8-9 and G1-14 compared to rhMPV are as follows: 16 h, P=0.00073, P=0.51720, P=0.00091; 24 h, P=0.00045, P=0.01806 P=5.7204E-6; 40 h, P=0.32383, P=0.87823, P=4.3492E-9. (F) Exact P values of rhMPV-G1-2, G8-9 and G1-14 compared to rhMPV are as follows: 16 h, P=0.02384, P=0.16134, P=0.00013; 24 h, P=0.93090, P=0.12806 P=00050; 40 h, P=0.00332, P=0.00029, P=0.00022. (G) Exact P values of rhMPV-G1-14, G(−)1-6 and hMPV(ALKBH5) compared to rhMPV are as follows: 16 h, P=0.01312, P=0.11129, P=2.0409E-5 24 h, P=2.13E-5, P=0.00166, P=1.9338E-5; 40 h, P=0.00128, P=6.9171E-5, P=0.29460. (H) Exact P values of rhMPV-G1-14, G(−)1-6 and hMPV(ALKBH5) compared to rhMPV are as follows: 16 h, P=0.00712, P=2.5255E-5, P=7.3829E-6; 24 h, P=0.00036, P=2.1905E-5, P=1.3462E-5; 40 h, P=0.00015, P=4.5883E-6, P=2.8804E-5. (I) Exact P values of rhMPV-G1-14, G(−)1-6 and hMPV(ALKBH5) compared to rhMPV are as follows: 16 h, P=0.01798, P=0.08709, P=0.02274; 24 h, P=0.00151, P=0.02996, P=0.00081; 40 h, P=0.88326, P=0.00073, P=0.77901.

FIG. 35A-D. IFN response in wild-type, RIG-I, MAVS, or MDA5-knockout A549-Dual™ cells infected by m⁶A deficient hMPVs. Confluent wild-type (A), MDA5 (B), RIG-I (C), or MAVS (D)-knockout A549 cells were infected by rhMPV, rhMPV-G8-14, or rhMPV-G1-14 at an MOI of 1.0, cell culture supernatants were harvested at 24 and 48 h post-inoculation. IFN-β in cell supernatants was measured by ELISA. Data shown are average of three independent experiments±standard deviation. P values of rhMPV-G8-14 or G1-14 compared to rhMPV are as follows: A549-Dual, 24 h, P=0.0000006, P=0.0000003; 48 h, P=0.011023, P=0.000516. A549-Dual MDA5 K.O., 24 h, P=0.000004, P=0.000004; 48 h, P=0.000074, P=0.00000002.

FIG. 36A-D. SEAP secretion in wild-type, RIG-I, MAVs, or MDA5-knockout A549-Dual™ cells infected by m⁶A deficient hMPV. Confluent wild-type (A), MDA5 (B), RIG-I (C), or MAVs (D)-knockout A549 cells were infected by rhMPV, rhMPV-G8-14, or rhMPV-G1-14 at an MOI of 1.0, cell culture supernatants were harvested at 24 and 48 h post-inoculation. SEAP secreted in cell supernatants was measured by colorimetric enzyme assay with substrate Quanti-Blue™ and read by microplate reader on OD value at 620 nm. Data shown are average of three independent experiments±standard deviation. P values of rhMPV-G8-14 or G1-14 compared to rhMPV are as follows: A549-Dual, 24, P=0.000022, P=0.000365; 48 h, P=0.028380. A549-Dual MDA5 K.O., 24 h, P=0.000036, P=0.000001; 48 h, P=0.000135, P=0.000556. A549-Dual RIG-I K.O., 24 h, P=0.000003, P=0.000001; 48 h, P=0.007404, P=0.000925. A549-Dual MAVS K.O., 24 h, P=0.000211, P=0.000016; 48 h, P=0.000656, P=0.000028.

FIG. 37A-D. IFN response in wild-type, RIG-I, MAVS, or MDA5-knockout A549-Dual™ cells transfected by virion RNA. Confluent wild-type (A), MDA5 (B), RIG-I (C), or MAVs (D)-knockout A549 cells were transfected with 10⁷ antigenomic RNA copies of virion RNA of rhMPV, rhMPV-G8-14, or rhMPV-G1-14, cell culture supernatants were harvested at 24 and 40 h post-inoculation. IFN-β in cell supernatants was measured by ELISA. Data shown are average of three independent experiments±standard deviation. P values of rhMPV-G8-14 or G1-14 compared to rhMPV are as follows: A549-Dual, 24 h, P=0.003259, P=0.000450; 48 h, P=0.001945, P=0.000037. A549-Dual MDA5 K.O., 24 h, P=0.000755, P=0.000263; 48 h, P=0.001200, P=0.000008. A549-Dual RIG-I K.O., 24 h, P=0.000593, P=0.000524; 48 h, P=0.007623 P=0.00000007.

FIG. 38A-D. SEAP secretion in wild-type, RIG-I, MAVS, or MDA5-knockout A549-Dual™ cells transfected by virion RNA. Confluent wild-type (A), MDA5 (B), RIG-I (C), or MAVs (D)-knockout A549 cells were transfected with 10⁷ antigenomic RNA copies of virion RNA of rhMPV, rhMPV-G8-14, or rhMPV-G1-14 with or without CIP treatment, cell culture supernatants were harvested at 16, 24, and 40 h post-inoculation. SEAP secretion in cell supernatants was measured by colorimetric enzyme assay with substrate Quanti-Blue™ and read by microplate reader on OD value at 620 nm. Data shown are average of three independent experiments. P values of rhMPV-G8-14 or G1-14 compared to rhMPV are as follows: A549-Dual, 16 h, P=0.00283, P=0.00408; 24 h, P=0.00103, P=0.00288; 40 h, P=0.00332, P=0.03540. A549-Dual MDA5 K.O., 16 h, P=0.00565, P=0.00826; 24 h, P=0.00062, P=0.00366; 40 h, P=0.00083, P=0.01011. A549-Dual RIG-I K.O., 24 h, P=0.00421; 40 h, P=0.00102, P=0.00021. A549-Dual MAVS K.O., 40 h, P=0.01194.

FIG. 39A-H. m⁶A-deficient hMPVs and virion RNA induce a higher expression of RIG-I. (A) m⁶A-deficient rhMPVs stimulate a higher expression of RIG-I. Confluent A549 cells in 12-well plates were infected by of rhMPV, rhMPV-G8-14, or rhMPV-G1-14 at an MOI of 0.2, 1.0, and 5.0. At 8, 16, 24, and 32 h post-transfection, cells were lysed and 20 μl of cell lysates were subjected to SDS-PAGE and Western blot analysis using antibody specific to RIG-I, hMPV N, or β-actin. (B) m⁶A-deficient virion RNA induces a higher expression of RIG-I. Confluent A549 cells in 12-well plates were transfected with an increasing amount of poly (I:C) (Sigma-Aldrich, 0.5 and 2.0 μg/well) or virion RNAs (2×10⁵, 2×10⁶, or 2×10⁷ copies/well) of rhMPV, rhMPV-G8-14, or rhMPV-G1-14. At 8, 16, 24, and 32 h post-transfection, cells were lysed and 20 μl of cell lysates were subjected to SDS-PAGE, and Western blot analysis using antibody against RIG-I or β-actin. (C) m⁶A-deficient rhMPVs induce higher phosphorylation of IRF3. Confluent A549 cells were infected by of each hMPV at an MOI of 5.0. At 8, 16, 24, and 32 h post-transfection, cells were lysed and 20 μl of cell lysates were subjected to Western blot using antibody specific to IRF3 or phosphorylated IRF3 on site S386 or S396. (D) m⁶A-deficient virion RNA induce higher phosphorylation of IRF3. Confluent A549 cells were transfected with poly (I:C) or virion RNA from rhMPV, rhMPV-G8-14, and rhMPV-G1-14. At 8, 16, 24, and 32 h post-transfection, 20 μl of cell lysates were subjected to Western blot using antibody specific to IRF3 or phosphorylated IRF3 on site S386 or S396. (E) Comparison of RIG-I expression triggered by virion RNA of rhMPV mutants. Confluent A549 cells were transfected with increasing amounts (10⁵, 10⁶, and 10⁷ RNA copies) of virion RNA of rhMPV-G1-14, G1-2, G8-9, and rhMPV. At 8 and 16 h post-transfection, RIG-I was detected by Western blot. (F) Removal of 5′ triphosphate abolished RIG-I expression. A549 cells were transfected with virion RNA of rhMPV-G1-14, G8-14, and rhMPV with or without CIP treatment. At 8, 16, and 24 h post-transfection, RIG-I was detected by Western blot. (G) m⁶A-deficient rhMPVs induce higher RIG-I expression and IRF3 phosphorylation. A549 cells were infected by each hMPV at an MOI of 5.0. At indicated time points, RIG-I expression and IRF3 phosphorylation at sites S386 and S396 were detected by Western blot. (H) m⁶A-deficient virion RNA induces higher RIG-I expression. A549 cells were transfected with 10⁷ copies of each virion RNA. At indicated time points, RIG-I expression were detected by Western blot.

FIG. 40A-E. m⁶A-deficient antigenome increases binding affinity to RIG-I. (A) Biotinylated virion RNA pulldown RIG-I. 10⁹ copies of virion RNA with or without CIP treatment was biotinylated and incubated with MyOne™ Streptavidin C1 beads in the presence of RNase inhibitor. RNA-associated beads were then washed three times and incubated with 50 μl of A549 cell lysate containing overexpressed RIG-I. Beads were then washed for 3 times and subjected to SDS-PAGE. The pull-down RIG-I protein on Streptavidin beads was detected by Western blot using anti-RIG-I antibody. 5 μl of cell lysate from each sample was loaded as input. (B and C) RIG-I pulldown hMPV RNA. A549 cells in T75 flasks were transfected with 18 μg of pEF-BOS-RIG-I-Flag. At 22 h post-transfection, cell lysates were prepared and incubated with 450 μl of anti-Flag M2 Magnetic beads for 80 min. The mixture was then divided into 13 aliquots, 12 aliquots were incubated with 2×10⁸ copies of virion RNA (with or without CIP treatment) or 2×10⁹ copies of N or G mRNA respectively at 37° C. for 1 h, and the 13^{th} aliquot was washed and subjected for Western blot for RIG-I and β-actin (B). The magnetic beads were washed with TBS for three times. The bound RNA was extracted by Trizol, and quantified by real-time RT-PCR (C). P value for antigenome of rhMV-G8-14 and G1-14 compared to rhMPV: CIP, P=0.0083, P=0.07438; No-CIP, P=0.000909, P=0.0366. (D) Purification of RIG-I protein. HEK-293T cells in T150 flask were transfected with 30 μg of plasmid encoding Flag-tagged RIG-I (pEF-BOS-RIG-I-Flag), and RIG-I protein was purified. (E) Competitive binding of wt virion RNA and m⁶A-deficient virion RNA to RIG-I. Purified RIG-I protein was incubated with RNA mixtures consisted of different ratios of rhMPV-G1-14 and rhMPV RNA, which are biotinylated and conjugated to Dynabeads® MyOne™ Streptavidin C1 beads in the presence of AMP-PNP. The beads were washed three times with PBS and subjected to Western blot against RIG-I.

FIG. 41A-F. Analysis of RIG-I:RNA conformation by limited trypsin digestion. (A) Domain structure of RIG-I protein. CARD, caspase activation and recruitment domains; Helicase, helicase domain; CTD, C-terminal domain. (B) Model for mechanisms of enhanced RIG-I-mediated IFN signaling by m⁶A-deficient hMPV RNA. RIG-I is in an autorepressed conformation in the absence of ligand. RIG-I CTD recognizes and binds to 5′ppp of RNA. 5′-ppp-RNA without m⁶A has a higher binding affinity to helicase domain of RIG-I. RIG-I is an RNA translocase, moving from 5′-ppp to RNA chain. Internal m⁶A may serve as a “brake” to prevent RIG-I translocation. The RIG-I helicase domain binds the RNA, triggering RIG-I conformational change and subsequent oligomerization. RNAs without m⁶A more easily induce RIG-I conformational change. The released CARDs of the activated RIG-I:RNA complex are ubiquitinated for downstream signaling. (C-F) RIG-I fragments were detected by Western blotting with a monoclonal antibody to the helicase domain. (C) Limited trypsin digestion of RIG-I protein in the absence of RNA ligand. RIG-I was incubated with trypsin in the absence of RNA ligands. Aliquots were removed from the reaction at various time points and analyzed by Western blot. (D) Limited trypsin digestion of RIG-I protein with poly (I:C). RIG-I was incubated with 2-10 μg of poly (I:C) and 2 mM AMP-PNP. After 2 h of digestion by trypsin, samples were analyzed by Western blot. (E) Limited trypsin digestion of RIG-I protein with virion RNA. RIG-I was incubated with 10⁷ RNA copies of each virion RNA and 2 mM AMP-PNP. After 2 h of digestion by trypsin, samples were analyzed by Western blot. (F) Competition assay. RIG-I was incubated with a mixture containing different amount of RNA of rhMPV-G1-14 and rhMPV, and 2 mM AMP-PNP. After 2 h of digestion by trypsin, samples were analyzed by Western blot.

FIG. 42A-D. Kinetics of the replication of m⁶A deficient rhMPVs in wild-type, RIG-I, MAVS, or MDA5-knockout A549 cells. Confluent wild-type (A), MDA5 (B), RIG-I (C), or MAVS (D)-knockout A549 cells in 24-well plates were infected by rhMPV, rhMPV-G8-14, or rhMPV-G1-14 at an MOI of 1.0, total virus in cell culture supernatants and cell lysate was harvested at the indicated time, and viral titer was determined by an immunostaining plaque assay. Data represent two independent experiments (mean and standard deviation of six samples). The arrow indicates the degree of titer difference compared to rhMPV. P values of rhMPV-G8-14 or G1-14 compared to rhMPV are as follows: A549-Dual, 12 h, P=0.005677, P=0.020272; 24 h, P=0.000003, P=0.000018; 36 h, P=0.000002, P=0.000017; 48 h, P=0.000067, P=0.000450; 60 h, P=0.000120, P=0.001064; 72 h, P=0.000296, P=0.012931. A549-Dual MDA5-K.O., 12 h, P=0.000872, P=0.013116; 24 h, P=0.000010, P=0.000022; 36 h, P=0.000042, P=0.000195; 48 h, P=0.000007, P=0.000014; 60 h, P=0.000127, P=0.000062; 72 h, P=0.000014, P=0.000403. A549-Dual RIG-I-K.O., 12 h, P=0.013642, P=0.016926; 24 h, P=0.000057, P=0.000359; 36 h, P=0.000002, P=0.000023; 48 h, P=0.000085; P=0.004960, 60 h, P=0.000113, P=0.017314; 72 h, P=0.000019. A549-Dual MAVS-K.O., 12 h, P=0.001973, P=0.005915; 24 h, P=0.000107, P=0.000418; 36 h, P=0.000139, P=0.000366; 48 h, P=0.000318; 60 h, P=0.000937; 72 h, P=0.000566, P=0.026082.

FIG. 43A-E. Interferon response, pathogenicity, and immunogenicity of m⁶A-deficient rhMPVs in cotton rats. (A) Interferon response of rhMPV in cotton rats. Six-week-old specific-pathogen-free (SPF) female cotton rats (5 per group) were inoculated intranasally with 100 μl of PBS or PBS containing 2.0×10⁵ PFU of rhMPV-G8-14, rhMPV-G1-14 or rhMPV. At 48 h post-inoculation, cotton rats were sacrificed and bronchoalveolar lavage (BAL) from the right lung was collected for IFN-β bioactivity assay on CCRT cells as described in Materials and Methods. The IFN-β concentration of each BAL sample was calculated according to the highest dilution of samples and the lowest concentration of standard human IFN-β which inhibited rVSV-GFP replication therefore GFP expression. P value of rhMPV-G8-14 and G1-14 compared to rhMPV is P=0.000022 and P=0.00237 respectively. (B) hMPV titer in lungs and nasal turbinates. Four-week-old SPF cotton rats were inoculated intranasally with 2.0×10⁵ PFU of each rhMPV. At day 4 post-infection, the cotton rats were sacrificed, and lungs and nasal turbinates were collected for virus titration by an immunostaining plaque assay. Viral titers are the geometric mean titer (GMT) of 5 animals±standard deviation. Detection limit is 2.0 log PFU/g tissue. P values of rhMPV-G1-2, G8-9 or G1-14 compared to rhMPV are as follows: Lung, P=4.81×10⁻⁶, P=8.67×10⁻⁷, P=4.38×10⁻⁸; Nasal turbinate, P=0.02583, P=0.00331, P=0.01101. (C) m⁶A deficient rhMPVs had less lung histopathological changes compared to rhMPV. Representative pathological changes from each group are shown. Right lung lobe of each cotton rat was fixed in 4% neutral buffered formaldehyde, embedded in paraffin, sectioned at 5 μm, and stained with hematoxylin-eosin (HE) for the examination of histological changes by light microscopy. Micrographs with 20× magnification are shown. (D) m⁶A deficient rhMPV provides complete protection against hMPV challenge. Four-week-old SPF cotton rats were inoculated intranasally with 2.0×10⁵ PFU of each rhMPV. At week 4 post-immunization, cotton rats were challenged with 2.0×10⁵ PFU of hMPV. At day 4 post-challenge, the cotton rats were sacrificed, and lungs and nasal turbinates were collected for virus titration by an immunostaining plaque assay. Viral titers are the geometric mean titer (GMT) of 5 animals±standard deviation. The detection limit is 2.0 log PFU/g tissue. (E) m⁶A deficient rhMPV induced a high level of neutralizing antibody. Blood samples were collected from each rat weekly by retro-orbital bleeding. The hMPV-neutralizing antibody titer was determined using a plaque reduction neutralization assay, as described in Materials and Methods.

FIG. 44. Model for RIG-I mediated IFN signaling pathway. Upon hMPV entry, the RNP complex is delivered into the cytoplasm where RNA synthesis and viral replication occur. During replication, the RdRP initiates at the extreme 3′ end of the genome and synthesizes a full-length complementary antigenome, which subsequently serves as template for synthesis of full-length progeny genomes. The newly synthesized genome and antigenome was methylated by m⁶A writer proteins and encapsidated by viral N protein. Viral genome and antigenome are recognized by cytoplasmic RNA sensor RIG-I and induces signaling to the downstream adaptor protein MAVS which subsequently activates IRF3 and NF-κB pathways, leading to the production of type-I IFN and proinflammatory cytokines. The internal m⁶A methylation inhibits RIG-I mediated IFN signaling pathway.

FIG. 45A-C. hMPV genome and antigenome are packaged into virion. (A) Quantification of genome and antigenome in hMPV virions by real-time RT-PCR. Wild type hMPV was grown in T150 flasks of A549 cells and cell culture supernatant was collected at 40 h. hMPV was purified by 20% sucrose cushion, the virion pellet was resuspended in 100 μL NTE buffer, treated with RNase at room temperature for 30 min. The reaction system was diluted in 5 ml NTE buffer and the virion was pelleted down in SW55 Ti ultracentrifugation tube on 10% sucrose cushion and resuspended in 100 μL NTE buffer. (B) Pulldown of N-RNA complex by N antibody. RNase-treated virion was disrupted by 10× Disruption Buffer, mixed with Protein A/G Magnetic beads (MilliPore, LSKMAGAG02) bound with mouse anti-hMPV N antibody (MiiliPore, MAB80138) or mouse IgG (Sigma Aldrich, #I5381) and incubated at room temperature for 2 h and washed with TBS buffer for 3 times. The beads were then subjected for Western blot. (C) Quantification of genome and antigenome in N-RNA complex by real-time RT-PCR. Total RNA was extracted from N-complex pulled down by magnetic beads and subjected to real-time RT-PCR.

FIG. 46A-H. The effects of writer and eraser proteins on host RNA m⁶A methylation and host mRNA translation. (A) The effects of knockdown of writer proteins on total host RNA. A549 cells were transfected with siRNA targeting METTL3 and METTL14 or control siRNA. At 24 h post-transfection, total RNA was extracted from these cells. The m⁶A content was quantified by m⁶A RNA Methylation Assay Kit. Data are the average of three independent experiments±standard deviation. (B) The effects of knockdown of writer proteins on host mRNA. Polyadenylated mRNA from panel A was isolated using poly-A beads, and m⁶A content was quantified by m⁶A RNA Methylation Assay Kit. Data are the average of three independent experiments±standard deviation. (C) The effects of overexpression of writer proteins on host RNA. A549 cells were transfected with plasmids encoding METTL3 and METTL14 or control vector. At 24 h post-transfection, total RNA was extracted from these cells. The m⁶A content was quantified by m⁶A RNA Methylation Assay Kit. Data are the average of three independent experiments±standard deviation. (D) The effects of overexpression of eraser proteins on host RNA. A549 cells were transfected with plasmids encoding FTO and ALKBH5 or control vector. At 24 h post-transfection, total RNA was extracted from these cells. The m⁶A content was quantified by m⁶A RNA Methylation Assay Kit. Data are the average of three independent experiments±standard deviation. (E) The effects of knockdown of writer proteins on host protein translation. A549 cells were transfected with siRNA against METTL3 and METTL14 or control siRNA. After 24 h, cells were incubated in methionine- and cysteine-free media for 1 h, and 50 μCi of [³⁵S]-methionine was added. At 0.5, 1, 2 h, cells were washed with PBS, lysed in lysis buffer, analyzed by SDS-PAGE and exposed to film. The gel is the representative of two independent experiments. (F) Quantification of protein bands. Quantification of protein bands in panel E was done using ImageJ software. Data were the average of two independent experiments. (G) [³⁵S] incorporation by scintillation counting. 5 μl of each sample from panel E was used for measuring [³⁵S] incorporation by scintillation counting. Data were the average of two independent experiments. (F) Percent of [³⁵S] incorporation relative to control siRNA. Percentage was calculated from panel G. Data were the average of two independent experiments.

FIG. 47A-B. The effects of hMPV infection on distribution of m⁶A writer proteins in cells. A549 cells were infected by rhMPV at an MOI of 5.0. At 24 h post-infection, mock- or rhMPV-infected cells were stained with anti-reader or writer protein antibody and anti-hMPV N protein antibody, and were analyzed by confocal microscope. Nuclei were labeled with DAPI. (A) METTL14; (B) METTL3. Representative results from three independent experiments are shown.

FIG. 48. The effects of hMPV infection on distribution of m⁶A eraser proteins in cells. A549 cells were infected by rhMPV at an MOI of 5.0. At 24 h post-infection, mock- or rhMPV-infected cells were stained with anti-ALKBH5 antibody and anti-hMPV N protein antibody, and were analyzed by confocal microscope. Nuclei were labeled with DAPI. Representative results from three independent experiments are shown.

FIG. 49A-C. Distribution of m⁶A reader proteins in mock and hMPV-infected A549 cells. A549 cells were infected with rhMPV at an MOI of 5.0. At 24 h post-infection, mock- or rhMPV-infected cells were stained with anti-reader or writer antibody and anti-hMPV N protein antibody, and analyzed by confocal microscopy. Nuclei were labeled with DAPI. (A) YTHDF1; (B) YTHDF2; and (C) YTHDF 3.

FIG. 50. Mutagenesis strategy in putative m⁶A site in the G gene region in hMPV antigenome and mRNA. Schematic diagram of the hMPV genome with the mutations for altering the critical A or C residues in the m⁶A motifs to produce rhMPV lacking that putative m⁶A modification site in the G gene. A total of 14 putative m⁶A site, G1-G14, are shown. G gene sequence of hMPV strain (subtype A strain NL/1/00, GenBank accession number AF371337) is shown. Shown is SEQ ID NO:11

FIG. 51. Mutagenesis strategy in putative m⁶A site in the G gene in hMPV genome. Schematic diagram of the hMPV genome with the mutations for altering the critical A or C residues in the m⁶A motifs to produce rhMPV lacking that putative m⁶A modification site in the G gene. A total of 6 putative m⁶A site, G1-G6, are shown. Shown in FIG. 51 is SEQ ID NO:12.

FIG. 52. CPE produced by m⁶A-deficient hMPVs in A549 cells. Confluent A549 cells in 24-well plates were infected by each rhMPV mutant at an MOI of 5.0. CPE was imaged at 16, 24, 40 and 48 h post-infection.

FIG. 53A-B. m⁶A-deficient rhMPV RNA has reduced binding efficiency to reader proteins. A549 cells in T25 flasks were transfected with plasmid pYTHDF1-HA or pYTHDF2-HA, and were lysed in 650 μL 1× lysis buffer (abeam, ab152163) at 24 h post transfection. Cell lysate was divided into 3 tube (200 μL/tube) and incubated with 2×10⁸ copies of virion RNA (rhMPV-G8-14, rhMPV-G1-14, or rhMPV) and then incubated with 50 μL Pierce anti-HA Magnetic beads at room temperature for 30 min. Reader protein: RNA complex pulled down by the beads was subjected to Western blot (A). The amount of virion RNA captured by the YTHDF1 or YTHDF2 was quantified by real-time RT-PCR. Percent of bound RNA of hMPV mutants relative to rhMPV was calculated (B). Results are from three independent experiments. Western blots shown are the representatives of three independent experiments. Input equals to 5 μL of original lysate, and each I.P. sample equals to 40 μL of original lysate. P values of rhMPV-G8-14 or rhMPV-G1-14 compared to rhMPV are as follows: YTHDF1, P=0.01361, P=0.00099; YTHDF2, P=0.0000005, P=0.000005.

FIG. 54. Cell-death triggered by virion RNA of m⁶A-deficient hMPVs. Virion RNA was extracted from purified hMPV virions, the level of antigenome was quantified by real-time RT-PCR. A549 cells in 24-well plates were transfected with 2×10⁷ antigenome copies of virion RNA of each hMPV. Images were taken at 16, 24, 40 post-transfection.

FIG. 55. Cell-death triggered by virion RNA of m⁶A-deficient hMPVs with or without CIP. Virion RNA was extracted from purified hMPV virions, the level of antigenome was quantified by real-time RT-PCR. A549 cells in 24-well plates were transfected with 2×10⁷ antigenome copies of virion RNA of each hMPV either with or without CIP treatment. Images were taken at 48 post-transfection.

FIG. 56. CPE produced by m⁶A-deficient hMPVs in parental and knockout A549 cells. Confluent wild-type, MDA5, RIG-I, or MAVs-knockout A549 cells in 24-well plates were infected by rhMPV, rhMPV-G8-14, or rhMPV-G1-14 at an MOI of 5.0. CPE was imaged at 16 post-infection.

FIG. 57A-E. m⁶A enhances G protein expression. (A) Transient expression of m⁶A reader proteins enhances G expression. A549 cells were transfected with 1 μg of plasmids encoding YTHDF1, 2, 3, or YTHDC1. At 24 h post-transfection, cells were further transfected with 1 μg of pCAGGS-G. At 24 h post-transfection, total cell extracts were harvested and subjected to Western blot using antibody against hMPV G protein. (B) siRNA knockdown of m⁶A reader proteins reduces G expression. A549 cells were transfected with siRNA targeting YTHDF1, 2, 3, or YTHDC1. At 24 h post-transfection, cells were further transfected with 1 μg of pCAGGS-G. At 24 h post-transfection, cell lysates were harvested for Western blot analysis. (C) Transient expression of m⁶A writer proteins enhances G expression. A549 cells were transfected with 1 μg of plasmids encoding METTLE3 and/or METTL14. At 24 h post-transfection, cells were further transfected with 1 μg of pCAGGS-G. At 24 h post-transfection, total cell extracts were harvested and subjected to Western blot using antibody against hMPV G protein. (D) siRNA knockdown of m⁶A writer proteins reduces G expression. A549 cells were transfected with siRNA targeting METTLE3 and/or METTL14. At 24 h post-transfection, cells were further transfected with 1 μg of pCAGGS-G. At 24 h post-transfection, cell lysates were harvested for Western blot analysis. (E) Mutations in m6A sites in G reduces G expression. A549 cells were transfected with 1 μg of each plasmid. At 48 h post-transfection, cell lysates were harvested for Western blot analysis.

### DETAILED DESCRIPTION OF THE INVENTION

N⁶-methyladenosine (m6A or m⁶A, which are interchangeable) is the most prevalent internal modification of mRNAs in most eukaryotes. RNAs produced in these cells during virus replication may also acquire m6A methylation. The Examples demonstrate that RNAs of human respiratory syncytial virus (RSV), a medically important non-segmented negative-sense (NNS) RNA virus, are modified by m6A within discreet regions and that these modifications enhance viral replication and pathogenesis. Described herein are recombinant RSV variants that are highly attenuated yet retain high immunogenicity. Therefore, the RSV variants described herein can be used for rational design of live attenuated vaccine candidates and for novel antiviral therapeutic agents for RSV.

### I. Nucleic Acids

In certain embodiments, the disclosure concerns recombinant polynucleotides containing or encoding the m6A consensus sites.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene”, “polynucleotide”, or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges therebetween, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode all or segments of a gene from RSV, such as the G gene. The term “recombinant” may be used in conjunction with a polynucleotide or polypeptide and generally refers to a polypeptide or polynucleotide produced and/or manipulated in vitro or that is a replication product of such a molecule.

In other embodiments, the disclosure concerns isolated nucleic acid segments and recombinant vectors incorporating nucleic acid sequences that RSV genes or variants thereof to generate an immune response in a subject or to generate attenuated virus useful in the compositions and methods described herein.

The nucleic acid segments of the disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain other embodiments, the disclosure concerns isolated nucleic acid segments that include within their sequence a contiguous nucleic acid sequence from SEQ ID NO:1 (G gene RSV mRNA) or the complement thereof:

(SEQ ID NO: 1)
ATGTCCAAAAACAAGGACCAACGCACCGCTAAGACATTAGAAAGGACCTG
GGACACTCTCAATCATTTATTATTCATATCATCGTGCTTATATAAGTTAA
ATCTTAAATCTGTAGCACAAATCACATTATCCATTCTGGCAATGATAATC
TCAACTTCACTTATAATTGCAGCCATCATATTCATAGCCTCGGCAAACCA
CAAAGTCACACCAACAACTGCAATCATACAAGATGCAACAAGCCAGATCA
AGAACACAACCCCAACATACCTCACCCAGAATCCTCAGCTTGGAATCAGT
CCCTCTAATCCGTCTGAAATTACATCACAAATCACCACCATACTAGCTTC
AACAACACCAGGAGTCAAGTCAACCCTGCAATCCACAACAGTCAAGACCA
AAAACACAACAACAACTCAAACACAACCCAGCAAGCCCACCACAAAACAA
CGCCAAAACAAACCACCAAGCAAACCCAATAATGATTTTCACTTTGAAGT
GTTCAACTTTGTACCCTGCAGCATATGCAGCAACAATCCAACCTGCTGGG
CTATCTGCAAAAGAATACCAAACAAAAAACCAGGAAAGAAAACCACTACC
AAGCCCACAAAAAAACCAACCCTCAAGACAACCAAAAAAGATCCCAAACC
TCAAACCACTAAATCAAAGGAAGTACCCACCACCAAGCCCACAGAAGAGC
CAACCATCAACACCACCAAAACAAACATCATAACTACACTACTCACCTCC
AACACCACAGGAAATCCAGAACTCACAAGTCAAATGGAAACCTTCCACTC
AACTTCCTCCGAAGGCAATCCAAGCCCTTCTCAAGTCTCTACAACATCCG
AGTACCCATCACAACCTTCATCTCCACCCAACACACCACGCCAGTAG

In certain other embodiments, the disclosure concerns isolated nucleic acid segments and recombinant vectors, as RNA or DNA, that include within their sequence a contiguous nucleic acid sequence from SEQ ID NO:2 (RSV antigenome shown with thymine (T) instead of uracil (U)) or the RNA complement thereof (RSV genome):

(SEQ ID NO: 2)
TTGCATAAACCAAAAAAATGGGGCAAATAAGAATTTGATAAGTACCACTTAAAT
TTAACTCCCTTGGTTAGAGATGGGCAGCAATTCATTGAGTATGATAAAAGTTAGA
TTACAAAATTTGTTTGACAATGATGAAGTAGCATTGTTAAAAATAACATGCTATA
CTGATAAATTAATACATTTAACTAATGCTTTGGCTAAGGCAGTGATACATACAAT
CAAATTGAATGGCATTGTGTTTGTGCATGTTATTACAAGTAGTGATATTTGCCCTA
ATAATAATATTGTAGTAAAATCCAATTTCACAACAATGCCAGTACTACAAAATGG
AGGTTATATATGGGAAATGATGGAATTAACACATTGCTCTCAACCTAATGGTCTA
CTAGATGACAATTGTGAAATTAAATTCTCCAAAAAACTAAGTGATTCAACAATGA
CCAATTATATGAATCAATTATCTGAATTACTTGGATTTGATCTTAATCCATAAATT
ATAATTAATATCAACTAGCAAATCAATGTCACTAACACCATTAGTTAATATAAAA
CTTAACAGAAGACAAAAATGGGGCAAATAAATCAATTCAGCCAACCCAACCATG
GACACAACCCACAATGATAATACACCACAAAGACTGATGATCACAGACATGAGA
CCGTTGTCACTTGAGACCATAATAACATCACTAACCAGAGACATCATAACACACA
AATTTATATACTTGATAAATCATGAATGCATAGTGAGAAAACTTGATGAAAGAC
AGGCCACATTTACATTCCTGGTCAACTATGAAATGAAACTATTACACAAAGTAGG
AAGCACTAAATATAAAAAATATACTGAATACAACACAAAATATGGCACTTTCCC
TATGCCAATATTCATCAATCATGATGGGTTCTTAGAATGCATTGGCATTAAGCCT
ACAAAGCATACTCCCATAATATACAAGTATGATCTCAATCCATAAATTTCAACAC
AATATTCACACAATCTAAAACAACAACTCTATGCATAACTATACTCCATAGTCCA
GATGGAGCCTGAAAATTATAGTAATTTAAAACTTAAGGAGAGATATAAGATAGA
AGATGGGGCAAATACAACCATGGCTCTTAGCAAAGTCAAGTTGAATGATACACT
CAACAAAGATCAACTTCTGTCATCCAGCAAATACACCATCCAACGGAGCACAGG
AGATAGTATTGATACTCCTAATTATGATGTGCAGAAACACATCAATAAGTTATGT
GGCATGTTATTAATCACAGAAGATGCTAATCATAAATTCACTGGGTTAATAGGTA
TGTTATATGCGATGTCTAGGTTAGGAAGAGAAGACACCATAAAAATACTCAGAG
ATGCGGGATATCATGTAAAAGCAAATGGAGTAGATGTAACAACACATCGTCAAG
ACATTAATGGAAAAGAAATGAAATTTGAAGTGTTAACATTGGCAAGCTTAACAA
CTGAAATTCAAATCAACATTGAGATAGAATCTAGAAAATCCTACAAAAAAATGC
TAAAAGAAATGGGAGAGGTAGCTCCAGAATACAGGCATGACTCTCCTGATTGTG
GGATGATAATATTATGTATAGCAGCATTAGTAATAACTAAATTAGCAGCAGGGG
ACAGATCTGGTCTTACAGCCGTGATTAGGAGAGCTAATAATGTCCTAAAAAATG
AAATGAAACGTTACAAAGGCTTACTACCCAAGGACATAGCCAACAGCTTCTATG
AAGTGTTTGAAAAACATCCCCACTTTATAGATGTTTTTGTTCATTTTGGTATAGCA
CAATCTTCTACCAGAGGTGGCAGTAGAGTTGAAGGGATTTTTGCAGGATTGTTTA
TGAATGCCTATGGTGCAGGGCAAGTGATGTTACGGTGGGGAGTCTTAGCAAAAT
CAGTTAAAAATATTATGTTAGGACATGCTAGTGTGCAAGCAGAAATGGAACAAG
TTGTTGAGGTTTATGAATATGCCCAAAAATTGGGTGGTGAAGCAGGATTCTACCA
TATATTGAACAACCCAAAAGCATCATTATTATCTTTGACTCAATTTCCTCACTTCT
CCAGTGTAGTATTAGGCAATGCTGCTGGCCTAGGCATAATGGGAGAGTACAGAG
GTACACCGAGGAATCAAGATCTATATGATGCAGCAAAGGCATATGCTGAACAAC
TCAAAGAAAATGGTGTGATTAACTACAGTGTACTAGACTTGACAGCAGAAGAAC
TAGAGGCTATCAAACATCAGCTTAATCCAAAAGATAATGATGTAGAGCTTTGAGT
TAATAAAAAATGGGGCAAATAAATCATCATGGAAAAGTTTGCTCCTGAATTCCAT
GGAGAAGATGCAAACAACAGGGCTACTAAATTCCTAGAATCAATAAAGGGCAAA
TTCACATCACCCAAAGATCCCAAGAAAAAAGATAGTATCATATCTGTCAACTCAA
TAGATATAGAAGTAACCAAAGAAAGCCCTATAACATCAAATTCAACTATTATCA
ACCCAACAAATGAGACAGATGATACTGCAGGGAACAAGCCCAATTATCAAAGAA
AACCTCTAGTAAGTTTCAAAGAAGACCCTACACCAAGTGATAATCCCTTTTCTAA
ACTATACAAAGAAACCATAGAAACATTTGATAACAATGAAGAAGAATCCAGCTA
TTCATACGAAGAAATAAATGATCAGACAAACGATAATATAACAGCAAGATTAGA
TAGGATTGATGAAAAATTAAGTGAAATACTAGGAATGCTTCACACATTAGTAGT
GGCAAGTGCAGGACCTACATCTGCTCGGGATGGTATAAGAGATGCCATGGTTGG
TTTAAGAGAAGAAATGATAGAAAAAATCAGAACTGAAGCATTAATGACCAATGA
CAGATTAGAAGCTATGGCAAGACTCAGGAATGAGGAAAGTGAAAAGATGGCAA
AAGACACATCAGATGAAGTGTCTCTCAATCCAACATCAGAGAAATTGAACAACC
TATTGGAAGGGAATGATAGTGACAATGATCTATCACTTGAAGATTTCTGATTAGT
TACCAATCTTCACATCAACACACAATACCAACAGAAGACCAACAAACTAACCAA
CCCAATCATCCAACCAAACATCCATCCGCCAATCAGCCAAACAGCCAACAAAAC
AACCAGCCAATCCAAAACTAACCACCCGGAAAAAATCTATAATATAGTTACAAA
AAAAGGAAAGGGTGCGCGCTGGGGCAAATATGGAAACATACGTGAACAAGCTTC
ACGAAGGCTCCACATACACAGCTGCTGTTCAATACAATGTCTTAGAAAAAGACG
ATGACCCTGCATCACTTACAATATGGGTGCCCATGTTCCAATCATCTATGCCAGC
AGATTTACTTATAAAAGAACTAGCTAATGTCAACATACTAGTGAAACAAATATCC
ACACCCAAGGGACCTTCACTAAGAGTCATGATAAACTCAAGAAGTGCAGTGCTA
GCACAAATGCCCAGCAAATTTACCATATGCGCTAATGTGTCCTTGGATGAAAGAA
GCAAACTAGCATATGATGTAACCACACCCTGTGAAATCAAGGCATGTAGTCTAA
CATGCCTAAAATCAAAAAATATGTTGACTACAGTTAAAGATCTCACTATGAAGAC
ACTCAACCCTACACATGATATTATTGCTTTATGTGAATTTGAAAACATAGTAACA
TCAAAAAAAGTCATAATACCAACATACCTAAGATCCATCAGTGTCAGAAATAAA
GATCTGAACACACTTGAAAATATAACAACCACTGAATTCAAAAATGCTATCACA
AATGCAAAAATCATCCCTTACTCAGGATTACTATTAGTCATCACAGTGACTGACA
ACAAAGGAGCATTCAAATACATAAAGCCACAAAGTCAATTCATAGTAGATCTTG
GAGCTTACCTAGAAAAAGAAAGTATATATTATGTTACCACAAATTGGAAGCACA
CAGCTACACGATTTGCAATCAAACCCATGGAAGATTAACCTTTTTCCTCTACATC
AGTGTGTTAATTCATACAAACTTTCTACCTACATTCTTCACTTCACCATCACAATC
ACAAACACTCTGTGGTTCAACCAATCAAACAAAACTTATCTGAAGTCCCAGATCA
TCCCAAGTCATTGTTTATCAGATCTAGTACTCAAATAAGTTAATAAAAAATATAC
ACATGGCGATCGACATGGGGCAAATAATCATTGGAGGAAATCCAACTAATCACA
ATATCTGTTAACATAGACAAGTCCACACACCATACAGAATCAACCAATGGAAAA
TACATCCATAACAATAGAATTCTCAAGCAAATTCTGGCCTTACTTTACACTAATA
CACATGATCACAACAATAATCTCTTTGCTAATCATAATCTCCATCATGATTGCAA
TACTAAACAAACTTTGTGAATATAACGTATTCCATAACAAAACCTTTGAGTTACC
AAGAGCTCGCGTCAACACATAGCATTAGTTAATTAAAAATTAGGGCCCAACAAT
GAACTAGGATATCAAGACTAACAATAACATTGGGGCAAATGCAAACATGTCCAA
AAACAAGGACCAACGCACCGCTAAGACATTAGAAAGGACCTGGGACACTCTCAA
TCATTTATTATTCATATCATCGTGCTTATATAAGTTAAATCTTAAATCTGTAGCAC
AAATCACATTATCCATTCTGGCAATGATAATCTCAACTTCACTTATAATTGCAGC
CATCATATTCATAGCCTCGGCAAACCACAAAGTCACACCAACAACTGCAATCAT
ACAAGATGCAACAAGCCAGATCAAGAACACAACCCCAACATACCTCACCCAGAA
TCCTCAGCTTGGAATCAGTCCCTCTAATCCGTCTGAAATTACATCACAAATCACC
ACCATACTAGCTTCAACAACACCAGGAGTCAAGTCAACCCTGCAATCCACAACA
GTCAAGACCAAAAACACAACAACAACTCAAACACAACCCAGCAAGCCCACCAC
AAAACAACGCCAAAACAAACCACCAAGCAAACCCAATAATGATTTTCACTTTGA
AGTGTTCAACTTTGTACCCTGCAGCATATGCAGCAACAATCCAACCTGCTGGGCT
ATCTGCAAAAGAATACCAAACAAAAAACCAGGAAAGAAAACCACTACCAAGCC
CACAAAAAAACCAACCCTCAAGACAACCAAAAAAGATCCCAAACCTCAAACCAC
TAAATCAAAGGAAGTACCCACCACCAAGCCCACAGAAGAGCCAACCATCAACAC
CACCAAAACAAACATCATAACTACACTACTCACCTCCAACACCACAGGAAATCC
AGAACTCACAAGTCAAATGGAAACCTTCCACTCAACTTCCTCCGAAGGCAATCC
AAGCCCTTCTCAAGTCTCTACAACATCCGAGTACCCATCACAACCTTCATCTCCA
CCCAACACACCACGCCAGTAGTTACTTAAAAACATATTATCACAAAAGGCCTTGA
CCAACCGCGGAGAATCAAAATAAACTCTGGGGCAAATAACAATGGAGTTGCTAA
TCCTCAAAGCAAATGCAATTACCACAATCCTCACTGCAGTCACATTTTGTTTTGCT
TCTGGTCAAAACATCACTGAAGAATTTTATCAATCAACATGCAGTGCAGTTAGCA
AAGGCTATCTTAGTGCTCTGAGAACTGGTTGGTATACCAGTGTTATAACTATAGA
ATTAAGTAATATCAAGAAAAATAAGTGTAATGGAACAGATGCTAAGGTAAAATT
GATAAAACAAGAATTAGATAAATATAAAAATGCTGTAACAGAATTGCAGTTGCT
CATGCAAAGCACACAAGCAACAAACAATCGAGCCAGAAGAGAACTACCAAGGT
TTATGAATTATACACTCAACAATGCCAAAAAAACCAATGTAACATTAAGCAAGA
AAAGGAAAAGAAGATTTCTTGGTTTTTTGTTAGGTGTTGGATCTGCAATCGCCAG
TGGCGTTGCTGTATCTAAGGTCCTGCACCTAGAAGGGGAAGTGAACAAGATCAA
AAGTGCTCTACTATCCACAAACAAGGCTGTAGTCAGCTTATCAAATGGAGTTAGT
GTTTTAACCAGCAAAGTGTTAGACCTCAAAAACTATATAGATAAACAATTGTTAC
CTATTGTGAACAAGCAAAGCTGCAGCATATCAAATATAGAAACTGTGATAGAGT
TCCAACAAAAGAACAACAGACTACTAGAGATTACCAGGGAATTTAGTGTTAATG
CAGGCGTAACTACACCTGTAAGCACTTACATGTTAACTAATAGTGAATTATTGTC
ATTAATCAATGATATGCCTATAACAAATGATCAGAAAAAGTTAATGTCCAACAAT
GTTCAAATAGTTAGACAGCAAAGTTACTCTATCATGTCCATAATAAAAGAGGAA
GTCTTAGCATATGTAGTACAATTACCACTATATGGTGTTATAGATACACCCTGTT
GGAAACTACACACATCCCCTCTATGTACAACCAACACAAAAGAAGGGTCCAACA
TCTGTTTAACAAGAACTGACAGAGGATGGTACTGTGACAATGCAGGATCAGTAT
CTTTCTTCCCACAAGCTGAAACATGTAAAGTTCAATCAAATCGAGTATTTTGTGA
CACAATGAACAGTTTAACATTACCAAGTGAAGTAAATCTCTGCAATGTTGACATA
TTCAACCCCAAATATGATTGTAAAATTATGACTTCAAAAACAGATGTAAGCAGCT
CCGTTATCACATCTCTAGGAGCCATTGTGTCATGCTATGGCAAAACTAAATGTAC
AGCATCCAATAAAAATCGTGGAATCATAAAGACATTTTCTAACGGGTGCGATTAT
GTATCAAATAAAGGGGTGGACACTGTGTCTGTAGGTAACACATTATATTATGTAA
ATAAGCAAGAAGGTAAAAGTCTCTATGTAAAAGGTGAACCAATAATAAATTTCT
ATGACCCATTAGTATTCCCCTCTGATGAATTTGATGCATCAATATCTCAAGTCAA
CGAGAAGATTAACCAGAGCCTAGCATTTATTCGTAAATCCGATGAATTATTACAT
AATGTAAATGCTGGTAAATCCACCACAAATATCATGATAACTACTATAATTATAG
TGATTATAGTAATATTGTTATCATTAATTGCTGTTGGACTGCTCTTATACTGTAAG
GCCAGAAGCACACCAGTCACACTAAGCAAAGATCAACTGAGTGGTATAAATAAT
ATTGCATTTAGTAACTAAATAAAAATAGCACCTAATCATGTTCTTACAATGGTTT
ACTATCTGCTCATAGACAACCCATCTGTCATTGGATTTTCTTAAAATCTGAACTTC
ATCGAAACTCTCATCTATAAACCATCTCACTTACACTATTTAAGTAGATTCCTAGT
TTATAGTTATATAAAACACAATTGCATGCCACTCGAGCTTACCATCTGTAAAAAT
GAAAACTGGGGCAAATATGTCACGAAGGAATCCTTGCAAATTTGAAATTCGAGG
TCATTGCTTAAATGGTAAGAGGTGTCATTTTAGTCATAATTATTTTGAATGGCCAC
CCCATGCACTGCTTGTAAGACAAAACTTTATGTTAAACAGAATACTTAAGTCTAT
GGATAAAAGTATAGATACCTTATCAGAAATAAGTGGAGCTGCAGAGTTGGACAG
AACAGAAGAGTATGCTCTTGGTGTAGTTGGAGTGCTAGAGAGTTATATAGGATC
AATAAACAATATAACTAAACAATCAGCATGTGTTGCCATGAGCAAACTCCTCACT
GAACTCAATAGTGATGATATCAAAAAGCTGAGGGACAATGAAGAGCTAAATTCA
CCCAAGATAAGAGTGTACAATACTGTCATATCATATATTGAAAGCAACAGGAAA
AACAATAAACAAACTATCCATCTGTTAAAAAGATTGCCAGCAGACGTATTGAAG
AAAACCATCAAAAACACATTGGATATCCATAAGAGCATAACCATCAACAACCCA
AAAGAATCAACTGTTAGTGATACAAATGACCATGCCAAAAATAATGATACTACC
TGACAAATATCCTTGTAGTATAACTTCCATACTAATAACAAGTAGATGTAGAGTT
ACTATGTATAATCAAAAGAACACACTATATTTCAATCAAAACAACCCAAATAAC
CATATGTACTCACCGAATCAAACATTCAATGAAATCCATTGGACCTCTCAAGAAT
TGATTGACACAATTCAAAATTTTCTACAACATCTAGGTATTATTGAGGATATATA
TACAATATATATATTAGTGTCATAACACTCAATTCTAACACTCACCACATCGTTA
CATTATTAATTCAAACAATTCAAGTTGTGGGACAAAATGGATCCCATTATTAATG
GAAATTCTGCTAATGTTTATCTAACCGATAGTTATTTAAAAGGTGTTATCTCTTTC
TCAGAGTGTAATGCTTTAGGAAGTTACATATTCAATGGTCCTTATCTCAAAAATG
ATTATACCAACTTAATTAGTAGACAAAATCCATTAATAGAACACATGAATCTAAA
GAAACTAAATATAACACAGTCCTTAATATCTAAGTATCATAAAGGTGAAATAAA
ATTAGAAGAACCTACTTATTTTCAGTCATTACTTATGACATACAAGAGTATGACC
TCGTCAGAACAGATTGCTACCACTAATTTACTTAAAAAGATAATAAGAAGAGCT
ATAGAAATAAGTGATGTCAAAGTCTATGCTATATTGAATAAACTAGGGCTTAAA
GAAAAGGACAAGATTAAATCCAACAATGGACAAGATGAAGACAACTCAGTTATT
ACGACCATAATCAAAGATGATATACTTTCAGCTGTTAAAGATAATCAATCTCATC
TTAAAGCAGACAAAAATCACTCTACAAAACAAAAAGACACAATCAAAACAACAC
TCTTGAAGAAATTGATGTGTTCAATGCAACATCCTCCATCATGGTTAATACATTG
GTTTAACTTATACACAAAATTAAACAACATATTAACACAGTATCGATCAAATGAG
GTAAAAAACCATGGGTTTACATTGATAGATAATCAAACTCTTAGTGGATTTCAAT
TTATTTTGAACCAATATGGTTGTATAGTTTATCATAAGGAACTCAAAAGAATTAC
TGTGACAACCTATAATCAATTCTTGACATGGAAAGATATTAGCCTTAGTAGATTA
AATGTTTGTTTAATTACATGGATTAGTAACTGCTTGAACACATTAAATAAAAGCT
TAGGCTTAAGATGCGGATTCAATAATGTTATCTTGACACAACTATTCCTTTATGG
AGATTGTATACTAAAGCTATTTCACAATGAGGGGTTCTACATAATAAAAGAGGTA
GAGGGATTTATTATGTCTCTAATTTTAAATATAACAGAAGAAGATCAATTCAGAA
AACGATTTTATAATAGTATGCTCAACAACATCACAGATGCTGCTAATAAAGCTCA
GAAAAATCTGCTATCAAGAGTATGTCATACATTATTAGATAAGACAGTGTCCGAT
AATATAATAAATGGCAGATGGATAATTCTATTAAGTAAGTTCCTTAAATTAATTA
AGCTTGCAGGTGACAATAACCTTAACAATCTGAGTGAACTATATTTTTTGTTCAG
AATATTTGGACACCCAATGGTAGATGAAAGACAAGCCATGGATGCTGTTAAAAT
TAATTGCAATGAGACCAAATTTTACTTGTTAAGCAGTCTGAGTATGTTAAGAGGT
GCCTTTATATATAGAATTATAAAAGGGTTTGTAAATAATTACAACAGATGGCCTA
CTTTAAGAAATGCTATTGTTTTACCCTTAAGATGGTTAACTTACTATAAACTAAAC
ACTTATCCTTCTTTGTTGGAACTTACAGAAAGAGATTTGATTGTGTTATCAGGACT
ACGTTTCTATCGTGAGTTTCGGTTGCCTAAAAAAGTGGATCTTGAAATGATTATA
AATGATAAAGCTATATCACCTCCTAAAAATTTGATATGGACTAGTTTCCCTAGAA
ATTACATGCCATCACACATACAAAACTATATAGAACATGAAAAATTAAAATTTTC
CGAGAGTGATAAATCAAGAAGAGTATTAGAGTATTATTTAAGAGATAACAAATT
CAATGAATGTGATTTATACAACTGTGTAGTTAATCAAAGTTATCTCAACAACCCT
AATCATGTGGTATCATTGACAGGCAAAGAAAGAGAACTCAGTGTAGGTAGAATG
TTTGCAATGCAACCGGGAATGTTCAGACAGGTTCAAATATTGGCAGAGAAAATG
ATAGCTGAAAACATTTTACAATTCTTTCCTGAAAGTCTTACAAGATATGGTGATC
TAGAACTACAAAAAATATTAGAACTGAAAGCAGGAATAAGTAACAAATCAAATC
GCTACAATGATAATTACAACAATTACATTAGTAAGTGCTCTATCATCACAGATCT
CAGCAAATTCAATCAAGCATTTCGATATGAAACGTCATGTATTTGTAGTGATGTG
CTGGATGAACTGCATGGTGTACAATCTCTATTTTCCTGGTTACATTTAACTATTCC
TCATGTCACAATAATATGCACATATAGGCATGCACCCCCCTATATAGGAGATCAT
ATTGTAGATCTTAACAATGTAGATGAACAAAGTGGATTATATAGATATCACATGG
GTGGCATCGAAGGGTGGTGTCAAAAACTATGGACCATAGAAGCTATATCACTAT
TGGATCTAATATCTCTCAAAGGGAAATTCTCAATTACTGCTTTAATTAATGGTGA
CAATCAATCAATAGATATAAGCAAACCAATCAGACTCATGGAAGGTCAAACTCA
TGCTCAAGCAGATTATTTGCTAGCATTAAATAGCCTTAAATTACTGTATAAAGAG
TATGCAGGCATAGGCCACAAATTAAAAGGAACTGAGACTTATATATCACGAGAT
ATGCAATTTATGAGTAAAACAATTCAACATAACGGTGTATATTACCCAGCTAGTA
TAAAGAAAGTCCTAAGAGTGGGACCGTGGATAAACACTATACTTGATGATTTCA
AAGTGAGTCTAGAATCTATAGGTAGTTTGACACAAGAATTAGAATATAGAGGTG
AAAGTCTATTATGCAGTTTAATATTTAGAAATGTATGGTTATATAATCAGATTGC
TCTACAATTAAAAAATCATGCATTATGTAACAATAAACTATATTTGGACATATTA
AAGGTTCTGAAACACTTAAAAACCTTTTTTAATCTTGATAATATTGATACAGCAT
TAACATTGTATATGAATTTACCCATGTTATTTGGTGGTGGTGATCCCAACTTGTTA
TATCGAAGTTTCTATAGAAGAACTCCTGACTTCCTCACAGAGGCTATAGTTCACT
CTGTGTTCATACTTAGTTATTATACAAACCATGACTTAAAAGATAAACTTCAAGA
TCTGTCAGATGATAGATTGAATAAGTTCTTAACATGCATAATCACGTTTGACAAA
AACCCTAATGCTGAATTCGTAACATTGATGAGAGATCCTCAAGCTTTAGGGTCTG
AGAGACAAGCTAAAATTACTAGCGAAATCAATAGACTGGCAGTTACAGAGGTTT
TGAGTACAGCTCCAAACAAAATATTCTCCAAAAGTGCACAACATTATACTACTAC
AGAGATAGATCTAAATGATATTATGCAAAATATAGAACCTACATATCCTCATGGG
CTAAGAGTTGTTTATGAAAGTTTACCCTTTTATAAAGCAGAGAAAATAGTAAATC
TTATATCAGGTACAAAATCTATAACTAACATACTGGAAAAAACTTCTGCCATAGA
CTTAACAGATATTGATAGAGCCACTGAGATGATGAGGAAAAACATAACTTTGCTT
ATAAGGATACTTCCATTGGATTGTAACAGAGATAAAAGAGAGATATTGAGTATG
GAAAACCTAAGTATTACTGAATTAAGCAAATATGTTAGGGAAAGATCTTGGTCTT
TATCCAATATAGTTGGTGTTACATCACCCAGTATCATGTATACAATGGACATCAA
ATATACTACAAGCACTATATCTAGTGGCATAATTATAGAGAAATATAATGTTAAC
AGTTTAACACGTGGTGAGAGAGGACCCACTAAACCATGGGTTGGTTCATCTACAC
AAGAGAAAAAAACAATGCCAGTTTATAATAGACAAGTCTTAACCAAAAAACAGA
GAGATCAAATAGATCTATTAGCAAAATTGGATTGGGTGTATGCATCTATAGATAA
CAAGGATGAATTCATGGAAGAACTCAGCATAGGAACCCTTGGGTTAACATATGA
AAAGGCCAAGAAATTATTTCCACAATATTTAAGTGTCAATTATTTGCATCGCCTT
ACAGTCAGTAGTAGACCATGTGAATTCCCTGCATCAATACCAGCTTATAGAACAA
CAAATTATCACTTTGACACTAGCCCTATTAATCGCATATTAACAGAAAAGTATGG
TGATGAAGATATTGACATAGTATTCCAAAACTGTATAAGCTTTGGCCTTAGTTTA
ATGTCAGTAGTAGAACAATTTACTAATGTATGTCCTAACAGAATTATTCTCATAC
CTAAGCTTAATGAGATACATTTGATGAAACCTCCCATATTCACAGGTGATGTTGA
TATTCACAAGTTAAAACAAGTGATACAAAAACAGCATATGTTTTTACCAGACAA
AATAAGTTTGACTCAATATGTGGAATTATTCTTAAGTAATAAAACACTCAAATCT
GGATCTCATGTTAATTCTAATTTAATATTGGCACATAAAATATCTGACTATTTTCA
TAATACTTACATTTTAAGTACTAATTTAGCTGGACATTGGATTCTGATTATACAAC
TTATGAAAGATTCTAAAGGTATTTTTGAAAAAGATTGGGGAGAGGGATATATAA
CTGATCATATGTTTATTAATTTGAAAGTTTTCTTCAATGCTTATAAGACCTATCTC
TTGTGTTTTCATAAAGGTTATGGCAAAGCAAAGCTGGAGTGTGATATGAACACTT
CAGATCTTCTATGTGTATTGGAATTAATAGACAGTAGTTATTGGAAGTCTATGTC
TAAGGTATTTTTAGAACAAAAAGTTATCAAATACATTCTTAGCCAAGATGCAAGT
TTACATAGAGTAAAAGGATGTCATAGCTTCAAATTATGGTTTCTTAAACGTCTTA
ATGTAGCAGAATTCACAGTTTGCCCTTGGGTTGTTAACATAGATTATCATCCAAC
ACATATGAAAGCAATATTAACTTATATAGATCTTGTTAGAATGGGATTGATAAAT
ATAGATAGAATACACATTAAAAATAAACACAAATTCAATGATGAATTTTATACTT
CTAATCTCTTCTACATTAATTATAACTTCTCAGATAATACTCATCTATTAACTAAA
CATATAAGGATTGCTAATTCTGAATTAGAAAATAATTACAACAAATTATATCATC
CTACACCAGAAACCCTAGAGAATATACTAGCCAATCCGATTAAAAGTAATGACA
AAAAGACACTGAATGACTATTGTATAGGTAAAAATGTTGACTCAATAATGTTACC
ATTGTTATCTAATAAGAAGCTTATTAAATCGTCTGCAATGATTAGAACCAATTAC
AGCAAACAAGATTTGTATAATTTATTCCCTATGGTTGTGATTGATAGAATTATAG
ATCATTCAGGCAATACAGCCAAATCCAACCAACTTTACACTACTACTTCCCACCA
AATATCCTTAGTGCACAATAGCACATCACTTTACTGCATGCTTCCTTGGCATCATA
TTAATAGATTCAATTTTGTATTTAGTTCTACAGGTTGTAAAATTAGTATAGAGTAT
ATTTTAAAAGATCTTAAAATTAAAGATCCCAATTGTATAGCATTCATAGGTGAAG
GAGCAGGGAATTTATTATTGCGTACAGTAGTGGAACTTCATCCTGACATAAGATA
TATTTACAGAAGTCTGAAAGATTGCAATGATCATAGTTTACCTATTGAGTTTTTA
AGGCTGTACAATGGACATATCAACATTGATTATGGTGAAAATTTGACCATTCCTG
CTACAGATGCAACCAACAACATTCATTGGTCTTATTTACATATAAAGTTTGCTGA
ACCTATCAGTCTTTTTGTCTGTGATGCCGAATTGTCTGTAACAGTCAACTGGAGTA
AAATTATAATAGAATGGAGCAAGCATGTAAGAAAGTGCAAGTACTGTTCCTCAG
TTAATAAATGTATGTTAATAGTAAAATATCATGCTCAAGATGATATTGATTTCAA
ATTAGACAATATAACTATATTAAAAACTTATGTATGCTTAGGCAGTAAGTTAAAG
GGATCGGAGGTTTACTTAGTCCTTACAATAGGTCCTGCGAATATATTCCCAGTAT
TTAATGTAGTACAAAATGCTAAATTGATACTATCAAGAACCAAAAATTTCATCAT
GCCTAAGAAAGCTGATAAAGAGTCTATTGATGCAAATATTAAAAGTTTGATACCC
TTTCTTTGTTACCCTATAACAAAAAAAGGAATTAATACTGCATTGTCAAAACTAA
AGAGTGTTGTTAGTGGAGATATACTATCATATTCTATAGCTGGACGTAATGAAGT
TTTCAGCAATAAACTTATAAATCATAAGCATATGAACATCTTAAAATGGTTCAAT
CATGTTTTAAATTTCAGATCAACAGAACTAAACTATAACCATTTATATATGGTAG
AATCTACATATCCTTACCTAAGTGAATTGTTAAACAGCTTGACAACCAATGAACT
TAAAAAACTGATTAAAATCACAGGTAGTCTGTTATACAACTTTCATAATGAATAA
TGAATAAAGATCTTATAATAAAAATTCCCATAGCTATACACTAACACTGTATTCA
ATTATAGTTATTAAAAATTAAAAATCGTACGATTTTTTAAATAACTTTTAGTGAA
CTAATCCTAAAGTTATCATTTTAATCTTGGAGGAATAAATTTAAACCCTAATCTA
ATTGGTTTATATGTGTATTAACTAAATTACGAGATATTAGTTTTTGACACTTTTTT
TCTCGT.

In certain other embodiments, the disclosure concerns isolated nucleic acid segments that include within their sequence a contiguous nucleic acid sequence from SEQ ID NO:3 (G gene antigenome of hMPV) or the complement thereof (G gene genome sequence):

(SEQ ID NO: 3)
ATGGAGGTGAAAGTGGAGAACATTCGAACAATAGATATGCTCAAAGCAAG
AGTAAAAAATCGTGTGGCACGCAGCAAATGCTTTAAAAATGCCTCTTTGG
TCCTCATAGGAATAACTACATTGAGTATTGCCCTCAATATCTATCTGATC
ATAAACTATAAAATGCAAAAAAACACATCTGAATCAGAACATCACACCAG
CTCATCACCCATGGAATCCAGCAGAGAAACTCCAACGGTCCCCACAGACA
ACTCAGACACCAACTCAAGCCCACAGCATCCAACTCAACAGTCCACAGAA
GGCTCCACACTCTACTTTGCAGCCTCAGCAAGCTCACCAGAGACAGAACC
AACATCAACACCAGATACAACAAACCGCCCGCCCTTCGTCGACACACACA
CAACACCACCAAGCGCAAGCAGAACAAAGACAAGTCCGGCAGTCCACACA
AAAAACAACCCAAGGACAAGCTCTAGAACACATTCTCCACCACGGGCAAC
GACAAGGACGGCACGCAGAACCACCACTCTCCGCACAAGCAGCACAAGAA
AGAGACCGTCCACAGCATCAGTCCAACCTGACATCAGCGCAACAACCCAC
AAAAACGAAGAAGCAAGTCCAGCGAGCCCACAAACATCTGCAAGCACAAC
AAGAATACAAAGGAAAAGCGTGGAGGCCAACACATCAACAACATACAACC
AAACTAGTTAA

In certain other embodiments, the disclosure concerns isolated nucleic acid segments and recombinant vectors, as RNA or DNA, that include within their sequence a contiguous nucleic acid sequence from SEQ ID NO:4 (hMPV antigenome shown with thymine (T) instead of uracil (U)) or the RNA complement thereof (hMPV genome):

(SEQ ID NO: 4)
GTATAAATTAGATTCCAAAAAAATATGGGACAAGTGAAAATGTCTCTTCAAGGG
ATTCACCTGAGTGATTTATCATACAAGCATGCTATATTAAAAGAGTCTCAGTACA
CAATAAAAAGAGATGTGGGTACAACAACTGCAGTGACACCCTCATCATTGCAAC
AAGAAATAACACTGTTGTGTGGAGAAATTCTGTATGCTAAACATGCTGACTACAA
ATATGCTGCAGAAATAGGAATACAATATATTAGCACAGCTTTAGGATCAGAGAG
AGTGCAGCAGATTCTGAGGAACTCAGGCAGTGAAGTCCAAGTGGTCTTAACCAG
AACGTACTCTCTGGGGAAAATTAAAAACAATAAAGGAGAAGATTTACAGATGTT
AGACATACACGGGGTAGAGAAGAGCTGGGTAGAAGAGATAGACAAAGAAGCAA
GGAAAACAATGGCAACCTTGCTTAAGGAATCATCAGGTAATATCCCACAAAATC
AGAGGCCCTCAGCACCAGACACACCCATAATCTTATTATGTGTAGGTGCCTTAAT
ATTCACTAAACTAGCATCAACCATAGAAGTGGGACTAGAGACCACAGTCAGAAG
GGCTAACCGTGTACTAAGTGATGCACTCAAGAGATACCCTAGAATGGACATACC
AAAGATTGCCAGATCCTTCTATGACTTATTTGAACAAAAAGTGTATCACAGAAGT
TTGTTCATTGAGTATGGCAAAGCATTAGGCTCATCATCTACAGGCAGCAAAGCAG
AAAGTCTATTTGTTAATATATTCATGCAAGCTTATGGGGCCGGTCAAACAATGCT
AAGGTGGGGGGTCATTGCCAGGTCATCCAACAATATAATGTTAGGACATGTATCC
GTCCAAGCTGAGTTAAAACAGGTCACAGAAGTCTATGACTTGGTGCGAGAAATG
GGCCCTGAATCTGGACTTCTACATTTAAGGCAAAGCCCAAAAGCTGGACTGTTAT
CACTAGCCAACTGTCCCAACTTTGCAAGTGTTGTTCTCGGAAATGCCTCAGGCTT
AGGCATAATCGGTATGTATCGAGGGAGAGTACCAAACACAGAATTATTTTCAGC
AGCTGAAAGTTATGCCAAAAGTTTGAAAGAAAGCAATAAAATAAATTTCTCTTC
ATTAGGACTTACAGATGAAGAGAAAGAGGCTGCAGAACATTTCTTAAATGTGAG
TGACGACAGTCAAAATGATTATGAGTAATTAAAAAAGTGGGACAAGTCAAAATG
TCATTCCCTGAAGGAAAAGATATTCTTTTCATGGGTAATGAAGCAGCAAAATTAG
CAGAAGCTTTCCAGAAATCATTAAGAAAACCAGGTCATAAAAGATCTCAATCTA
TTATAGGAGAAAAAGTGAATACTGTATCAGAAACATTGGAATTACCTACTATCA
GTAGACCTGCAAAACCAACCATACCGTCAGAACCAAAGTTAGCATGGACAGATA
AAGGTGGGGCAACCAAAACTGAAATAAAGCAAGCAATCAAAGTCATGGATCCCA
TTGAAGAAGAAGAGTCTACCGAGAAGAAGGTGCTACCCTCCAGTGATGGGAAAA
CCCCTGCAGAAAAGAAACTGAAACCATCAACTAACACCAAAAAGAAGGTTTCAT
TTACACCAAATGAACCAGGGAAATATACAAAGTTGGAAAAAGATGCTCTAGATT
TGCTCTCAGATAATGAAGAAGAAGATGCAGAATCTTCAATCTTAACCTTTGAAGA
AAGAGATACTTCATCATTAAGCATTGAGGCCAGATTGGAATCAATAGAGGAGAA
ATTAAGCATGATATTAGGGCTATTAAGAACACTCAACATTGCTACAGCAGGACCC
ACAGCAGCAAGAGATGGGATCAGAGATGCAATGATTGGCGTAAGAGAGGAATT
AATAGCAGACATAATAAAGGAAGCTAAAGGGAAAGCAGCAGAAATGATGGAAG
AGGAAATGAGTCAACGATCAAAAATAGGAAATGGTAGTGTAAAATTAACAGAA
AAAGCAAAAGAGCTCAACAAAATTGTTGAAGATGAAAGCACAAGTGGAGAATC
CGAAGAAGAAGAAGAACCAAAAGACACACAAGACAATAGTCAAGAAGATGACA
TTTACCAGTTAATTATGTAGTTTAATAAAAATAAACAATGGGACAAGTAAAAATG
GAGTCCTACCTAGTAGACACCTATCAAGGCATTCCTTACACAGCAGCTGTTCAAG
TTGATCTAATAGAAAAGGACCTGTTACCTGCAAGCCTAACAATATGGTTCCCTTT
GTTTCAGGCCAACACACCACCAGCAGTGCTGCTCGATCAGCTAAAAACCCTGAC
AATAACCACTCTGTATGCTGCATCACAAAATGGTCCAATACTCAAAGTGAATGCA
TCAGCCCAAGGTGCAGCAATGTCTGTACTTCCCAAAAAATTTGAAGTCAATGCGA
CTGTAGCACTCGATGAATATAGCAAACTGGAATTTGACAAACTCACAGTCTGTGA
AGTAAAAACAGTTTACTTAACAACCATGAAACCATACGGGATGGTATCAAAATT
TGTGAGCTCAGCCAAATCAGTTGGCAAAAAAACACATGATCTAATCGCACTATGT
GATTTTATGGATCTAGAAAAGAACACACCTGTTACAATACCAGCATTCATCAAAT
CAGTTTCAATCAAAGAGAGTGAGTCAGCTACTGTTGAAGCTGCTATAAGCAGTG
AAGCAGACCAAGCTCTAACACAGGCCAAAATTGCACCTTATGCGGGATTAATTA
TGATCATGACTATGAACAATCCCAAAGGCATATTCAAAAAGCTTGGAGCTGGGA
CTCAAGTCATAGTAGAACTAGGAGCATATGTCCAGGCTGAAAGCATAAGCAAAA
TATGCAAGACTTGGAGCCATCAAGGGACAAGATATGTCTTGAAGTCCAGATAAC
AACCAAGCACCTTGGCCAAGAGCTACTAACCCTATCTCATAGATCATAAAGTCAC
CATTCTAGTTATATAAAAATCAAGTTAGAACAAGAATTAAATCAATCAAGAACG
GGACAAATAAAAATGTCTTGGAAAGTGGTGATCATTTTTTCATTGTTAATAACAC
CTCAACACGGTCTTAAAGAGAGCTACTTAGAAGAGTCATGTAGCACTATAACTG
AAGGATATCTCAGTGTTCTGAGGACAGGTTGGTACACCAATGTTTTTACACTGGA
GGTAGGCGATGTAGAGAACCTTACATGTGCCGATGGACCCAGCTTAATAAAAAC
AGAATTAGACCTGACCAAAAGTGCACTAAGAGAGCTCAGAACAGTTTCTGCTGA
TCAACTGGCAAGAGAGGAGCAAATTGAAAATCCCAGACAATCTAGATTCGTTCT
AGGAGCAATAGCACTCGGTGTTGCAACTGCAGCTGCAGTTACAGCAGGTGTTGC
AATTGCCAAAACCATCCGGCTTGAAAGTGAAGTAACAGCAATTAAGAATGCCCT
CAAAAAGACCAATGAAGCAGTATCTACATTGGGGAATGGAGTTCGTGTGTTGGC
AACTGCAGTGAGAGAGCTGAAAGATTTTGTGAGCAAGAATCTAACACGTGCAAT
CAACAAAAACAAGTGCGACATTGCTGACCTGAAAATGGCCGTTAGCTTCAGTCA
ATTCAACAGAAGGTTCCTAAATGTTGTGCGGCAATTTTCAGACAACGCTGGAATA
ACACCAGCAATATCTTTGGACTTAATGACAGATGCTGAACTAGCCAGAGCTGTTT
CCAACATGCCAACATCTGCAGGACAAATAAAACTGATGTTGGAGAACCGTGCAA
TGGTAAGAAGAAAAGGGTTCGGATTCCTGATAGGAGTTTACGGAAGCTCCGTAA
TTTACATGGTGCAACTGCCAATCTTTGGGGTTATAGACACGCCTTGCTGGATAGT
AAAAGCAGCCCCTTCTTGTTCAGGAAAAAAGGGAAACTATGCTTGCCTCTTAAGA
GAAGACCAAGGATGGTATTGTCAAAATGCAGGGTCAACTGTTTACTACCCAAAT
GAAAAAGACTGTGAAACAAGAGGAGACCATGTCTTTTGCGACACAGCAGCAGGA
ATCAATGTTGCTGAGCAGTCAAAGGAGTGCAACATAAACATATCTACTACTAATT
ACCCATGCAAAGTTAGCACAGGAAGACATCCTATCAGTATGGTTGCACTATCTCC
TCTTGGGGCTTTGGTTGCTTGCTACAAGGGAGTGAGCTGTTCCATTGGCAGCAAC
AGAGTAGGGATCATCAAGCAACTGAACAAAGGCTGCTCTTATATAACCAACCAA
GACGCAGACACAGTGACAATAGACAACACTGTATACCAGCTAAGCAAAGTTGAA
GGCGAACAGCATGTTATAAAAGGAAGGCCAGTGTCAAGCAGCTTTGACCCAGTC
AAGTTTCCTGAAGATCAATTCAATGTTGCACTTGACCAAGTTTTCGAGAGCATTG
AGAACAGTCAGGCCTTGGTGGATCAATCAAACAGAATCCTAAGCAGTGCAGAGA
AAGGAAACACTGGCTTCATCATTGTAATAATTCTAATTGCTGTCCTTGGCTCTACC
ATGATCCTAGTGAGTGTTTTTATCATAATAAAGAAAACAAAGAAACCCACAGGA
GCACCTCCAGAGCTGAGTGGTGTCACAAACAATGGCTTCATACCACATAATTAGT
TAATTAAAAATAAAGTAAATTAAAATAAATTAAAATTAAAAATAAAAATTTGGG
ACAAATCATAATGTCTCGCAAGGCTCCGTGCAAATATGAAGTGCGGGGCAAATG
CAATAGAGGAAGTGAGTGCAAGTTTAACCACAATTACTGGAGTTGGCCAGATAG
ATACTTATTAATAAGATCAAATTATTTATTAAATCAACTTTTAAGGAACACTGAT
AGAGCTGATGGCTTATCAATAATATCAGGAGCAGGCAGAGAAGATAGGACACAA
GATTTTGTCCTAGGTTCCACCAATGTGGTTCAAGGTTATATTGATGATAACCAAA
GCATAACAAAAGCTGCAGCCTGTTACAGTCTACATAATATAATCAAACAACTAC
AAGAAGTTGAAGTTAGGCAGGCTAGAGATAACAAACTATCTGACAGCAAACATG
TAGCACTTCACAACTTAGTCCTATCTTATATGGAGATGAGCAAAACTCCTGCATC
TTTAATCAACAATCTCAAGAGACTGCCGAGAGAGAAACTGAAAAAATTAGCAAA
GCTCATAATTGACTTATCAGCAGGTGCTGAAAATGACTCTTCATATGCCTTGCAA
GACAGTGAAAGCACTAATCAAGTGCAGTGAGCATGGTCCAGTTTTCATTACTATA
GAGGTTGATGACATGATATGGACTCACAAGGACTTAAAAGAAGCTTTATCTGAT
GGGATAGTGAAGTCTCATACTAACATTTACAATTGTTATTTAGAAAACATAGAAA
TTATATATGTCAAGGCTTACTTAAGTTAGTAAAAACACATCAGAGTGGGATAAAT
GACAATGATAACATTAGATGTCATTAAAAGTGATGGGTCTTCAAAAACATGTACT
CACCTCAAAAAAATAATTAAAGACCACTCTGGTAAAGTGCTTATTGTACTTAAGT
TAATATTAGCTTTACTAACATTTCTCACAGTAACAATCACCATCAATTATATAAA
AGTGGAAAACAATCTGCAAATATGCCAGTCAAAAACTGAATCAGACAAAAAGGA
CTCATCATCAAATACCACATCAGTCACAACCAAGACTACTCTAAATCATGATATC
ACACAGTATTTTAAAAGTTTGATTCAAAGGTATACAAACTCTGCAATAAACAGTG
ACACATGCTGGAAAATAAACAGAAATCAATGCACAAATATAACAACATACAAAT
TTTTATGTTTTAAATCTGAAGACACAAAAACCAACAATTGTGATAAACTGACAGA
TTTATGCAGAAACAAACCAAAACCAGCAGTTGGAGTGTATCACATAGTAGAATG
CCATTGTATATACACAGTTAAATGGAAGTGCTATCATTACCCAACCGATGAAACC
CAATCCTAAATGTTAACACCAGATTAGGATCCATCCAAGTCTGTTAGTTCAACAA
TTTAGTTATTTAAAAATATTTTGAAAACAAGTAAGTTTCTATGATACTTCATAATA
ATAAGTAATAATTAATTGCTTAATCATCATCACAACATTATTCGAAACCATAACT
ATTCAATTTAAAAAGTAAAAAACAATAACATGGGACAAGTAGTTATGGAGGTGA
AAGTGGAGAACATTCGAACAATAGATATGCTCAAAGCAAGAGTAAAAAATCGTG
TGGCACGCAGCAAATGCTTTAAAAATGCCTCTTTGGTCCTCATAGGAATAACTAC
ATTGAGTATTGCCCTCAATATCTATCTGATCATAAACTATAAAATGCAAAAAAAC
ACATCTGAATCAGAACATCACACCAGCTCATCACCCATGGAATCCAGCAGAGAA
ACTCCAACGGTCCCCACAGACAACTCAGACACCAACTCAAGCCCACAGCATCCA
ACTCAACAGTCCACAGAAGGCTCCACACTCTACTTTGCAGCCTCAGCAAGCTCAC
CAGAGACAGAACCAACATCAACACCAGATACAACAAACCGCCCGCCCTTCGTCG
ACACACACACAACACCACCAAGCGCAAGCAGAACAAAGACAAGTCCGGCAGTC
CACACAAAAAACAACCCAAGGACAAGCTCTAGAACACATTCTCCACCACGGGCA
ACGACAAGGACGGCACGCAGAACCACCACTCTCCGCACAAGCAGCACAAGAAA
GAGACCGTCCACAGCATCAGTCCAACCTGACATCAGCGCAACAACCCACAAAAA
CGAAGAAGCAAGTCCAGCGAGCCCACAAACATCTGCAAGCACAACAAGAATAC
AAAGGAAAAGCGTGGAGGCCAACACATCAACAACATACAACCAAACTAGTTAAC
AAAAAATACAAAATAACTCTAAGATAAACCATGCAGACACCAACAATGGAGAA
GCCAAAAGACAATTCACAATCTCCCCAAAAAGGCAACAACACCATATTAGCTCT
GCCCAAATCTCCCTGGAAAAAACACTCGCCCATATACCAAAAATACCACAACCA
CCCCAAGAAAAAAACTGGGCAAAACAACACCCAAGAGACAAATAACAATGGAT
CCTCTCAATGAATCCACTGTTAATGTCTATCTTCCTGACTCATATCTTAAAGGAGT
GATTTCCTTTAGTGAGACTAATGCAATTGGTTCATGTCTCTTAAAAAGACCTTACC
TAAAAAATGACAACACTGCAAAAGTTGCCATAGAGAATCCTGTTATCGAGCATG
TTAGACTCAAAAATGCAGTCAATTCTAAGATGAAAATATCAGATTACAAGATAG
TAGAGCCAGTAAACATGCAACATGAAATTATGAAGAATGTACACAGTTGTGAGC
TCACATTATTAAAACAGTTTTTAACAAGGAGTAAAAATATTAGCACTCTCAAATT
AAATATGATATGTGATTGGCTGCAGTTAAAGTCTACATCAGATGATACCTCAATC
CTAAGTTTTATAGATGTAGAATTTATACCTAGCTGGGTAAGCAATTGGTTTAGTA
ATTGGTACAATCTCAACAAGTTGATTCTGGAATTCAGGAAAGAAGAAGTAATAA
GAACTGGTTCAATCTTGTGTAGGTCATTGGGTAAATTAGTTTTTGTTGTATCATCA
TATGGATGTATAGTCAAGAGCAACAAAAGCAAAAGAGTGAGCTTCTTCACATAC
AATCAACTGTTAACATGGAAAGATGTGATGTTAAGTAGATTCAATGCAAATTTTT
GTATATGGGTAAGCAACAGTCTGAATGAAAATCAAGAAGGGCTAGGGTTGAGAA
GTAATCTGCAAGGCATATTAACTAATAAGCTATATGAAACTGTAGATTATATGCT
TAGTTTATGTTGCAATGAAGGTTTCTCACTTGTGAAAGAGTTCGAAGGCTTTATT
ATGAGTGAAATTCTTAGGATTACTGAACATGCTCAATTCAGTACTAGATTTAGAA
ATACTTTATTAAATGGATTAACTGATCAATTAACAAAATTAAAAAATAAAAACA
GACTCAGAGTTCATGGTACCGTGTTAGAAAATAATGATTATCCAATGTACGAAGT
TGTACTTAAGTTATTAGGAGATACTTTGAGATGTATTAAATTATTAATCAATAAA
AACTTAGAGAATGCTGCTGAATTATACTATATATTTAGAATATTCGGTCACCCAA
TGGTAGATGAAAGAGATGCAATGGATGCTGTCAAATTAAACAATGAAATCACAA
AAATCCTTAGGTGGGAGAGCTTGACAGAACTAAGAGGGGCATTCATATTAAGGA
TTATCAAAGGATTTGTAGACAACAACAAAAGATGGCCCAAAATTAAAAACTTAA
AAGTGCTTAGTAAGAGATGGACTATGTACTTCAAAGCAAAAAGTTACCCCAGTC
AACTTGAATTAAGCGAACAAGATTTTTTAGAGCTTGCTGCAATACAGTTTGAACA
AGAGTTTTCTGTCCCTGAAAAAACCAACCTTGAGATGGTATTAAATGATAAAGCT
ATATCACCTCCTAAAAGATTAATATGGTCTGTGTATCCAAAAAATTACTTACCTG
AGAAAATAAAAAATCGATATCTAGAAGAGACTTTCAATGCAAGTGATAGTCTCA
AAACAAGAAGAGTACTAGAGTACTATTTGAAAGATAATAAATTCGACCAAAAAG
AACTTAAAAGTTATGTTGTTAAACAAGAATATTTAAATGATAAGGATCATATTGT
CTCGCTAACTGGAAAAGAAAGAGAATTAAGTGTAGGTAGAATGTTTGCTATGCA
ACCAGGAAAACAGCGACAAATACAAATATTGGCTGAAAAATTGTTAGCTGATAA
TATTGTACCTTTTTTCCCAGAAACCTTAACAAAGTATGGTGATCTAGATCTTCAGA
GAATAATGGAAATCAAATCGGAACTTTCTTCTATTAAAACTAGAAGAAATGATA
GTTATAATAATTACATTGCAAGAGCATCCATAGTAACAGATTTAAGTAAGTTCAA
CCAAGCCTTTAGGTATGAAACTACAGCGATCTGTGCGGATGTAGCAGATGAACT
ACATGGAACACAAAGCCTATTCTGTTGGTTACATCTTATCGTCCCTATGACAACA
ATGATATGTGCCTATAGACATGCACCACCAGAAACAAAAGGTGAATATGATATA
GATAAGATAGAAGAGCAAAGTGGTTTATATAGATATCATATGGGTGGTATTGAA
GGATGGTGTCAAAAACTCTGGACAATGGAAGCTATATCTCTATTAGATGTTGTAT
CTGTAAAAACACGATGTCAAATGACATCTTTATTAAACGGTGACAACCAATCAAT
AGATGTAAGTAAACCAGTTAAGTTATCTGAGGGTTTAGATGAAGTGAAAGCAGA
TTATAGCTTGGCTGTAAAAATGTTAAAAGAAATAAGAGATGCATACAGAAATAT
AGGCCATAAACTTAAAGAAGGGGAAACATATATATCAAGAGATCTTCAGTTTAT
AAGTAAGGTGATTCAATCTGAAGGAGTAATGCATCCTACCCCTATAAAAAAGAT
CTTAAGAGTGGGACCATGGATAAACACAATATTAGATGACATTAAAACCAGTGC
AGAGTCAATAGGGAGTCTATGTCAGGAATTAGAATTTAGGGGGGAAAGCATAAT
AGTTAGTCTGATATTAAGGAATTTTTGGCTGTATAATTTATACATGCATGAATCA
AAGCAACACCCCCTAGCAGGGAAGCAGTTATTCAAACAACTAAATAAAACATTA
ACATCAGTGCAGAGATTTTTTGAAATAAAAAAGGAAAATGAAGTAGTAGATCTA
TGGATGAACATACCAATGCAGTTTGGAGGAGGAGATCCAGTAGTCTTCTATAGAT
CTTTCTATAGAAGGACCCCTGATTTTTTAACTGAAGCAATCAGTCATGTGGATAT
TCTGTTAAGAATATCAGCCAACATAAGAAATGAAGCGAAAATAAGTTTCTTCAA
AGCCTTACTGTCAATAGAAAAAAATGAACGTGCTACACTGACAACACTAATGAG
AGATCCTCAAGCTGTTGGCTCAGAGCGACAAGCAAAAGTAACAAGTGATATCAA
TAGAACAGCAGTTACCAGCATCTTAAGTCTTTCTCCAAATCAACTTTTCAGCGAT
AGTGCTATACACTACAGTAGAAATGAAGAAGAGGTCGGAATCATTGCTGACAAC
ATAACACCTGTTTATCCTCATGGACTGAGAGTTTTGTATGAATCATTACCTTTTCA
TAAAGCTGAAAAAGTTGTGAATATGATATCAGGAACGAAATCCATAACCAACTT
ATTACAGAGAACATCTGCTATTAATGGTGAAGATATTGACAGAGCTGTATCCATG
ATGCTGGAGAACCTAGGATTATTATCTAGAATATTGTCAGTAGTTGTTGATAGTA
TAGAAATTCCAACCAAATCTAATGGTAGGCTGATATGTTGTCAGATATCTAGAAC
CCTAAGGGAGACATCATGGAATAATATGGAAATAGTTGGAGTAACATCCCCTAG
CATCACTACATGCATGGATGTCATATATGCAACTAGCTCTCATTTGAAAGGGATA
ATCATTGAAAAGTTCAGCACTGACAGAACTACAAGAGGTCAAAGAGGTCCAAAG
AGCCCTTGGGTAGGGTCGAGCACTCAAGAGAAAAAATTAGTTCCTGTTTATAACA
GACAAATTCTTTCAAAACAACAAAGAGAACAGCTAGAAGCAATTGGAAAAATGA
GATGGGTATATAAAGGGACACCAGGTTTAAGACGATTACTCAATAAGATTTGTCT
TGGAAGTTTAGGCATTAGTTACAAATGTGTAAAACCTTTATTACCTAGGTTTATG
AGTGTAAATTTCCTACACAGGTTATCTGTCAGTAGTAGACCTATGGAATTCCCAG
CATCAGTTCCAGCTTATAGAACAACAAATTACCATTTTGACACTAGTCCTATTAA
TCAAGCACTAAGTGAGAGATTTGGGAATGAAGATATTAATTTGGTCTTCCAAAAT
GCAATCAGCTGTGGAATTAGCATAATGAGTGTAGTAGAACAATTAACTGGTAGG
AGTCCAAAACAGTTAGTTTTAATACCTCAATTAGAAGAAATAGACATTATGCCAC
CACCAGTGTTTCAAGGGAAATTCAATTATAAGCTAGTAGATAAGATAACTTCTGA
TCAACATATCTTCAGTCCAGACAAAATAGATATGTTAACACTGGGGAAAATGCTC
ATGCCCACTATAAAAGGTCAGAAAACAGATCAGTTCCTGAACAAGAGAGAGAAT
TATTTCCATGGGAATAATCTTATTGAGTCTTTGTCAGCAGCGTTAGCATGTCATTG
GTGTGGGATATTAACAGAGCAATGTATAGAAAATAATATTTTCAAGAAAGACTG
GGGTGACGGGTTCATATCGGATCATGCTTTTATGGACTTCAAAATATTCCTATGT
GTCTTTAAAACTAAACTTTTATGTAGTTGGGGGTCCCAAGGGAAAAACATTAAAG
ATGAAGATATAGTAGATGAATCAATAGATAAACTGTTAAGGATTGATAATACTTT
TTGGAGAATGTTCAGCAAGGTTATGTTTGAATCAAAGGTTAAGAAAAGGATAAT
GTTATATGATGTAAAATTTCTATCATTAGTAGGTTATATAGGGTTTAAGAATTGG
TTTATAGAACAGTTGAGATCAGCTGAGTTGCATGAGGTACCTTGGATTGTCAATG
CCGAAGGTGATCTGGTTGAGATCAAGTCAATTAAAATCTATTTGCAACTGATAGA
GCAAAGTTTATTTTTAAGAATAACTGTTTTGAACTATACAGATATGGCACATGCT
CTCACAAGATTAATCAGAAAGAAGTTGATGTGTGATAATGCACTATTAACTCCGA
TTCCATCCCCAATGGTTAATTTAACTCAAGTTATTGATCCTACAGAACAATTAGCT
TATTTCCCTAAGATAACATTTGAAAGGCTAAAAAATTATGACACTAGTTCAAATT
ATGCTAAAGGAAAGCTAACAAGGAATTACATGATACTGTTGCCATGGCAACATG
TTAATAGATATAACTTTGTCTTTAGTTCTACTGGATGTAAAGTTAGTCTAAAAAC
ATGCATTGGAAAACTTATGAAAGATCTAAACCCTAAAGTTCTGTACTTTATTGGA
GAAGGGGCAGGAAATTGGATGGCCAGAACAGCATGTGAATATCCTGACATCAAA
TTTGTATACAGAAGTTTAAAAGATGACCTTGATCATCATTATCCTTTGGAATACC
AGAGAGTTATAGGAGAATTAAGCAGGATAATAGATAGCGGTGAAGGGCTTTCAA
TGGAAACAACAGATGCAACTCAAAAAACTCATTGGGATTTGATACACAGAGTAA
GCAAAGATGCTTTATTAATAACTTTATGTGATGCAGAATTTAAGGACAGAGATGA
TTTTTTTAAGATGGTAATTCTATGGAGGAAACATGTATTATCATGCAGAATTTGC
ACTACTTATGGGACAGACCTCTATTTATTCGCAAAGTATCATGCTAAAGACTGCA
ATGTAAAATTACCTTTTTTTGTGAGATCAGTAGCCACCTTTATTATGCAAGGTAGT
AAACTGTCAGGCTCAGAATGCTACATACTCTTAACACTAGGCCACCACAACAATT
TACCCTGCCATGGAGAAATACAAAATTCTAAGATGAAAATAGCAGTGTGTAATG
ATTTTTATGCTGCAAAAAAACTTGACAATAAATCTATTGAAGCCAACTGTAAATC
ACTTTTATCAGGGCTAAGAATACCGATAAATAAGAAAGAATTAAATAGACAGAG
AAGGTTATTAACACTACAAAGCAACCATTCTTCTGTAGCAACAGTTGGAGGTAGC
AAGGTCATAGAGTCTAAATGGTTAACAAACAAGGCAAACACAATAATTGATTGG
TTAGAACATATTTTAAATTCTCCAAAAGGTGAATTAAATTATGATTTTTTTGAAGC
ATTAGAAAATACTTACCCTAATATGATTAAACTAATAGATAATCTAGGGAATGCA
GAGATAAAAAAACTGATCAAAGTAACTGGATATATGCTTGTAAGTAAAAAATGA
AAAATGATAAAAATGATAAAATAGGTGACAACTTCATACTATTCCAAAGTAATC
ATTTGATTATGCAATTATGTAATAGTTAATTAAAAACTAAAAATCAAAAGTTAGA
AACTAACAACTGTCATTAAGTTTATTAAAAATAAGAAATTATAATTGGATGTATA
CG.

In certain embodiments, the current disclosure provides polynucleotide variants having substantial identity to the sequences disclosed herein; those comprising at least 50%, 60%, 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

Certain embodiments relate to a nucleotide of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1950, 2000, 2050, 2100, 2150, 2200, 2250, 2300, 2350, 2400, 2450, 2500, 2550, 2600, 2650, 2700, 2750, 2800, 2850, 2900, 2950 contiguous nucleic acids (or any derivable range therein) having at least 10, 20, 30, 40, 50, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity (or any derivable range therein) to a nucleic acid of the disclosure or segment thereof, such as to SEQ ID NO:1-4 (RNA or DNA version), or a segment thereof or a complement of SEQ ID NO:1-4, or a complementing segment thereof. Throughout this disclosure, an RNA molecule may specifically be an mRNA molecule in some embodiments.

Certain embodiments relate to a nucleic acid of the disclosure, such as a nucleic acid (RNA or DNA) of SEQ ID NO:1-4 (or its complement or a complementing segment thereof), wherein the nucleic acid is modified to have at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) substitutions, such as substitutions of a guanine for a uracil, an adenine for a uracil, a cytosine for a uracil, a cytosine for a guanine, an adenine for a guanine, a uracil for a guanine, or combinations thereof.

In some embodiments, the disclosure relates to a nucleic acid comprising exactly or at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112,113, 114, 115, 116, 117, 118, 119, 120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 225, 250, 275, or 300 (or any derivable range therein) contiguous nucleic acids of SEQ ID NO:1-4 (or its complement or an RNA or DNA version thereof), wherein at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 (or any derivable range therein) of the uracils, guanines, or m6A modification sites are substituted.

In some embodiments, the disclosure relates to a nucleic acid comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106,107, 108, 109, 110, 111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 225, 250, 275, or 300 (or any derivable range therein) contiguous nucleic acids of SEQ ID NO:1-4 (RNA or DNA, as well as its complement or a complimenting segment thereof), wherein at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) of the m6A consensus sites are modified.

The disclosure also contemplates the use of polynucleotides which are complementary to all the above described polynucleotides.

Nucleic acids of the disclosure may be modified through recombinant DNA technology to include the variants described herein. The DNA is converted to RNA by an RNA polymerase, provided with the RSV nucleocapsid protein, the polymerase protein, phosphoprotein and M2-1 protein to assemble a functional capsid that can replicate and produce complete RSV for use as a modified virus, as further described herein.

A nucleic acid sequence can be “heterologous,” which means that it is in a context of a cell or amid a nucleic acid sequence in which it is not found in nature. Instead, the heterologous nucleic acid is foreign to the cell in which the nucleic acid is being introduced or to the nucleic acid in which is incorporated, which includes a sequence homologous to a sequence in the cell or nucleic acid but in a position within the host cell or nucleic acid where it is ordinarily not found.

### II. Host Cells

As used herein, the terms “cell”, “cell line”, and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for heterologous nucleic acids or viruses. A host cell may be “infected”, “transfected”, “transformed”, or “transduced,” which refers to a process by which exogenous nucleic acid, such as a recombinant protein-encoding sequence, is transferred or introduced into the host cell. A transformed cell includes the primary subject cell and its progeny. A transduced cell has received nucleic acid via a virus vector such as a lentivirus or adeno-associated virus vector. In the case of a lentivirus vector, the transduced gene is integrated into a chromosome of the cell.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### III. Immune Response and Assays

The current disclosure concerns evoking or inducing an immune response in a subject against RSV. In one embodiment, the immune response can protect a subject at risk of developing RSV disease.

### A. Immunoassays

Embodiments include the implementation of serological assays to evaluate whether and to what extent an immune response is induced or evoked by compositions of the disclosure. There are many types of immunoassays that can be implemented. Immunoassays encompassed by some embodiments include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### B. Diagnosis of RSV

In addition to the use of compositions of the disclosure comprising viral particles, proteins, polypeptides, and/or peptides, as well as antibodies binding these polypeptides, proteins, and/or peptides, to treat or prevent infection as described above, the current disclosure contemplates the use of these compositions in a variety of ways, including the detection of the presence of RSV to diagnose an infection in a patient. One method of detecting the presence of infections involves the steps of obtaining a sample suspected of being infected by RSV, such as a sample taken from an individual, for example, from one's nasal discharges, nasal swab/wash, blood, saliva, tissues, bone, muscle, cartilage, or skin. Following isolation of the sample, diagnostic assays utilizing the polypeptides, proteins, peptides, and/or antibodies of the present invention may be carried out to detect the presence of RSV, and such assay techniques for determining such presence in a sample are well known to those skilled in the art and include methods such as radioimmunoassay, western blot analysis and ELISA assays. In general, in accordance with the invention, a method of diagnosing an infection is contemplated wherein a sample suspected of being infected with RSV has added to it the polypeptide, protein, peptide, antibody, or monoclonal antibody in accordance with the present invention, and RSV infection is indicated by antibody binding to the polypeptides, proteins, and/or peptides, or polypeptides, proteins, and/or peptides binding to the antibodies in the sample.

Accordingly, antibodies in accordance with the invention may be used for the prevention of infection from RSV (i.e., passive immunization), for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. Specific examples of the generation of an antibody to a bacterial protein can be found in U.S. Patent Application Pub. No. 20030153022, which is incorporated herein by reference in its entirety.

Any of the above described polypeptides, proteins, peptides, and/or antibodies may be labeled directly with a detectable label for identification and quantification of staphylococcal bacteria. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

### C. Protective Immunity

In some embodiments of the disclosure, the compositions, particularly those comprising attenuated RSV particles, confer protective immunity to a subject with respect to the disease associated with an RSV infection. Protective immunity refers to a body's ability to mount a specific immune response that protects the subject from developing a particular disease or condition that involves the agent against which there is an immune response. An immunogenically effective amount is capable of conferring protective immunity to the subject. In some embodiments, a patient population may have fewer symptoms, less severe symptoms, less risk of morbidity, reduced duration of symptoms (for example, by days or weeks), and/or reduced complications from an RSV infection. In some embodiments, the reduction may be expressed as a reduction of 10, 20, 30, 40, 50, 60, 70, 80, 90% or more as compared to a patient population not receiving the attenuated RSV.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the invention in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

Methods may be employed with respect to individuals who have tested positive for previous exposure to RSV or who are deemed to be at high risk for complications related to RSV, including infants of less than one year old, children younger than 12, premature infants of less than one year old, immunocompromised individuals, and the elderly, such as those over 70 years old.

In particular, the disclosure encompasses a method of protection from RSV infection and disease. The immunogenic compositions and vaccines of the disclosure are also advantageous to use to inoculate health care workers.

### D. Formulations and Modes of Administration

The present disclosure includes methods for preventing or inhibiting RSV infections. As such, the disclosure contemplates vaccines for use in active immunization embodiments. Immunogenic compositions, proposed to be suitable for use as a vaccine, may be prepared from host cells.

The vaccines of the disclosure may be prepared for delivery as nose drop or aerosols to be delivered intra-nasally. They may also be delivered as injectables either as liquid solutions or suspensions: solid forms suitable for solution in or suspension in liquid prior to injection may also be prepared. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be administered intra-nasally by drops or aerosol or parenterally, by injection, for example, either subcutaneously or intramuscularly. Oral formulations include such normally employed excipients as, for example, pharmaceutical grades of mannitol, lactose, starch, magnesium stearate, sodium saccharine, cellulose, magnesium carbonate and the like.

The compositions may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens, as described in U.S. Pat. Nos. 3,791,932; 4,174,384 and 3,949,064.

In some embodiments, pharmaceutical compositions are administered to a subject. Different aspects of the current disclosure involve administering an effective amount of a composition to a subject.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present disclosure will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### E. In Vitro, Ex Vivo, or In Vivo Administration

As used herein, the term in vitro administration refers to manipulations performed on cells removed from or outside of a subject, including, but not limited to cells in culture. The term ex vivo administration refers to cells which have been manipulated in vitro, and are subsequently administered to a subject. The term in vivo administration includes all manipulations performed within a subject.

In certain aspects of the present disclosure, the compositions may be administered either in vitro, ex vivo, or in vivo. In certain in vitro embodiments, autologous B-lymphocyte cell lines are incubated with a virus of the instant invention for 24 to 48 hours and/or any other composition described herein for two hours. The transduced cells can then be used for in vitro analysis, or alternatively for ex vivo administration. U.S. Pat. Nos. 4,690,915 and 5,199,942, both incorporated herein by reference, disclose methods for ex vivo manipulation of blood mononuclear cells and bone marrow cells for use in therapeutic applications.

### IV. Kits

The disclosure additionally provides kits for detecting modified adenosines in RNA, kits for detecting RSV, and kits comprising compositions of the disclosure. Each kit may also include additional components that are useful for amplifying the nucleic acid, or sequencing the nucleic acid, or other applications of the present invention as described herein. The kit may optionally provide additional components that are useful in the procedure. These optional components include buffers, capture reagents, developing reagents, labels, reacting surfaces, means for detection, control samples, instructions, and interpretive information. The kit may also include reagents for RNA isolation and/or purification.

### V. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments, are provided as an example, and are not intended as limitations on the scope of the invention. Changes therein and other uses which are encompassed within the spirit of the invention as defined by the scope of the claims will occur to those skilled in the art.

### Example 1: Viral N⁶-methyladenosine Upregulates Replication and Pathogenesis of Human Respiratory Syncytial Virus

### A. Results

The RSV genome and antigenome/mRNAs are m⁶A methylated in both HeLa and A549 cells. RSV has a non-segmented negative-sense (NNS) RNA genome of 15,222 nucleotides (RSV A2 strain). As is typical for NNS RNA viruses, replication of the viral genomic RNA (vgRNA) produces an exact, positive-sense full-length complementary RNA (cRNA) antigenome [39]. Both the genome and antigenome are encapsidated by the nucleocapsid (N) protein and both nucleocapsids can be packaged into virions, as for many NNS RNA viruses [47]. Using linear sucrose density gradient ultracentrifugation, the inventors first purified RSV virions from supernatants of RSV-infected human cervical carcinoma cells (HeLa), as this cell line is commonly used in RSV research. To investigate whether RSV RNA contains m⁶A, RNA was extracted from virions grown in HeLa cells and the presence of m⁶A in viral RNA from virions was quantified by liquid chromatography-tandem mass spectrometry (LC-MS/MS). It was found that approximately 0.7% of the A bases were m⁶A methylated in RSV viral RNAs, a somewhat higher level than the host mRNAs (0.1-0.4%).

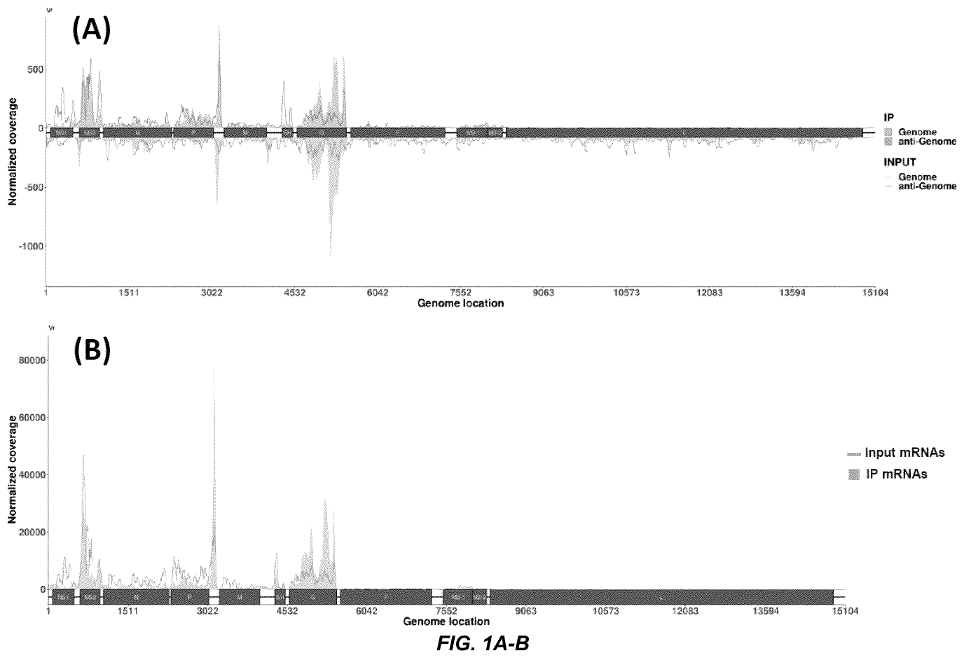

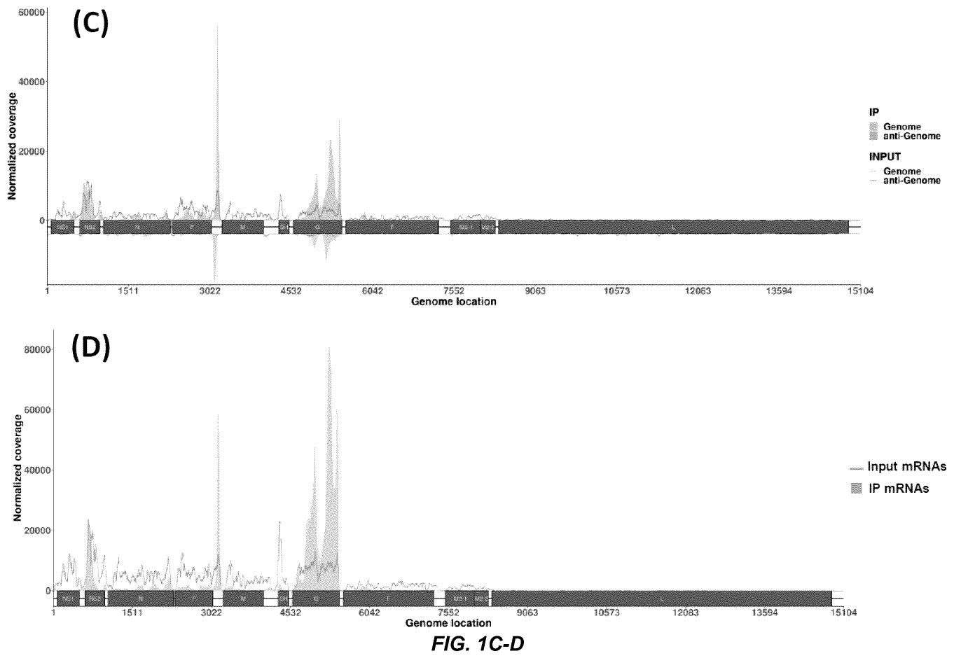

To locate the m⁶A sites on RSV viral RNA, the inventors sonicated virion RNA and subjected it to m⁶A-specific antibody immunoprecipitation followed by high throughput sequencing (m⁶A-seq), then mapped all the reads onto either the genome or antigenome sequence. Several m⁶A peaks were identified on both strands of the viral RNA (FIG. 1A). The RSV antigenomic RNA contained major m⁶A peaks in the regions complementary to the N, P, G, and F genes and in two regulatory elements, the gene end (ge) sequence of N and the intergenic (ig) sequence between the P and M genes (FIG. 1A and Supplementary Table 1). In the genomic RNA, eleven m⁶A peaks were detected in the NS2, N, P, M, G, and L genes and four regulatory elements including the gene start (gs) of NS2, ig between P and M, ge of M, ig between M and G, ge of G, and ig between G and L (FIG. 1A and Supplementary Table 1). The G gene regions from both genome and antigenome have the strongest m⁶A enrichment with peak size of 822 nt and 672 nt, respectively. Despite the sequences being reverse complementary, the locations of m⁶A peaks identified from both strands largely overlap (FIG. 1A). Together, these results confirm that both RSV genome and antigenome RNAs contain m⁶A.

The inventors also mapped m⁶A peaks in mRNAs purified from RSV-infected cells. To do this, total RNA was isolated from rgRSV-infected HeLa cells, enriched for mRNA by binding to oligo dT, and subjected to m⁶A-seq. As RSV mRNAs also contain poly(A) and are subsequently detected from poly(dT)-enriched m⁶A-seq, the inventors identified 16 m⁶A peaks from RSV mRNAs (FIG. 1B, Supplementary Table 2) which were largely overlapped with those of antigenome (FIG. 1A, Supplementary Table 1), indicating the cellular m⁶A machinery methylates both types of positive-strand viral RNAs similarly. Interestingly, the G gene transcript has the strongest m⁶A enrichment with 846 bp peak size.

The inventors next performed m⁶A-seq of rgRSV grown in tumor-derived human lung alveolar epithelial cells (A549 cells), a relevant cell line for RSV infection. RNAs of virions and RSV-infected A549 cells were prepared as described above and were m⁶A sequenced. Similar to HeLa cells, it was found that genome, antigenome, and mRNAs were m⁶A methylated in A549 cells (FIGS. 1C and D). For virion RNAs, a total of 9 and 15 m⁶A peaks were identified in the genome and antigenome respectively (FIG. 1C and Supplementary Table 3). Similar to virions grown in HeLa cells, the location of m⁶A peaks identified from genome and antigenome largely overlap.

G gene regions from both genome and antigenome have the strongest m^{6A} enrichment with 696 and 846 bp peak size, respectively. For the RNAs extracted from virus-infected cells, a total of 18 m⁶A peaks were identified in mRNAs (FIG. 1D and Supplementary Table 4). Again, the G gene transcript has the strongest m⁶A enrichment with 1046 bp peak size.

The inventors next analyzed the overlapping regions based on m⁶A-seq data from HeLa and A549 cells (Supplementary Table 5 and 6). For virion RNA, six and four overlapping regions were identified in the genome (gs of NS2, NS2, N, P, ig between P and M, and G) and antigenome (N, M, G, and F), respectively (Supplementary Table 5). For RNAs purified from RSV-infected cells, 11 overlapping m⁶A peaks were also found in mRNAs, respectively (Supplementary Table 6). Although there are some differences, the majority of m⁶A peaks are highly conserved between the two cell lines suggesting that RSV utilizes the host m⁶A machinery to methylate these specific sites.

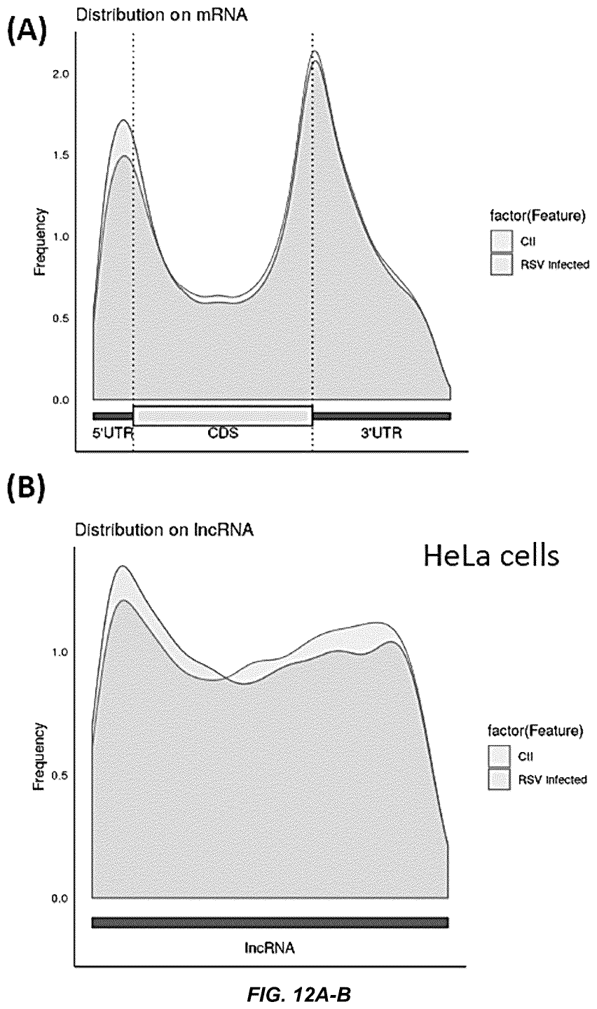

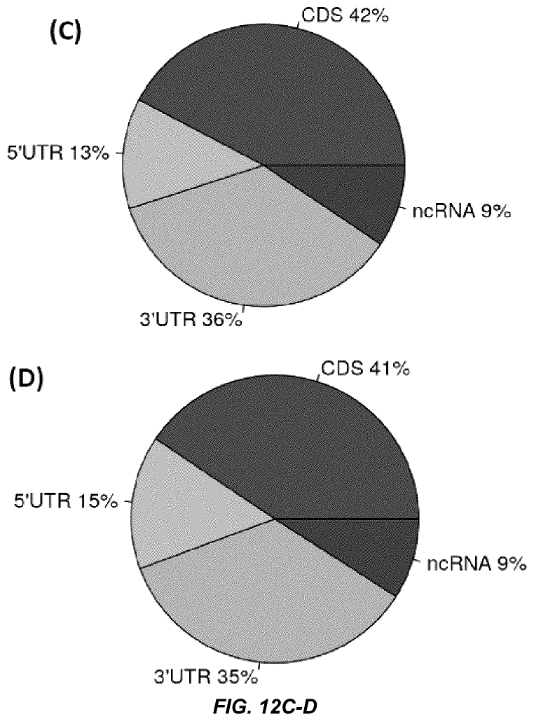

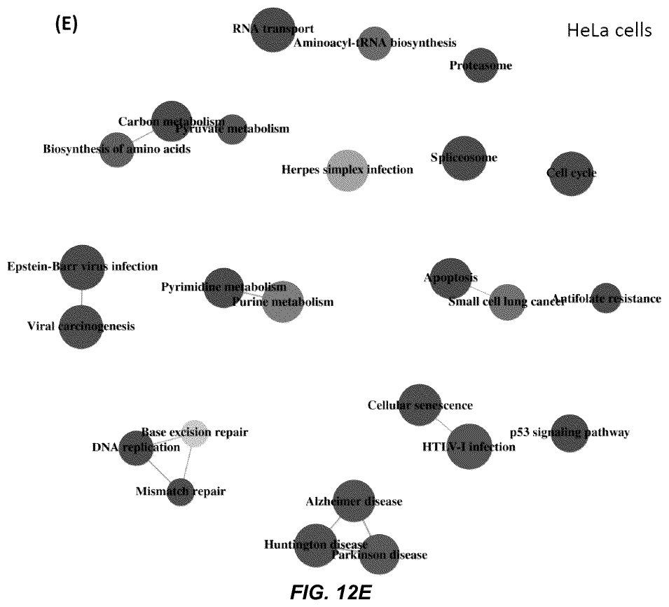

RSV infection alters the m⁶A distribution and gene expression of host cell transcripts. The inventors next determined the effects of RSV infection on the abundance and distribution of m⁶A on cellular transcripts. To do this, total RNA was isolated from mock-infected and rgRSV-infected HeLa cells, enriched for mRNA by binding to oligo dT, and subjected to m⁶A-seq. Metagene analysis showed that RSV-infected and mock-infected HeLa cells have m⁶A peaks enriched near the start and stop codons of open reading frames (FIG. 12A), which is consistent with the known distribution of m⁶A sites on transcripts [9-11]. Unlike the distribution of m⁶A peaks on mRNA, the peaks are mostly uniformly distributed on lncRNA with slightly more enrichment at its 5′ end (FIG. 12B). The distribution of m⁶A peaks in each annotation also recapitulate m⁶A site distribution [9-11] with the majority of peaks residing in the CDS and 3′ UTR regions (FIGS. 12C and D). Differential peak analysis using the count based QNB test [48] identified 2256 differentially methylated peaks (Supplementary Table 7). Analysis of RNA-seq data from the host cell (HeLa) revealed over 9,000 differentially expressed genes at an adjusted P value cutoff of 0.05 (Supplementary Table 8). These data suggest RSV infection significantly altered both the epitranscriptome and the transcriptome of the host cells. Pathway enrichment analysis shows differentially expressed genes are enriched in pathways including cell cycle, metabolism, RNA synthesis and transport, and response to viral infection (FIG. 12E).

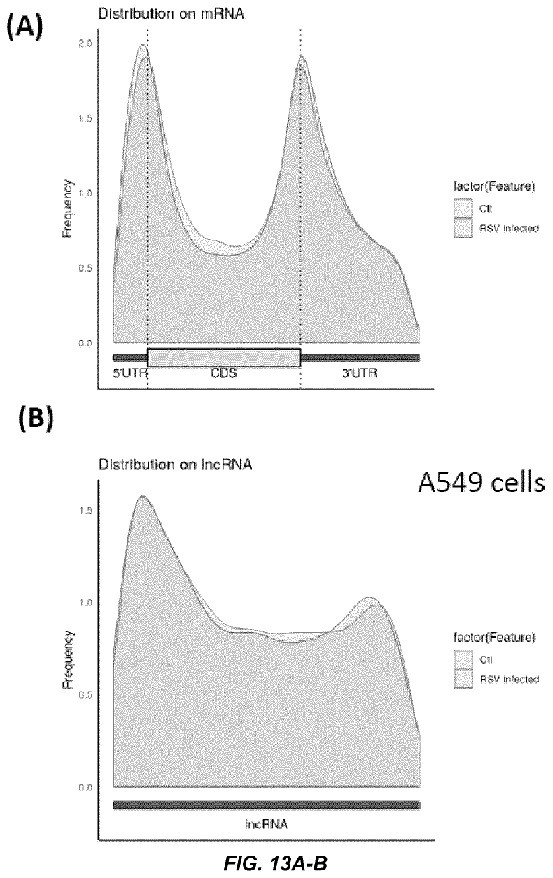

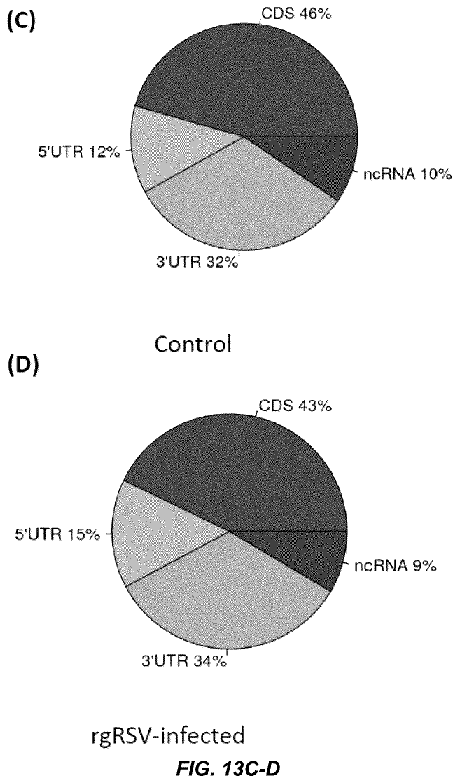

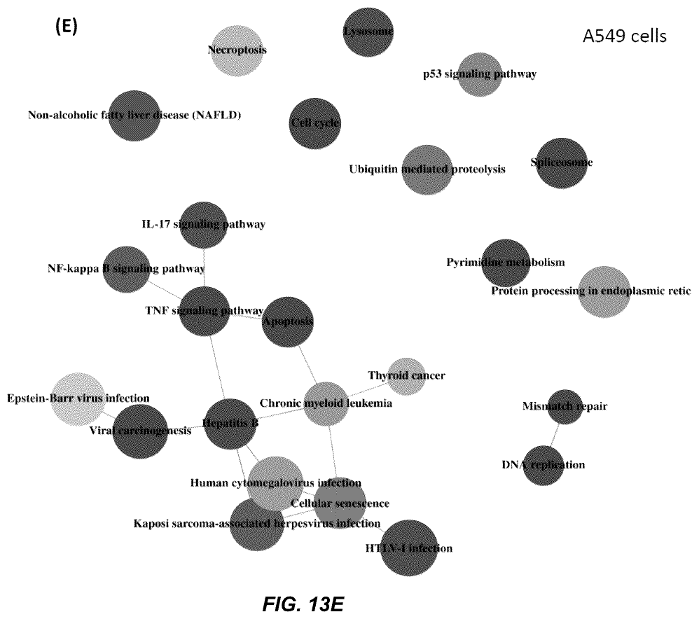

As expected, the distribution of m⁶A sites was highly conserved between cell lines (FIG. 13A-D). RSV infection altered the expression of over 7,000 host cell genes in A549 cells (Supplementary Table 9) involved in a series of signal pathways (FIG. 13E) despite very few m⁶A peaks were found to be differentially methylated (Supplementary Table 10). Therefore, RSV infection may have widespread effects on host gene expression partially attributed to alteration of the deposition of m⁶A. Collectively, RSV infection seems to be sensed and actively responded to by the host m⁶A regulators to trigger broad changes in post-transcriptional methylation profiles of host mRNAs in some cell lines, which in turn may impact the expression of key genes in multiple functional pathways relevant to the host response to viral infection.

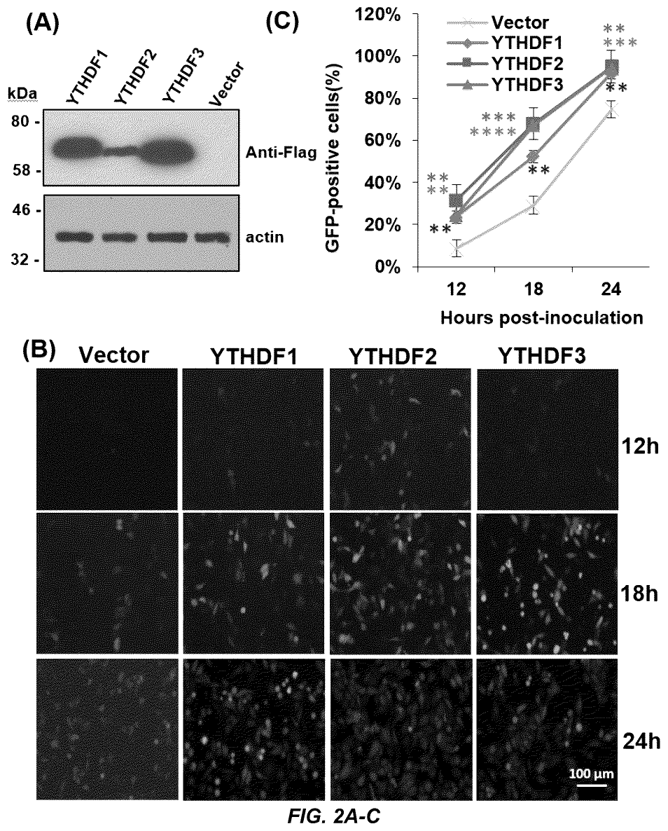

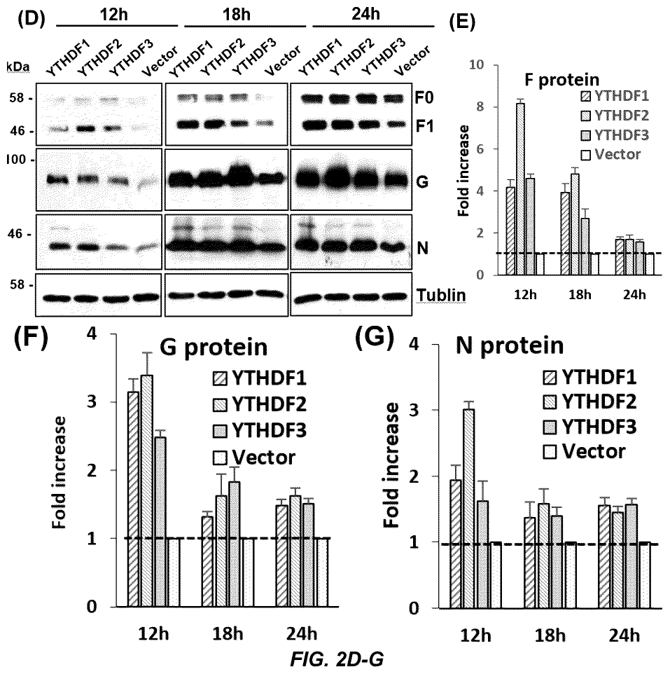

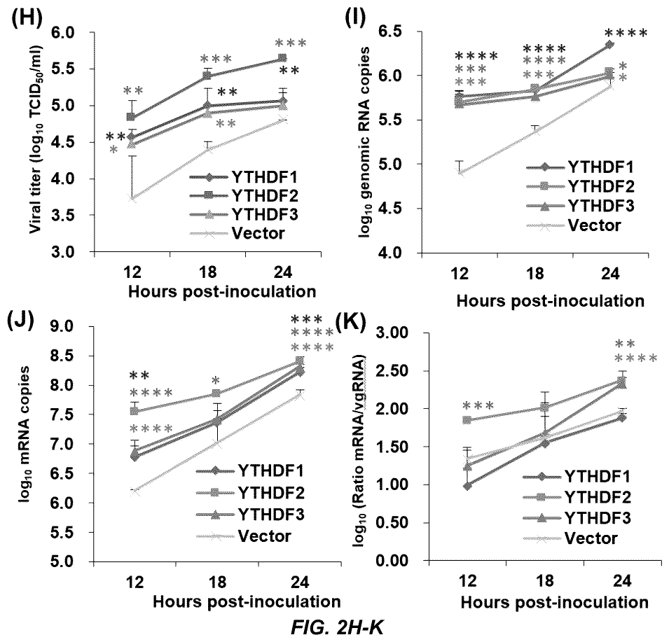

m⁶A reader proteins positively regulate RSV replication, gene expression, and virus production. To begin to explore the role of m⁶A modification in RSV replication and gene expression, the inventors first took advantage of HeLa cells that stably overexpress m⁶A “reader” proteins, YTHDF1, YTHDF2, and YTHDF3, which are m⁶A-binding proteins (FIG. 2A). Briefly, HeLa cells were infected with rgRSV at an MOI of 0.1, and viral protein expression, RNA synthesis, and virus production were monitored at 12, 18, and 24 h post-infection. As shown in FIG. 2B, stronger GFP expression (more green cells, brighter cells) was observed in HeLa cells overexpressing YTHDF1-3 compared to the vector control. Quantification by flow cytometry showed that significantly more GFP-positive cells were detected in HeLa cells overexpressing m⁶A reader proteins at 12, 18, and 24 h post-infection than in the vector control (P<0.05) (FIG. 2C).

The inventors next measured the expression of RSV F and G proteins (the two major surface glycoproteins) and N protein (the major component of the nucleocapsid complex). As shown in FIG. 2D, more F, G, and N proteins were detected in all three YTHDF-overexpressing HeLa cell lines at all three time points. Quantitative analysis of the protein bands showed a dramatic increase in viral protein expression during the first 12 h. Later time points were not as large possibly because the virus had already reached maximal levels of replication (FIGS. 2E, F, and G). Next, the inventors measured the release of infectious virus particles in a single step growth curve. The RSV titer was significantly increased in all three YTHDF-overexpressing cell lines at 12, 18, and 24 h post-inoculation (FIG. 2H) (P<0.05 or 0.01). Overexpression of YTHDF2 had the most dramatic impact on virus production, increasing RSV titer by 1-2 logs at all three time points compared to the vector control HeLa cells (P<0.05 or 0.01) (FIG. 2H).

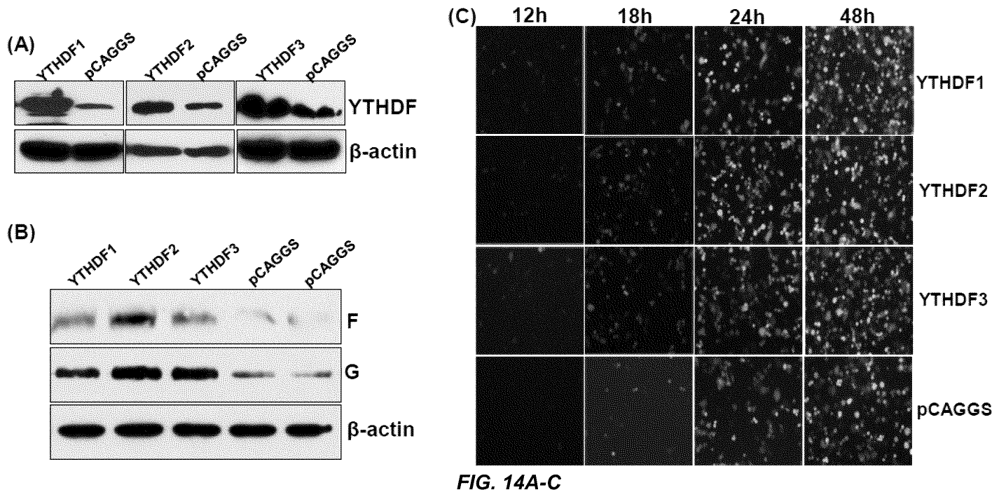

The upregulating role of m⁶A reader proteins on RSV replication was also confirmed in HeLa cells transiently expressing YTHDF1-3 (FIG. 14A). HeLa cells were transfected with plasmids expressing YTHDF1, YTHDF2, or YTHDF3, and were infected by rgRSV. Compared to HeLa cells stably overexpressing YTHDF1-3 (FIG. 2D), transient overexpression of YTHDF1-3 led to a more robust enhancement of F and G protein synthesis (FIG. 14B) and GFP expression (FIG. 14C).

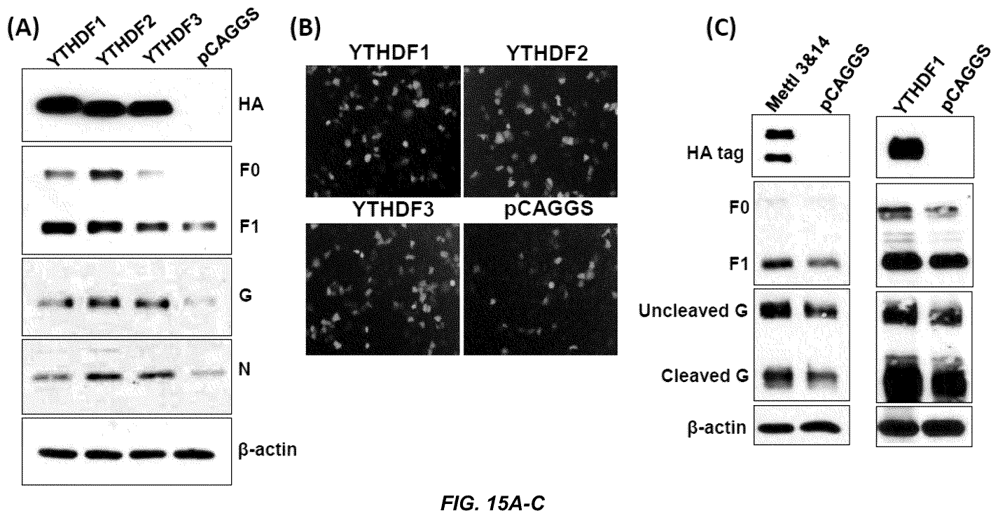

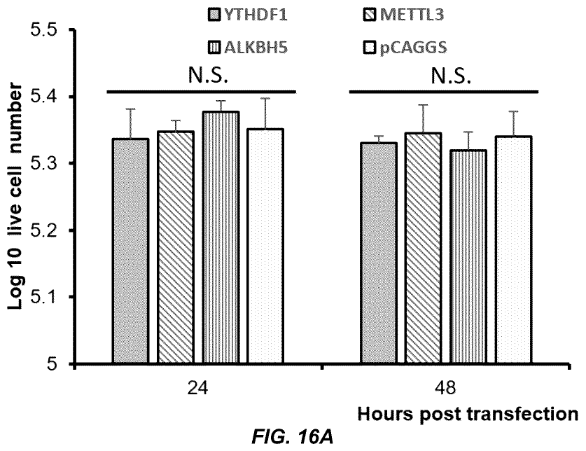

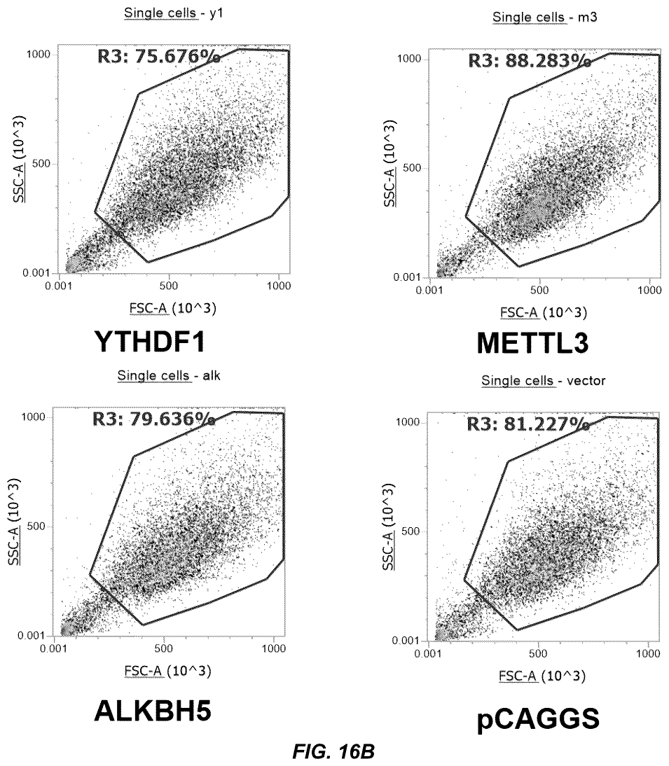

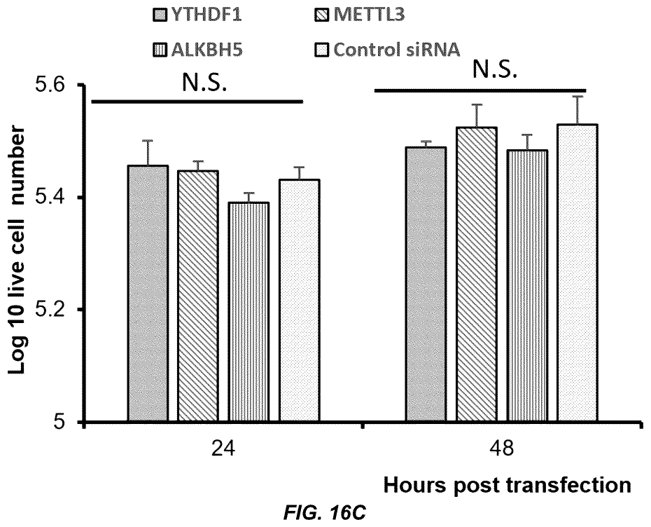

Currently, whether m⁶A machinery plays pro- or anti-viral function is controversial for some viruses (eg. HIV) [30, 31, 49]. In the case of KSHV, m⁶A machinery has a pro- or anti-viral effect depending on the cell line [34-36]. Thus, the inventors further analyzed viral replication and gene expression in A549 cells, a physiologically relevant cell line for RSV. Similar to the observations in HeLa cells, enhanced F, G, and N protein synthesis (FIG. 15A) and GFP expression (FIG. 15B) was detected when YTHDF1-3 proteins were transiently overexpressed in A549 cells. The inventors also tested RSV replication in Vero cells, the WHO-approved cell line for production of RSV live attenuated vaccine candidates. Similarly, m⁶A reader proteins (YTHDF1-3) enhanced RSV protein synthesis in Vero cells (FIG. 15C). Thus, a pro-viral function for m⁶A was observed in all three cell lines. It should be noted that overexpression of YTHDF1-3 proteins in all three cell lines did not significantly affect the growth or survival of the cells (FIGS. 16A and B)

One of the unique features of RSV and other viruses of the Mononegavirales order is that the genome RNA is completely encapsidated by the N protein and this complex serves as the template for two distinct RNA syntheses: genomic/anti-genomic RNA replication and mRNA transcription [50]. Both processes are carried out by a single RNA dependent RNA polymerase (RdRp) complex [50]. Thus, the RSV genomic RNA (the replication product) and mRNAs (the transcription product) were measured by real-time RT-PCR. Overexpression of YTHDF1-3 significantly increased both RSV genomic RNA (FIG. 21) and mRNA synthesis (FIG. 2J) in virus-infected cells. Overexpression of YTHDF1 and 3 did not alter the balance between the synthesis of genomic RNA and mRNA whereas overexpression of YTHDF2 led to a more dramatic increase in replication than transcription (FIG. 2K). It appears that overexpression of YTHDF1-3 enhanced the ability of the RSV RdRp to both replicate and transcribe.

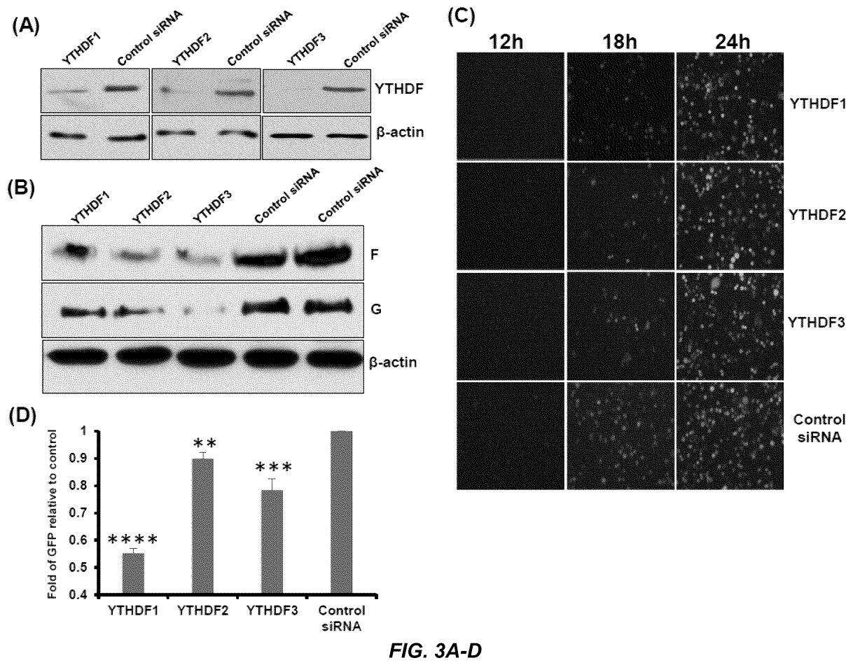

As a complementary approach, the inventors also tested RSV replication and gene expression in HeLa cells when m⁶A reader proteins were knocked down by siRNA. The inventors first examined cell survival when they were transfected with control siRNA or YTHDF1-3 siRNA. Counting live cells by flow cytometry showed that siRNA targeting YTHDF1-3 did not significantly alter cell survival (FIGS. 16C and D). Knockdown of individual, endogenous YTHDF1-3 proteins (FIG. 3A) did significantly reduced viral F and G protein synthesis (FIG. 3B) and GFP expression (FIGS. 3C and D) relative to the control siRNA transfected cells. Collectively, these results demonstrate that m⁶A binding proteins promote RSV genome replication, mRNA transcription, and as a result, viral protein expression, and progeny virus production.

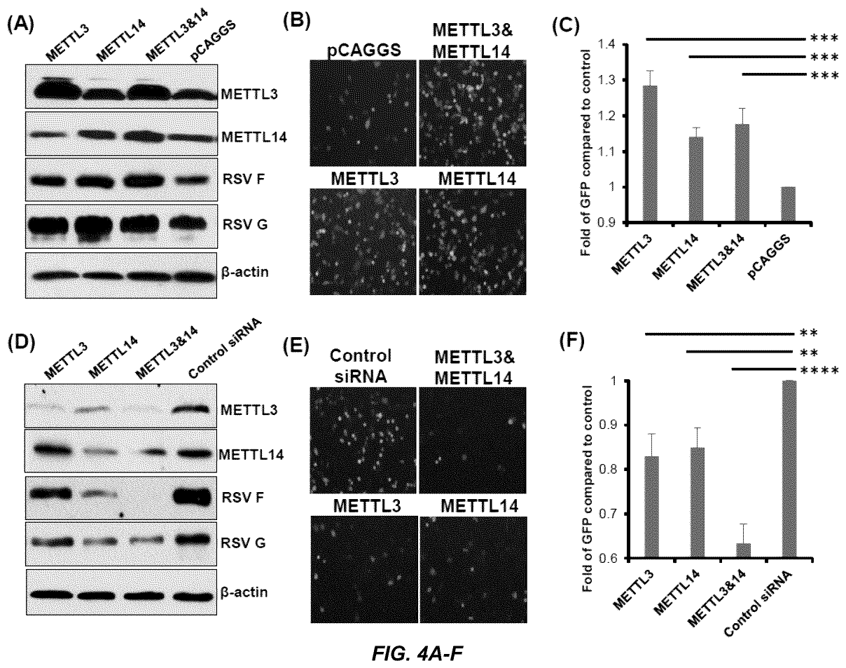

m⁶A writer proteins positively regulate RSV replication and gene expression. The internal m⁶A addition is catalyzed by host methyltransferases termed m⁶A writer proteins [17]. Next the role of the m⁶A writer proteins in RSV replication and protein expression was examined. To do this, HeLa cells were transfected with plasmids encoding the m⁶A writer proteins, METTL3 or METTL14, or both, followed by rgRSV infection. More F and G protein synthesis (FIG. 4A) and GFP expression (FIGS. 4B and C) were observed when METTL3 and METTL14 were overexpressed in HeLa cells. In contrast, less F and G proteins were synthesized (FIG. 4D) and less GFP was expressed (FIGS. 4E and F) when endogenous METTL3, METTL14, or both, were knocked down in HeLa cells using siRNA. These results suggest that modification of RSV RNA by m⁶A writers facilitates RSV replication and gene expression.

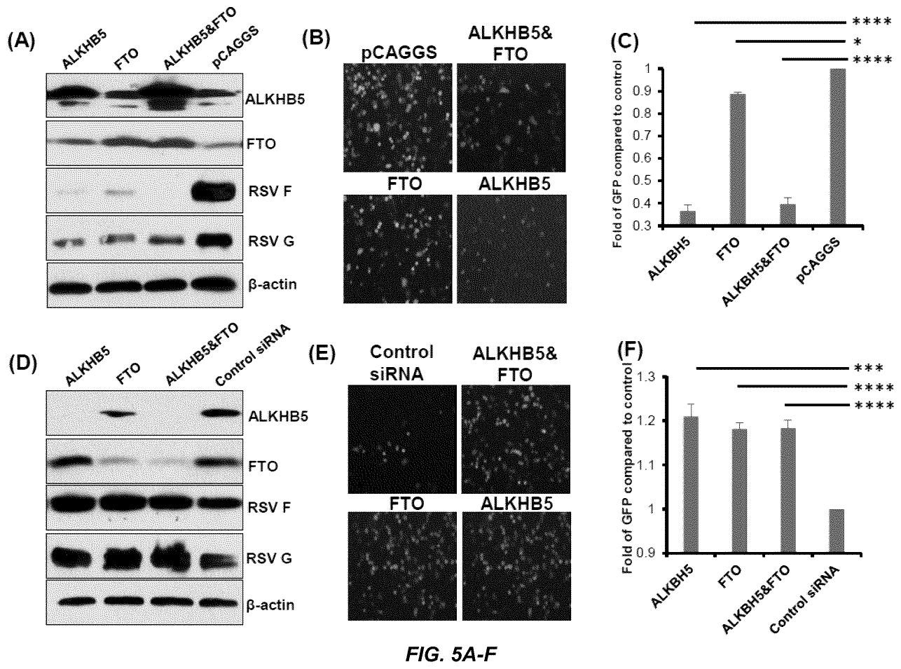

m⁶A eraser proteins downregulated RSV replication and gene expression. Internal m⁶A modifications are reversible and can be removed by m⁶A eraser proteins [7, 8]. Thus, the effects of overexpression of eraser proteins by transfection of HeLa cells with plasmids encoding m⁶A eraser proteins AlkBH5 or FTO, or both was examined (FIG. 5). Overexpression of eraser proteins dramatically reduced RSV F and G protein expression by 80- and 20-fold, respectively (FIG. 5A), and GFP expression by 20-50 times (FIGS. 5B and C). Next, AlkBH5 or FTO, or both, was knocked down in HeLa cells, followed by rgRSV infection. Knockdown of AlkBH5 and FTO enhanced the expression of F protein by 3-fold, G protein by 5-fold (FIG. 5D), and GFP expression by 10-fold (FIGS. 5E and F) compared to the cells transfected with control siRNA. Therefore, over-expression of m⁶A eraser proteins negatively regulated RSV replication and gene expression.

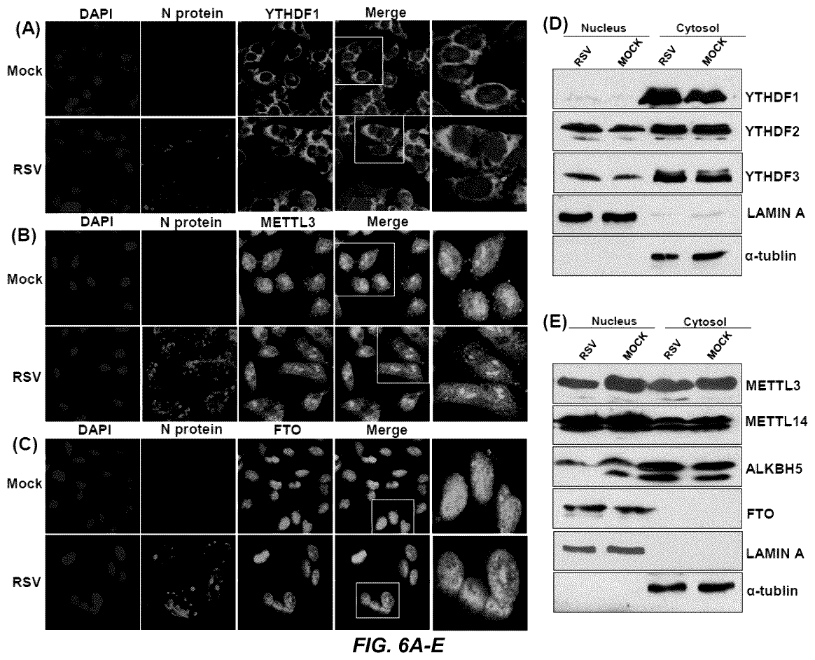

RSV infection does not alter the translocation of m⁶A-related proteins. The fact that the RNAs of RSV, a cytoplasmic replicating virus, are m⁶A modified suggests that m⁶A-related proteins are present in the cytoplasm and raises the possibility that they may shuttle from the nucleus into the cytoplasm in response to virus infection. To directly visualize the locations of the m⁶A reader, writer, and eraser proteins, mock and rgRSV infected HeLa cells were stained with antibodies specific to each m⁶A-related protein and analyzed by confocal microscopy. As shown in FIG. 6A and FIG. 17, m⁶A reader proteins (YTHDF1-3) were distributed in the cytoplasm in both mock and RSV-infected cells. In contrast, the majority of m⁶A writer proteins (METTL3 and METTL14) and eraser protein (AlkBH5) were distributed in the nucleus although a small fraction of these proteins was also found in the cytoplasm (FIG. 6B and FIGS. 18 and 19). Another eraser protein, FTO, was exclusively located in the nucleus (FIG. 6C). To quantify m⁶A-related proteins, nuclear and cytoplasmic fractions were isolated and analyzed by Western blot. Equal amounts of m⁶A related proteins were detected in the cytoplasmic and nuclear fractions (FIGS. 6D and E). Therefore, RSV infection does not significantly alter the distribution pattern of m⁶A-related proteins in HeLa cells. These results also suggest that the presence of a small fraction of cytoplasmic m⁶A writer proteins in the cytoplasm is sufficient for installing m⁶A on RSV RNAs.

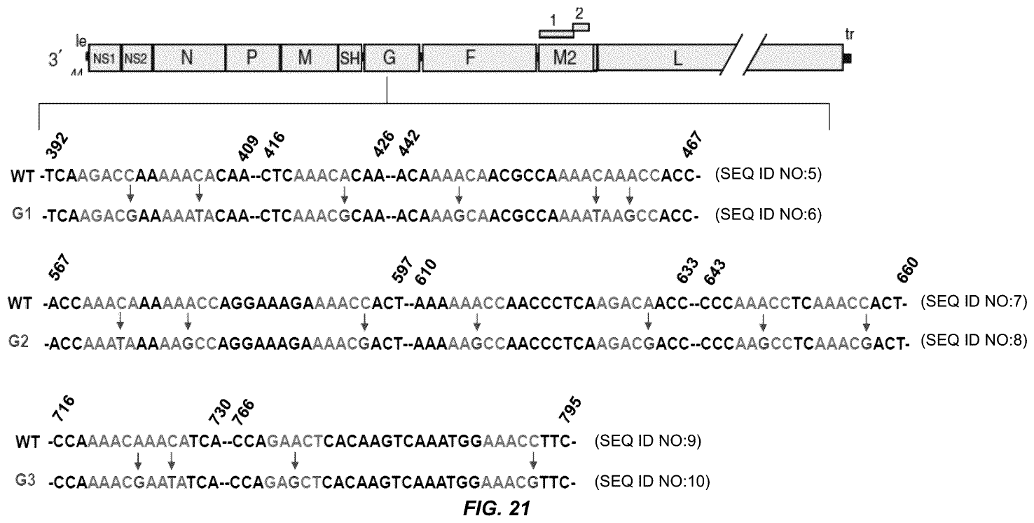

m⁶A reader proteins bind to both RSV genomic RNA and mRNA. Since the biological function of m⁶A is mediated by m⁶A binding proteins, it was next determined whether YTHDF2 can directly bind to RSV RNAs in virus-infected cells. Briefly, HeLa cells were infected with rgRSV, cell lysates were harvested and a specific antibody against YTHDF2 was used to precipitate YTHDF2, and any bound RSV genomic RNA and N mRNA were detected by real-time RT-PCR. As expected, YTHDF2 was detected by YTHDF2-specific antibody (FIG. 20A). Both RSV genomic RNA and N mRNA were efficiently precipitated as complexes, with YTHDF2 (FIG. 20B). This result was further confirmed by pulling down HA-tagged YTHDF2 from total cell lysates of HeLa cells overexpressing YTHDF2 with HA antibody (FIG. 20C). Similarly, significant amounts of RSV genomic RNA and N mRNA bound to YTHDF2 (FIG. 20D).

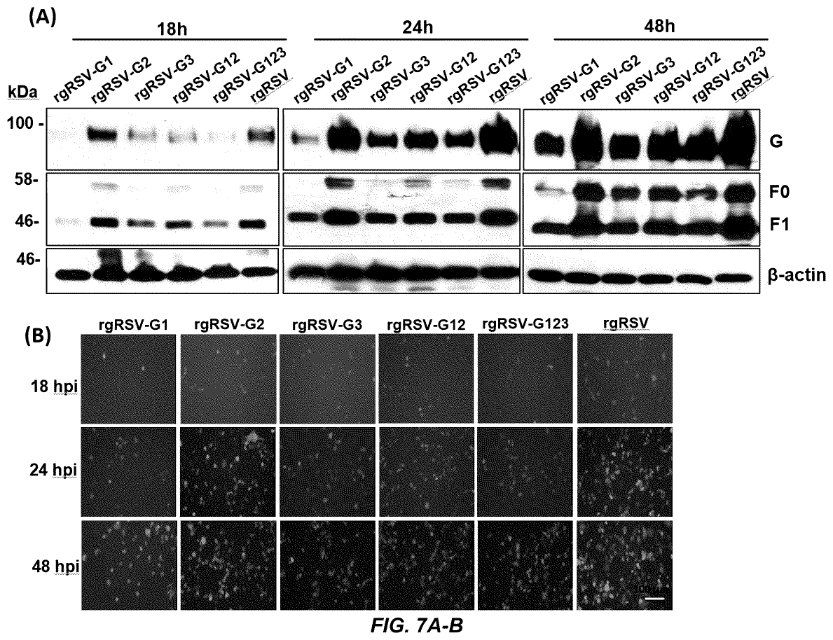

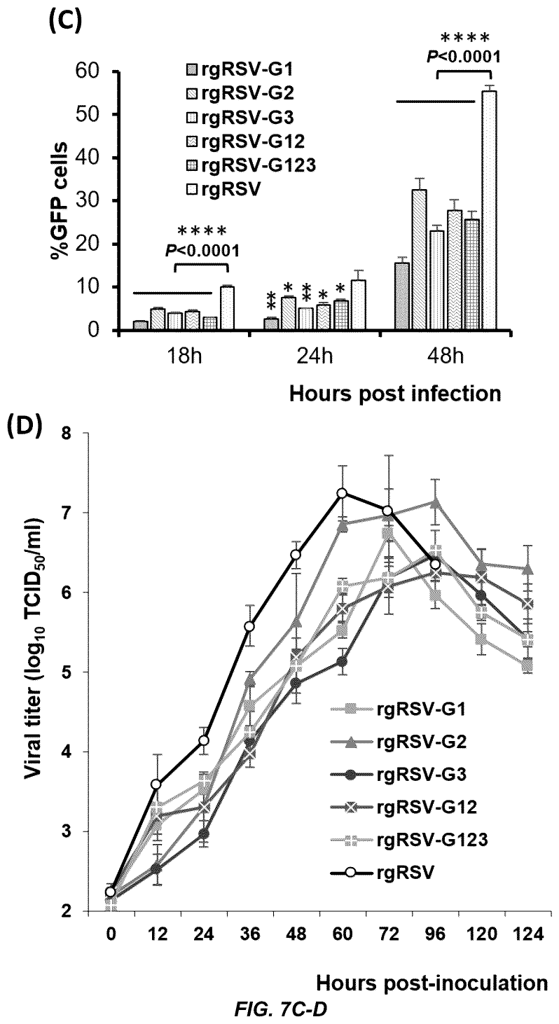

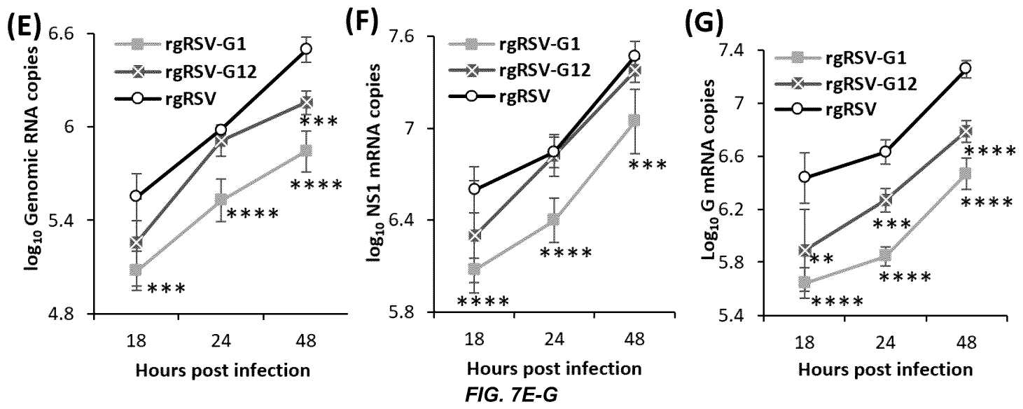

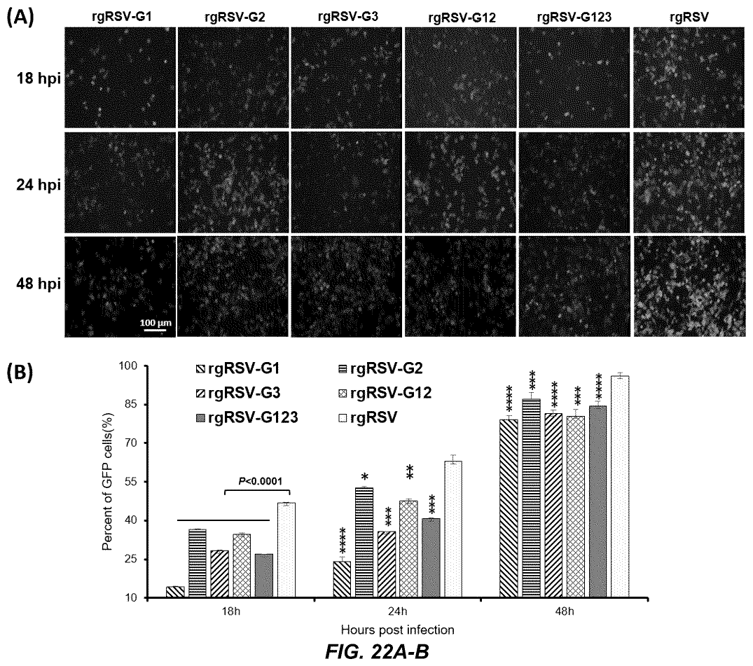

Abrogation of m⁶A sites in G mRNA results in attenuation of RSV in cell culture. Based on m⁶A-seq of mRNA, G mRNA has the most abundant m⁶A enrichment among RSV mRNAs in both HeLa and A549 cells (FIG. 1). In addition, the most abundant m⁶A enrichments in genome and antigenome are also located in the region covering the G gene. Thus, the inventors decided to mutate the m⁶A sites in the G gene which was found to be conserved in the m⁶A-seq from both HeLa and A549 cells. Those m⁶A peaks in the G gene are clustered in three regions, 392-467 nt, 567-660 nt, and 716-795 nt. Since it is known that m⁶A modified sites in RNA contain the conserved Pu [G>A]m⁶AC[A/C/U] motif (Pu represents purine) [1], the inventors searched for this motif in these three regions in G mRNA and identified 6, 7, and 4 potential m⁶A sites in regions 1, 2, and 3 respectively (FIG. 21). Subsequently, the inventors mutated the A or C within the consensus site to a T or G in these sites without changing the amino acid they encoded (FIG. 21). In addition, M-fold and Genscript software was used to predict that these mutations did not alter RNA secondary structure or codon usage. These mutations were predicted to remove the m⁶A sites in the G mRNA and in the G gene region of the antigenome. To determine the role of G mRNA m⁶A sites in the RSV life cycle, these G mutations were introduced into an infectious cDNA clone of the RSV A2 strain and recovered a panel of m⁶A-deficient rgRSV mutants. First, the potential m⁶A sites in regions 1, 2, and 3 were mutated individually to produce rgRSV-G1, G2, and G3, respectively. Second, mutations of m⁶A sites in regions 1 and 2 were combined to produce rgRSV-G12. Third, m⁶A sites in regions 1, 2, and 3 were combined to produce rgRSV-G123. Next, the RSV replication and gene expression in A549 cells infected by each rgRSV mutant was monitored. All m⁶A-deficient rgRSVs had various degrees of reduction in viral N, F, and G protein synthesis in A549 cells compared to the parental rgRSV (FIG. 7A). Significantly less GFP expression was observed in m⁶A-deficient rgRSVs compared to rgRSV at 48 h post-infection (FIGS. 7B and C, and FIG. 22). Single-step growth curves showed that m⁶A-deficient rgRSVs had delayed replication kinetics and had 0.5-1.5 log reductions in peak titer compared to rgRSV (FIG. 7D). Overall, m⁶A-deficient rgRSVs had variable degrees of attenuation in replication in immortalized cell culture. Mutants rgRSV-G1, G3, G12, and G123 had a moderate defect whereas rgRSV-G2 had a mild defect in replication. It was next determined the amount of RSV genome, NS1 mRNA, and G mRNA synthesized by rgRSV-G1 and G12 in A549 cells. Both rgRSV-G1 and G12 had defects in genome (FIG. 7E), NS1 (FIG. 7F), and G (FIG. 7G) mRNA synthesis compared to rgRSV, and rgRSV-G1 had more defects than rgRSV-G12. Next, the percentage of reduction for NS1 and G mRNA was calculated. It was found that NS1 mRNA had significantly less reduction than G mRNA, suggesting that removal of the m⁶A from the G mRNA may accelerate its decay.

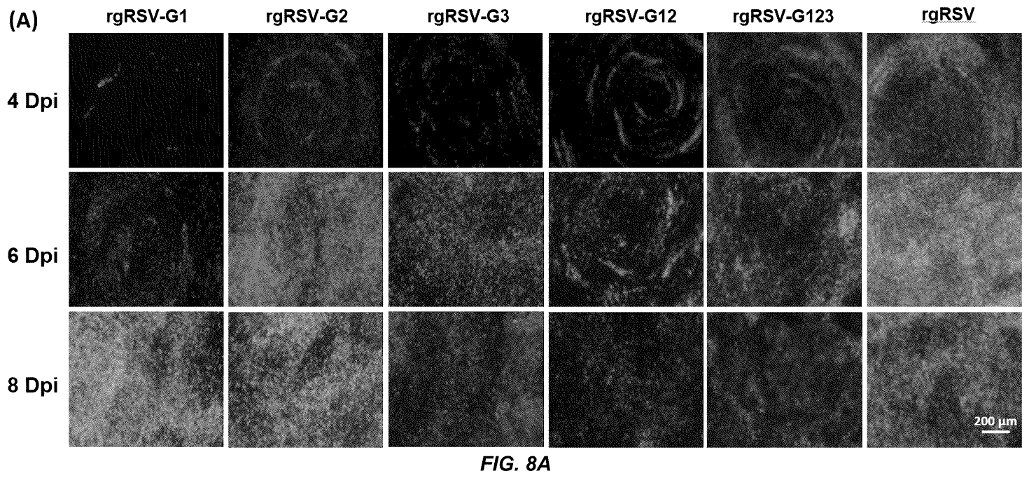

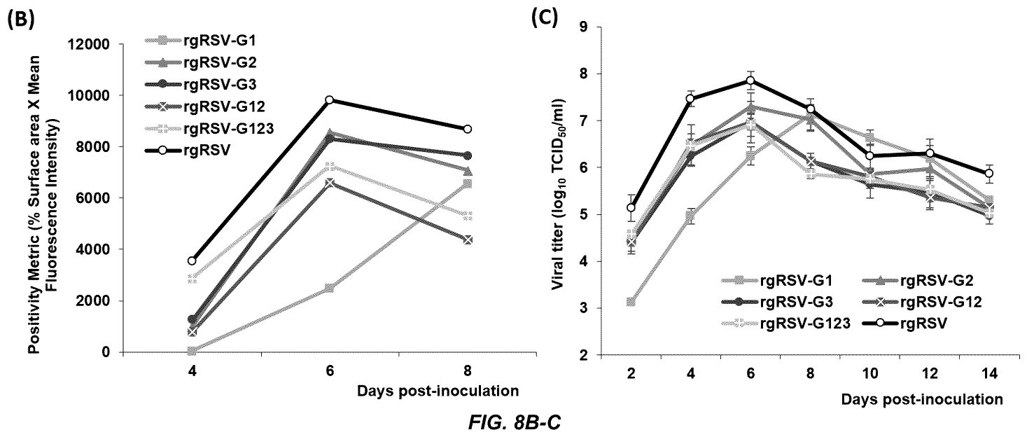

m⁶A-deficient rgRSVs are defective in replication and spread in primary well differentiated human airway epithelial (HAE) cultures. The inventors next tested the replication and spread of m⁶A-deficient rgRSVs in HAE cultures, a near in vivo model for lower airway infection. These cultures are pseudostratified and polarized, closely resembling the in vivo airway epithelium morphology and function, including mucus production and ciliary motion. RSV infects the ciliated cells on the apical surface where it attaches to its receptor, CX3CR1, on the cilia [51]. Infection spreads from an infected ciliated cell to neighboring ciliated cells, usually in a counter-clockwise fashion, due to the concerted ciliary beat, likely mimicking RSV infection and spread in human airways [51]. Briefly, HAE cultures were infected with 800 TCID₅₀ (equal to 400 pfu) of each recombinant virus, and viral release and spread was monitored. As in A549 cells, m⁶A-deficient rgRSVs had a delay in viral gene expression (GFP production) and spread (FIGS. 8A and B). At day 4 post-inoculation, m⁶A-deficient rgRSVs had fewer green cells compared to rgRSV. Although several rgRSV mutants gradually increased at days 6 and 8, the density of green cells remained less than for rgRSV. rgRSV-G1 was delayed in spreading but eventually spread to most susceptible cells at day 8. rgRSV-G2 had a delay at the early time point (day 4) but had wild type level of spreading at later times. Recombinant rgRSV-G12 was the most defective virus in HAE cultures, displaying a weak GFP signal during the entire experimental period. In addition, m⁶A-deficient rgRSVs had delays in virus release in HAE culture with 1-2 log defects in virus yield (FIG. 8C). These results demonstrate that m⁶A-deficient rgRSVs were defective in replication and spread in this near in vivo lung infection model.

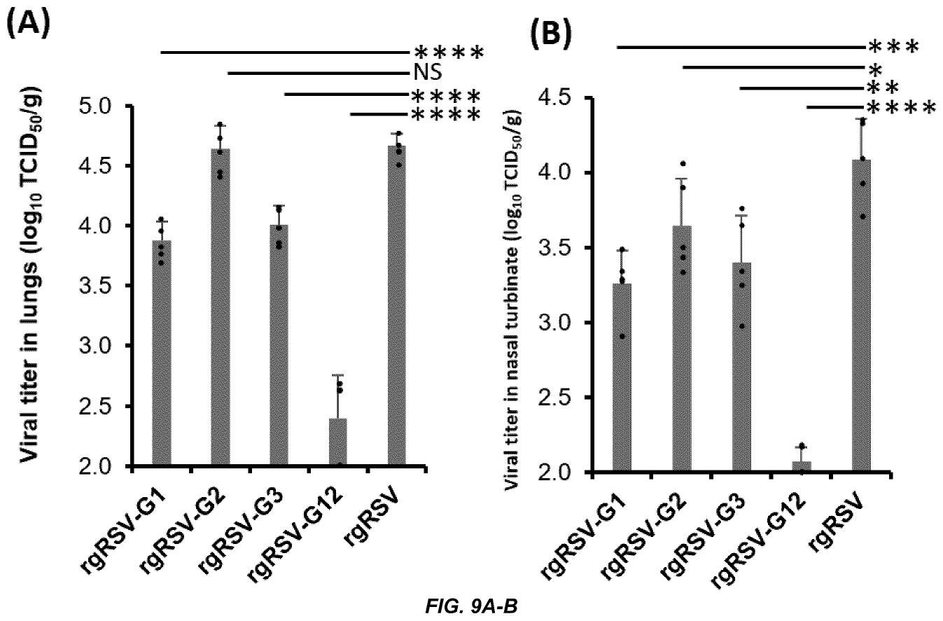

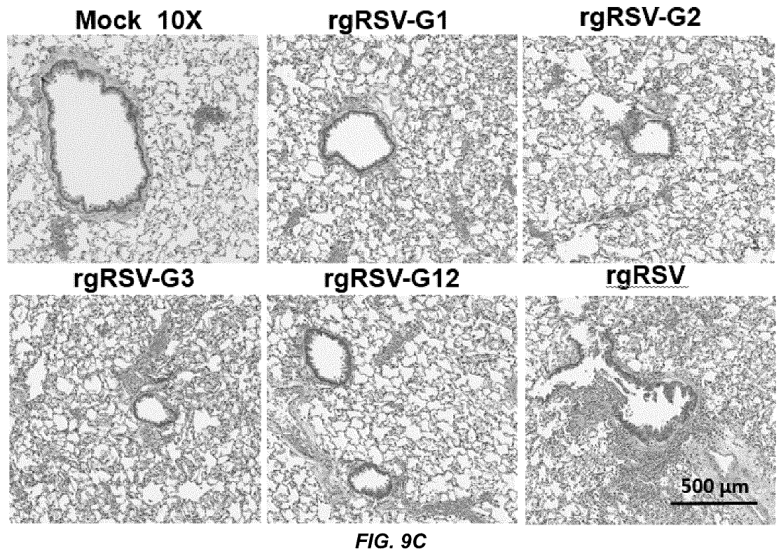

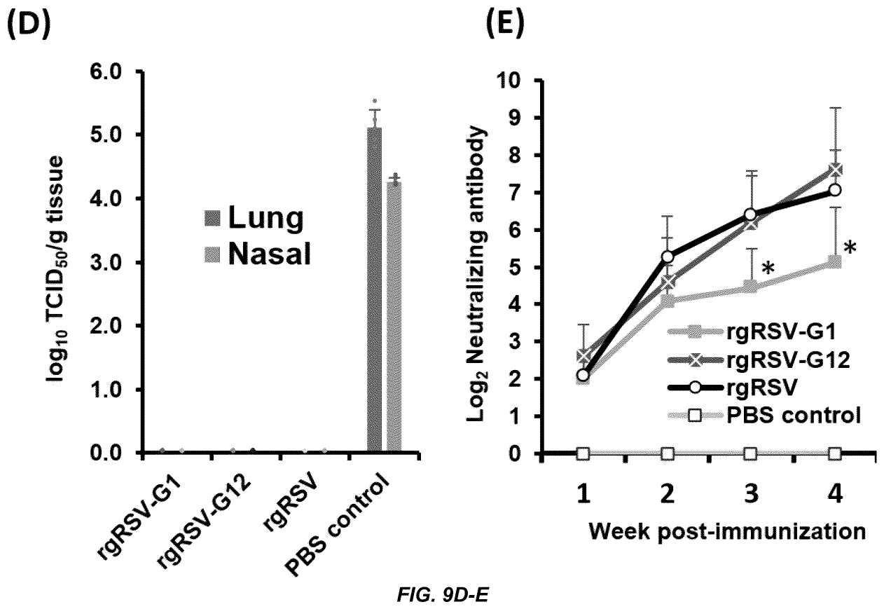

Abrogation of m⁶A sites in G mRNA results in rgRSVs that have defects in replication in cotton rats. The inventors tested replication and pathogenesis of four m⁶A-deficient rgRSV mutants, rgRSV-G1, G2, G3, and G12, in cotton rats, the best available small animal model for RSV infection. Based on replication and spread in immortalized cells and HAE culture, rgRSV-G2 exhibited mild attenuation whereas rgRSV-G1, G3, and G12 represent moderate to high attenuation. Briefly, cotton rats were inoculated intranasally with 2×10⁵ TCID50 of each rgRSV mutant. At day 4 postinoculation, cotton rats were sacrificed, and viral replication in the nasal turbinates and lungs, and pulmonary histology, were determined. Parental rgRSV replicated efficiently in the lungs (FIG. 9A) and nasal turbinates (FIG. 9B) with average viral titers of 4.70±0.10 log₁₀ TCID50/g and 4.10±0.10 log₁₀ TCID50/g, respectively. Mutant rgRSV-G1 with m⁶A mutations in peak 1 of the G mRNA had a 7-fold reduction in replication in nasal turbinate and lung titers, respectively (P<0.05). Mutant rgRSV-G2 with m⁶A mutations in peak 2 of the G mRNA had no significant reduction in replication in lung (P>0.05) but 3-fold reductions in nasal turbinate (P<0.05). Mutant rgRSV-G12, which is a combination of all mutations in m⁶A peaks 1 and 2 in G mRNA, had the most dramatic defect in replication, with reductions of 100- and 200-fold in viral titer in nasal turbinate and lung, respectively. It should be noted that 4 out of 5 cotton rats had below detection limit level of RSV replication in the nasal turbinate and 3 out of 5 cotton rats had below detection limit level of RSV replication in lungs, suggesting that rgRSV-G12 is highly attenuated in vivo. The rgRSV-G3 with m⁶A mutations in peak 3 of the G mRNA had 5-fold reductions in replication in nasal turbinate and lung (P<0.05). Histologic examination showed that rgRSV caused moderate pulmonary histopathological changes, including interstitial pneumonia and peribronchial lymphoplasmocytic infiltrates (FIG. 9C). In contrast, m⁶A-deficient rgRSV mutants only had mild and less pulmonary histopathological changes compared to rgRSV (FIG. 9C). These results showed that m⁶A-deficient rgRSV mutants had significant reductions in viral replication in both the upper and lower respiratory tracts in cotton rats and were less pathogenic compared to rgRSV. These results indicate that viral m⁶A upregulates viral replication and pathogenesis in vivo.

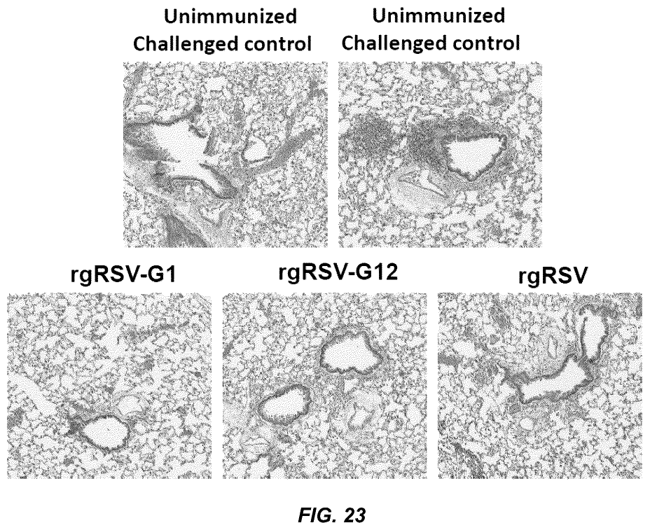

m⁶A-deficient rgRSVs provide complete protection against challenge with parental RSV. To determine whether defects in viral m⁶A methylation impair the immunogenicity of the virus, the protection efficacy of a partially attenuated (rgRSV-G1) and highly attenuated (rgRSV-G12) virus in cotton rats was evaluated. The parental rgRSV served as a control. An ideal vaccine candidate should retain similar or higher immunogenicity compared to the parental virus. To do this, six-week-old female SPF cotton rats were immunized intranasally with 2×10⁵ TCID₅₀ of each recombinant virus. Serum samples were collected weekly for detection of antibody response. At week 4 post-inoculation, animals were challenged with 2×10⁵ TCID₅₀ of parental rgRSV. At day 4 post-challenge, all the animals were sacrificed and nasal turbinate and lung tissue samples were collected for virus detection and pathological examination. Cotton rats immunized with parental rgRSV or m⁶A-deficient rgRSVs did not have any detectable infectious virus in either the nasal turbinate or lung tissue after challenge with rgRSV (FIG. 9D). In contrast, unvaccinated challenged controls had average titers of 5.12±0.28 and 4.27±0.07 log₁₀ PFU/g in the lung and nasal turbinate, respectively (FIG. 9D). These results demonstrate that immunization with the rgRSV-G1 and G12 provided complete protection from challenge with rgRSV. Lung histology showed that unvaccinated challenged controls had moderate histologic lesions (FIG. 23). However, the vaccinated challenged groups had only mild lesions in lungs. In addition, no enhanced lung damage was observed for m⁶A-deficient rgRSV immunized cotton rats upon reinfection with rgRSV (FIG. 23). The two m⁶A-deficient rgRSV triggered similar levels of neutralizing antibody compared to rgRSV (P>0.05) (FIG. 9E). Antibody was detectable at week 1 postimmunization, and the levels gradually increased during weeks 2 to 4. No RSV-specific antibody was detected in the unvaccinated control. These results demonstrate that m⁶A-deficient rgRSV retained high immunogenicity and provided complete protection against RSV infection in cotton rats.

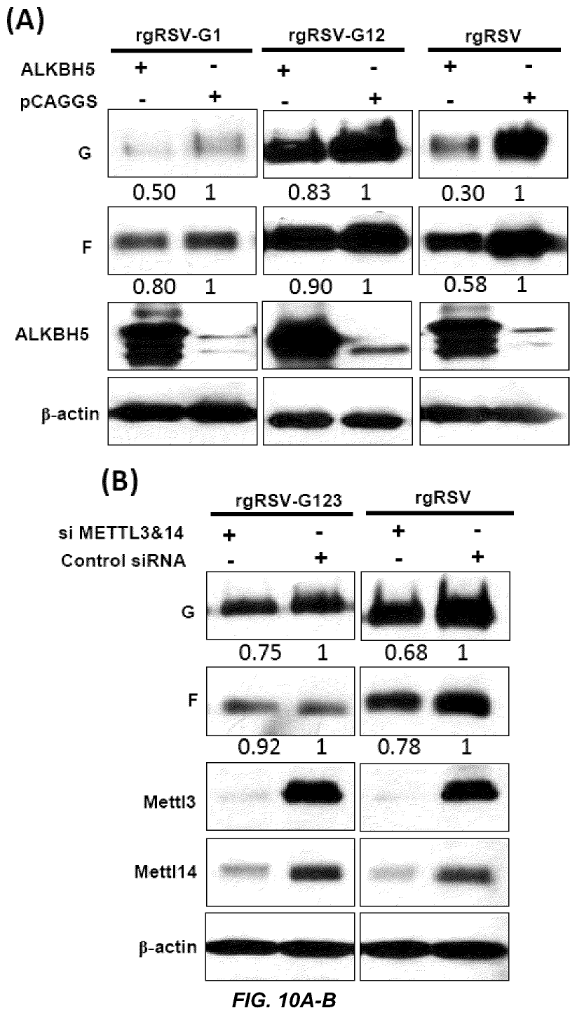

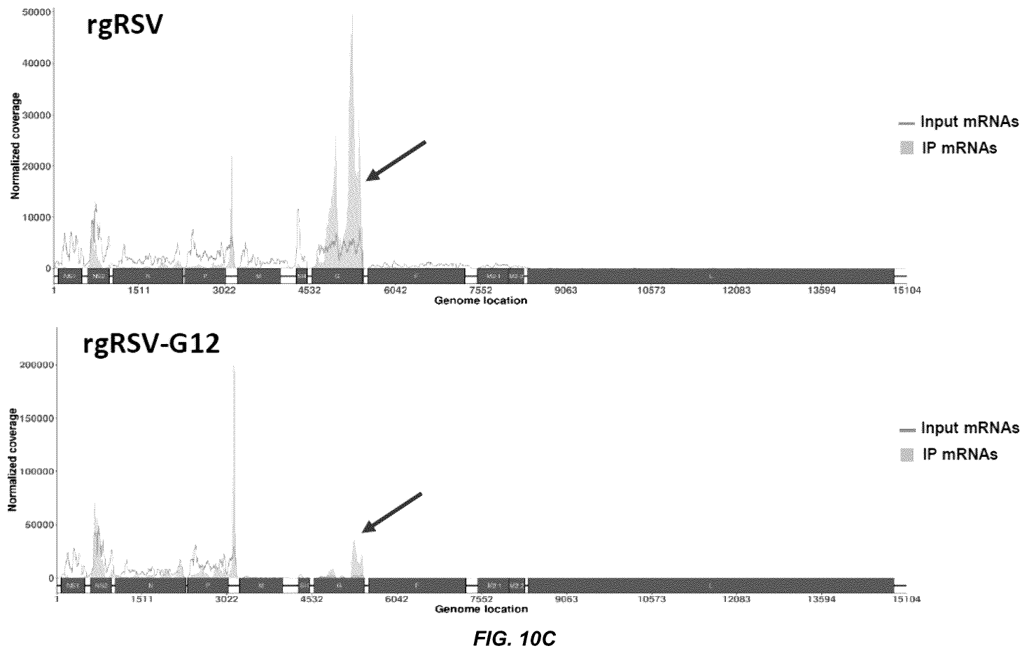

Replication and gene expression of m⁶A-deficient rgRSVs are less dependent on host m⁶A machinery. If the attenuated phenotype of m⁶A-deficient rgRSVs is indeed m⁶A-dependent, alteration of host m⁶A machinery would have no or less of an impact on replication and gene expression since major m⁶A sites have been removed from the G mRNA in these m⁶A-deficient viruses. To address this question, the inventors tested replication of rgRSV-G1 and G12 in A549 cells overexpressing AlkBH5 which is an m⁶A eraser protein. Consistent with previous results, overexpression of AlkBH5 led to 70% and 42% reduction in RSV G and F protein synthesis in rgRSV-infected cells compared to vector control cells (FIG. 10A). The inventors also observed a reduction in replication and protein expression of the m⁶A-deficient rgRSVs in AlkBH5 overexpressing cells, but the level of reduction was much less compared to the parental rgRSV. For example, only 17% and 10% reduction in RSV G and F protein synthesis was observed for rgRSV-G12, and 50% and 20% reduction in G and F protein was observed for rgRSV-G1, respectively. The inventors also tested the replication of rgRSV-G123 in m⁶A writer protein-depleted A549 cells. Knockdown of MELL3 and METTL14 led to 32 and 22% reduction in RSV G and F protein in rgRSV-infected A549 cells whereas only 25% and 8% reduction in G and F in rgRSV-G123-infected A549 cells (FIG. 10B). Thus, these results showed that replication and gene expression of m⁶A-deficient rgRSVs were less dependent on host m⁶A machinery, suggesting that the attenuated phenotype of these mutants is likely due to the deficiency in m⁶A methylation of the viral RNA.

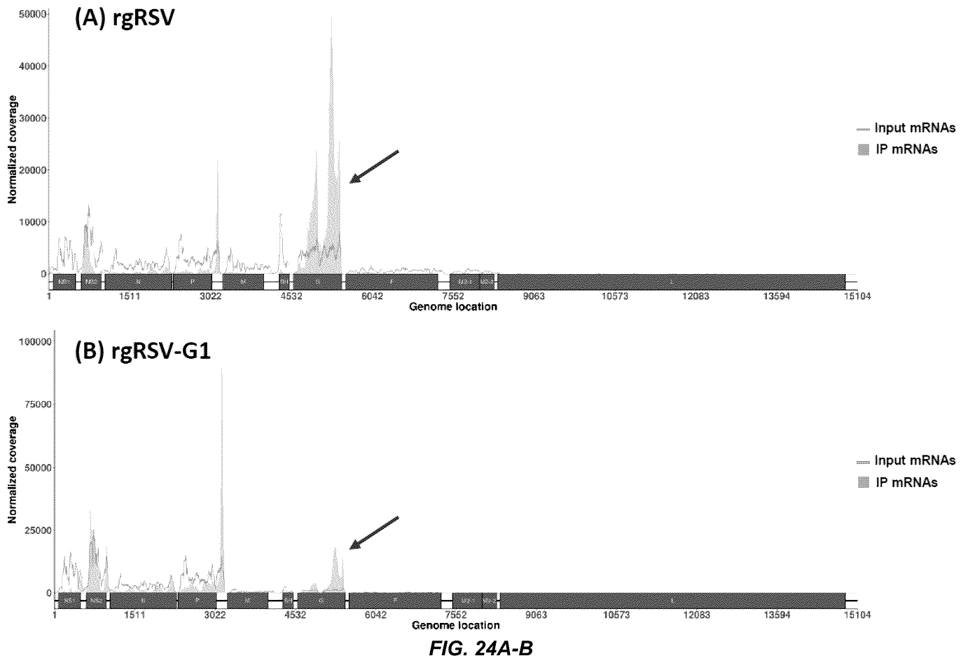

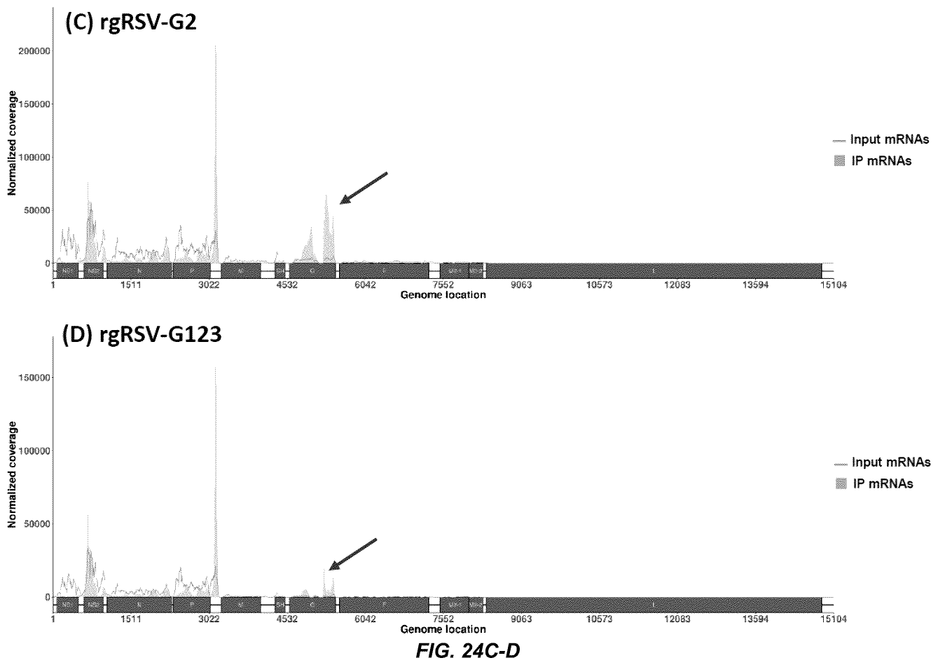

m⁶A-deficient rgRSVs had significant reductions in m⁶A enrichment specifically in G mRNA. To determine whether m⁶A sites are indeed missing from the G gene, A549 cells were infected by each m⁶A-deficient rgRSV, and polyadenylated mRNAs were isolated and subjected to m⁶A-seq. As shown in FIG. 10C and FIG. 24, the enrichment of m⁶A in the G mRNA of each m⁶A-deficient rgRSV significantly decreased compared to the G mRNA from the parental rgRSV, confirming that m⁶A methylation in the G mRNA has indeed been significantly reduced.

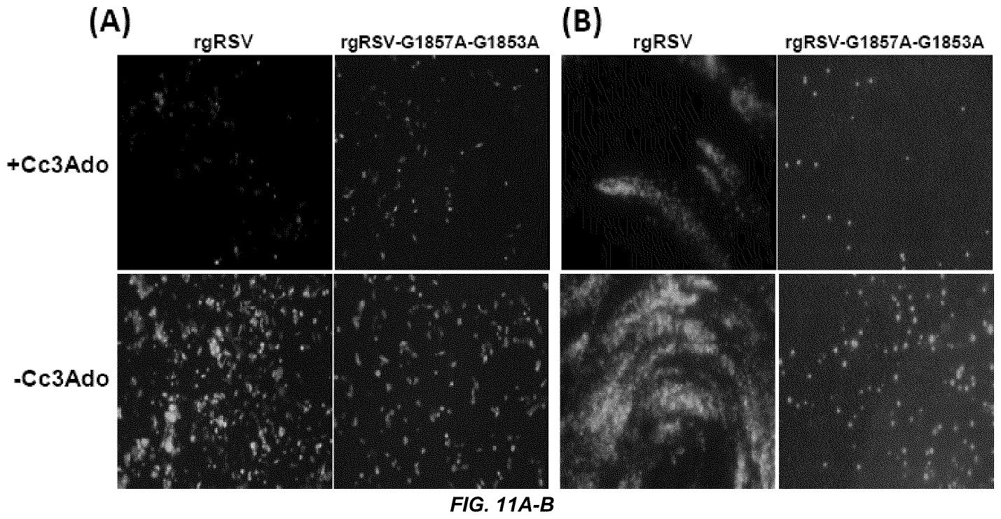

Carbocyclic 3-deazaadenosine (Cc3Ado) inhibited viral m⁶A which in turn inhibited RSV replication. Cc3Ado is an inhibitor of S-adenosyl-L-homocysteine (SAH) hydrolase, which catalyzes the reversible hydrolysis of SAH to adenosine and homocysteine [52, 53]. Inhibition of SAH hydrolase leads to an accumulation of SAH in cells, which in turn leads to a perturbation of methylation reactions. Since inhibition of SAH hydrolase will likely inhibit both mRNA cap methylation and m⁶A methylation, the inventors generated a RSV mutant that was completely defective in mRNA cap G-N-7 and ribose 2′-O methylations, which allows one to test the effect of Cc3Ado on m⁶A methylation. To do this, two mutations (G1853A and G1857A) were introduced into the SAM binding site in the L gene, and generated an RSV mutant (rgRSV-G1853A-G1857A) which inactivated the SAM binding site and is completely defective in mRNA cap methylation. As expected, rgRSV-G1853A-G1857A was defective in replication in HEp-2 cells, producing significantly less GFP compared to rgRSV (FIG. 11A). Interestingly, Cc3Ado inhibited rgRSV spread over 3 days in both rgRSV and rgRSV-G1853A-G1857A-infected cells (FIG. 11A), suggesting that inhibition of m⁶A of both cap methylation and m⁶A methylation by Cc3Ado does inhibit RSV replication and gene expression.

Next, the inventors tested the effects of Cc3Ado on RSV spread in HAE cultures, mimicking the testing of an antiviral treatment of RSV infection in human airways. Similar to HEp-2 cells, rgRSV-G1853A-G1857A was significantly attenuated for replication in HAE cells compared to rgRSV (FIG. 11B). After treatment with Cc3Ado, the GFP signal was further reduced in rgRSV-G1853A-G1857A-infected HAE cells (FIG. 11B). These results demonstrate that inhibition of m^{6A} by Cc3Ado decreases RSV replication and gene expression, suggesting that compounds that target m⁶A addition may have potential as antiviral drugs for RSV.

The biological function of m⁶A methylation in viral RNAs has remained uncertain since its discovery 40 years ago. Here, it is shown, for the first time, that the genome, antigenome, and mRNAs of RSV, an NNS RNA virus, are m⁶A methylated in both HeLa and A549 cells. The inventors showed that m⁶A modification positively regulates each step in the RSV replication cycle ranging from genome replication, mRNA transcription and viral protein synthesis, to progeny infectious particle production. Consistent with the positive effect of viral m⁶A methylation, m⁶A-deficient rgRSVs were significantly attenuated in viral replication, gene expression, and spread in A549 cells and HAE cultures. The inventors demonstrated for the first time that m⁶A regulates RSV replication and pathogenesis in an animal model. Furthermore, this example demonstrates that m⁶A could be a target for the development of live attenuated vaccine candidates as well as broad-spectrum antiviral drugs. Altogether, this work reveals that viral m⁶A has pro-viral functions in the RSV life cycle, virulence, and pathogenesis.

The m⁶A methylation of RNAs is modulated by writers, erasers, and readers in host cells. It should be noted that m⁶A methylation and its reader proteins may play distinct roles in a virus life cycle. In this study, it shown that overexpression of both m⁶A reader and writer proteins positively regulated RSV replication while knockdown inhibited RSV gene expression and replication. The opposite was true for eraser proteins: overexpression decreased RSV gene expression and replication whereas knockdown increased them. Overall, the biological functions of writers, erasers, and readers in regulating RSV replication and gene expression are consistent with each other.

In contrast, m⁶A writer and m⁶A reader proteins have been found to negatively regulate HCV production [37], opposite to RSV and influenza virus. Depletion of m⁶A writers increased infectious HCV particle production [37]. The m⁶A reader proteins relocalize to lipid droplets, the sites of HCV assembly, and suppress the packaging of HCV RNA into infectious viral particles [37]. Currently, the role of m⁶A reader proteins in the HIV life cycle is controversial [29-31]. One group found that YTHDF overexpression enhanced HIV-1 protein and RNA expression, and virus replication in CD4+ T cells [29], but others found that overexpression of m⁶A reader proteins inhibited HIV-1 infection by decreasing HIV-1 reverse transcription [31]. In a separate study, it was shown that the m⁶A sites within the Rev-response element (RRE) RNA structure alter nuclear export of HIV RNA [30]. Thus, m⁶A readers have distinct effects on the life cycles of different viruses, as they are multifunctional and play many important biological roles ranging from RNA stability, decay, and transport, to protein translation.

The inventors performed m⁶A sequencing of viral RNAs from HeLa and A549 cells. Majority of viral m⁶A peaks identified in these two cell lines overlap although there are also differences in m⁶A peak distributions. This finding suggests that different host cells may modify viral RNAs somewhat differently. These results with RSV indicate that the conserved, high density m⁶A sites are the ones that are functionally most important. Overall, the host m⁶A machinery promotes RSV replication and gene expression in both HeLa and A549 cells. The m⁶A-seq also found that the viral G mRNA has the most abundant m⁶A peaks among the 10 RSV mRNAs in both HeLa and A549 cells. In addition, the strongest m⁶A peaks in both the genome and the antigenome are located in the G gene region. As expected, the m⁶A peaks in the two positive strand RNA species, the G mRNA and G gene region of the antigenome, are largely identical. Another interesting finding was that the positions of the m⁶A modifications in the genome and antigenome largely overlapped despite the fact that the sequence of the antigenome is complementary to the genome.

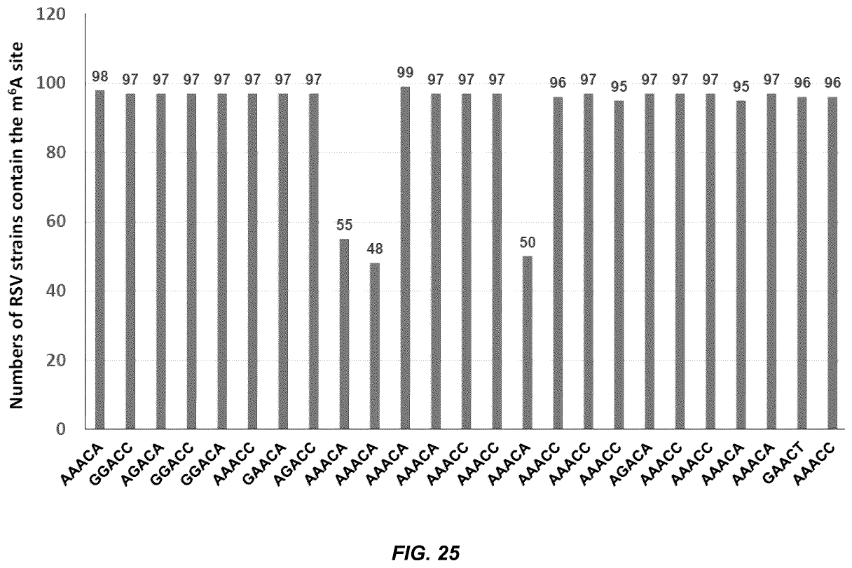

Since G mRNA has the strongest m⁶A enrichment, the inventors searched the three peaks in the G sequence for m⁶A motifs, identifying a total of 18 putative m⁶A sites. It is known that the G gene is the most genetically diverse RSV gene. However, bioinformatics analysis of 100 RSV strains (FIG. 25) found that those 18 m⁶A sites are highly conserved in the G gene, suggesting that m⁶A sites in the G gene may provide an evolutionary advantage for virus infection, replication, and spreading. Mutations in these three m⁶A peaks in the G mRNA showed that peaks 1 and 3 play a major role in regulating RSV replication whereas peak 2 plays a minor role, as recombinant rgRSV mutants in peak 1 and 3 (rgRSV-G1 and G3) had greater deficits in replication compared to mutants in peak 2 (rgRSV-G2).

The G protein is primarily responsible for the attachment of RSV to host cells and plays a role in modulating innate immune responses [51, 54]. Although it is not essential for the production of infectious RSV, RSV G is necessary for full infectivity [55, 56]. The G protein also plays an important role in the assembly of filamentous virions which have been shown to be the equivalent of virions [57]. It is likely that the abundant m⁶A modifications of the G mRNA enhances its stability, enabling more translation, insertion into virions and enhanced production of infectious virions. However, a portion of the G protein produced in a cell is released in a soluble form that affects leukocyte migration [64]. Enhanced G protein expression could enhance the production of soluble G protein, thereby affecting the immune response to RSV. It is also possible that m⁶A modification of viral RNAs facilitate the virus to escape the surveillance of host innate immunity to allow for efficient gene expression and virus replication.

Accumulating evidence suggests that m⁶A modification of cellular RNAs is important for diverse biological processes in vivo, including embryo development, cancer, and disease physiology [1, 16]. Importantly, it was found that viral m⁶A also modulates viral replication and pathogenesis in vivo. It was found that abrogating m⁶A peaks in the G gene resulted in rgRSV mutants that had significant reductions in viral replication in both the upper and lower respiratory tract of cotton rats and were less pathogenic in cotton rats.

However, the degree of attenuation in cell culture did not always match that in vivo. For example, rgRSV-G1 and G12 had similar levels of attenuation in immortalized cells (A549 and HeLa cells). In cotton rats, rgRSV-G1 replication was 7-fold reduced in the lung and nasal turbinate, respectively, whereas rgRSV-G12 had more than 100-fold reductions. Recombinant rgRSV-G2 was only mildly attenuated in cell culture. This recombinant had similar level of replication in lungs (P>0.05), and only had 3-fold reduction in nasal turbinates (P<0.05). Therefore, it appears that m⁶A sites in peaks 1 and 2 contributed synergistically to the highly attenuated phenotype of rgRSV-G12 in vivo. The phenotype of these mutants in primary differentiated HAE culture seems to correlate better with the phenotype in cotton rats than in HeLa and A549 cells. For example, rgRSV-G1 and G2 had delayed spreading in HAE culture but had spread robustly by late time points whereas rgRSV-G12 had much less spread during the entire experimental period. From this perspective, HAE culture may be better system to predict virus replication in vivo. Parental rgRSV caused changes in lung histology ranging from peribronchiolar mononuclear cell infiltrates to interstitial pneumonia. In contrast, m⁶A-deficient rgRSVs had significantly less histopathology. These results demonstrate that m⁶A not only modulates the virus life cycle in vitro but also regulates viral replication and pathogenesis in vivo.

In this study, the inventors designed mutations in predicted m⁶A sites to avoid as much as possible alterations to the predicted mRNA secondary structure and to avoid changes in the efficiency of translation of the new codon relative to the original codon. The inventors also confirmed the loss of m⁶A in the predicted region of the G mRNA by m⁶A sequencing and tested the functional consequences of reducing the m⁶A modifications. Functional loss of m⁶A modifications was examined by comparing replication of the mutant rgRSV in A549 cells overexpressing or depleted of m⁶A-related proteins. The m⁶A-deficient rgRSVs (G1, G12, and G123) were much less dependent on host m⁶A enzyme compared to the parental rgRSV, confirming that the attenuated phenotype of m⁶A-deficient rgRSVs is due to the reduction of m⁶A sites in G mRNA. Removal of m⁶A sites in the mRNA also removes them from the antigenome, but not from other viral mRNAs, other locations in the antigenome, or sites in the genome. Therefore, rgRSVs lacking particular m⁶A peaks in the G gene would be partially but not fully independent of host m⁶A enzymes. It is not clear if or how previous studies that mutated putative m⁶A sites in the genes of other viruses (HIV, influenza virus, and HCV) [30, 32, 37] confirmed that the phenotypes of the recombinant viruses were indeed due to the lack of m⁶A sites in viral genes.

A potentially important application of this study is in the rational design of live attenuated RSV vaccine candidates by inhibiting m⁶A addition to the mRNA and antigenome, or perhaps the viral genome. Currently, there is no FDA-approved vaccine for RSV despite the fact that it was first isolated in 1953. For decades, approaches to generate RSV vaccines employing inactivated virus or viral proteins have failed either due to a lack of immunogenicity or the potential for causing enhanced pulmonary disease upon natural infection with the same virus [43]. A live attenuated vaccine, similar to the effective vaccines for the related measles and mumps viruses, would seem to be one of the most promising methods for protection from RSV disease. However, it has been a challenge to strike the right balance between attenuation and immunogenicity [43].

Although mutations in individual m⁶A peaks in the G mRNA were not sufficient to achieve complete attenuation of RSV replication in vivo, the combination of m⁶A mutations in peaks 1 and 2 resulted in a recombinant virus that was sufficiently attenuated both in vitro and in vivo. Importantly, cotton rats vaccinated with rgRSV-G12 had similar neutralizing antibody response levels compared to parental rgRSV and were completely protected from rgRSV challenge. In addition, no enhanced lung damage was observed. Thus, rgRSV-G12 may be a good live attenuated vaccine candidate for RSV. This study demonstrates that inhibition of m⁶A methylation may be a novel method for rationally designing live attenuated vaccines.

Since m⁶A methylation occurred in the genome, antigenome, and mRNAs, one approach would be to combine multiple m⁶A mutations in selected gene regions (such as G gene, N gene, ig, and ge sequences) to generate a panel of RSV mutants with various degrees of attenuation in vivo. This approach would allow one to identify an RSV mutant that is sufficiently attenuated yet retains optimal immunogenicity. Another distinct advantage is that combinations of multiple m⁶A mutations in viral RNAs will enhance the genetic stability of a vaccine strain, because reversion to wild type at any nucleotide should not provide a major fitness gain. In fact, no revertant was found when rgRSV-G12 was blindly passed in A549 cells for 15 passages, suggesting that m⁶A-deficient rgRSV is genetically stable. These m⁶A-deficient rgRSVs would also provide invaluable tools to understand the roles of m⁶A modification in the innate immune response. In fact, it has been shown that internal m⁶A modification of in vitro synthesized RNAs prevents recognition of the RNA by the host pattern recognition receptors TLR3 and RIG-I [58]. From this prospective, m⁶A modification may provide an additional molecular signature for the host to discriminate self from non-self RNA by innate immunity, similar to RNA ribose 2′-O methylation of the mRNA cap.

This study also provides a novel approach for enhancing viral titers in cell culture, an important consideration in the production of live attenuated vaccines. Attenuated viruses typically grow to lower titers than wild-type virus. In the case of RSV, a relatively large dose of vaccine candidate is required to induce a protective immune response in humans, making vaccine production expensive. One strategy includes producing live attenuated vaccines in cells overexpressing one or more m⁶A reader or writer proteins, since overexpression of these host m⁶A machinery components enhance virus yield at least 10-fold. Such a boost in the production of a vaccine should greatly enhance its economic feasibility.

This study also provides an approach for developing novel antiviral drugs by targeting m⁶A methylation and m⁶A-related enzymes. It was found that a small molecule inhibitor of m⁶A methylation inhibited RSV replication. Previously, it was shown that an SAH hydrolase inhibitor, 3-deazaadenosine (DAA), is capable of inhibiting the replication of diverse viruses, including Rous sarcoma virus, HIV-1, RSV, parainfluenza virus type 3, VSV, measles virus, and reovirus [52, 53, 59]. Interestingly, mRNAs of all these viruses are capped, G-N-7 and 2′-O methylated, and polyadenylated. Translation of viral proteins likely follows a cap methylation-dependent translation mechanism. Thus, these studies could not discriminate between the antiviral effect of DAA on mRNA cap methylation and internal m⁶A methylation. To overcome this obstacle, a recombinant virus (rgRSV-G1853A-G1857A) that was completely defective in mRNA cap methylation was generated, allowing for the independent analysis of the inhibitory effect of m⁶A on virus replication. It was found that replication of rgRSV-G1857A-G1853A was further inhibited in the presence of Cc3Ado, suggesting that this drug also inhibits RSV replication by another mechanism, likely the only other known methylation of RNA, that of m⁶A methylation. The demonstration of the antiviral effect of SAM-dependent methylase inhibitors suggests that inhibition of mRNA cap methylation and m⁶A methylation could collectively contribute to the inhibition of RSV infection. Alternatively, it will be interesting to test the antiviral effect of methylase inhibitors on viruses (such as caliciviruses) which do not require cap-dependent translation machinery. If m⁶A positively regulates viral replication for a wide range of viruses, inhibition of m⁶A methylation or perturbation of m⁶A-related enzymes may serve as novel broad-spectrum antiviral drugs.

In summary, the inventors mapped the internal m⁶A modifications in RSV RNAs and showed that m⁶A enhances RSV replication, gene expression, and virus production. In addition, evidence that m⁶A upregulates RSV pathogenesis and virulence in vivo is provided. These findings highlight viral m⁶A machinery as a possible novel target for rational design of live attenuated vaccines, for enhanced production of live attenuated vaccines, and for broad-spectrum antiviral drug discovery.

### B. Materials and Methods

The animal study was conducted in strict accordance with USDA regulations and the recommendations in the Guide for the Care and Use of Laboratory Animals of the National Research Council and was approved by The Ohio State University Institutional Animal Care and Use Committee (IACUC; animal protocol no. 2009A0221). The animals were housed within the University Laboratory Animal Resources (ULAR) facilities of The Ohio State University according to the guidelines of the Institutional Animal Care and Use Committee (IACUC). The animal care facilities at The Ohio State University are AAALAC accredited. Every effort was made to minimize potential distress, pain, or discomfort to the animals throughout all experiments.

Cell lines. HeLa (ATCC CCL-2), A549 (ATCC CCL-185), Vero (ATCC CRL-CCL81), and HEp-2 (ATCC CCL-23) cell lines were purchased from the American Type Culture Collection (Manassas, VA) and were grown in Dulbecco's modified Eagle's medium (DMEM; Life Technologies) supplemented with 10% FBS. HeLa cells overexpressing the empty vector (pPB-CAG), YTHDF1, YTHDF2, or YTHDF3 were maintained in DMEM, 10% FBS and 1 μg/ml of puromycin every passage to select for YTHDF1-3 overexpressing cells. Primary, well-differentiated human airway epithelial (HAE) cultures were grown on collagen coated Transwell inserts (Corning Incorporated, Corning, NY) at an air-liquid interface, as previously described [51]. Upon reaching confluency and forming tight junctions, the apical medium was removed and cultures were maintained at the air-liquid interface for 4 to 6 weeks to generate well-differentiated, polarized cultures. All cell lines used in this study were free of mycoplasma, as confirmed by the LookOut Mycoplasma PCR Detection Kit (Sigma).

Virus stocks and purification. Recombinant RSV containing a green fluorescence protein (GFP) gene between the leader sequence and NS1 gene (rgRSV) [51] was propagated and titered in HeLa cells or A549 cells. To prepare purified rgRSV, 20 T150 flasks of HeLa cells or A549 cells were infected by rgRSV at an MOI of 0.1, and cell culture supernatants harvested at 48 or 72 h post-infection were clarified by centrifugation at 10,000×g for 30 min. Virus was concentrated through a 35% (wt/vol) sucrose cushion by centrifugation at 30,000×g for 2 h at 4° C. in a Ty 50.2 rotor (Beckman). The pellet was resuspended in DMEM with 10% trehalose and further purified through a sucrose gradient (20-55%) by centrifugation at 35,000×g for 2 h at 4° C. in an SW55 rotor (Beckman). The final pellet was resuspended in 0.5 ml of DMEM with 10% trehalose.

m⁶A-seq. High-throughput sequencing of the RSV and host methylome was carried out using m⁶A-seq as described previously [19]. For m⁶A-seq of the rgRSV genome and antigenome, RNAs were extracted from purified rgRSV virions and purified with the RiboMinus Eukaryote System v2 kit (Thermo Fisher). For m⁶A-seq of host transcripts, total RNAs were extracted from mock or rgRSV-infected HeLa or A549 cells and polyadenylated RNAs were isolated using Dynabeads mRNA DIRECT Purification kit (Thermo Fisher). Purified RNAs were sonicated with Bioruptor Pico (Diagenode) with 30 s ON 30 s OFF for 30 cycles, mixed with 1 μl of affinity purified anti-m⁶A monoclonal antibody (NEB) in IPP buffer (150 mM NaCl, 0.1% NP-40, 10 mM Tris-HCl, pH 7.4) and incubated for 2 h at 4° C. Enriched mRNA fragments were purified with RNA Clean & Concentrator kit (Zymo) and used for library generation with TruSeq Stranded mRNA Library Prep kit (Illumina). Sequencing was carried out on Illumina HiSeq 4000 according to the manufacturer's instructions. Two replicates of RNA samples from virions, virus-infected cells, and mock-infected cells were subjected to m⁶A-seq. For data analysis, after removing the adapter sequences, the reads were mapped to the human genome (hg38) and rgRSV genome and antigenome by using Hisat2 [60] with peak calling as described [61]. Metagene analysis was performed by R package Guitar [62]. Differential methylation analysis was performed with count based negative binomial model implemented in QNB test [48].

Quantification of RSV RNA m⁶A level using liquid chromatography-mass spectrometry (LC-MS/MS). RSV RNA (250 mg) was extracted from highly purified rgRSV virions using an RNeasy Mini kit (Qiagen) and purified twice with RiboMinus Eukaryote System v2 kit (Thermo Fisher). Purified RNA was digested and subjected to a quantitative analysis of the m⁶A level using LC-MS/MS as previously described [7].

Host Cell Gene Differential Expression analysis. Host cell differential gene expression was analyzed by R package DESeq2 [62] using wald-test. The significantly differentially expressed genes were reported at adjusted P value cutoff of 0.05.

Gene Ontogeny (GO) analysis. GO analysis was performed using the R package cluster Profiler [62]. Specifically, enrichKEGG function was called to analyze for enriched pathway and enrichMap function was called to generate network plot of enriched pathway.

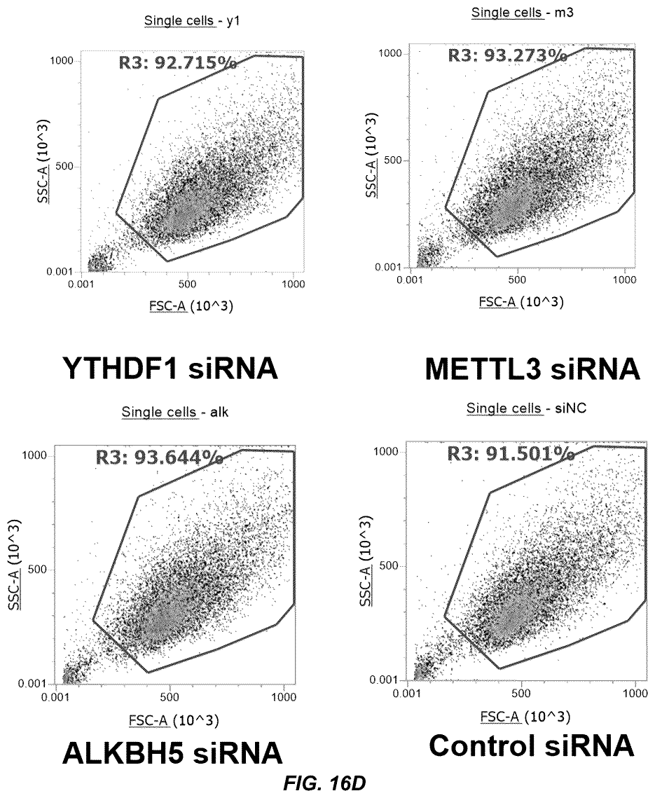

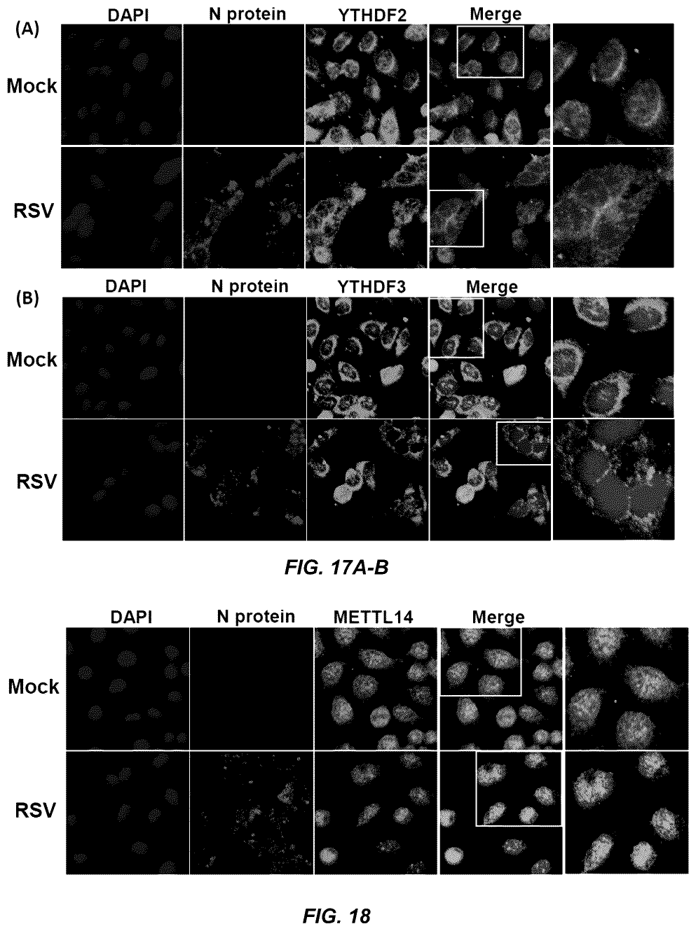

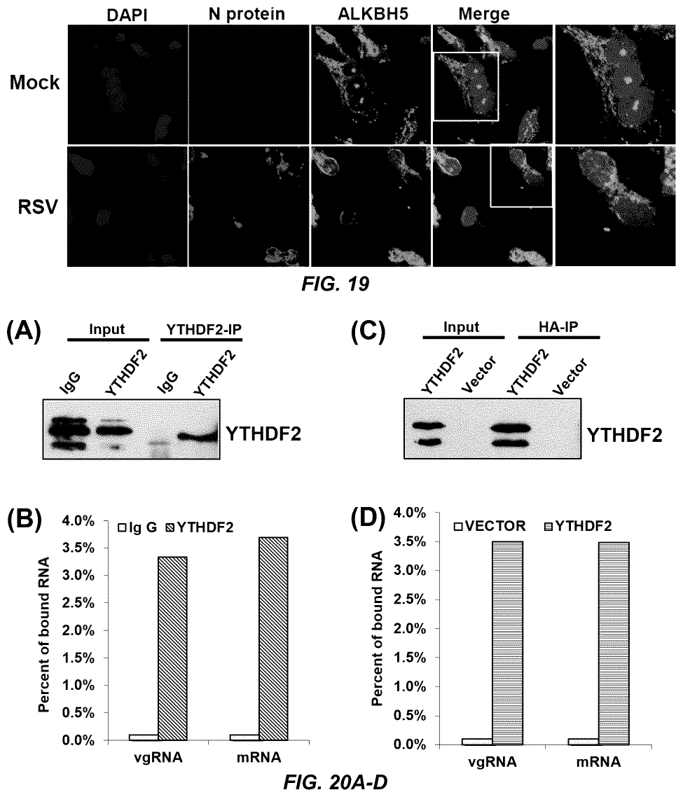

Plasmids and site-directed mutagenesis. The pPB-CAG plasmid vector was used to overexpress the readers (YTHDF1-3), writers (METTL3, METTL14), and erasers (FTO, ALKBH5) as described previously [31]. Plasmid (RW30) encoding the full-length antigenomic cDNA of RSV strain A2 with GFP inserted between the leader and the NS1 gene, and support plasmids expressing RSV A2 strain N protein (pTM1-N), P protein (pTM1-P), L protein (pTM1-L), and M2-1 protein (pTM1-M2-1) were generously provided by Dr. P. L. Collins, NIAID, Bethesda, MD. Mutations to the potential m⁶A sites in G gene were introduced into the RW30 plasmids using QuikChange site-directed mutagenesis kit (Stratagene, La Jolla, CA). There are 3 m⁶A peaks, G1, G2, and G3 in G gene which has 6, 7, and 4 putative m⁶A sites, respectively. The potential m⁶A sites mutants in G1 peak include 394-AGm⁶ACC-400; 401-AAm6ACA-407; 418-AAm⁶ACA-424; 444-AAm⁶ACA-450; 455-AAm⁶ACA-461; 459-AAm⁶ACC-465; mutants in G2 peak include 569-AAm⁶ACA-575; 576-AAm⁶ACC-582; 589-AAm⁶ACC-595; 612-AAm⁶ACC-618; 625-AGm⁶ACA-631; 645-AAm⁶ACC-651; 652-AAm⁶ACC-658; and mutants in G3 peak include 718-AAm⁶ACA-724; 722-AAm⁶ACA-728; 768-GAm⁶ACT-774; 787-AAm⁶ACC-793 (FIG. 19). The A or C within the consensus m⁶A sites was mutated to a T or G in these sites without changing the encoded amino acid. Mutant G12 combined the mutations from G1 and G2. Mutant G123 was a combined the mutations from G1, G2, and G3. To generate an rgRSV mutant lacking cap methylation, residues G1853 and G1857 in the S-adenosyl-L-methionine (SAM) binding site in the L gene were mutated to alanine in the RW30 backbone. All plasmids and mutations were confirmed by DNA sequencing.

siRNA and siRNA transfection. siRNAs against METTL3, METTL14, FTO, ALKBH5, YTHDF1, YTHDF2, YTHDF3 or non-targeting AllStars negative control siRNA were purchased from Qiagen (Valencia, CA, sequences listed in Supplementary Table 11). All siRNA transfections were performed using the Lipofectamine 3000 transfection reagent (Thermo-Fisher) according to the manufacturer's instructions.

Antibodies and Western blotting. The antibodies used in this study were anti-YTHDF1 (Proteintech, Rosemont, IL), anti-YTHDF2 (Abcam, Cambridge, MA), anti-YTHDF3 (Abcam), anti-METTL 3 (Proteintech), anti-METTL 14 (Proteintech), anti-ALKBH5 (Sigma-Aldrich), anti-FTO (Abcam), and anti-RSV serum (Virostat), F (Abcam), G (Abcam), anti-FLAG (Sigma-Aldrich), anti-Actin (Proteintech) and anti-Tubulin (Abcam). Cells were harvested and lysed in RIPA buffer (Abcam) supplemented with protease inhibitor cocktail (Sigma-Aldrich). Western blotting was performed as described. Tubulin or actin was used as a loading control.

Immunofluorescence analysis and confocal microscopy. Mock or rgRSV-infected cells were fixed in acetone and methanol at the ratio of 1:1 for 30 min, and blocked with 5% milk in PBST. Slides were stained with all primary antibodies (1:100), washed 3 times with PBST, and stained with conjugated Alexa Fluor secondary antibodies Alexa Fluor 488/594 (Thermo-Fisher; 1:300), and mounted with SlowFade™ Diamond Antifade Mountant with DAPI (Thermo-Fisher). Imaging was performed on an Olympus FV 1000 confocal microscopy system at The Ohio State University Campus Microscopy & Imaging Facility.

Real-time RT-PCR. RSV genomic RNA and mRNA were quantified by real-time RT-PCR. HeLa or A549 cells were infected with rgRSV or an rgRSV mutant at an MOI of 0.1. At 12, 18 and 24 post-infection, total RNA was isolated from cells using TRIzol (Life Technologies). Viral genomic RNA copies were quantified by real-time RT-PCR using two primers specifically targeting the RSV leader sequence and GFP gene. Poly (A)-containing viral mRNAs were isolated from total RNA using a Dynabead mRNA isolation kit (Life Technologies) according to the manufacturer's recommendations. Using the viral mRNAs as the template, the NS1 and G mRNA copies were quantified by real-time RT-PCR using two primers targeting the viral NS1 and G genes, respectively.

RNA-immunoprecipitation (RIP). The RIP assay was performed as described previously [37]. Briefly, HeLa cells were infected with rgRSV at MOI of 1.0 and cell extracts were harvested in polysome lysis buffer after 36 h post-infection. RNP complexes were immunoprecipitated with anti-HA antibody conjugated to magnetic beads (Sigma) or anti-YTHDF2 antibody overnight at 4° C., and washed five times with ice-cold NT2 buffer. For the RIP with anti-YTHDF2 antibody, additional secondary antibody was added. After the final wash, 10% of the beads were used for immunoblotting and the remaining 90% were used for RNA extraction using TRIzol (ThermoFisher).

Recovery of RSV from the full-length cDNA clones. rgRSV mutants were rescued from the full-length cDNA of the RSV A2 strain [63]. HEp-2 cells were infected with MVA-T7 at an MOI of 10, then transfected with 1.2 pg of plasmid RW30 or RW30 mutant, 0.4 pg of pTM1-N, 0.2 pg of pTM1-P, 0.1 pg of pTM1-M2-1, and 0.1 pg of pTM1-L using the Lipofectamine 3000 reagent (Life Technologies). At day 4 post-transfection, the cells were harvested using scrapers and were co-cultured with new flask of HEp-2 cells at 50 to 60% confluence. When an extensive cytopathic effect (CPE) was observed, the cells were subjected to three freeze-thaw cycles, followed by centrifugation at 4,000×g for 10 min. The supernatant was subsequently used to infect new HEp-2 cells. The successful recovery of the rgRSV was confirmed by the presence of green fluorescent cells, followed by RT-PCR and sequencing. Recombinants rgRSV carrying mutations in m⁶A sites were designated as rgRSV-G1, G2, G3, G12, and G123. Recombinant rgRSV carrying double mutations in the SAM binding site of L gene was designated as rgRSV-G1853A-G1857A.

RT-PCR and sequencing. All plasmids, viral mutants and stocks, and virus isolates from the nasal turbinates and lungs of cotton rats were sequenced to confirm virus identity. Viral RNA was extracted from 100 μl of each recombinant virus using an RNeasy minikit (Qiagen, Valencia, CA). A 1.5-kb DNA fragment spanning the RSV G gene was amplified by RT-PCR. The PCR products were purified and sequenced using a sequencing primer at The Ohio State University Plant Microbe Genetics Facility to confirm the presence of the designed mutations.

Viral replication kinetics. Confluent HeLa or A549 cells in 6-well-plate were infected with wild-type rgRSV or mutant rgRSV at an MOI of 0.1. After 1 h of adsorption, the inoculum was removed and the cells were washed three times with DMEM. Fresh DMEM (supplemented with 2% FBS) was added, and the infected cells were incubated at 37° C. At different time points post-inoculation, the supernatant and cells were harvested by three freeze-thaw cycles, followed by centrifugation at 1,500×g at room temperature for 15 min. The virus titer was determined by TCID50 assay in HEp-2 cells [51].

Genetic stability of rgRSV mutants in cell culture. Confluent Vero cells in T25 flasks were infected with each rgRSV mutant at an MOI of 0.1. At day 3 post-inoculation, the cell culture supernatant was harvested and used for the next passage in Vero cells. Using this method, each rgRSV mutant was repeatedly passaged 15 times in Vero cells. At each passage, the G gene was amplified by RT-PCR and sequenced. At passage 15, the entire genome of each recombinant virus was amplified by RT-PCR and sequenced.

Replication and pathogenesis of rgRSV mutants in cotton rats. Thirty 6-week-old specific-pathogen-free (SPF) male cotton rats (Envigo, Indianapolis, IN) were randomly divided into 6 groups (5 cotton rats per group). Prior to virus inoculation, the cotton rats were anesthetized with isoflurane. The cotton rats in group 1 were inoculated with 2.0×10⁵ TCID₅₀ of parental rgRSV and served as positive controls. The cotton rats in groups 2 to 5 were inoculated with 2.0×10⁵ TCID₅₀ of four m⁶A deficient rgRSV mutants, rgRSV-G1, G2, G3, and G12. Each cotton rat was inoculated intranasally with a volume of 100 μl. At day 4 post-infection, the cotton rats were sacrificed via carbon dioxide inhalation. The left lung and nasal turbinates were collected for virus titration and the right lung was collected for histological analysis.

Immunogenicity of rgRSV in cotton rats. For the immunogenicity study, twenty 6-week-old female cotton rats (Envigo) were randomly divided into five groups (5 cotton rats per group). Cotton rats in groups 1, 2, and 3 were intranasally inoculated with 2.0×10⁵ TCID₅₀ of two m⁶A deficient rgRSV mutants (rgRSV-G1 and G12) and rgRSV, respectively. Cotton rats in groups 4 were mock-infected with DMEM and served as unvaccinated challenged control. After immunization, the cotton rats were evaluated daily for any possible abnormal reaction and blood samples were collected from each cotton rat weekly by facial vein retro-orbital plexus sampling, and serum was used for detection of neutralizing antibodies. At 4 weeks post-immunization, the cotton rats in groups 2 to 5 were challenged with 2.0×10⁵ TCID₅₀ of parental rgRSV via intranasal route, and evaluated twice daily for the presence of any clinical symptoms. At 4 days post-challenge, all cotton rats were euthanized by CO2 asphyxiation, and their lungs and nasal turbinates were collected for virus titration. The immunogenicity of rgRSV mutants was assessed based on their ability to trigger neutralizing antibody, the ability to prevent rgRSV replication in lungs and nose, and the ability to protect lung from pathological changes.

Pulmonary histology. After sacrifice, the right lung of each animal was removed, inflated, and fixed with 4% neutral buffered formaldehyde. Fixed tissues were embedded in paraffin and a microtome used to generate 5 m sections. Slides were then stained with hematoxylin-eosin (H&E) for the examination of histological changes by light microscopy. Histopathological changes were evaluated based on the extent of interstitial inflammation, edema, and peribronchiolar inflammation.

Determination of viral titer in lung and nasal turbinate. The nasal turbinate and the left lung from each cotton rat were removed, weighed, and homogenized in either 3 ml or 2 ml of DMEM. The lung was homogenized using a Precellys 24 tissue homogenizer (Bertin, MD) by following the manufacturer's recommendations. The nasal turbinates were homogenized by hand with a 15 mL capacity PYREX® homogenizer (Corning, NY). The presence of infectious virus was determined by TCID50 assay in HEp-2 cells.

Determination of RSV-neutralizing antibody. RSV-specific neutralizing antibody titers were determined using a plaque reduction neutralization assay. Briefly, cotton rat sera were collected by retro-orbital plexus sampling weekly until challenge. The serum samples were heat inactivated at 56° C. for 30 min. Twofold dilutions of the serum samples were mixed with an equal volume of DMEM containing approximately 50 TCID50/well rgRSV in a 96-well plate, and the plate was incubated at room temperature for 1 h with constant rotation. The mixtures were then transferred to confluent HEp-2 cells in a 96-well plate in triplicate. After 1 h of incubation at 37° C., the virus-serum mixtures were removed and the cells were overlaid with 0.75% methylcellulose in overlay media (1×MEM, 2% FBS, Sodium bicarbonate, 25 mM HEPES, 1% L-Glutamine, 1% Pen Strep) and incubated for another 3 days before counting the fluorescent foci. The numbers of foci at each serum dilution were plotted and the 50% plaque reduction titer was used as the RSV-specific neutralizing antibody titer.

Statistical analysis. Quantitative analysis was performed by either densitometric scanning of autoradiographs or by using a phosphorimager (Typhoon; GE Healthcare, Piscataway, NJ) and ImageQuant TL software (GE Healthcare, Piscataway, NJ). Statistical analysis was performed by one-way multiple comparisons using SPSS (version 8.0) statistical analysis software (SPSS Inc., Chicago, IL). A P value of <0.05 was considered statistically significant.

### C. Tables

SUPPLEMENTARY TABLE 1
m⁶A peaks in RSV RNAs purified from virions grown in HeLa cells

| | Peak | Peak range | Gene | Peak size | Enrichment | Enrichment |
|---|---|---|---|---|---|---|
| RSV RNAs | no. | (nt) ^{A} | location ^{B} | (nt) | Score | Fold ^{C} |
| Genome | 1 | 599-972 | gs, NS2 | 373 | 5.07 | 2.34 |
| | 2 | 1571-1645 | N | 74 | 3.42 | 1.77 |
| | 3 | 1795-1944 | N | 149 | 2.89 | 1.53 |
| | 4 | 2617-2766 | P | 149 | 3.65 | 1.87 |
| | 5 | 3066-3215 | ig | 149 | 8.81 | 3.13 |
| | 6 | 3963-4262 | M, ge, ig | 299 | 3.22 | 1.68 |
| | 7 | 4711-5533 | G, ge, ig | 822 | 6.68 | 2.74 |
| | 8 | 11291-1365 | L | 74 | 3.39 | 1.76 |
| | 9 | 13459-3533 | L | 74 | 3.29 | 1.71 |
| | 10 | 13758-3832 | L | 74 | 2.70 | 1.43 |
| | 11 | 13908-3982 | L | 74 | 3.44 | 1.78 |
| Antigenome | 1 | 1645-1719 | N | 74 | 2.39 | 1.26 |
| | 2 | 2543-2991 | P | 448 | 3.07 | 1.62 |
| | 3 | 4786-5458 | G | 672 | 5.49 | 2.45 |
| | 4 | 5833-5907 | F | 74 | 4.66 | 2.22 |

^{A} Nucleotide sequence is referred to RSV A2 strain. Nucleotide ranges are indicated. m⁶A peaks in G gene region are highlighted by yellow color.
^{B} The RSV genes and regulatory elements are covered by m⁶A peaks. These regions may contain m⁶A sites. However, whether these regions indeed contain m⁶A sites will require to search the presence of m⁶A motif, Pu [G > A]m⁶AC[A/C/U] motif (Pu represents purine). RSV gene start, gene end, and intergenic sequence are indicated by gs, ge, and ig, respectively.
^{C} log2 enrichment of the m⁶A peaks identified in RSV antigenome and genome.

SUPPLEMENTARY TABLE 2
m⁶A peaks in RSV mRNAs from rgRSV-infected HeLa cells

| | Peak | Peak range | Gene | Peak size | Enrichment | Enrichment |
|---|---|---|---|---|---|---|
| RSV RNAs | no. | (nt) ^{A} | location ^{B} | (nt) | Score | Fold ^{C} |
| mRNAs | 1 | 1-50 | Leader | 49 | 3.87 | 1.95 |
| | 2 | 450-648 | NS1, ge, ig, gs, | 198 | 3.45 | 1.78 |
| | | | NS2 | | | |
| | 3 | 898-997 | NS2, ge, ig | 99 | 5.45 | 2.44 |
| | 4 | 1895-1944 | N | 49 | 2.79 | 1.48 |
| | 5 | 2194-2243 | N | 49 | 5.73 | 2.52 |
| | 6 | 2444-2542 | P | 98 | 3.24 | 1.69 |
| | 7 | 2743-2792 | P | 49 | 4.04 | 2.01 |
| | 8 | 3042-3190 | P, ge, ig | 148 | 16.05 | 4.01 |
| | 9 | 3740-3789 | M | 49 | 3.70 | 1.88 |
| | 10 | 3989-4138 | M, ge, ig | 149 | 5.95 | 2.57 |
| | 11 | 4288-4337 | gs, SH | 49 | 3.81 | 1.93 |
| | 12 | 4388-4437 | SH | 49 | 7.81 | 2.96 |
| | 13 | 4687-5533 | G, ge, ig | 846 | 15.38 | 3.94 |
| | 14 | 7279-7328 | ge, ig | 49 | 4.48 | 2.16 |
| | 15 | 8326-8375 | ig, gs | 49 | 8.32 | 3.05 |
| | 16 | 13909-3958 | L | 49 | 5.43 | 2.44 |

^{A} Nucleotide sequence is referred to RSV A2 strain. Nucleotide ranges are indicated. m⁶A peaks in G gene region are highlighted by yellow color.
^{B} The RSV mRNAs and regulatory elements are covered by m⁶A peaks. These regions may contain m⁶A sites. However, whether these regions indeed contain m⁶A sites will require to search the presence of m⁶A motif, Pu [G > A]m⁶AC[A/C/U] motif (Pu represents purine). RSV gene start, gene end, and intergenic sequence are indicated by gs, ge, and ig, respectively.
^{C} log2 enrichment of the m⁶A peaks identified in RSV mRNAs.

SUPPLEMENTARY TABLE 3
m⁶A peaks in RSV RNAs purified virions grown in A549 cells

| | Peak | Peak range | Gene | Peak size | Enrichment | Enrichment |
|---|---|---|---|---|---|---|
| RSV RNAs | no. | (nt) | location | (nt) | Score | Fold |
| Genome | 1 | 599-798 | gs, NS2 | 199 | 13.73 | 3.77 |
| | 2 | 1746-1944 | N | 198 | 21.94 | 4.45 |
| | 3 | 2194-2243 | N | 49 | 10.15 | 3.34 |
| | 4 | 2444-2542 | P | 98 | 7.62 | 2.93 |
| | 5 | 2593-2841 | P | 248 | 9.65 | 3.27 |
| | 6 | 2992-3240 | P, ge, ig, gs | 248 | 178.31 | 7.47 |
| | 7 | 4737-5085 | G | 348 | 35.42 | 5.14 |
| | 8 | 5135-5483 | G | 348 | 90.27 | 6.49 |
| | 9 | 5883-6082 | F | 199 | 6.30 | 2.65 |
| Antigenome | 1 | 400-848 | NS1, ge, ig, gs, | 448 | 9.89 | 3.31 |
| | | | NS2 | | | |
| | 2 | 898-997 | NS2, ge, ig | 99 | 6.66 | 2.73 |
| | 3 | 1347-1446 | N | 99 | 4.57 | 2.19 |
| | 4 | 1496-1695 | N | 199 | 6.86 | 2.77 |
| | 5 | 2095-2243 | N | 148 | 14.49 | 3.85 |
| | 6 | 2344-2393 | P | 49 | 3.07 | 1.62 |
| | 7 | 2444-3290 | P, ge, ig, gs, M | 846 | 20.56 | 4.36 |
| | 8 | 4039-4138 | lg | 99 | 8.43 | 3.07 |
| | 9 | 4537-4686 | ig, gs, G | 149 | 3.64 | 1.86 |
| | 10 | 4737-5434 | G | 697 | 47.63 | 5.57 |
| | 11 | 5484-5533 | lg | 49 | 17.78 | 4.15 |
| | 12 | 5783-6082 | F | 299 | 12.5 | 3.64 |
| | 13 | 8326-8375 | ig, gs | 49 | 5.55 | 2.47 |
| | 14 | 12114-2263 | L | 149 | 5.78 | 2.53 |
| | 15 | 14806-4855 | L | 49 | 13.29 | 3.73 |

SUPPLEMENTARY TABLE 4
m⁶A peaks in RSV mRNAs purified from rgRSV-infected A549 cells

| | Peak | Peak range | Gene | Peak size | Enrichment | Enrichment |
|---|---|---|---|---|---|---|
| RSV RNAs | no. | (nt) | location | (nt) | Score | Fold |
| mRNAs | 1 | 1-50 | leader | 49 | 3.86 | 1.95 |
| | 2 | 450-798 | NS1, ge, ig, gs, NS2 | 348 | 12.19 | 3.61 |
| | 3 | 898-997 | NS2, ge, ig | 99 | 3.58 | 1.84 |
| | 4 | 1048-1246 | N | 198 | 2.58 | 1.37 |
| | 5 | 1297-1396 | N | 99 | 3.03 | 1.60 |
| | 6 | 1496-1695 | N | 199 | 5.12 | 2.35 |
| | 7 | 1796-1994 | N | 198 | 5.87 | 2.55 |
| | 8 | 2045-2642 | N, ge, ig, gs, P | 597 | 5.21 | 2.38 |
| | 9 | 2743-3589 | P, ge, ig, gs, M | 846 | 22.23 | 4.47 |
| | 10 | 3989-4138 | M, ge, ig | 149 | 4.34 | 2.11 |
| | 11 | 4188-4337 | ig, gs, SH | 149 | 2.46 | 1.30 |
| | 12 | 4487-5533 | ig, gs, G, ge, ig | 1046 | 85.69 | 6.42 |
| | 13 | 5783-6082 | F | 299 | 6.61 | 2.72 |
| | 14 | 6481-6580 | F | 99 | 2.42 | 1.27 |
| | 15 | 7628-7876 | M2-1 | 248 | 2.86 | 1.51 |
| | 16 | 7977-8026 | M2-1 | 49 | 2.90 | 1.53 |
| | 17 | 8326-8424 | ig, gs, L | 98 | 2.54 | 1.34 |
| | 18 | 12114-12163 | L | 49 | 5.10 | 2.35 |

SUPPLEMENTARY TABLE 5
Overlapping m⁶A peaks in RSV RNAs purified
virions grown in HeLa and A549 cells

| | Overlapping | Peak range | Peak size | |
|---|---|---|---|---|
| RSV RNAs | Peak no. | (nt) | (nt) | Gene location |
| Genome | 1 | 599-798 | 199 | gs, NS2 |
| | 2 | 1795-1944 | 149 | N |
| | 3 | 2617-2766 | 149 | P |
| | 4 | 3066-3215 | 149 | ig |
| | 5 | 4737-5085 | 348 | G |
| | 6 | 5135-5483 | 348 | G |
| Antigenome | 1 | 1645-1695 | 50 | N |
| | 2 | 2543-2991 | 448 | P |
| | 3 | 4786-5434 | 648 | G |
| | 4 | 5833-5907 | 74 | F |

SUPPLEMENTARY TABLE 6
Overlapping m⁶A peaks in RSV mRNAs purified
from rgRSV-infected HeLa and A549 cells

| | Overlapping | Peak range | Peak size | |
|---|---|---|---|---|
| RSV RNAs | Peak no. | (nt) | (nt) | Gene location |
| mRNAs | 1 | 1-50 | 49 | Leader |
| | 2 | 450-648 | 198 | NS1, ge, ig, gs, |
| | | | | NS2 |
| | 3 | 898-997 | 99 | NS2, ge, ig |
| | 4 | 1895-1944 | 49 | N |
| | 5 | 2194-2243 | 49 | N |
| | 6 | 2444-2542 | 98 | P |
| | 7 | 2743-2792 | 49 | P |
| | 8 | 3989-4138 | 149 | M, ge, ig |
| | 9 | 4288-4337 | 49 | gs, SH |
| | 10 | 4687-5533 | 846 | G, ge, ig |
| | 11 | 8326-8375 | 49 | ig, gs |

Supplementary Table 7 siRNA used for knocking
down host m⁶A machinery

| SiRNA | Sequences (5'-3') |
|---|---|
| YTHDF1 | 5'-CCGCGTCTAGTTGTTCATGAA-3' |
| | (SEQ ID NO: 24) |
| YTHDF2 | 5'-AAGGACGTTCCCAATAGCCAA-3' |
| | (SEQ ID NO: 25) |
| YTHDF3 | 5'-ATGGATTAAATCAGTATCTAA-3' |
| | (SEQ ID NO: 26) |
| METTL3 | 5'-CTGCAAGTATGTTCACTATGA-3' |
| | (SEQ ID NO: 27) |
| METTL14 | 5'-AAGGATGAGTTAATAGCTAAA-3' |
| | (SEQ ID NO: 28) |
| ALKBH5 | 5'-AAACAAGTACTTCTTCGGCGA-3' |
| | (SEQ ID NO: 29) |
| FTO | 5'-AAATAGCCGCTGCTTGTGAGA-3' |
| | (SEQ ID NO: 30) |
| Control siRNA | 5' ACGTGACACGTTCGGAGAA-3' |
| | (SEQ ID NO: 31) |

### Example 2: N⁶-methyladenosine is a Molecular Signature for Discrimination of Self and Non-Self RNA by Cytoplasmic RNA Sensor RIG-I

Internal N⁶-methyladenosine (m⁶A) modification of RNA is one of the most common and abundant modifications in eukaryotic cells as well as in viruses. However, the biological role(s) of RNA m⁶A in virus-host interaction remains elusive. Using human metapneumovirus (hMPV), a medically important non-segmented negative-sense RNA virus as a model, the inventors demonstrate that m⁶A serves as a molecular marker for innate immune discrimination self and nonself RNAs. The inventors show that hMPV RNAs are m⁶A methylated and that viral m⁶A methylation promotes hMPV replication and gene expression. HMPV infection leads to differential expression of interferon-related genes involved in innate immune signaling pathways. Inactivating these m⁶A sites with synonymous mutations resulted in m⁶A deficient recombinant hMPVs that induced significantly higher expression of type I interferon that restricted viral replication. Notably, the induction of type I interferons by m⁶A-deficient rhMPVs and virion RNA was dependent on the cytoplasmic RNA sensor RIG-I, not MDA5. Mechanistically, m⁶A-deficient virion RNA induces higher expression of RIG-I, enhances its binding affinity to RIG-I, and facilitates the conformational change of RIG-I, leading to enhanced induction of type I IFN expression. The replication of m⁶A-deficient rhMPVs was attenuated in wild type A549 cells but was restored in cells knocked out for RIG-I and MAVS. Furthermore, m⁶A-deficient rhMPVs triggered higher type I interferon in vivo and were significantly attenuated in the lower respiratory tract yet retained high immunogenicity in cotton rats. Collectively, these results highlight that (i) virus acquires m⁶A in their RNAs as a means of mimicking cellular RNA to avoid the detection by innate immunity; and (ii) viral m⁶A RNA can serve as a novel target to attenuate hMPV for vaccine purposes.

Here, the inventors demonstrate that m⁶A modification serves as a molecular marker for innate sensing by cells to discriminate self and nonself RNA and that m⁶A regulates viral pathogenesis. The inventors found that the genome, antigenome, and mRNAs of human metapneumovirus (hMPV) are m⁶A modified and that m⁶A modification in hMPV RNAs positively regulated each step in the hMPV replication cycle, including RNA replication, mRNA transcription, protein synthesis, and progeny virus production. Next, the inventors generated recombinant (r)hMPVs lacking various m⁶A sites in the G gene region of the antigenome and the G gene in the genome and found that replication of the m⁶A-deficient rhMPVs was significantly reduced in cell culture while inducing an elevated type I interferon (IFN-I) response. The inventors showed that the m⁶A-deficient hMPV antigenome and/or genome, but not the viral mRNA, was responsible for the enhanced IFN response. Mechanistically, m⁶A-deficient hMPV virion RNA enhances its binding affinity to RIG-I, facilitates the conformational change of RIG-I, and induces higher RIG-I expression. Depletion of RIG-I and the mitochondrial antiviral signaling (MAVS) but not MDA5 completely abrogated the rhMPV-induced type I IFN responses. Furthermore, the inventors demonstrated that in a cotton rat model m⁶A-deficient rhMPVs were highly attenuated in replication in the lungs and provided complete protection against hMPV reinfection. These results suggest that the m⁶A modification serves as a molecular signature for host innate immunity to discriminate self from non-self RNA, and that inactivating the m⁶A modification could serve as a means to attenuate hMPV and perhaps other NNS RNA viruses for the vaccine purposes.

### A. Materials and Methods

Ethics statement. The animal study was conducted in strict accordance with the Guide for the Care and Use of Laboratory Animals of the National Research Council and was approved by The Ohio State University Institutional Animal Care and Use Committee (IACUC; animal protocol no. 2009A0221). The animal care facilities at The Ohio State University are AAALAC accredited. Every effort was made to minimize potential distress, pain, or discomfort to the animals throughout all experiments.

Cell lines. Vero E6 cells (ATCC CRL-1586), A549 cells (ATCC CCL-185), and THP-1 (ATCC TIB-202) were purchased from the American Type Culture Collection (Manassas, VA). A549-Dual™, A549-Dual™ KG-RIG-I, A549-Dual™ KO-MDA5, and A549-Dual™ KO-MAVS knockout cells were purchased from InvivoGen (San Diego, CA). BHK-SR19-T7 cells were kindly provided by Apath, LLC, Brooklyn, NY. All cell lines were grown in Dulbecco's modified Eagle's medium (DMEM; Life Technologies) supplemented with 10% FBS. The medium for the BHK-SR19-T7 cells was supplemented with 10 μg/ml puromycin (Life Technologies) during every other passage to select for T7 polymerase-expressing cells. A549-Dual™ and knockout cell lines were supplemented with Normocin™ (100 μg/ml), blasticidin (10 μg/ml) and Zeocin™ (100 μg/ml). HeLa cells overexpressing the empty vector (pPB-CAG), YTHDF1, YTHDF2, or YTHDF3 were maintained in DMEM supplemented with 10% FBS and 1 μg/ml of puromycin every passage to select for YTHDF1-3 overexpressing cells. All cell lines used in this study were free of mycoplasma, as confirmed by the LookOut Mycoplasma PCR Detection Kit (Sigma).

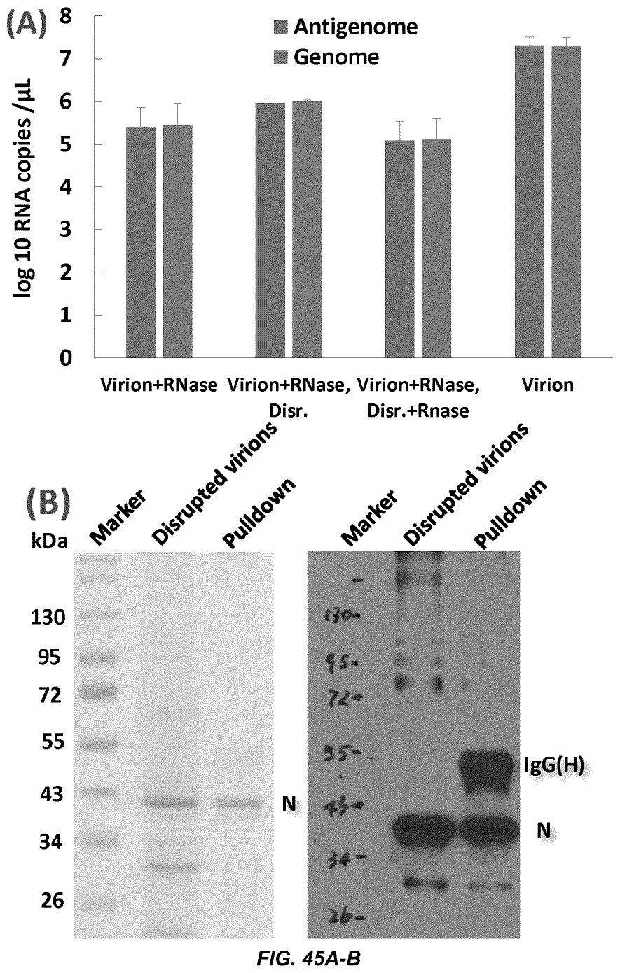

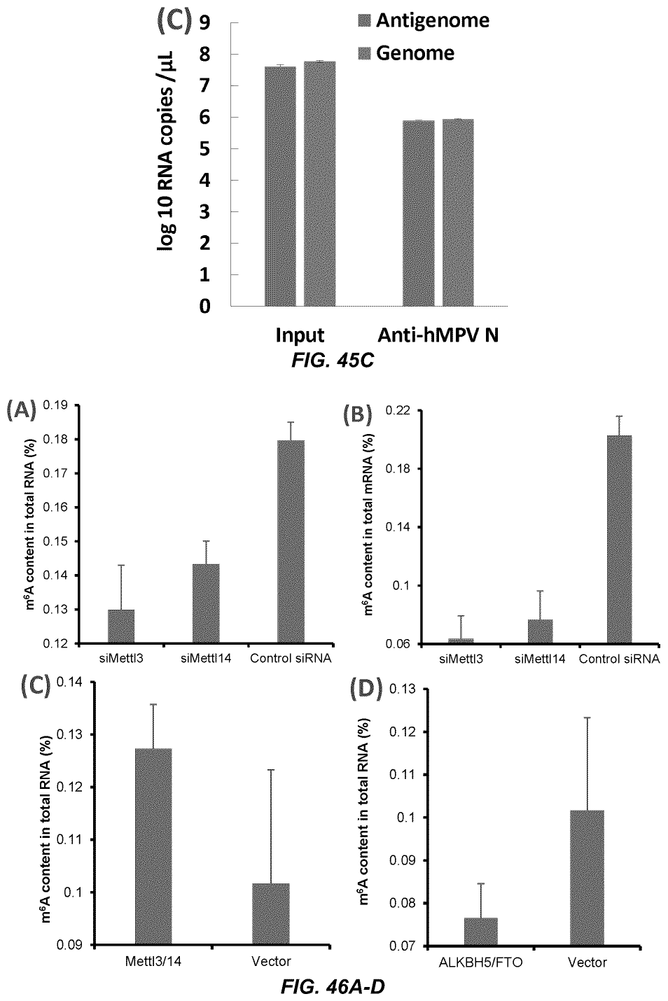

Plasmids and site-directed mutagenesis. Plasmids encoding the full-length genomic cDNA of hMPV strain NL/1/00 (phMPV), and support plasmids expressing hMPV N protein (pCITE-N), P protein (pCITE-P), L protein (pCITE-L), and M2-1 protein (pCITE-M2-1) were kindly provided by Ron A. M. Fouchier at the Department of Virology, Erasmus Medical Center, Rotterdam, The Netherlands (50). The F cleavage site in the genome of hMPV NL/1/00 was modified to a trypsin-independent F cleavage site, as described previously (51). A GFP gene was cloned into the gene junction between N and P in plasmid phMPV, resulted in the construction of phMPV-GFP. The G gene of hMPV strain NL/1/00 was cloned into pCAGGS resulted in the construction of pCAGGS-G. Mutations to the potential m⁶A sites in G gene were introduced into the plasmids pCAGGS-G and phMPV using QuikChange site-directed mutagenesis kit (Stratagene, La Jolla, CA). The pPB-CAG plasmid vector was used to overexpress the readers (YTHDF1-3 and YTHDC1), writers (METTL3, METTL14), and erasers (FTO, ALKBH5) as described previously (29, 31). These m⁶A sites mutants in G gene region of the antigenome include: site 1, 171-AAm⁶A [symbol] A-175; site 2, 187-GAm⁶ [symbol] CA-191; site 3, 227-AAm⁶AC [symbol] -231; site 4, 246-AGm⁶A [symbol] A-250; site 5, 255-AGm⁶A [symbol] A-259; site 6, 341-AGm⁶AC [symbol] -345; site 7, 346-GAm⁶ [symbol] CC-351; site 8, 422-GAm⁶AC [symbol] -426; site 9, 428-AGm⁶AC [symbol] -432; site 10, 453-AAm⁶A [symbol] A-457; site 11, 464-GGm⁶AC [symbol] -468; site 12, 476-GAm⁶AC [symbol] -480; site 13, 518-GAm⁶AC [symbol] -522; and site 14, 553-AGm⁶ [symbol] CC-557 (FIG. 45). The m⁶A sites mutants in the G gene of the genome include: site 1, 237- [symbol] Gm⁶T [symbol] C-241; site 2, 290-A [symbol] m⁶TC [symbol] -294; site 3, 433-AGm⁶ [symbol] CC-437; site 4, 441- [symbol] Gm⁶T [symbol] C-445; site 5, 570-AGm⁶ [symbol] CC-574; and site 6, 616-A [symbol] m⁶TC [symbol] -620. The A or C within the consensus m⁶A sites was mutated to a T or G in these sites without changing the encoded amino acid. Mutants G1-2, G8-9, G8-14, and G1-14 were a combination of mutations in sites 1 and 2, sites 8 and 9, sites 8 to 14, and sites 1 to 14, respectively. Mutant G(−)1-6 is the combined mutation of all six m⁶A sites in the G gene in the genome. All constructs were sequenced at The Ohio State University Plant Microbe Genetics Facility.

Virus stocks and purification. Parental hMPV strain NL/1/00 was propagated and titrated in Vero E6 cells. To prepare highly purified hMPV for m⁶A sequencing, 20 T150 flasks of A549 cells were infected by hMPV at an MOI of 0.5, and cell culture supernatants harvested at 72 h post-infection were clarified by centrifugation at 5,000×g for 30 min. Virus was concentrated by centrifugation at 30,000×g for 2 h at 4° C. in a Ty 50.2 rotor (Beckman). The pellet was resuspended in NTE buffer (0.05 M Tris-HCl, 0.15 M NaCl, 15 mM CaCl₂ [pH 6.5]) supplemented with 10% trehalose and further purified through a sucrose gradient by centrifugation at 35,000×g for 18 h at 4° C. in an SW55 rotor (Beckman). Solution layer containing virus was extracted with syringe, diluted with NTE buffer and centrifuged at 30,000×g for 2 h at 4° C. in SW55 rotor. The final pellet was resuspended in 0.5 ml of NTE buffer.

m⁶A-seq. High-throughput sequencing of the hMPV and host methylome was carried out using m⁶A-seq as described previously (38). For m⁶A-seq of the hMPV genome and antigenome, RNAs were extracted from highly purified hMPV virions and purified with the RiboMinus Eukaryote System v2 kit (Thermo Fisher). For m⁶A-seq of host transcripts, total RNAs were extracted from mock or hMPV-infected A549 cells and polyadenylated RNAs were isolated using Dynabeads mRNA DIRECT Purification kit (Thermo Fisher). Purified RNAs were sonicated with Bioruptor Pico (Diagenode) with 30 s ON 30 s OFF for 30 cycles, mixed with 2.5 mg of affinity purified anti-m⁶A polyclonal antibody (NEB cat.E1610) in IPP buffer (150 mM NaCl, 0.1% NP-40, 10 mM Tris-HCl, pH 7.4) and incubated for 2 h at 4° C. Enriched mRNA fragments were purified with RNA Clean & Concentrator kit (Zymo) and used for library generation with Kapa RNA HyperPrep kit (Roche). Sequencing was carried out on Illumina HiSeq 4000 at SE50 bp mode according to the manufacturer's instructions. Two replicates of RNA samples from virions, virus-infected cells, and mock-infected cells were subjected to m⁶A-seq. For data analysis, after removing the adapter sequences, the reads were mapped to the human genome (hg38) and hMPV genome by using Hisat2 (52). Peak calling for the viral genome RNA was done by first dividing the hMPV genome into 30 bp consecutive bins where read count was quantified. Then the inventors applied Fisher's exact test to assess enrichment of coverage by m⁶A-IP in that bin. The odds ratio was computed by (IP/overall IP)/(Input/overall Input) where overall IP/Input were represented by median of read counts of bins across the same strand of the whole virus genome. Note, when calling peaks for mRNAs of the hMPV, the overall IP/Input were represented by the median of bins across the gene instead of the whole virus genome. Finally, the inventors merged all neighboring bins that are significant (at FDR<0.05 cutoff) in all replicates and report them as consistent peaks.

Differential expression analysis of host cells. The input of m⁶A-seq is equivalent to regular RNA-seq, therefore the inventors quantified the gene-level read count of input samples that aligned to hg38 for differential gene expression analysis. DESeq2 was used to make an inferential test where differentially expressed genes were identified at FDR<0.1 cutoff.

Differential methylation analysis of host cell. To compare the m⁶A-methylome of the mock infected and hMPV infected cells, the inventors first called peaks using fisher's exact test on 50 bp consecutive bins as described in previous section. The inventors then used QNB package for differential methylation test with default setting.

Quantification of hMPV RNA m⁶A level using liquid chromatography-mass spectrometry (LC-MS/MS). hMPV RNA (250 mg) was extracted from highly purified rhMPV virions using an RNeasy Mini kit (Qiagen) and purified twice with RiboMinus Eukaryote System v2 kit (Thermo Fisher). To examine the purify of virion RNA, oligo d(T) was used for reverse transcription, followed by qPCR for quantification for β-actin and viral N and G mRNAs. Virion RNA which was free of contamination of host RNA and viral mRNAs was used for LC-MS/MS, m⁶A antibody pulldown assay, and m⁶A-seq. Purified RNA was digested and subjected to quantitative analysis of m⁶A level using LC-MS/MS as previously described.

Colorimetric quantification of viral m⁶A methylation. Virion RNA was extracted from sucrose gradient ultracentrifugation-purified wild type and mutant rhMPVs. Total m⁶A content on virion RNA was quantified by m⁶A RNA Methylation Assay Kit (Abcam, ab185912). Briefly, m⁶A was detected using a specific capture anti-m⁶A antibody and then quantified colorimetrically by reading the absorbance at 450 nm. A standard curve was generated using known m⁶A methylated RNA (range from 0.02 to 1 ng of m⁶A) as a positive control. The m⁶A content was calculated from each RNA samples based on their OD450 values. The percent change was calculated by dividing m⁶A contents in viral RNA from the treated group by those from the control group.

Gene Ontogeny (GO) analysis. GO analysis was performed using the online analysis software metascape www.metascape.org (53).

siRNA and plasmid transfection. siRNAs against METTL3, METTL14, FTO, ALKBH5, YTHDF1, YTHDF2, YTHDF3 or non-targeting AllStars negative control siRNA were purchased from Qiagen. All siRNA and plasmid transfections were performed using the Lipofectamine 3000 transfection reagent (Thermo-Fisher) according to the manufacturer's instructions. Briefly, ninety percent confluent A549 cells in 12-well plates were transfected with 1 μg of plasmid or 30 pmol of siRNA and 24 hours later infected with hMPV. At 12, 18, 24 and 48 hours post infection cells were lysed in RIPA buffer (Abcam) on ice and collected for Western blot.

Antibodies and Western blotting. The antibodies used in this study were anti-YTHDF1 (Proteintech, Rosemont, IL), anti-YTHDF2 (Abcam, Cambridge, MA), anti-YTHDF3 (Abcam), anti-METTL3 (Proteintech), anti-METTL 14 (Abcam), anti-ALKBH5 (Sigma-Aldrich), anti-FTO (Abcam), anti-hMPV serum (prepared in cotton rats), anti-hMPV N antibody (US Biological), anti-RIG-I (Abcam, ab180675), anti-MDA5 (Abcam), anti-FLAG (Sigma-Aldrich), anti-Actin (Abcam), and anti-HA antibody(Abcam). Cells were harvested and lysed in RIPA buffer (Abcam) supplemented with protease inhibitor cocktail (Sigma-Aldrich). Western blotting was performed as described. Actin was used as a loading control.

Immunofluorescence analysis and confocal microscopy. Mock or hMPV-infected cells were fixed in acetone and methanol at the ratio of 1:1 for 30 min, and blocked with goat serum (Sigma-Aldrich, G0923). Slides were stained with all primary antibodies (1:100), washed 3 times with PBS, and stained with conjugated Alexa Fluor secondary antibodies Alexa Fluor 488/594 (Thermo-Fisher; 1:300), and mounted with SlowFade™ Diamond Antifade Mountant with DAPI (Thermo-Fisher). Imaging was performed on an Olympus FV 1000 confocal microscopy system at The Ohio State University Campus Microscopy & Imaging Facility.

Recovery of rhMPVs from the full-length cDNA clones. rhMPVs or rghMPV (rhMPV expressing GFP) were rescued using a reverse genetics system as described previously (50, 54). Briefly, T25 flasks of BHK-SR19-T7 cells (kindly provided by Apath LLC), which stably express T7 RNA polymerase, were transfected with 3.75 μg of plasmid phMPV, 3.0 μg of pCITE-N, 1.5 μg of pCITE-P, 1.5 μg of pCITE-L, and 1.5 μg of pCITE-M2-1 using Lipofectamine 2000 (Life Technologies). At day 6 post-transfection, the cells were harvested using cell scrapers and were co-cultured with Vero-E6 cells at 50-60% confluence. When extensive cytopathic effects (CPE) were observed, the cells were subjected to three freeze-thaw cycles in the presence of 10% trehalose, followed by centrifugation at 3,000×g for 10 min. The supernatant was subsequently used to infect new Vero E6 cells. The successful recovery of the rhMPVs was confirmed by methylcellulose overlay plaque assay, immunostaining, and reverse transcription (RT)-PCR.

Immunostaining plaque assay. Vero E6 cells were seeded in 24-well plates, infected with serial dilutions of rhMPV, and overlayed with methylcellulose. At day 5 postinfection, cells were fixed with 10% neutral buffered formaldehyde at room temperature for 30 min and then the mixture of overlay and formaldehyde was removed. Cells were permeabilized in phosphate-buffered saline (PBS) containing 0.4% Triton X-100 at room temperature for 10 min and blocked at 37° C. for 1 h using 1% bovine serum albumin (BSA) in PBS. The cells were then incubated with anti-hMPV N-protein primary monoclonal antibody (Millipore, Billerica, MA) at a dilution of 1:2,000 overnight at 4° C., followed by incubation with horseradish peroxidase (HRP)-labeled rabbit anti-mouse secondary antibody (Thermo Scientific, Waltham, MA) at a dilution of 1:5,000. After incubation with 3-amino-9-ethylcarbazole (AEC) chromogen substrate (Sigma, St. Louis, MO), positive cells were visualized under a microscope. The viral titer was calculated as the number of PFU per ml.

Viral replication kinetics in A549 cells. Confluent A549 cells or knockout cells in 24-well plates were infected with parental rhMPV or rhMPV mutant at an MOI of 1.0 or 5.0. After 1 h of adsorption, the inoculum was removed and the cells were washed three times with PBS. Fresh DMEM (supplemented with 1% FBS) was added and the infected cells were incubated at 37° C. At different time points post-infection, the supernatant and cells were harvested by three freeze-thaw cycles, followed by centrifugation at 1,500×g at room temperature for 15 min. The virus titer was determined by an immunostaining assay in Vero E6 cells.

Quantification of viral genome, antigenome, and mRNA by real-time RT-PCR. Ninety percent confluent A549 cells were infected with each rhMPV mutant at an MOI of 1.0 or 5.0. At indicated time points, total RNA was isolated from virus-infected cells using the TRIzol reagent (Life Technologies). Poly(A)-containing viral mRNA was isolated from total RNA using a Dynabeads mRNA isolation kit (Life Technologies) according to the manufacturer's recommendations. The first strand of DNA was generated from genomic and antigenomic RNA with primers targeting leader and trailer sequence, respectively, and real-time PCR was performed in TB-Green premix Ex Taq™ (TaKaRa, Japan) with the primer pairs located on N and L gene, respectively. A cDNA pool was generated from total RNA with Oligo (dT)₂₃ (SEQ ID NO: 23) (Sigma-Aldrich), and hMPV N and G-mRNA copies were quantified with the primer pairs located on N and G gene, respectively. RNA and mRNA copies of each sample were normalized by respective mRNA copies of human GAPDH.

RT-PCR and sequencing. All plasmids, viral mutants and stocks, and virus isolates from the nasal turbinates and lungs of cotton rats were sequenced. Viral RNA was extracted from 100 μl of each recombinant virus using an RNeasy mini kit (Qiagen, Valencia, CA) and total RNA from infected tissue was extracted with TRizol reagent. A 1-kb DNA fragment spanning the hMPV G gene was amplified by RT-PCR. The PCR products were purified and sequenced using a sequencing primer at The Ohio State University Plant Microbe Genetics Facility to confirm the presence of the designed mutations.

Isolation of total viral RNA, virion RNA, and G mRNA. Confluent A549 cells in 150-mm dishes were mock infected or infected with wild-type or mutant rhMPV at an MOI of 0.5. At day 2 postinfection, total RNA was isolated from virus-infected cells using the TRIzol reagent (Life Technologies) and dissolved in RNase-free water. Subsequently, poly(A)-containing RNA was isolated from total RNA using a Dynabeads mRNA Direct™ kit (Life Technologies) according to the manufacturer's recommendations. Finally, hMPV G mRNA was isolated by Dynabeads MyOne™ Streptavidin C1 (ThermoFisher Scientific) conjugated with poly T-tailed G gene specific primer. Virion RNA was extracted from sucrose-gradient purified virions of rhMPV or rhMPV mutant. HMPV genome, antigenome, and G mRNA were quantified by real-time RT-PCR.

[³⁵S]-methionine metabolic labeling. A549 cells were transfected with siRNA against METTL3 and METTL14 or control siRNA. After 24 h, cells were incubated in methionine- and cysteine-free media for 1 h, and 50 μCi of [³⁵S]-methionine was added. At indicated time points, cells were washed with PBS and disrupted in lysis buffer. Cell lysates were resolved on SDS-PAGE and exposed to film. Quantification of [³⁵S]-labeled proteins was performed using ImageJ software. 5 μl of each protein sample was used for measuring total [³⁵S] incorporation by scintillation counting (Beckman).

MeRIP assay. MeRIP assay was carried out using a procedure provided by Millipore Magna MeRIP™ m6A kit (Catalog No. 17-10499). Magnetic Beads A/G blend (25 μl) was washed and incubated with anti-m⁶A antibody (5 μl) at room temperature for 30 min and washed three times to remove any unbound antibody. Total RNA (15 μg) was extracted from rhMPV or m⁶A deficient rhMPV-infected A549 cells. The RNA samples were treated at 85° C. for 5 min and chilled on ice immediately, and incubated with m⁶A antibody-associated beads at 4° C. for 2 h with rotation. The RNA-associated magnetic beads were then washed for 3 times. Total RNA was extracted from beads by TRizol reagent and was quantified by real-time RT-PCR using primers annealing to hMPV antigenome, genome, and G mRNA.

Measurement of interferon in virus-infected or RNA-transfected cells. For virus-infection, A549 cells or THP-1 cells infected by rhMPV or hMPV mutant at MOI of 1.0 or 4.0, cell supernatants were harvested at 16, 24, and 48 h post-infection and IFN-α and -β concentrations were determined by commercial enzyme-linked immunosorbent assays (ELISA) according to the manufacturer's instructions (PBL, Piscataway, NJ). A known concentration of human IFN-α and -β was used to generate the standard curve. Prior to RNA transfection, viral RNA was treated with or without calf intestinal alkaline phosphatase (CIP; Promega) at the dose of 10⁷ copies/10 U for 30 min at 37° C. After inactivation of CIP at 65° C. for 15 min, viral RNA was further purified by TRIzol reagent and quantified by real-time RT-PCR. A549 cells or A549-Dual cell lines in 24-well plates were transfected with CIP-treated or untreated viral RNA by Lipofectamine 3000. At 24 and 48 hours post-transfection, culture medium was harvested for IFN-β quantification by ELISA.

Immunoprecipitation assay of RIG-I and virion RNA. Confluent six-well-plates of A549 cells were transfected with 2 μg of plasmid pEF-BOS-RIG-I-Flag (kindly provided by Dr. Jacob Yount). At 24 h post-transfection, cells were lysed in lysis buffer (Abcam, ab152163). Cell lysates were harvested after centrifugation at 13,000×g for 10 min and incubated with Anti-FLAG® M2 magnetic beads (Sigma-Aldrich, M8823) at room temperature for 80 min. The mixture was then divided into 13 aliquots (150 μl/tube), and 12 aliquots were incubated with 2×10⁸ copies of virion RNA (with or without CIP treatment) or 2×10⁹ copies of hMPV mRNA respectively at 37° C. for 1 h. Beads associated RNA:protein complex were washed in lysis buffer for three times, and total RNA was extracted from beads by TRizol reagent and quantified by real-time RT-PCR. The 13^{th} aliquot was washed and subjected to Western blot.

RIG-I pull-down assay. 10⁹ copies of virion RNA with or without CIP treatment was biotinylated with Pierce™ RNA 3′ End Biotinylation Kit (Thermo Fisher Scientific) according to the product instruction. Purified 3′ end biotinylated RNA was incubated with MyOne™ Streptavidin C1 beads (Thermo Fisher Scientific) in the presence of RNase inhibitor at room temperature for 30 min with rotation. RNA-associated beads were then washed three times and incubated with 50 μl of A549 cell lysate containing overexpressed RIG-I and 1 unit of RNase inhibitor at room temperature for 1 h with rotation. Beads were then washed for 3 times and subjected to SDS-PAGE. The pull-down RIG-I protein on Streptavidin beads were detected by Western blot using anti-RIG-I antibody. For control, mixture of cell lysate and RNA-associated beads were loaded as input.

Limited trypsin digestion of RIG-I. Recombinant human RIG-I protein was purified from HEK-293T cells transfected with a plasmid encoding Flag-tagged RIG-I (pEF-BOS-RIG-I-Flag). The concentration of RIG-I protein was measured by Bradford assay. Recombinant RIG-I (50 nM) was incubated with 2×10⁷ copies of virion RNA of wild type or mutant hMPV in 30 μL MOPS buffered reaction system (10 mM MOPS pH 7.4, 1 mM DTT, 1 mM MgCl₂, 0.002% Tween20) in the presence of RNase inhibitor and AMP-PNP (2 mM). The reaction mixtures were incubated at 37° C. for 30 min to permit RIG-I:RNA complex formation and mixed with 10 μL of tosylsulfonyl phenylalanyl chloromethyl ketone (TPCK)-trypsin (2.5 ng/μL) and incubated at room temperature. At indicated time points (0 to 120 min), 10 μL was removed, mixed with 5×SDS-PAGE loading dye, and boiled for Western blot probed with an anti-RIG-I helicase antibody (Abcam). Poly(I:C) (2-10 pg) was used as a positive control. For the competition assay, wild type and rhMPV-G1-14 virion RNA were diluted to 2×10⁶ copies/μL and different ratios of these two RNAs were mixed (10:0, 7.5:2.5, 5:5, 2.5:7.5, and 0:10), and were incubated with purified RIG-I and AMP-PNP and the conformation of RIG-I was examined by limited trypsin digestion, as described above.

Interferon response of rhMPV and mutants in cotton rats. Six-week-old specific-pathogen-free (SPF) female cotton rats (Envigo, Indianapolis, IN) were inoculated intranasally with 100 μl of PBS or PBS containing 2.0×10⁵ PFU of rhMPV-G8-14, rhMPV-G1-14 or rhMPV. Each group contains 5 cotton rats. Forty-eight hours post-inoculation, cotton rats were sacrificed and 1 ml of PBS was injected into the right lung of each cotton rats. Approximately 1 ml of bronchoalveolar lavage (BAL) was collected for IFN-β bioactivity assay on CCRT cells. Briefly, CCRT cells were cultured in 96-well plates with 100 μl of DMEM medium supplemented with 2% FBS. BAL supernatant (250 μl) was mixed with the same volume of DMEM containing 0.1 M HCl, incubated at room temperature for 2 h to destroy type II interferon, and neutralized with 27.8 μl of DMEM containing 7.5% NaHCO₃. The treated BAL mixture was 2-fold serially diluted (1:2-1:128) and added to CCRT cell culture medium in duplicate in a volume of 100 μl/well. A known concentration of human IFN-β was 2-fold serially diluted (250 U-7.8 U/ml) and used to generate a standard curve. Cells were incubated at 37° C. in 5% CO₂ for another 24 h and infected by 10⁴ PFU of recombinant vesicular stomatitis virus expressing GFP reporter (rVSV-GFP) per well. GFP positive cells were observed under UV microscope at 24 h post-infection. The IFN-β concentration of each BAL sample was calculated according to the highest dilution of samples and the lowest concentration of standard human IFN-β which inhibited rVSV-GFP replication therefore GFP expression.

Replication and pathogenesis of rhMPV in cotton rats. Twenty-five 6-week-old female SPF cotton rats (Envigo, Indianapolis, IN) were randomly divided into 5 groups (5 cotton rats per group). Prior to virus inoculation, the cotton rats were anesthetized with isoflurane. The cotton rats in groups 1-4 were intranasally inoculated with 2.0×10 PFU of rhMPV, rhMPV-G1-2, rhMPV-G8-9, and rhMPV-G1-14. The cotton rats in group 5 were mock infected with 100 μl of PBS and served as uninfected controls. Each cotton rat was inoculated intranasally with a volume of 100 μl. After inoculation, the animals were evaluated on a daily basis for any clinical signs. At day 4 postinfection, the cotton rats were sacrificed, and lungs and nasal turbinates were collected for both virus isolation and histological analysis.

Immunogenicity of rhMPV in cotton rats. For the immunogenicity study, thirty 4-week-old cotton rats (Envigo) were randomly divided into four groups (5 cotton rats per group). Cotton rats in groups 1 were mock-infected with PBS and served as uninfected unchallenged control. Cotton rats in groups 2-5 were intranasally inoculated with 2.0×10⁵ PFU of rhMPV, rhMPV-G1-2, rhMPV-G8-9 and rhMPV-G1-14, respectively. Cotton rats in groups 4 were mock-infected with PBS and served as uninfected challenged control. After immunization, the cotton rats were evaluated daily for any possible abnormal reaction and blood samples were collected from each cotton rat weekly by facial vein retro-orbital bleeding, and serum was used for detection of neutralizing antibodies. At 4 weeks post-immunization, the cotton rats in groups 2 to 6 were challenged with 2.0×10⁵ PFU of parental rhMPV via intranasal route, and evaluated twice daily for the presence of any clinical symptoms. At 4 days post-challenge, all cotton rats were euthanized by CO₂ inhalation, and their lungs and nasal turbinates were collected for virus titration. The immunogenicity of rhMPV mutant was assessed based on their ability to trigger neutralizing antibody, the ability to prevent hMPV replication in lungs and nasal turbinates, and the ability to protect lung from pathological changes.

Genetic stability of rhMPV mutants in cell culture. Confluent Vero-E6 cells in T25 flasks were infected with each rhMPV mutant at an MOI of 0.1. At day 3 post-inoculation, the cell culture supernatant was harvested and used for the next passage in Vero-E6 cells. Using this method, each rhMPV mutant was repeatedly passaged 15 times in Vero-E6 cells. At each passage, the G gene was amplified by RT-PCR and sequenced. At passage 15, the entire genome of each recombinant virus was amplified by RT-PCR and sequenced.

Pulmonary histology. After sacrifice, the right lung of each animal was removed, inflated, and fixed with 4% neutral buffered formaldehyde. Fixed tissues were embedded in paraffin and sectioned at 5 m. Slides were then stained with hematoxylin-eosin (H&E) for the examination of histological changes by light microscopy. Histopathological changes were evaluated based on the extent of interstitial inflammation, edema, and peribronchiolar inflammation.

Determination of viral titer in lung and nasal turbinate. The nasal turbinate and the left lung from each cotton rat were removed, weighed, and homogenized in 1 ml of PBS solution using a Precellys 24 tissue homogenizer (Bertin, MD) following the manufacturer's recommendations. The presence of infectious virus was determined by an immunostaining plaque assay in Vero E6 cells, as described above.

Determination of hMPV-neutralizing antibody. hMPV-specific neutralizing antibody titers were determined using a plaque reduction neutralization assay (55). Briefly, cotton rat sera were collected by retro-orbital bleeding weekly until challenge. The serum samples were heat inactivated at 56° C. for 30 min. Twofold dilutions of the serum samples were mixed with an equal volume of DMEM containing approximately 100 PFU/well rhMPV in a 96-well plate, and the plate was incubated at 37° C. for 1 h with constant rotation. The mixtures were then transferred to confluent Vero-E6 cells in a 24-well plate in triplicate. After 1 h of incubation at 37° C., the virus-serum mixtures were removed and the cells were overlaid with 0.75% methylcellulose in DMEM and incubated for another 5 days before immunostaining plaque titration. The plaques were counted, and 50% plaque reduction titers were calculated as the hMPV-specific neutralizing antibody titers.

Statistical analysis. Quantitative analysis was performed by either densitometric scanning of autoradiographs or by using a phosphorimager (Typhoon; GE Healthcare, Piscataway, NJ) and ImageQuant TL software (GE Healthcare, Piscataway, NJ). Statistical analysis was performed by one-way multiple comparisons using SPSS (version 8.0) statistical analysis software (SPSS Inc., Chicago, IL). A P value of <0.05 was considered statistically significant.

### B. Results

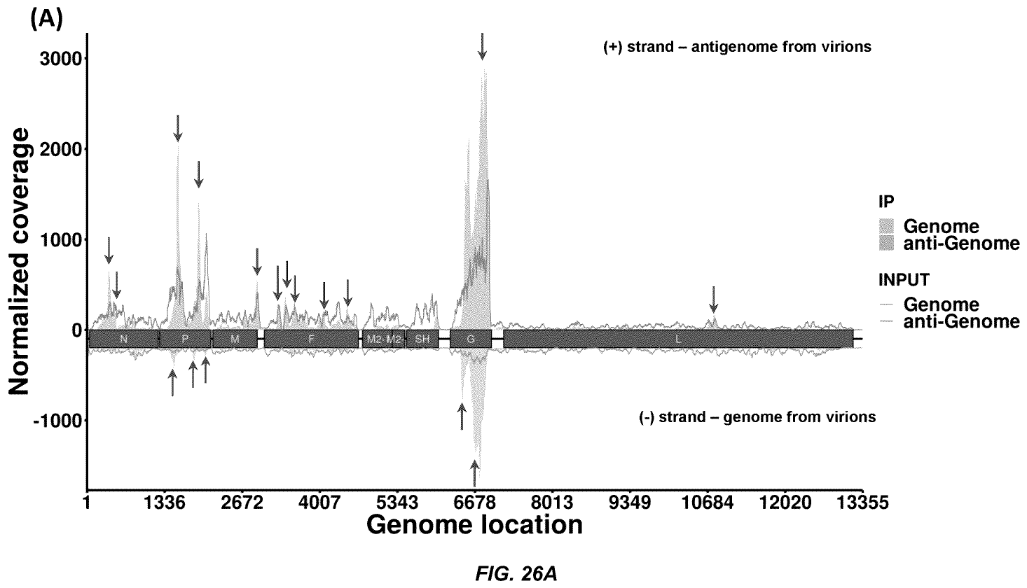

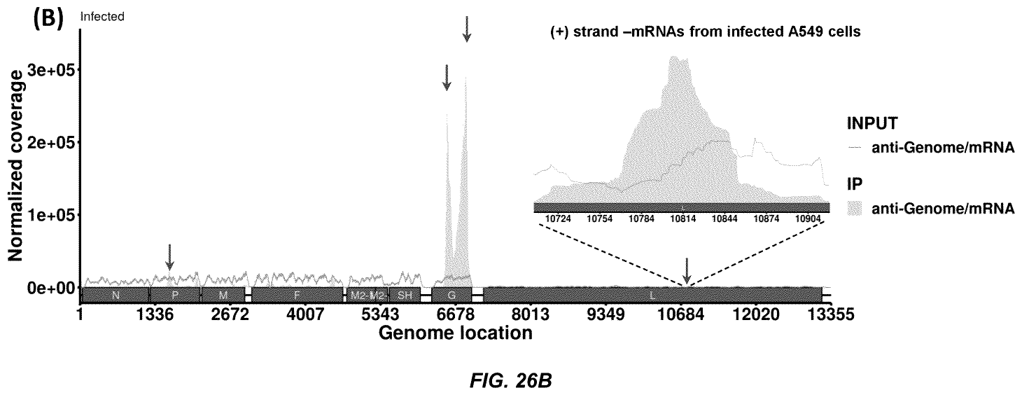

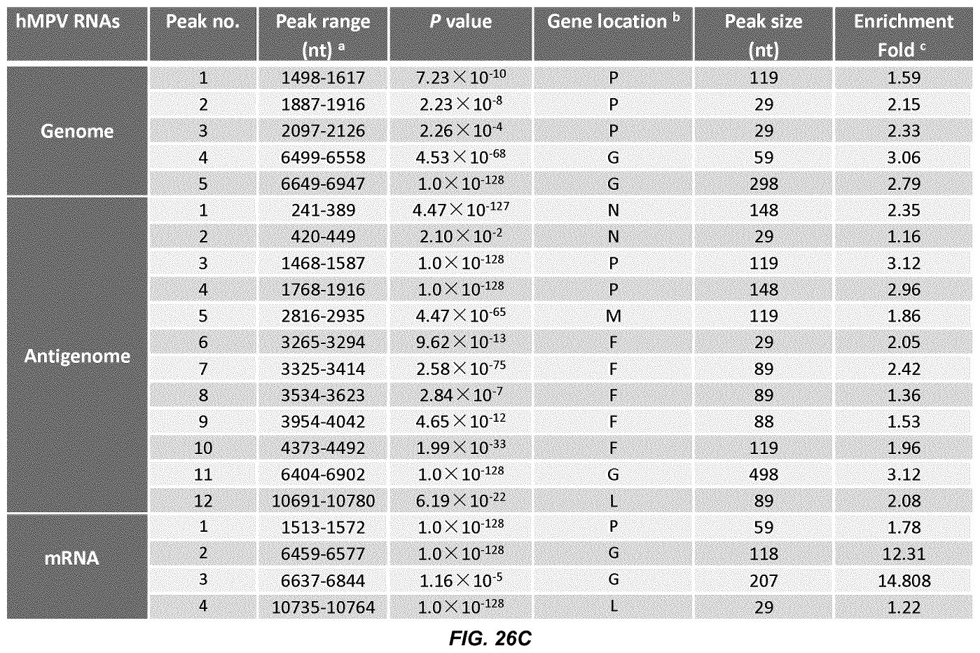

The hMPV genome, antigenome, and mRNAs contain m⁶A modifications. HMPV virions contain its NNS RNA genome of 13,350 nucleotides (subtype A strain NL/1/00, GenBank accession number AF371337). During replication, the viral RNA dependent RNA polymerase copies the negative-sense genomic RNA (vgRNA) to produce an exact, positive-sense full-length complementary RNA (cRNA) antigenome. To determine whether hMPV vgRNA contains m⁶A, hMPV was grown in A549 cells (a lung epithelial cell line) and viral vgRNA was extracted from purified hMPV virions, sonicated, and subjected to m⁶A-specific antibody immunoprecipitation followed by high throughput sequencing (m⁶A-seq). Although it has not been reported for hMPV, previous studies with several other NNS RNA viruses have shown that both the genome and the antigenome can be packaged into virions (56). Interestingly, the inventors found that sequencing reads from m⁶A-seq aligned to both the genome and antigenome, indicating that hMPV virions contain both the genome and the antigenome. To prove this, purified hMPV virions were disrupted by detergent, digested with RNase, and the RNase-resistant viral nucleocapsid (N-RNA complex) was pulled down by hMPV N antibody. Both genome and antigenome were detected in N-RNA complex by real-time RT-PCR (FIG. 45A-D). This demonstrates that both the genome and the antigenome are indeed encapsidated by N protein and packaged into hMPV virions. The m⁶A-seq found m⁶A peaks on both the genome and antigenome. In the genomic RNA, a total of 5 m⁶A peaks were detected spread through the P and G gene regions (FIGS. 26A and C). The largest m⁶A peak was found in the G gene with a log 2 enrichment of 3.06, followed by the next largest peak in the P gene with a log 2 enrichment of 2.33. Interestingly, a total of 12 m⁶A peaks were also found in the antigenome RNA, including regions complementary to the N, P, M, F, G, and L genes (FIGS. 26A and C). Similar to the genome RNA, the strongest m⁶A peaks were found in the G gene with a log 2 enrichment of 3.12. Interestingly, the regions of m⁶A peaks identified in both strands are largely overlapping, despite the genome being complementary to the antigenome. Thus, these results show that both the genome and the antigenome of hMPV are m⁶A modified.

In virus-infected cells, hMPV produces three types of RNAs, genome and antigenome (the replication products) which are neither capped nor polyadenylated, and 8 species of mRNAs (transcription products) which are capped and polyadenylated. The inventors next determined whether hMPV mRNAs were m⁶A modified by performing m⁶A-seq of polyadenylated mRNAs from virus-infected cells. This analysis revealed m⁶A peaks in 3 of the 8 mRNA species, P, G, and L (FIGS. 26B and C). Notably, several m⁶A-modified regions in hMPV mRNAs largely overlapped with those found in the antigenome. Again, the G mRNA has by far the strongest m⁶A enrichments among all the hMPV mRNAs. This result suggests that the host m⁶A machinery methylates positive-strand viral mRNAs and antigenomes similarly.

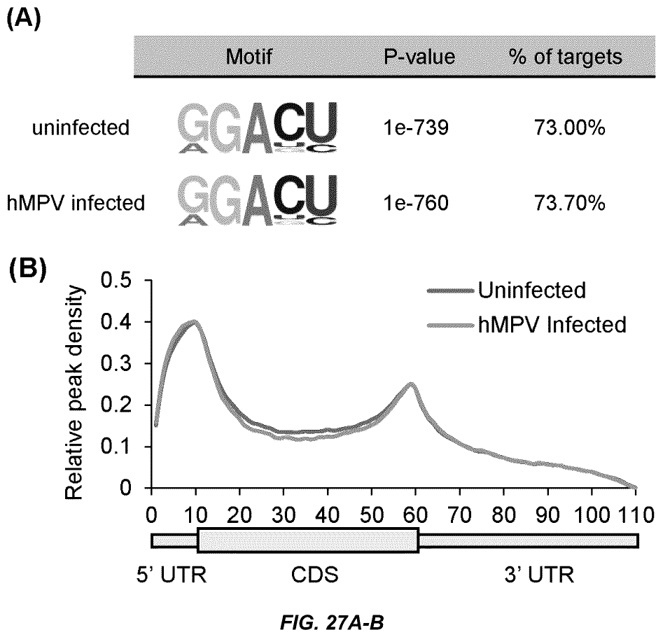

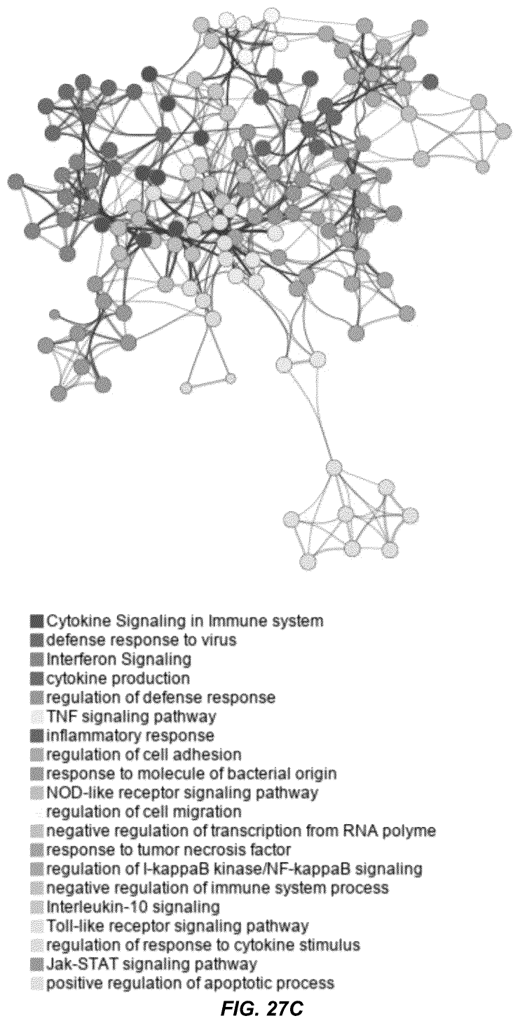

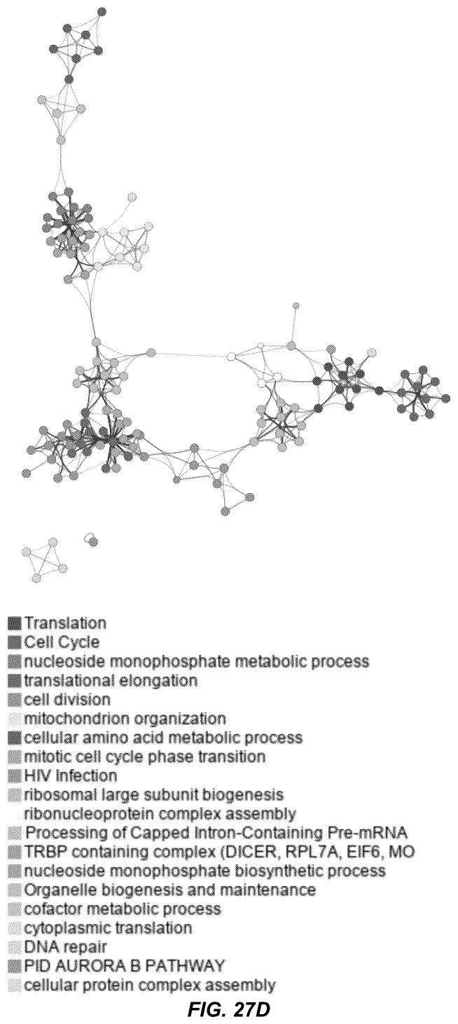

hMPV infection leads to differential expression of interferon-related genes involved in the innate immune response. The inventors next determined whether hMPV infection can alter the abundance and distribution of m⁶A on host transcripts. Total RNA was isolated from hMPV-infected or mock-infected A549 cells, and polyadenylated mRNAs were isolated and subjected to m⁶A-seq. High quality m⁶A peaks were detected in both hMPV-infected and mock-infected samples, as demonstrated by finding the m⁶A consensus sequence GGACU similarly enriched in both sets of samples (FIG. 27A). Metagene analysis showed that hMPV infection slightly increased m⁶A levels in the 5′ and 3′ UTR regions of the host transcriptome, but slightly decreased the m⁶A levels in the coding sequence (CDS) and noncoding sequence (NCS) regions of the host mRNAs (FIG. 27B). Differential methylation analysis using count-based model identified only 21 differentially methylated peaks at FDR<10%, suggesting that hMPV infection leads to little alteration of host epitranscriptome. In contrast, the inventors found a large number of genes differentially expressed in response to hMPV infection. Gene ontology (GO) analysis revealed that the upregulated genes are strikingly enriched in innate host defense transcripts including the cytokine and interferon signaling pathway and inflammatory responses (FIG. 27C). Numerous interferon encoded genes are upregulated including interferon lambda receptor 1, interferon beta 1, interferon lambda 2, interferon lambda 4, and genes involved in Pattern Recognition Receptor (PRR) including RIG-I, MDA5, LPG2, and multiple interferon-stimulated genes (ISGs). In contrast, the downregulated genes are enriched in the cell cycle, metabolism, and translation category (FIG. 27D). These results suggest that hMPV infection significantly alters the host gene expression but has a minimal impact on methylome of cellular mRNAs relevant to host responses to viral infection.

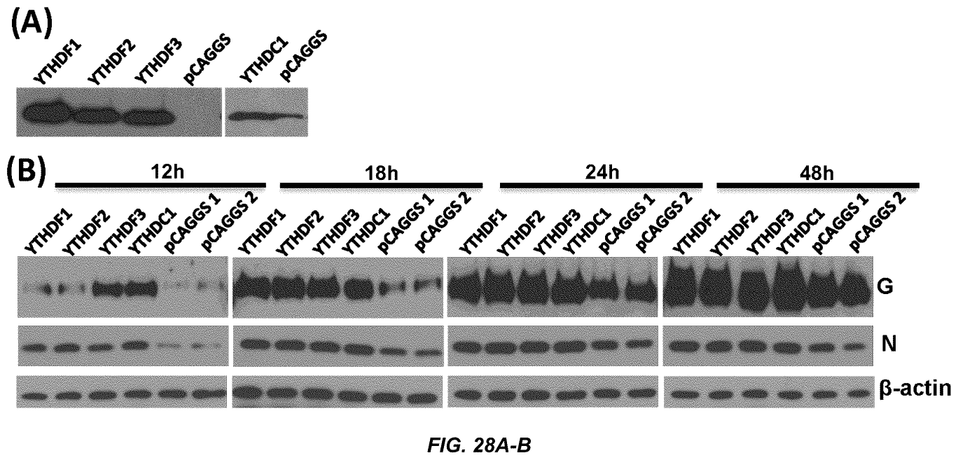

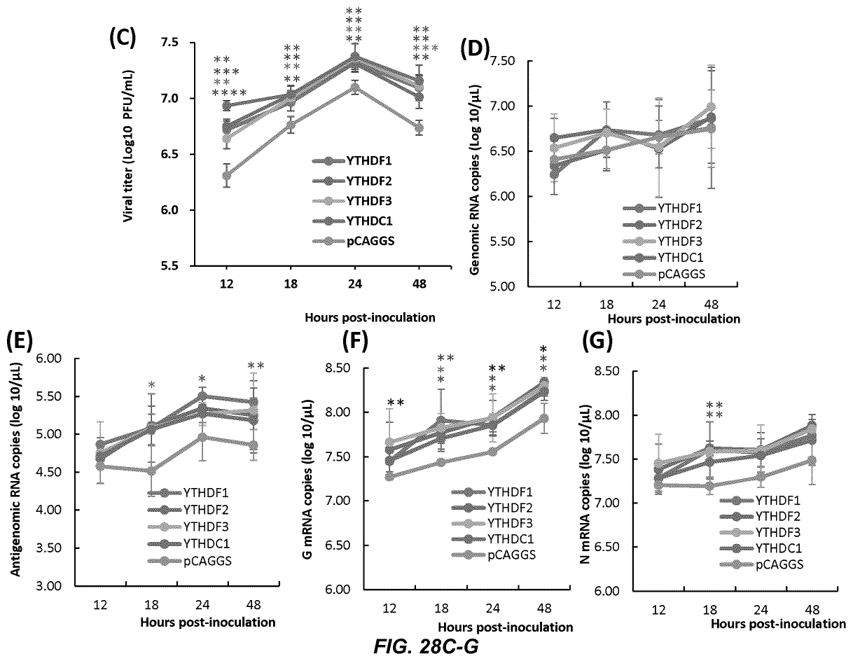

m⁶A reader proteins positively regulate hMPV replication, gene expression, and virus production. The biological functions of m⁶A modification are mediated by m⁶A-binding proteins with a YTH domain located in cytoplasmic (YTHDF1, YTHDF2, and YTHDF3) and nuclear (YTHDC1) compartments (21). The inventors first examined the effects of overexpression of m⁶A reader proteins on hMPV replication and gene expression. Briefly, A549 cells were transfected with plasmids expressing YTHDF1, YTHDF2, YTHDF3 or YTHDC1 (FIG. 28A), followed by infection with hMPV, and the expression of the hMPV G protein (one of the two major surface glycoproteins) and the N protein (the major component of the inner nucleocapsid complex) was measured by Western blot. As shown in FIG. 28B, viral G and N protein expression was significantly increased in A549 cells that transiently overexpressed YTHDF1-3 and YTHDC1, particularly at early time points post-inoculation. Next, the inventors measured the release of infectious virus in a single step growth curve. The hMPV titer was significantly increased in all four overexpressing cell lines at all time points (P<0.01, 0.001, or 0.0001) (FIG. 28C). Furthermore, viral replication intermediate (antigenomic RNA) and transcription products (N and G mRNAs) were significantly increased in overexpressing cell lines in some time points (P<0.05 or 0.01), as quantified by real-time RT-PCR (FIG. 28D-G). Thus, transient overexpression of m⁶A reader proteins promotes hMPV replication and gene expression in A549 cells.

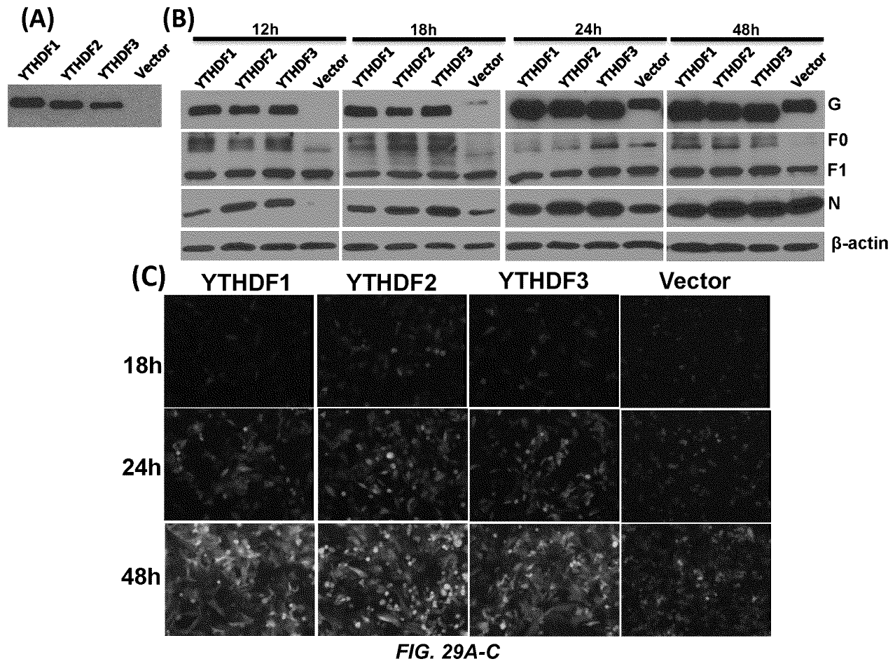

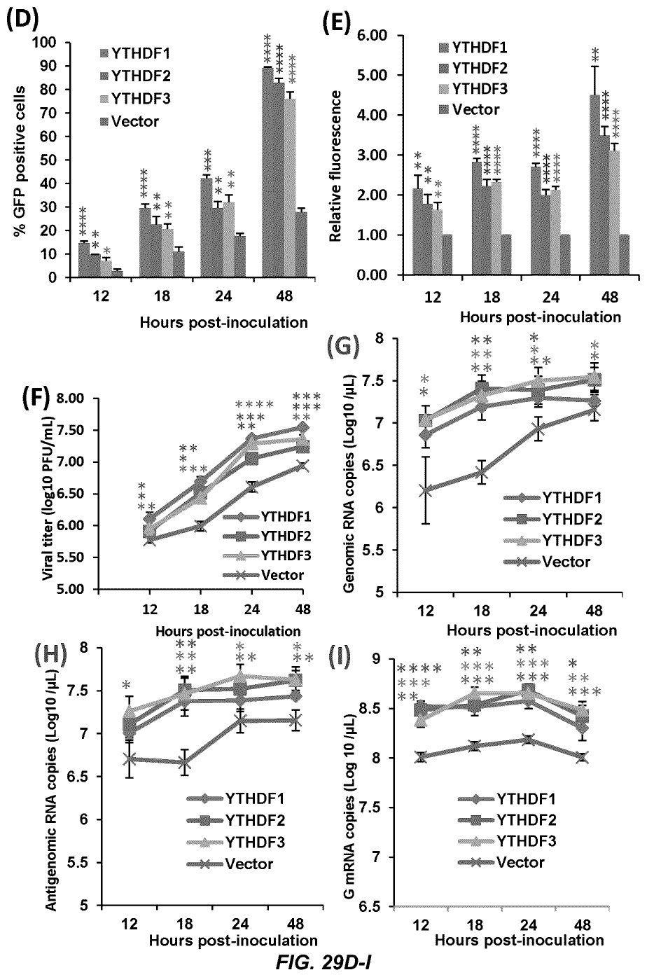

The inventors next examined whether the enhanced hMPV replication and gene expression by m⁶A reader proteins was cell type-specific. To do this, the inventors constructed HeLa cells stably overexpressing YTHDF1, YTHDF2, or YTHDF3 (FIG. 29A). Briefly, HeLa cells were infected with rghMPV (a recombinant hMPV expressing GFP) at an MOI of 0.5, and viral protein expression, RNA synthesis, and virus production were monitored. Similar to hMPV infection in A549 cells, significantly more hMPV F, G, and N proteins were detected in all three YTHDF-overexpressing HeLa cell lines (P<0.05) (FIG. 29B). In addition, significantly stronger GFP expression was observed in HeLa cells overexpressing YTHDF1-3 compared to the lentivirus vector control HeLa cells at 12, 18, 24, and 48 h post-infection (FIG. 29C). Flow cytometry analysis found significantly more GFP-positive cells (FIG. 29D) with stronger GFP intensity (FIG. 29E) in HeLa cells overexpressing m⁶A reader proteins (P<0.05). Finally, a single step growth curve assay showed that the rghMPV titer was significantly increased in all three YTHDF-overexpressing cell lines at 12, 18, 24, and 48 h post-inoculation (FIG. 29F). Thus, YTHDF1-3 promotes hMPV replication and gene expression in HeLa cells. Collectively, these results demonstrate that m⁶A binding proteins promote hMPV genome replication, mRNA transcription, viral protein expression, and progeny virus production in both A549 and HeLa cells.

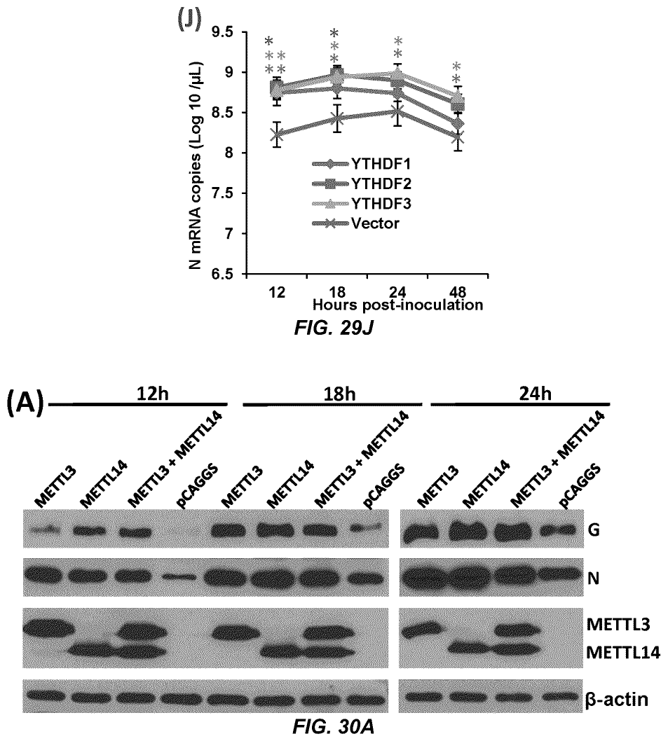

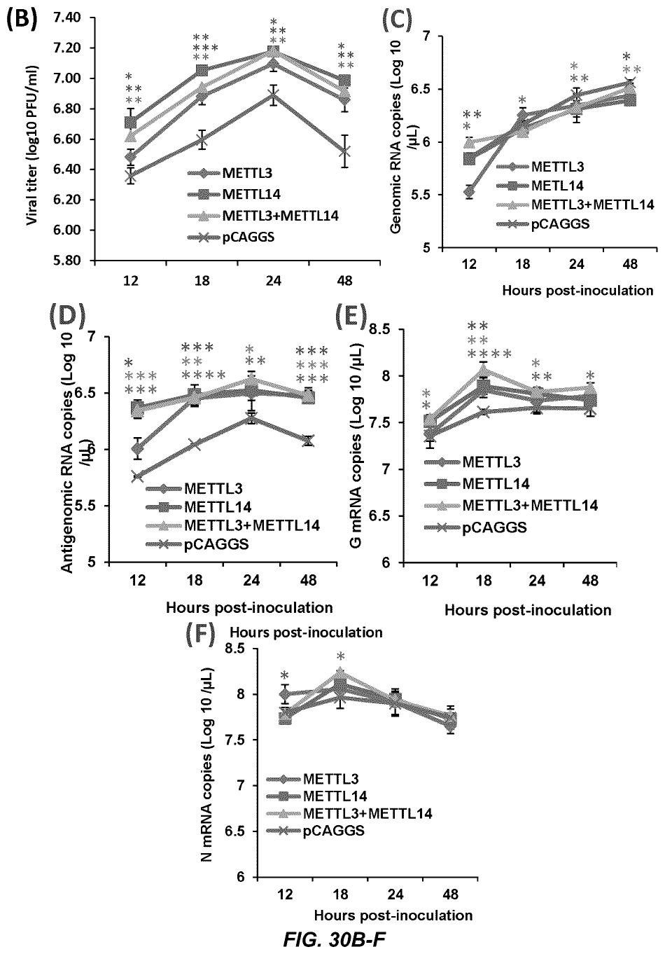

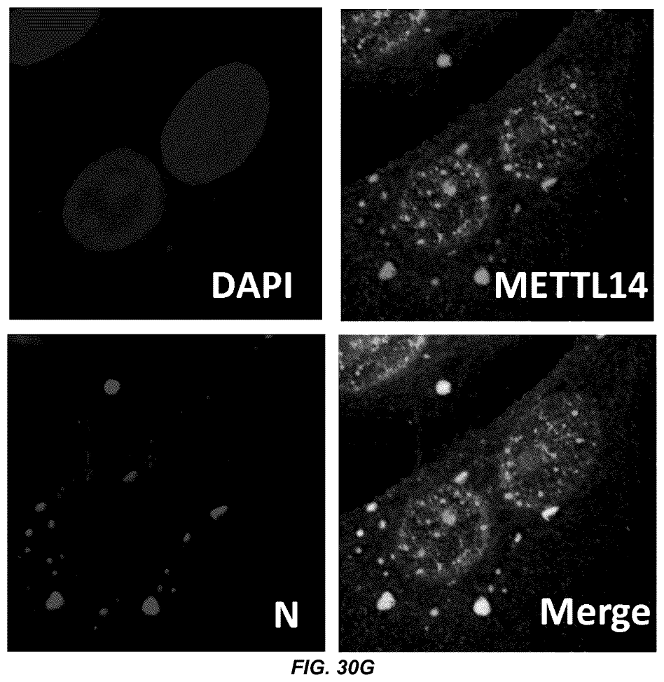

m⁶A writer proteins positively regulate hMPV replication and gene expression. The addition of internal m⁶A on RNA is catalyzed by m⁶A writer proteins composed of two host RNA methyltransferases, METTL3 and METTL14 (25). Thus, the role of the m⁶A writer proteins in hMPV replication and protein expression was examined. A549 cells were transfected with plasmids encoding HA-tagged METTL3, METTL14, or both, followed by rhMPV infection. Western blotting showed that METTL3, METTL14, or both were overexpressed in transfected A549 cells. Interestingly, the expression of hMPV G and N proteins was significantly increased in METTL3 and METTL14 overexpressing A549 cells (FIG. 30A). Consistent with this, the hMPV titer was significantly increased in METTL3 and METTL14 overexpressing A549 cells compared to the vector-transfected cells (FIG. 30B). Quantification of viral RNA in virus-infected cells showed that antigenome synthesis was significantly increased in METTL3 and METTL14 overexpressing A549 cells (FIG. 30D) although genome synthesis did not have a significant increase (FIG. 30C). In addition, G (FIG. 30E) and N (FIG. 30F) mRNA synthesis was significantly increased at some time points.

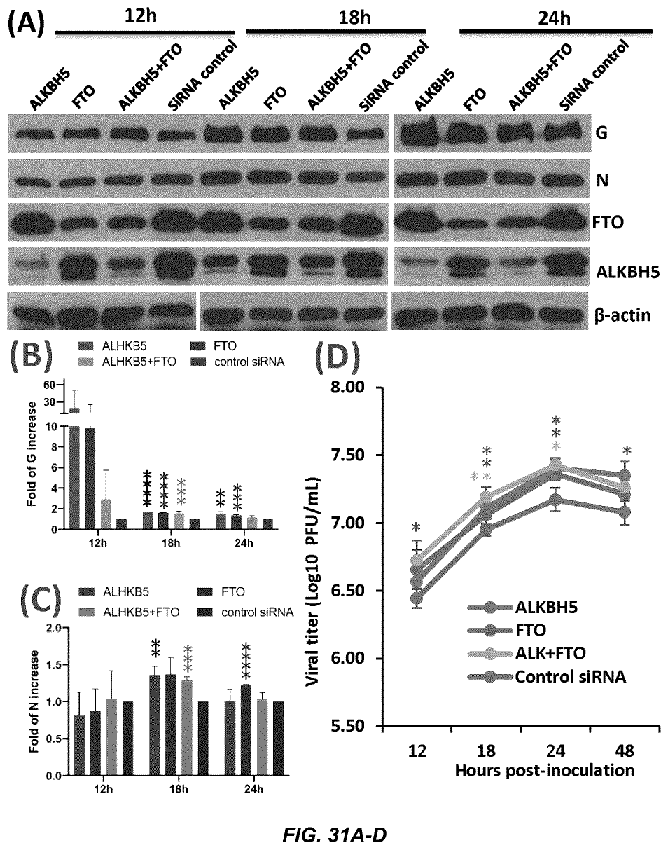

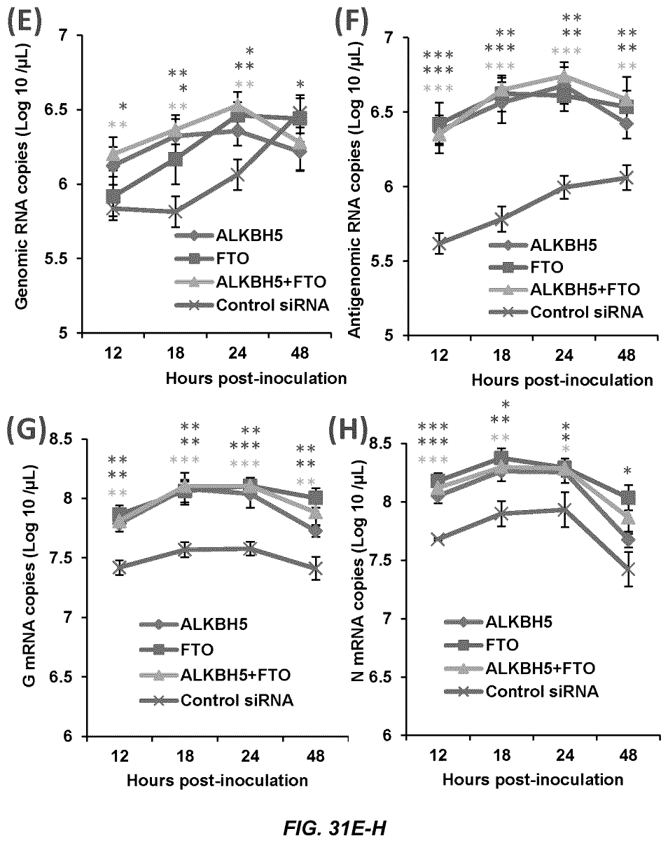

Internal m⁶A modifications are reversible and can be removed by m⁶A eraser proteins, AlkBH5 and FTO (27, 28). The inventors thus examined the effects of knockdown of eraser proteins by transfecting A549 cells with siRNA against AlkBH5 or FTO, or both (FIG. 31A). Knockdown of AlkBH5 and FTO increased viral G and N protein expression, most obviously at 18 hours post-infection. Quantification of Western blot gels from three independent experiment showed that G and N protein expression was significantly increased at 18 h post-infection (FIG. 31B). Viral protein expression was also increased but did not display a significant difference compared to the control siRNA-transfected cells (FIG. 31B) at some time points. In addition, genome (FIG. 31E), antigenome (FIG. 31F), N (FIG. 31H), and G (FIG. 31G) mRNA synthesis was significantly increased in eraser knockdown cells compared to the control siRNA-transfected cells. Taken together, these results demonstrated that modification of hMPV RNA by m⁶A writers enhances hMPV replication and gene expression.

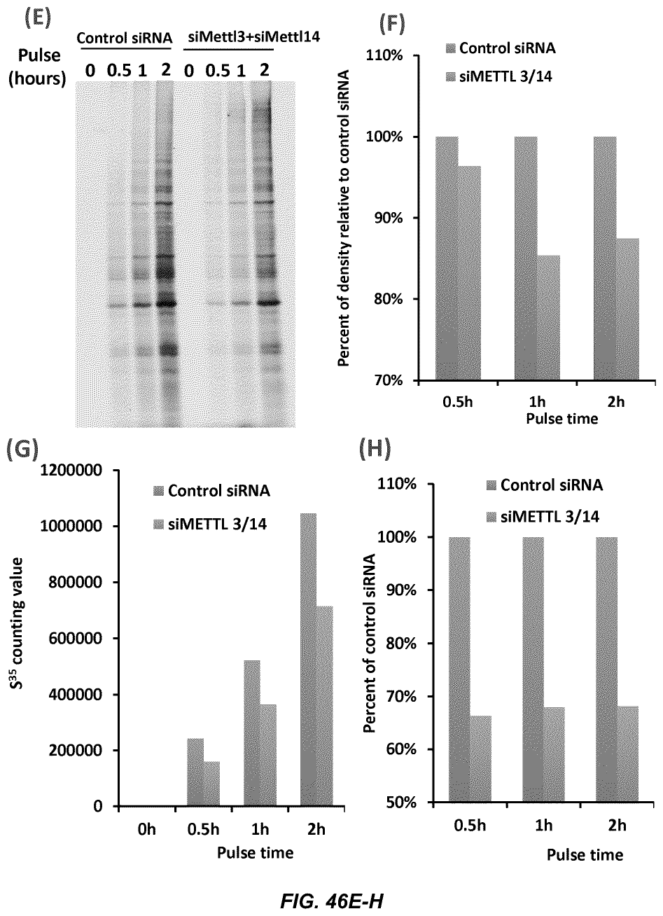

The inventors next examined whether manipulation of m⁶A eraser and writer proteins affect host RNA m⁶A methylation. As shown in FIG. 46A, siRNA knockdown of Mettl3 and Mettl14 led to 27.7% and 20.3% reduction in m⁶A contents in total RNA compared to the RNA from control siRNA-transfected cells. Next, polyadenylated mRNAs were isolated from total RNA. It was found that siRNA knockdown of Mettl3 and Mettl14 led to 68.7% and 62.2% reduction in m⁶A contents in host mRNA (FIG. 46B). Consistent with this finding, overexpression of Mettl3 and Mettl14 led to a 24.3% increase in host RNA m⁶A methylation (FIG. 46C). In addition, overexpression of eraser proteins (FTO and ALKBH5) led to a significant reduction (24.6%) in m⁶A content of host RNA (FIG. 46D). The inventors also found that siRNA knockdown of Mettl3 and Mettl14 reduced host protein translation relative to control siRNA (FIG. 46E-H). Quantification of protein density showed that knockdown of writer protein led to 4%, 14.6%, and 12.5% reduction in host protein translation at 0.5, 1, and 2 h after [S³⁵] labeling (FIG. 46F). Scintillation counting showed that 30-33% reduction in [S³⁵] incorporation (FIG. 46H). These results suggest that the altered viral replication and gene expression is likely due to the changes of both viral and host RNA m⁶A methylation.

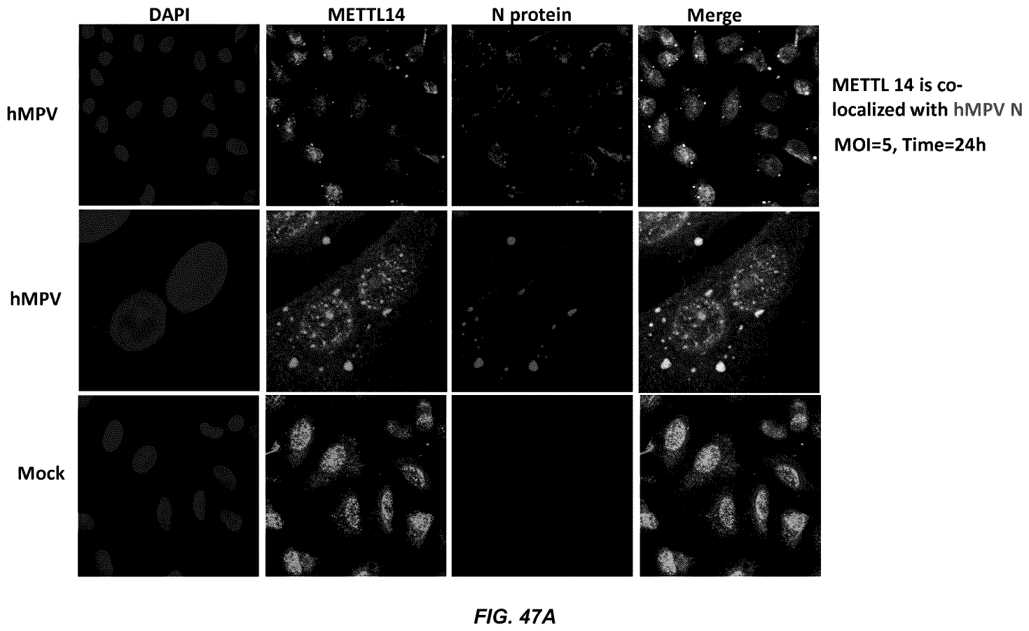

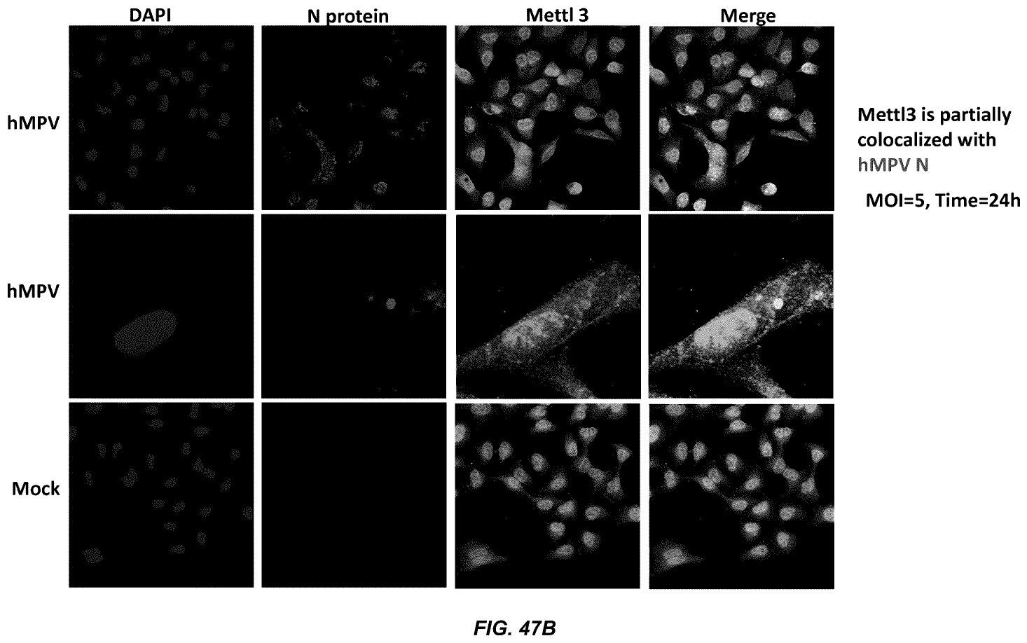

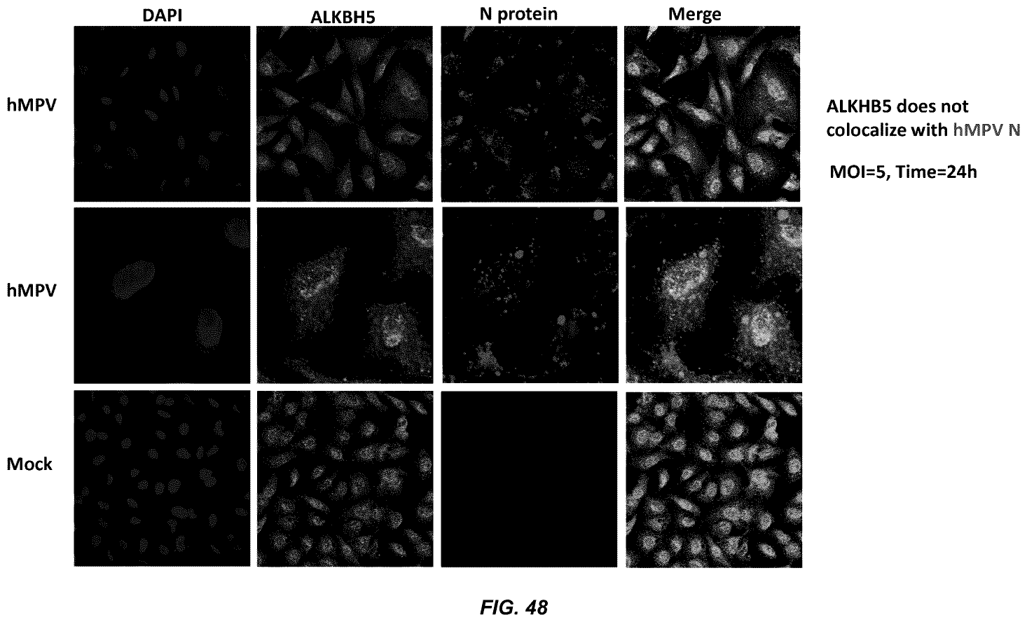

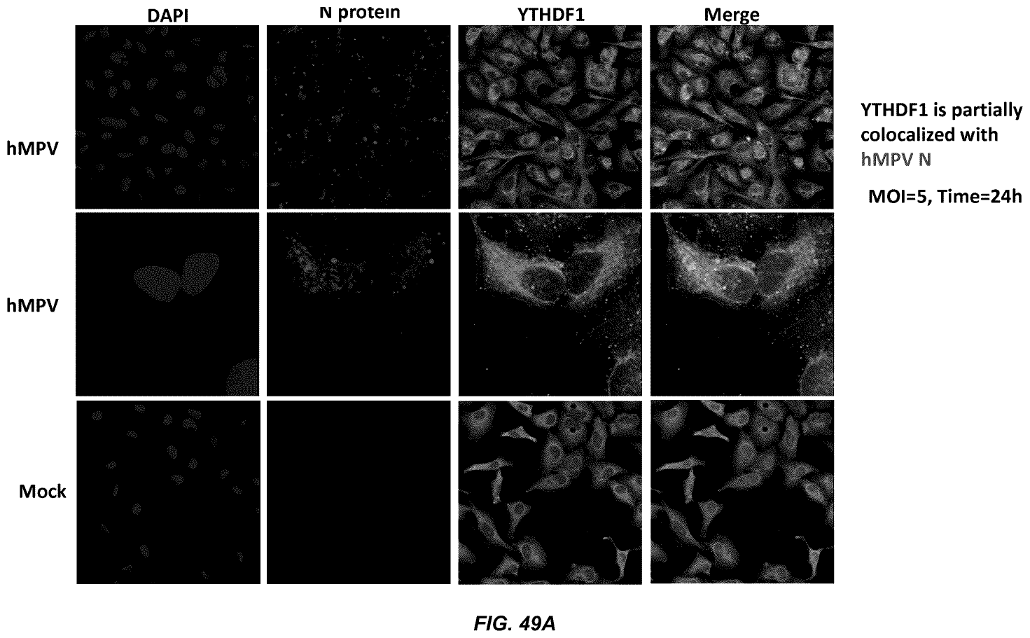

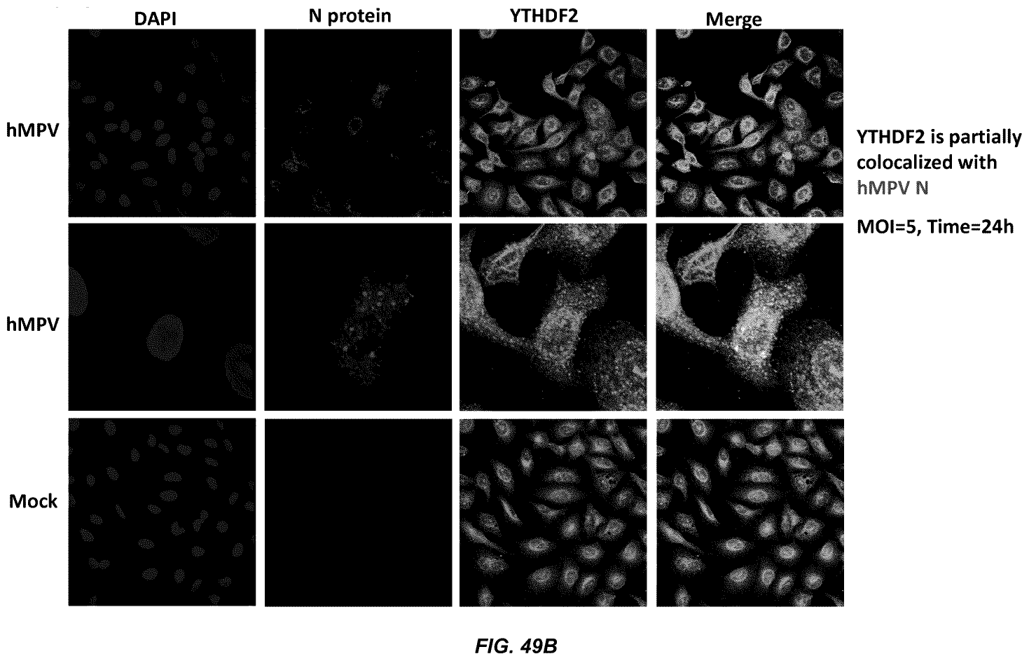

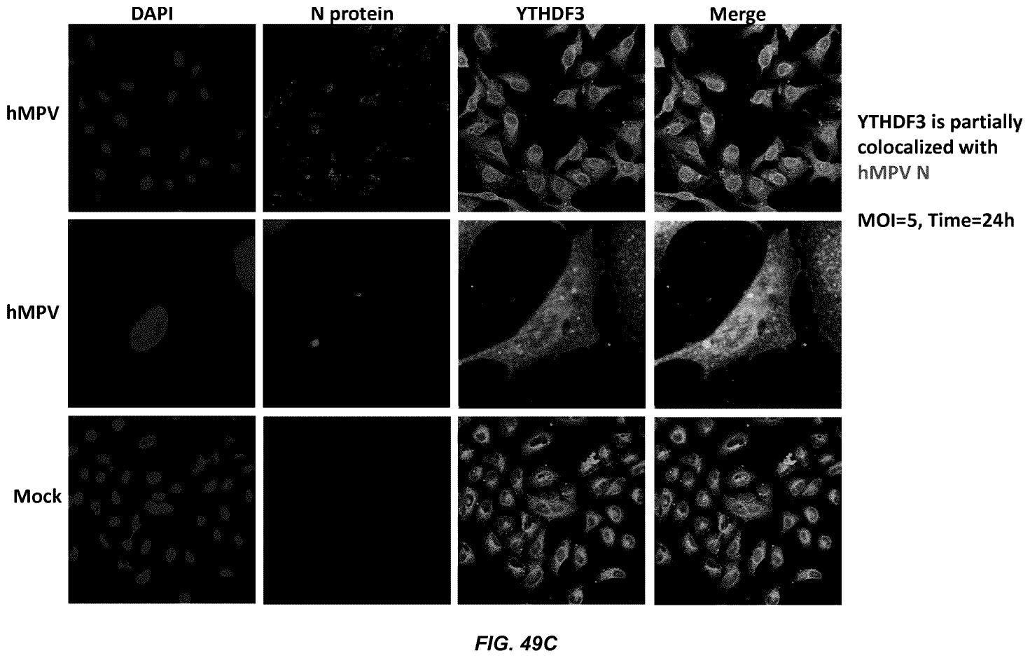

Localization of m⁶A writer, eraser, and reader proteins in hMPV-infected cells. It is generally believed that host RNA methyltransferases, METTL3 and METTL14, are localized in the nucleus (25). The fact that hMPV replicates entirely in the cytoplasm suggested that these proteins may also be present in the cytoplasm. To test this possibility, hMPV-infected A549 cells were stained with antibodies against the hMPV N protein together with individual m⁶A-related proteins, and analyzed by confocal microscopy. The majority of METTL3 and METTL14 proteins were localized in the nucleus (FIG. 30G and FIG. 47). However, a small fraction of these two proteins were detected in the cytoplasm of both mock and hMPV infected cells. This small amount of host RNA methyltransferases may be sufficient to catalyze m⁶A modification of the hMPV RNAs. Interestingly, viral N protein and Mettl14 had a strong co-localization in inclusion bodies, the site where hMPV replication and RNP assembly occurs (FIG. 30G and FIG. 47A). In addition, N protein was partially co-localized with METTL3 (FIG. 47B). However, viral N protein had little co-localization with the eraser proteins (ALKBH5) (FIG. 48). The inventors also examined the localization of m⁶A reader proteins in mock- and hMPV-infected cells. The majority of m⁶A binding proteins (YTHDF1, YTHDF2, and YTHDF3) were found in the cytoplasm of both mock- and hMPV-infected cells (FIG. 49). HMPV infection did not significantly alter the distribution of m⁶A writer and reader protein compared to mock-infected cells. In addition, viral N protein was partially co-localized with all three reader proteins (FIG. 49).

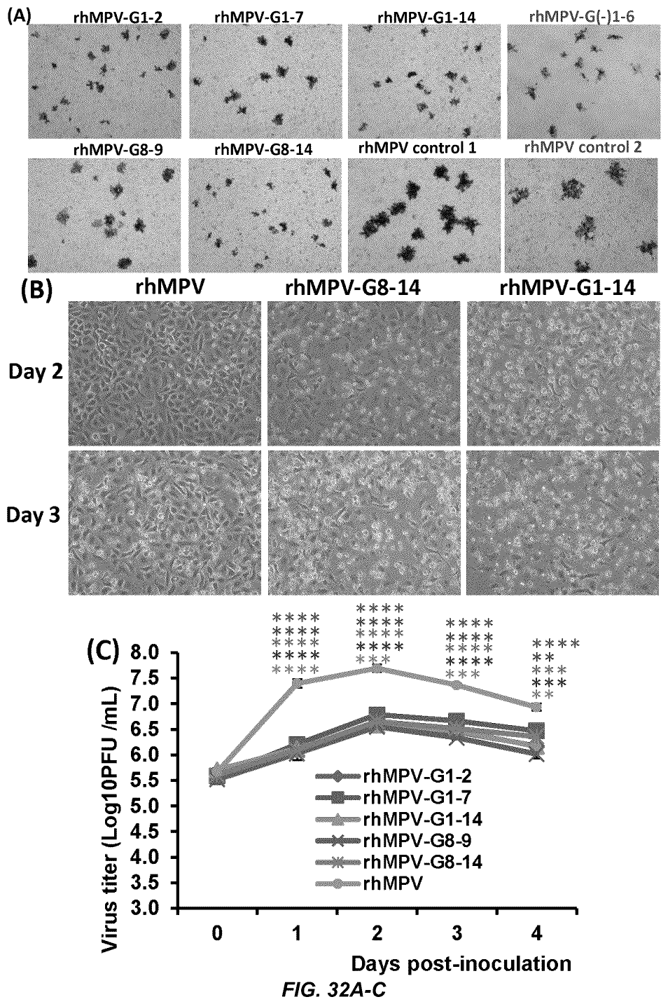

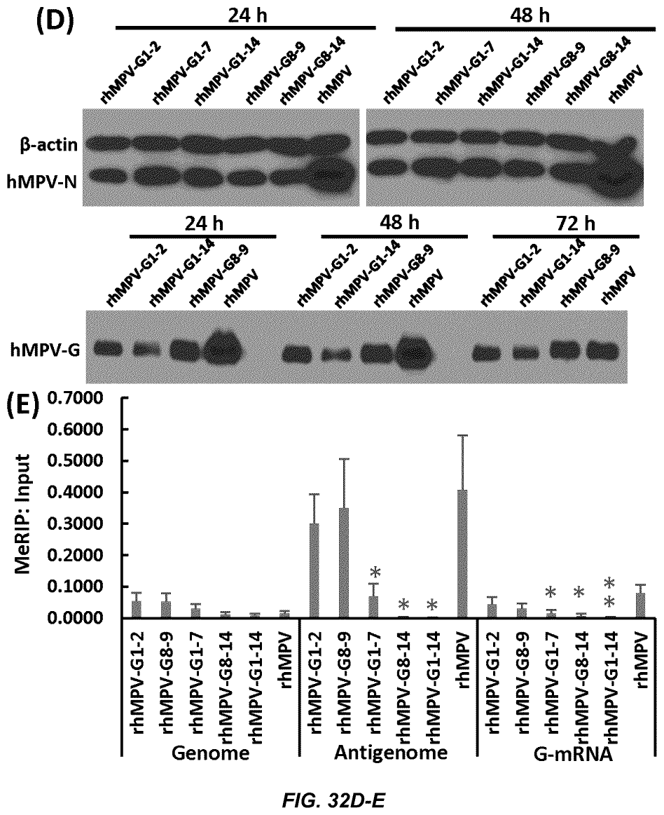

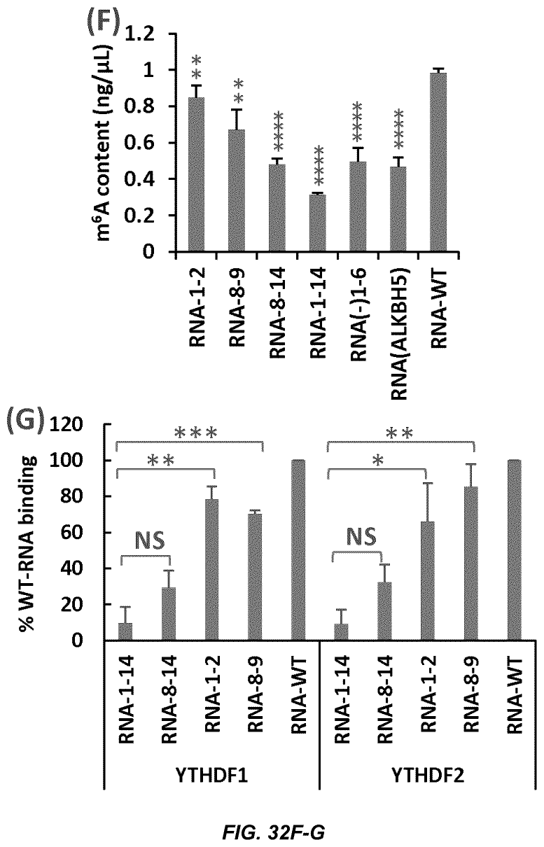

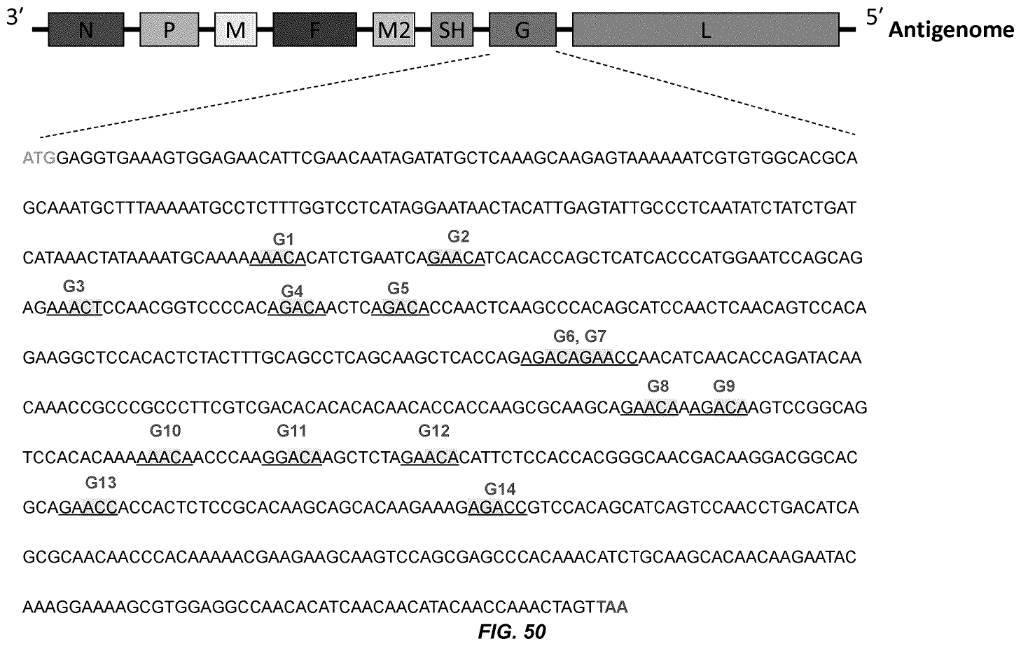

Abrogation of m⁶A sites in the G gene results in attenuation of hMPV in cell culture. Since the m⁶A-seq showed that the G gene has the strongest m⁶A enrichment in the antigenome, genome, and among hMPV mRNAs, the inventors decided to focus on the m⁶A sites in the G gene. In general, m⁶A sites occur at the consensus RRACH motif (R=G or A; H=A, C, or U) (20). From the m⁶A-seq result, the inventors searched for this motif in the G mRNA and identified a total of 14 potential m⁶A sites in the G gene region in the antigenome/G mRNA (FIG. 50) and identified 6 m⁶A sites in G gene in the genome (FIG. 51). The inventors mutated the A or C within the mRNA/antigenome or genome consensus sequence to a T or G in an infectious hMPV cDNA clone, lineage A strain NL/1/00, without changing the amino acids they encode. The inventors avoided changes that would alter the RNA secondary structures or codon usage, as predicted by the M-fold and Genscript software. Using a reverse genetics system, the inventors successfully recovered a total of 5 rhMPVs carrying mutations in m⁶A sites in the G gene region in antigenome/G mRNA: the first two potential m⁶A sites (G1 and G2) were combined to produce rhMPV-G1-2; the first 7 mutations of m⁶A sites (G1 to G7) were combined to produce rhMPV-G1-7; the 8^{th} and 9^{th} m⁶A sites (G8 and G9) were combined to produce rhMPV-G8-9; the last 7 m⁶A sites (G8 to G14) were combined to produce rhMPV-G8-14; and all 14 m⁶A sites were combined to produce rhMPV-G1-14. In addition, the inventors generated one recombinant virus [rhMPV-G(−)1-6] carrying 6 m⁶A site mutations in the G gene in genome. All hMPV mutants were plaque purified. The entire genome of each hMPV mutant was amplified and sequenced. All recombinant hMPVs retained the desired mutation in the G gene, and no additional mutations were found in the genome. The recombinant viruses were detected in Vero-E6 cells by an immunostaining assay using a monoclonal antibody against the N protein. The immunospots formed by these rhMPV mutants were significantly smaller than those formed by the parental rhMPV (FIG. 32A), indicating that m⁶A-deficient rhMPV had defects in viral replication and/or cell-to-cell spread.

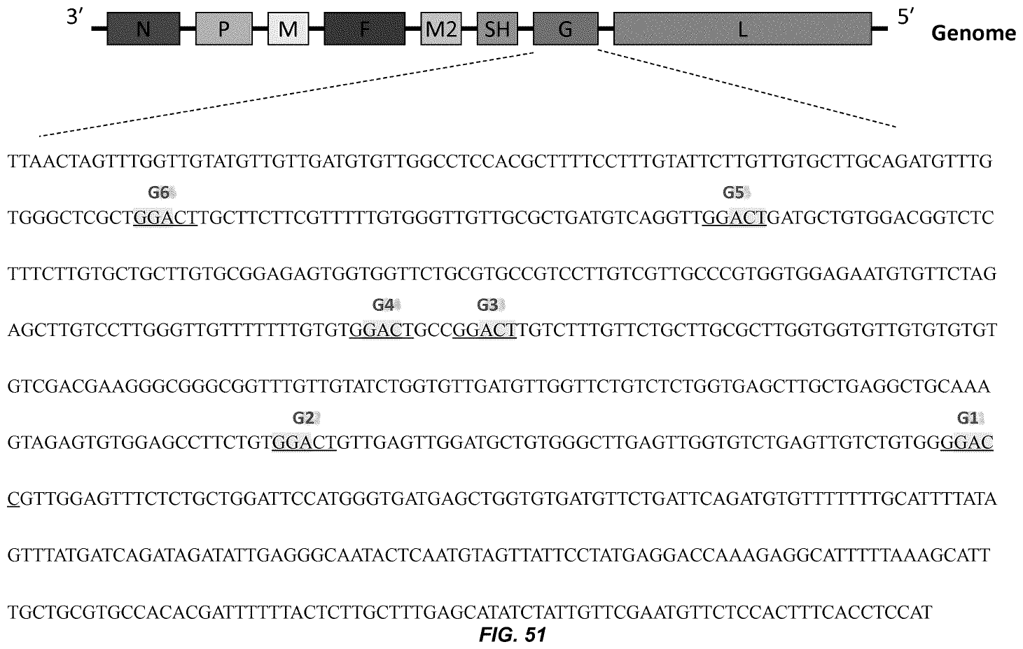

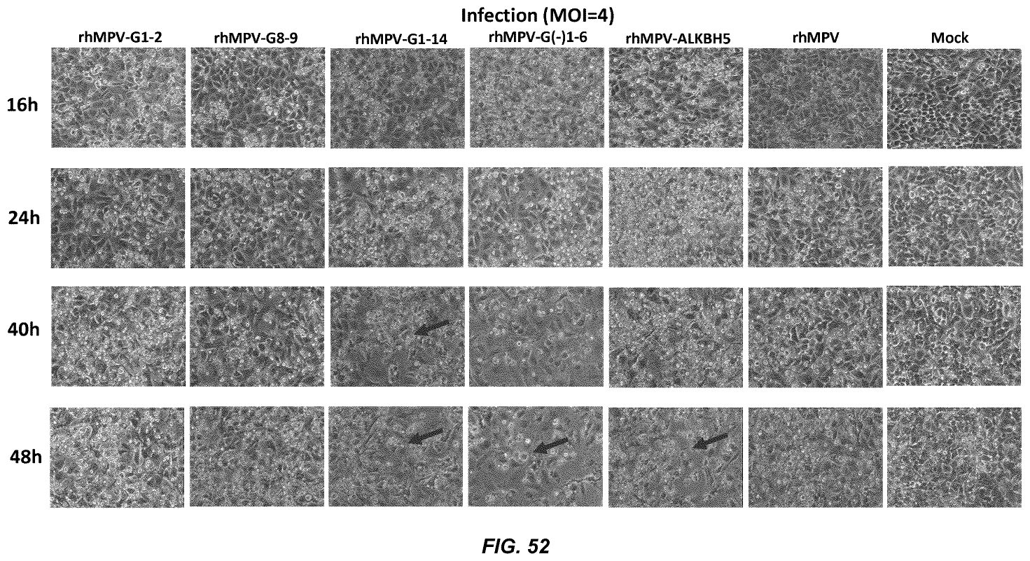

The replication and gene expression of m⁶A-deficient rhMPVs were monitored in A549 cells. The parental hMPV caused extensive cytopathic effects (CPE) at day 3 post-inoculation and most cells were killed by day 4. Interestingly, m⁶A-deficient rhMPV mutants developed CPE earlier in A549 cells. Extensive CPE was observed in hMPV mutants at day 2 post-infection and most cells were killed by day 3 (FIG. 32B and FIG. 52). Among these mutants, rhMPV-G1-14 caused the most obvious cell death at 40 h post-infection (FIG. 52). All m⁶A deficient rhMPVs had defects in replication kinetics in A549 cells compared to rhMPV (FIG. 32C). The parental rhMPV reached a peak titer of 10^{7.7} PFU/ml at day 2 post-inoculation whereas m⁶A-deficient rhMPV reached a peak titer of 10^{6.5}˜10^{6.7} PFU/ml. Next, the expression of viral proteins in virus-infected cells was analyzed by Western blot. Significantly less viral N and G proteins were detected in the m⁶A-deficient rhMPVs-infected cells compared to the parental rhMPV-infected cells (FIG. 32D). Thus, the m⁶A-deficient rhMPVs had significant replication defects in A549 cells.

To determine whether these hMPV mutants are defective in m⁶A methylation, total RNA was purified from rhMPV-infected A549 cells and pulled down using m⁶A antibody-conjugated magnetic beads by the MeRIP method. For rhMPVs with mutations in the antigenome, m⁶A methylation of both antigenome and G mRNA should be affected. As expected, antigenome and G mRNA from m⁶A-deficient rhMPVs had significant defects in binding to m⁶A-specific antibody compared to those from rhMPV (FIG. 32E). As controls, no significant difference in m⁶A binding was observed for genome (FIG. 32E). The inventors also quantified the m⁶A content in virion RNA extracted from each recombinant virus by m⁶A RNA Methylation Assay Kit. As expected, virion RNA of rhMPV-G1-14 containing 14 mutations in m⁶A sites had reduced m⁶A content compared to virion RNA of rhMPV-G1-2 and G8-9 containing only two m⁶A site mutations (FIG. 32F). These results suggest that m⁶A-deficient rhMPVs indeed produce antigenome, G mRNA, or genome that are deficient in m⁶A methylation.

The inventors also generated an important control virus (rhMPV-ALKBH5) that is naturally defective in m⁶A methylation. Briefly, A549 cells were transfected with a plasmid encoding AlkBH5 (a m⁶A eraser protein), followed by infection with rhMPV, and hMPV virions were harvested and purified. Virion RNA of rhMPV-ALKBH5 was significantly defective in m⁶A content (FIG. 32F), presumably because some of the m⁶A modifications in virion RNA have been removed by ALKBH5. Thus, these RNAs are ‘naturally’ deficient in m⁶A methylation, as their nucleotide sequences are not changed.

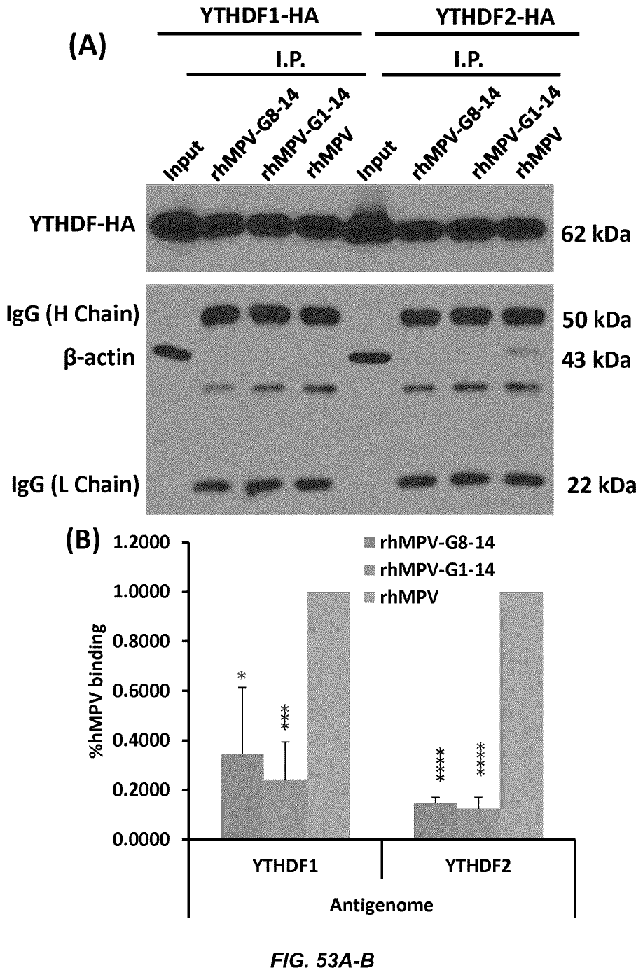

The function of m⁶A methylation is mediated by m⁶A reader proteins. Thus, the inventors also determined whether these hMPV mutants affect the binding efficiency of virion RNA to m⁶A reader proteins. For this purpose, RNA was isolated from highly purified virus particles. As shown in FIG. 32G and FIG. 53, the binding efficiency of antigenome from rhMPV-G1-14 and rhMPV-G8-14 to YTHDF1 and YTHDF2 was significantly reduced, only retaining approximately 12-34% of their binding relative to the rhMPV RNA. However, the antigenome of rhMPV-G1-2 and G8-9 retained 80-90% of their binding activity to YTHDF1 and YTHDF2. Therefore, the antigenome of rhMPVs with more m⁶A site mutations was more defective in binding to the m⁶A reader proteins. These results also provided additional evidence that the antigenome of m⁶A-deficient rhMPVs is deficient in m⁶A methylation.

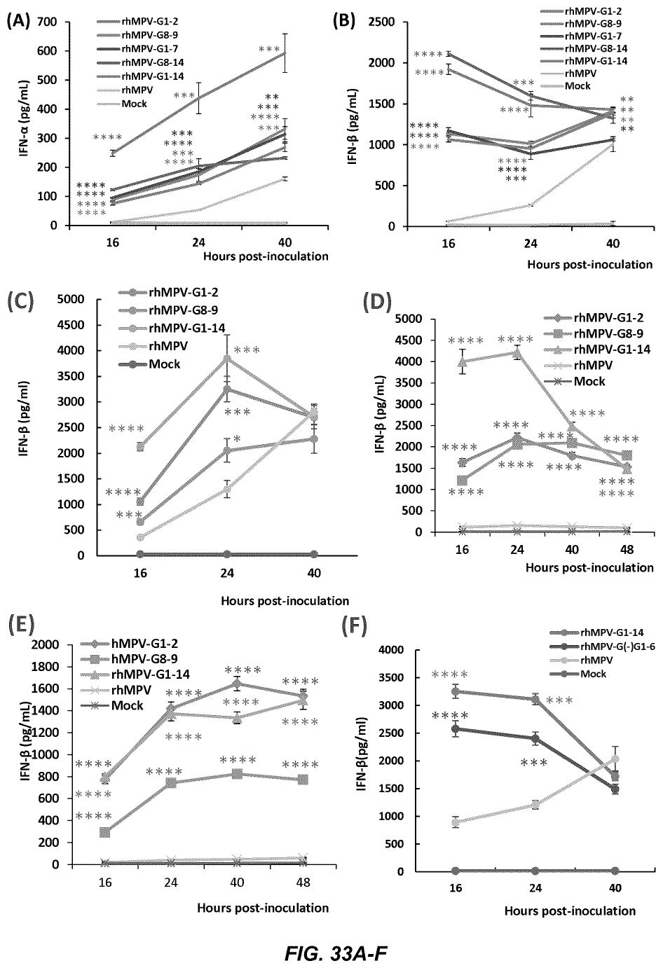

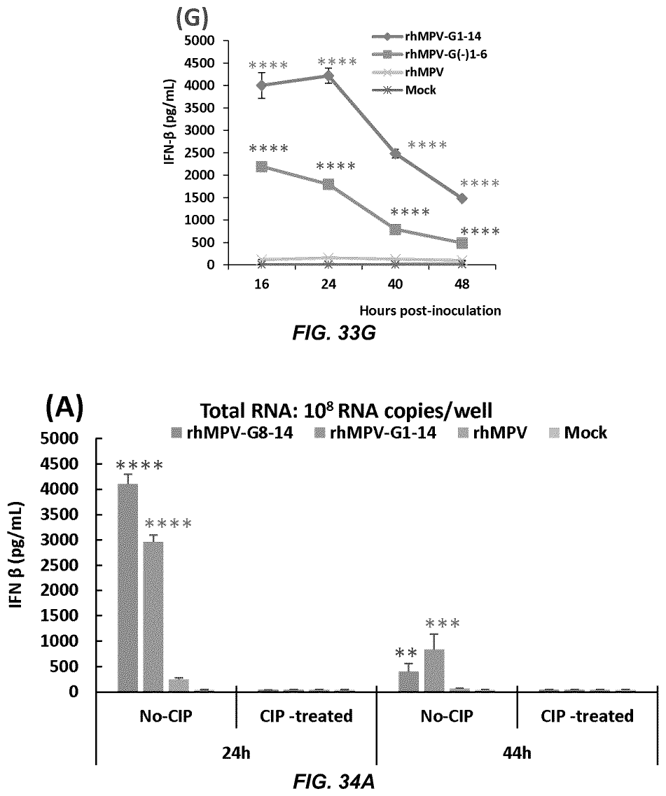

m⁶A-deficient rhMPVs trigger a significantly higher type I interferon response. The m⁶A-seq showed that hMPV infection caused broad alterations in the expression of genes involved in innate immunity. Thus, the inventors determined the dynamics of type I interferon (IFN-I) response of these m⁶A-deficient rhMPVs in virus-infected cells. Briefly, A549 cells were infected by each recombinant virus at an MOI of 4.0, and IFN-α and IFN-β protein levels in cell culture supernatants were determined at 16, 24, and 40 h post-inoculation by ELISA. All m⁶A-deficient rhMPVs induced significantly higher IFN-α (FIG. 33A) and IFN-β (FIG. 33B) than the parental rhMPV. IFN-α level gradually increased with late time points for all recombinant viruses. However, the largest differences on IFN-β level between m⁶A-deficient rhMPVs and parental rhMPV was observed at 16 h post-infection. Interestingly, it appears that rhMPV-G1-2 and G8-9 (with 2 m⁶A site mutations) had stronger IFN responses than rhMPV-G1-14 (with 14 m⁶A site mutations). Thus, the inventors decided to further characterize their IFN responses. The inventors compared the dynamics of IFN-β response of these hMPV mutants in A549 cells at a lower MOI (1.0) (FIG. 33C). Under this condition, rhMPV-G1-14 induced significantly more IFN-β than rhMPV-G1-2 and G8-9.

The inventors also determined IFN production in a human macrophage cell line (THP-1). The increase of IFN-α and IFN-β in TPH-1 was much more dramatic than in A549 cells: a 20-50-fold increase for both IFN-α (FIG. 33D) and IFN-β (FIG. 33E) in rhMPV-G1-14 and G8-14-infected cells compared to those infected with the parental rhMPV. The inventors compared the dynamics of the IFN-β response to rhMPV-G1-14, G1-2, and G8-9 in THP-1 at MOI of 4.0 (FIG. 33D) and 1.0 (FIG. 33E). At both MOIs, rhMPV-G1-14 induced significantly higher IFN-β than rhMPV-G1-2 and G8-9 at 16 and 24 h post-infection.

The inventors next compared the IFN responses to rhMPV-G(−)1-6 and rhMPV-G1-14. It was found that rhMPV-G(−)1-6 had significantly higher IFN responses in both A549 cells (FIG. 33F) and THP-1 cells (FIG. 33G) compared to rhMPV although their IFN responses were relatively less than that of rhMPV-G1-14.

Taken together, these results demonstrate that (i) inactivation of m⁶A sites in the G region in antigenome and G gene in genome of hMPV results in enhanced IFN-I induction; and (ii) hMPV with natural deficiency in m⁶A methylation leads to an enhanced IFN-I induction.

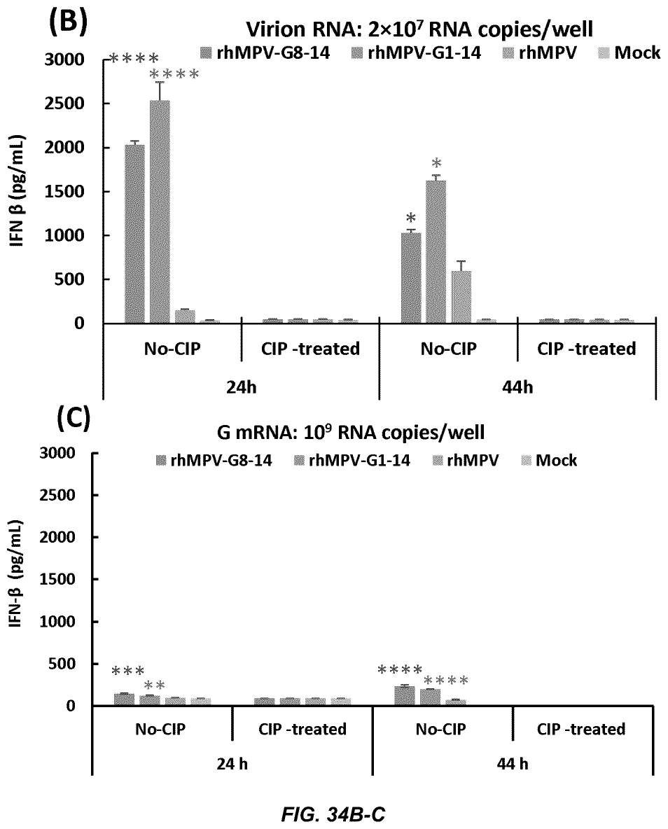

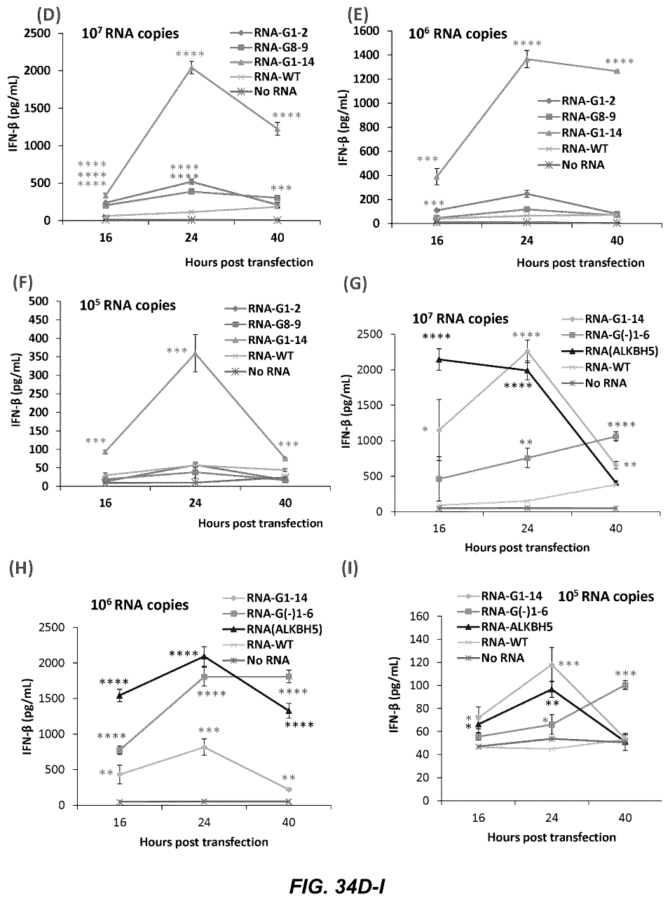

m⁶A-deficient hMPV antigenome and genome but not the G mRNA triggers a higher type I IFN response. To further investigate the mechanism underlying the enhanced IFN-I response associated with m⁶A-deficient hMPVs, the inventors transfected A549 cells with RNAs isolated from virus-infected cells or virions. Under these conditions, there would be no viral replication but there would be viral protein translation. Total RNAs purified from rhMPV, rhMPV-G8-14, rhMPV-G1-14-infected A549 cells would include viral genome, antigenome and mRNAs, and host RNAs. Equal amounts of total RNA isolated from rhMPV-G8-14 and rhMPV-G1-14-infected A549 cells which are deficient in viral RNA m⁶A modification triggered a significantly higher IFN response than those RNAs isolated from rhMPV-infected A549 cells (P<0.05) (FIG. 34A). No type I interferon response was detected in A549 cells transfected with mock-infected cell RNA. This IFN production was abrogated when the RNAs were dephosphorylated by CIP treatment (FIG. 34A), consistent with the previous observation that the 5′-phosphorylated viral genome RNA is the ligand for cytoplasmic RNA sensors. Since RNAs from hMPV or hMPV-infected cells cannot initiate replication, it is likely that the m⁶A-deficient viral RNAs contributed to the enhanced type I IFN response. Next, total RNAs from hMPV-infected cells were incubated with a polyadenylated G mRNA-specific probe, and viral G mRNA was isolated. Transfection of G mRNA samples into A549 cells only induced a minimal amount of IFN-β compared to virion RNA (FIG. 34C, compared to FIGS. 34A and B). To completely eliminate any effect of contamination of viral genome, antigenome or triphosphorylated RNA, the G mRNA was treated with CIP followed by transfection into A549 cells. As expected, G mRNA from rhMPV-G8-14, rhMPV-G1-14, and rhMPV failed to stimulate IFN induction, and there was no significant difference among these groups (P>0.05) (FIG. 34C), demonstrating that m⁶A-deficient G mRNA did not contribute to the enhanced type I IFN response.

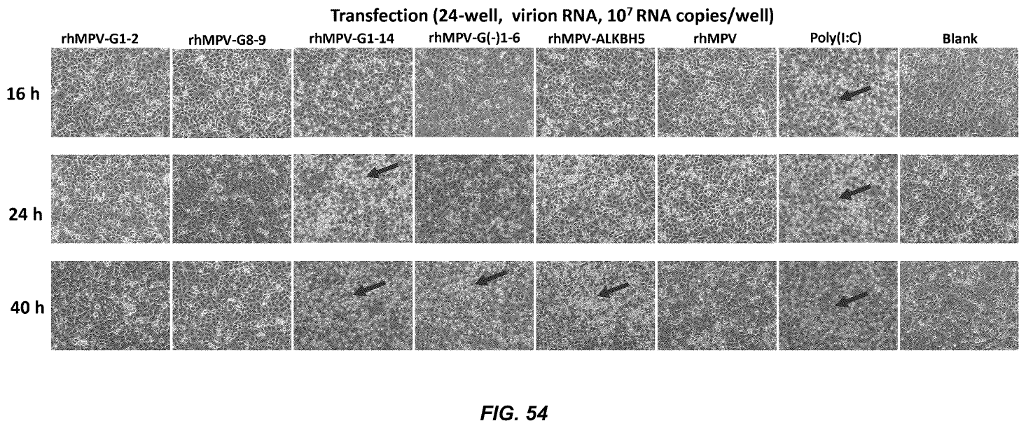

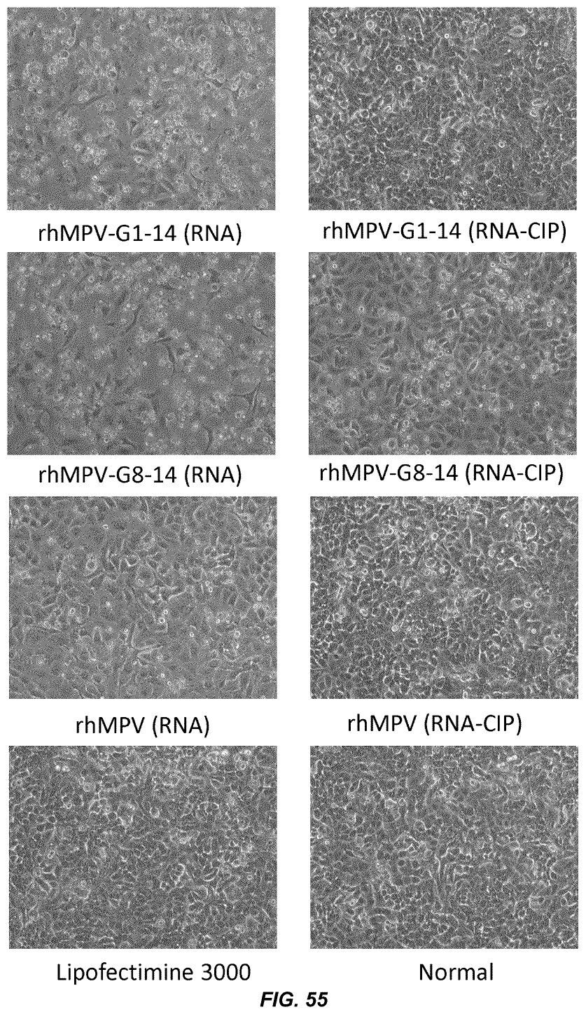

Finally, to directly demonstrate whether the m⁶A-deficient antigenome is involved in the enhanced IFN response, the inventors isolated hMPV genome and antigenome from highly purified virions and assessed it for the induction of IFN expression. Transfection of virion RNAs of rhMPV-G8-14 and rhMPV-G1-14 stimulated significantly higher IFN induction in A549 cells than RNAs of rhMPV (P<0.05) (FIG. 34B). IFN was completely abrogated when RNAs were dephosphorylated by CIP. In addition, cells transfected with virion RNAs of rhMPV-G8-14 and rhMPV-G1-14 triggered an earlier cell death compared to those from rhMPV (FIGS. 54 and 55). Together, these results demonstrate that m⁶A-deficient hMPV antigenome but not G mRNA are responsible for inducing higher type I IFN.

The inventors next directly compared the IFN responses of virion RNA derived from rhMPV-G1-14, G1-2 and G8-9. Briefly, A549 cells were transfected with three different amounts of each virion RNA, and the dynamics of IFN-β production was measured. RNA of rhMPV-G1-14 triggered significantly higher IFN-β than RNA of rhMPV-G1-2 and G8-9 at all three RNA concentrations (FIGS. 34D, E, and F). In addition, RNA of rhMPV-G1-2 and G8-9 induced significantly higher IFN-β than RNA of hMPV. Similarly, the inventors compared the IFN response of virion RNA derived from rhMPV-G1-14, rhMPV-G(−)1-6, and rhMPV-ALKBH5. RNA of rhMPV-G1-14 and rhMPV-ALKBH5 induced a significantly higher IFN-R than RNA of rhMPV-G(−)1-6 (FIGS. 34G, H, and I). Overall, these results demonstrated that m⁶A-deficient hMPV antigenome and genome induced a higher IFN response and the level of IFN response correlated with the degree of m⁶A deficiency.

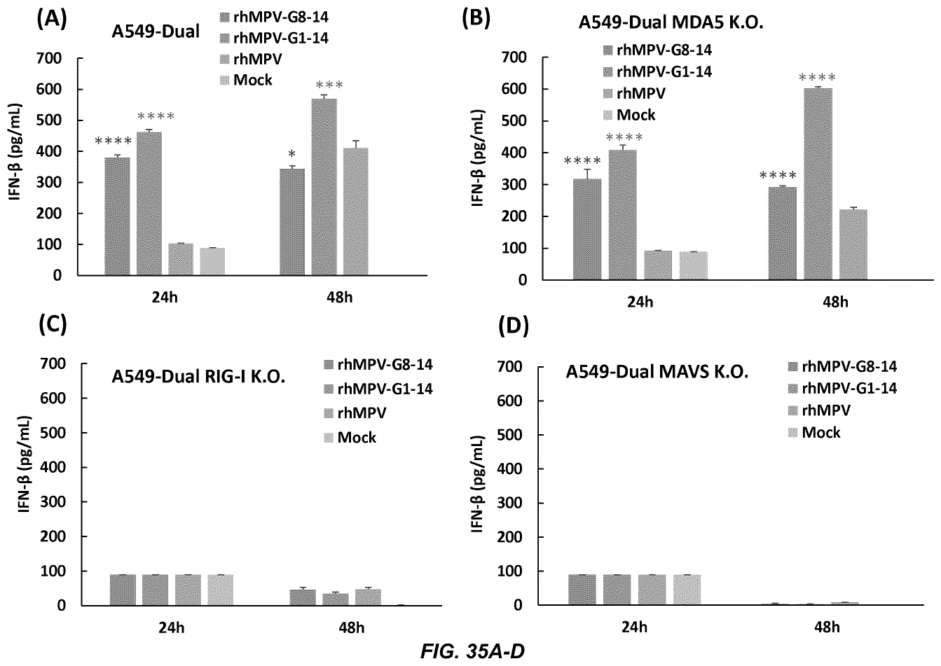

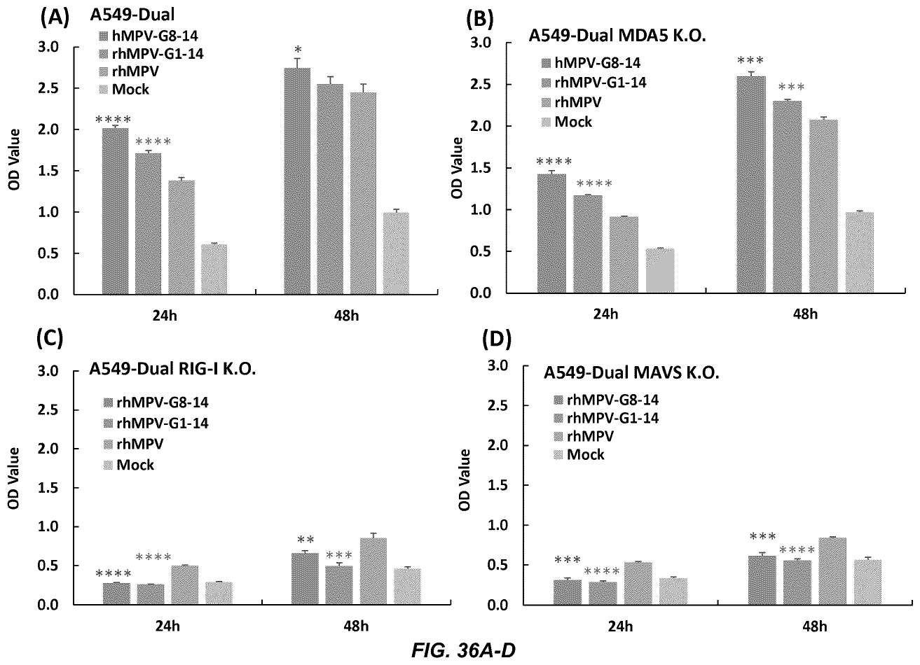

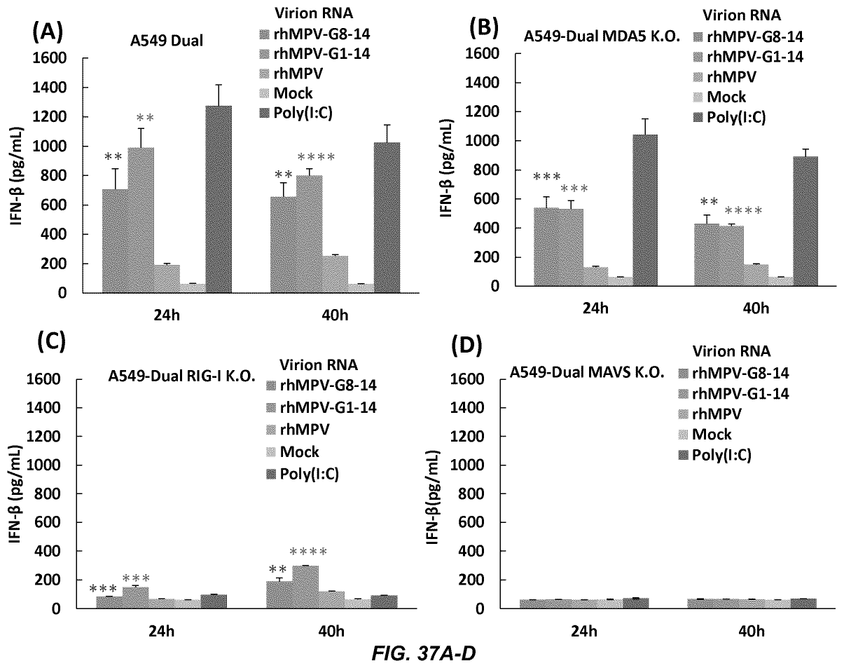

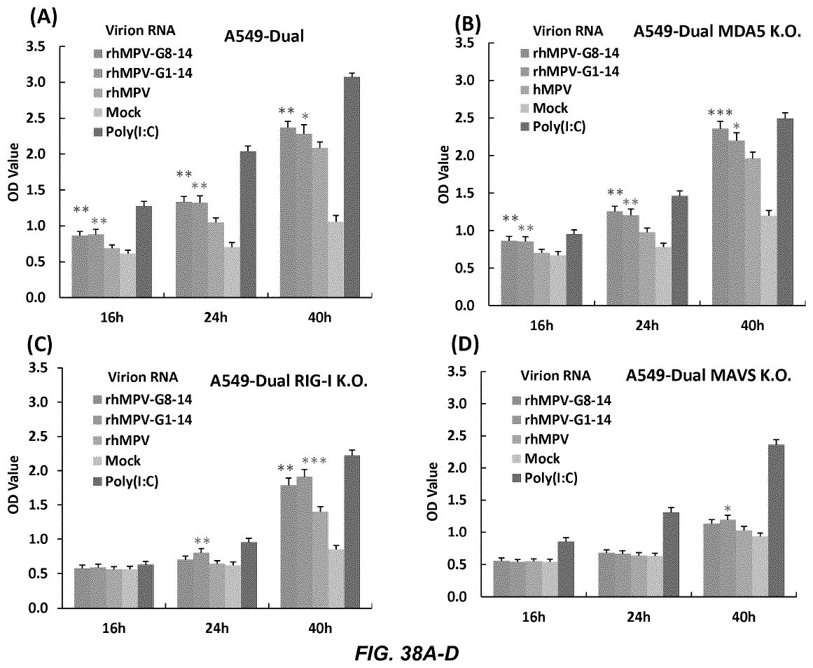

RIG-I plays a dominant role in the activation of higher interferon response and NF-κB. It is well established that RIG-I recognizes a 5′ triphosphorylated or diphosphorylated ssRNA, and short dsRNAs whereas MDA5 detects long dsRNAs (4, 9). The genome and antigenome of NNS RNA viruses are 5′ triphosphorylated ssRNA, thus detected by RIG-I. However, it remains possible that MDA5 may play a role in inducing a higher innate immunity of m⁶A-deficient rhMPVs, as it is known that rhMPV also activates the MDA5 pathway (57). In addition, it should be noted that there is cross-talk between the IFN and NF-κB pathways (3). Activation of downstream transcription factor of IRF3 leads to IFN production whereas activation of the transcription factor NF-κB leads to the expression of proinflammatory cytokines and chemokines. Thus, the inventors directly examined the IFN response of m⁶A-deficient rhMPVs in A549 cells lacking RIG-I, MDA5, or their downstream adaptor protein, MAVS. These cell lines also express a secreted embryonic alkaline phosphatase (SEAP) reporter gene under the control of the IFN-β minimal promoter fused to five NF-κB binding sites, allowing us to measure the activation of the NF-κB pathway. Briefly, RIG-I, MDA5, or MAVs-knockout A549 cells were infected by rhMPV or m⁶A-deficient rhMPVs, and IFN was measured. As expected, both rhMPV-G8-14 and rhMPV-G1-14 triggered a higher and earlier IFN-β response in wild type A549 cells than rhMPV (P<0.05) (FIG. 35A). Interestingly, IFN-R production of rhMPV-G8-14 and rhMPV-G1-14 was completely abrogated in RIG-I (FIG. 35C) and MAVS (FIG. 35D)-knockout A549 cells. In contrast, a high level of IFN-β was still detectable in MDA5-knock out A549 cells (FIG. 35B) although there was a significant reduction compared to the wild type A549 cells (P<0.05). A similar pattern was observed for SEAP activity (FIG. 36), which measures activation by NF-κB. The inventors also measured the IFN-β response in A549 knockout cells by transfection of virion RNA. Similar to virus infection, IFN-β production of virion RNAs from m⁶A-deficient rhMPVs was completely abrogated in RIG-I and MAVS but not in MDA5-knockout A549 cells (FIG. 37). Similarly, virion RNAs of m⁶A-deficient rhMPVs also induced higher SEAP activity (FIG. 38). Together, these results demonstrated that the cytoplasmic RNA sensor RIG-I played a dominant role in activation of the interferon response and NF-κB pathway by m⁶A-deficient rhMPVs and virion RNA. These results also suggest that m⁶A-deficient antigenome RNA may be recognized by the cytoplasmic RNA sensor RIG-I but not MDA5.

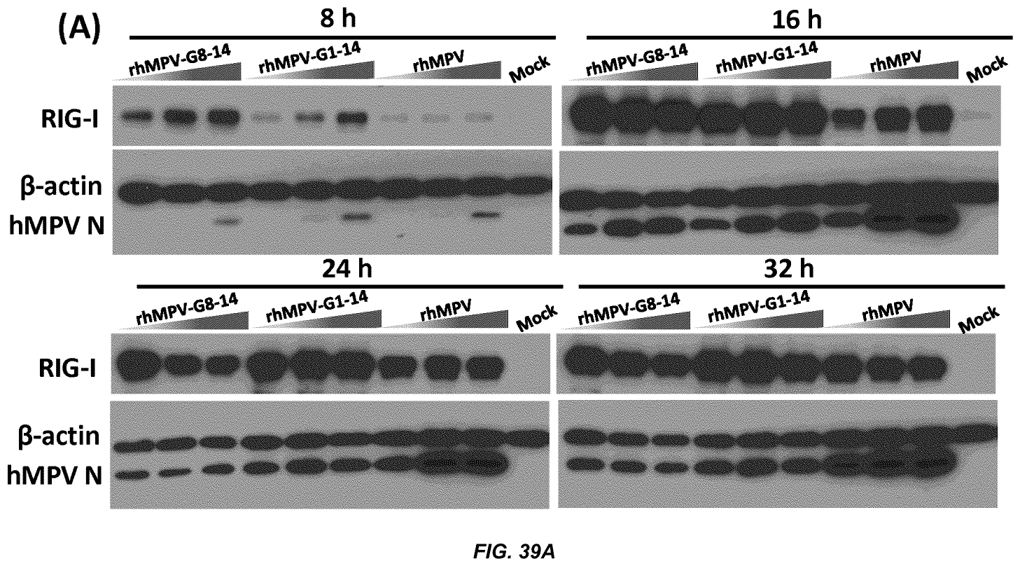

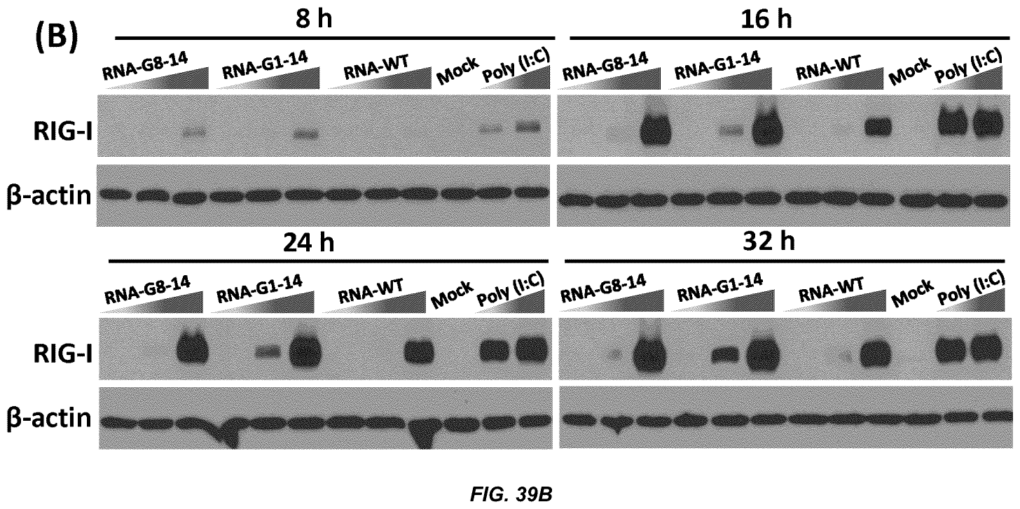

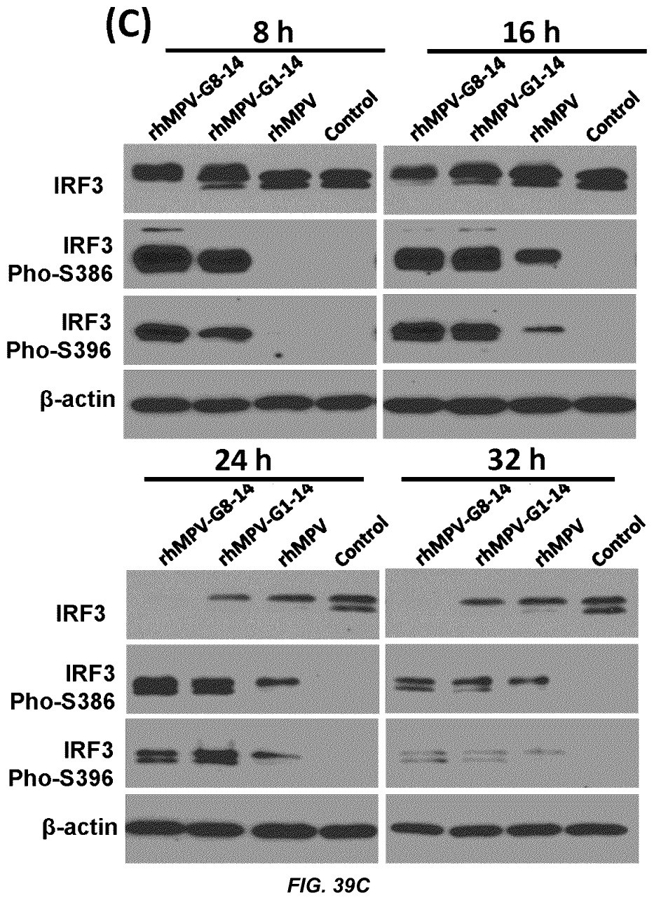

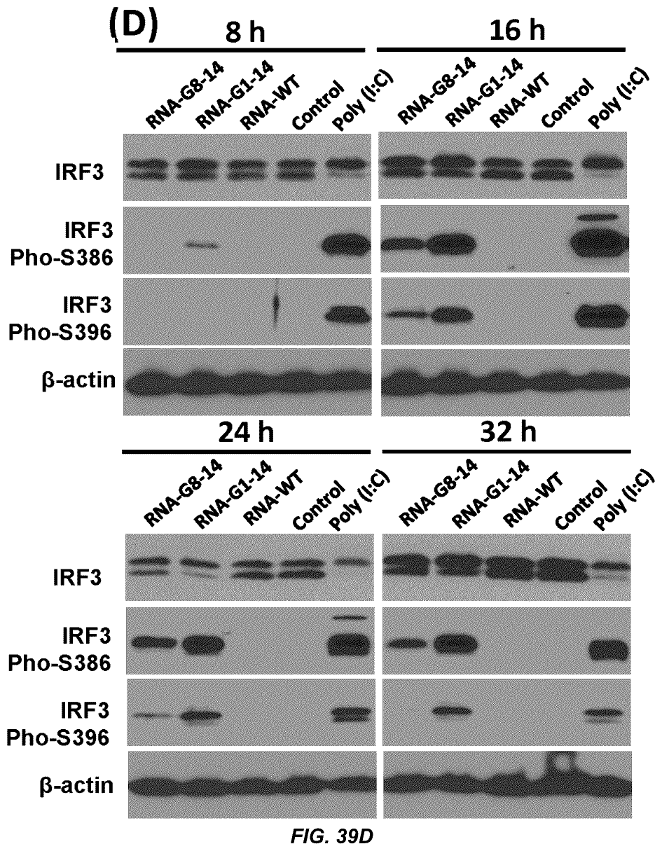

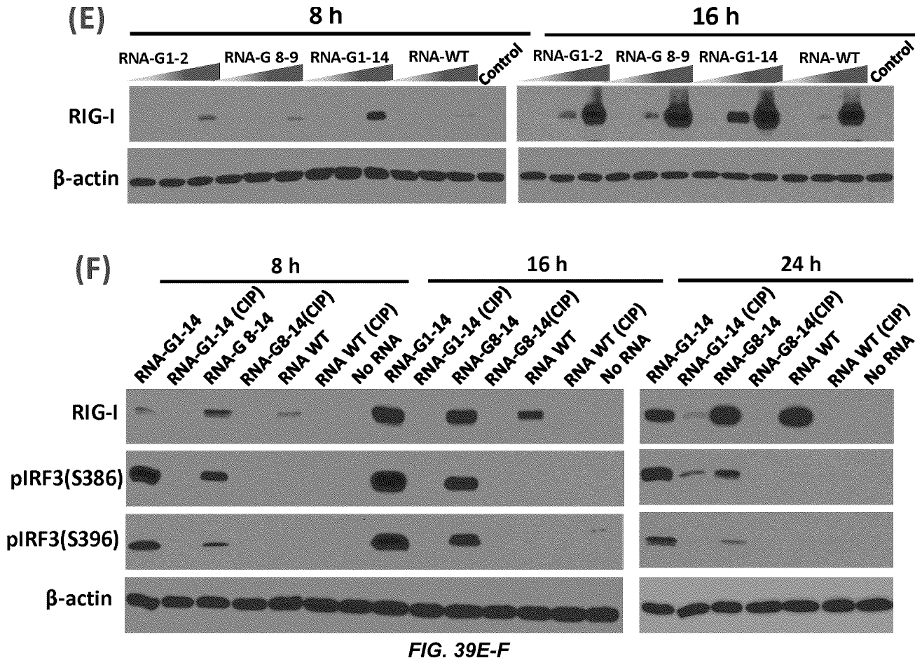

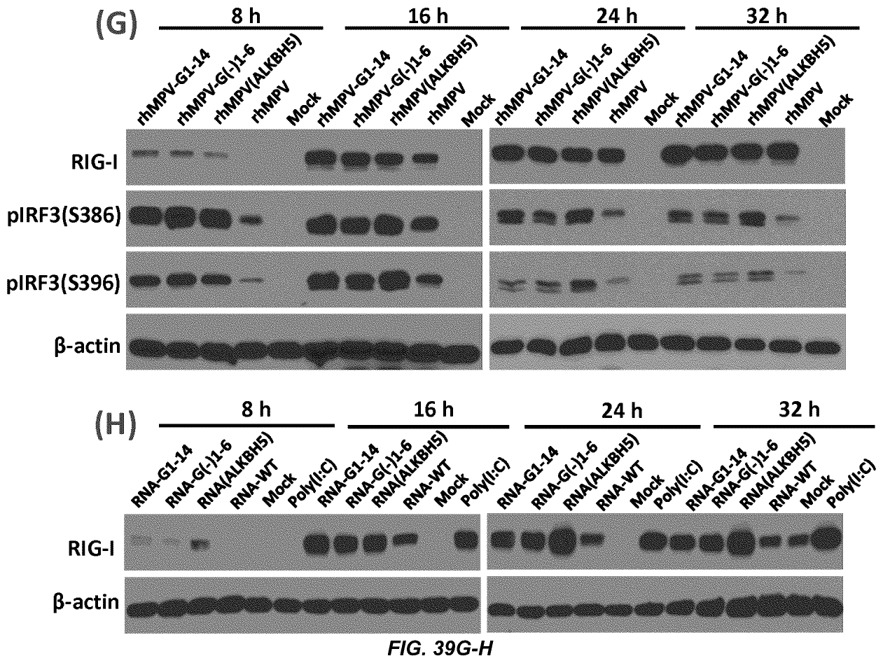

m⁶A-deficient antigenome and genome enhances expression of the cytoplasmic RNA sensor RIG-I. Having demonstrated that RIG-I plays a dominant role in inducing IFN, the inventors next asked whether m⁶A-deficient rhMPVs enhances RIG-I expression. To do this, A549 cells were infected by each recombinant virus at an MOI of 0.5, 2.0, and 5.0, RIG-I was detected at 8, 16, 24, and 32 h post-infection by Western blot. As shown in FIG. 39A, rhMPV-G8-14 and rhMPV-G1-14 induced a significantly higher expression of RIG-I compared to rhMPV. To investigate whether m⁶A-deficient antigenome directly activates RIG-I, A549 cells were transfected with an increasing amount of virion RNAs, and RIG-I was detected (FIG. 39B). As a positive control, Poly(I:C) activated RIG-I expression in a dose-dependent manner. Interestingly, m⁶A-deficient antigenome from rhMPV-G8-14 and rhMPV-G1-14 triggered significantly higher expression of RIG-I compared to that of rhMPV (P<0.05). The inventors next compared the RIG-I expression triggered by virion RNA of rhMPV-G1-14, G1-2, and G8-9. As shown in FIG. 39E, virion RNA of rhMPV-G1-14 induced higher expression of RIG-I than virion RNA of rhMPV-G1-2 and G8-9. Also, rhMPV-G(−)1-6 and rhMPV-ALKBH5 triggered higher expression of RIG-I compared to rhMPV in virus-infected cells. Similarly, RNA of rhMPV-G(−)1-6 and rhMPV-ALKBH5 triggered higher expression of RIG-I compared to RNA of rhMPV in RNA-transfected cells (FIG. 39H). Finally, the inventors determined whether 5′triphosphate of virion RNA is required for inducing RIG-I expression. Virion RNA of rhMPV-G1-14, G8-14, and rhMPV were treated with CIP, purified, and re-quantified by real-time RT-PCR. RNA with or without CIP treatment was transfected into A549 cells, and RIG-I was detected by Western blot. As shown in FIG. 39F, RIG-I protein was barely detectable when virion RNA was treated by CIP. These results suggest that 5′triphosphate of RNA is required for inducing RIG-I expression regardless of whether the RNA is m⁶A methylated or m⁶A deficient.

m⁶A-deficient antigenome enhances IRF3 phosphorylation. In the type I IFN signaling pathway, the activated RIG-I and MDA-5 interact with the downstream mitochondrial antiviral signaling (MAVS) adaptor molecule, leading to a signaling cascade that includes activation of TBK1 and IKKF protein kinases which phosphorylate and activate the transcription factors interferon regulatory factor 3 (IRF3) and NF-κB (3). Subsequently, IRF3 and NF-κB are translocated from the cytosol to the nucleus to induce transcription of a variety of innate immune and inflammatory response genes including IFNs. To demonstrate the activation of the downstream signaling cascade, the inventors measured the phosphorylation of IRF3 upon hMPV infection (FIG. 39C) or virion RNA transfection (FIG. 39D) by Western blot. A549 cells were infected by each rhMPV at an MOI of 5.0, phosphorylation of IRF3 was measured using antibody specific to two different phosphorylation sites, S386 and S396. Phosphorylation of IRF3 was significantly higher in rhMPV-G18-14 and G1-14-infected cells than the rhMPV-infected cells (FIG. 39C). Similarly, the inventors observed a significantly higher IRF3 phosphorylation in A549 cells transfected with virion RNA derived from rhMPV-G8-14 and G1-14 than those transfected with virion RNA from rhMPV (FIG. 39D). Higher IRF3 phosphorylation at S386 and S396 were also observed for rhMPV-G(−)1-6 and rhMPV-ALKBH5 compared to rhMPV (FIG. 39G). In addition, CIP treatment of virion RNA of rhMPV-G8-14, G1-14, and rhMPV completely abolished IRF3 phosphorylation (FIG. 39F), which is consistent with the results that CIP treatment abolished RIG-I expression and IFN production. Thus, these results showed that m⁶A deficient hMPVs led to a significantly higher amount of IRF3 phosphorylation, which is consistent with the fact that they induced higher expression of type I IFN.

Enhanced recognition of m⁶A-deficient antigenome by cytoplasmic RNA sensor RIG-I. The inventors next directly compared the binding affinity of m⁶A-containing and -deficient antigenome to RIG-I protein. The inventors first used biotinylated virion RNA to pull down endogenously expressed RIG-I in A549 cell extract. To do this, 10⁹ copies of virion RNA with or without CIP treatment was biotinylated and subsequently conjugated to the Streptavidin beads. The RNA-associated beads were washed three times and incubated with A549 cell lysates containing endogenously overexpressed RIG-I. RIG-I that co-isolated with the biotinylated RNA via Streptavidin beads was detected by Western blotting. As shown in FIG. 40A, virion RNA of rhMPV-G8-14 and rhMPV-G1-14 pulled down significantly more RIG-I protein compared to virion RNA of rhMPV. After removal of triphosphate by CIP, virion RNA from rhMPV and rhMPV mutants failed to pull down RIG-I. This result demonstrated that m⁶A-deficient virion RNA enhanced the ability to pull down RIG-I protein.

As a complementary approach, the inventors used RIG-I protein to pull down virion RNA. To do this, total cell lysates from A549 cells overexpressing Flag-tagged RIG-I was incubated with anti-Flag M2 magnetic beads. The RIG-I conjugated magnetic beads (FIG. 40B) were further incubated with 2×10⁸ copies of virion RNA (either with or without CIP treatment) or 2×10⁹ copies of N or G mRNA. The beads were washed three times and RNA bound to the RIG-I conjugated-beads was quantified by real-time RT-PCR. Antigenome (without CIP) of rhMPV-G1-14 and rhMPV-G8-14 had approximately 90- and 33-fold increases in binding to RIG-I compared to those of rhMPV (FIG. 40C). After removal of the triphosphate by CIP, the binding of antigenome to RIG-I was significantly abrogated, consistent with the fact that triphosphorylated RNA is the ligand for RIG-I (FIG. 40C). As controls, the capped viral mRNAs (N and G) had a minimal level of binding to RIG-I. In addition, N and G mRNAs from rhMPV-G1-14 and rhMPV-G8-14 had a similar level of binding efficiency compared to those of rhMPV (FIG. 40C). This observation is in agreement with the fact that capped mRNA is not recognized by RIG-I. Therefore, these results demonstrated that m⁶A-deficient antigenome RNA significantly increases binding affinity to RIG-I.

To further confirm that m⁶A-deficient RNA has an increased binding affinity to RIG-I, the inventors performed an in vitro RNA competitive binding assay. Briefly, highly purified RIG-I protein (FIG. 40D) were incubated with virion RNA mixture consisting of different ratios of biotinylated rhMPV1-14- and rhMPV-RNA (conjugated to Dynabeads® MyOne™ Streptavidin C1 beads) in the presence of AMP-PNP, an analogue of ATP. The beads were washed three times and subjected to Western blot against RIG-I. As shown in FIG. 40E, the amount of pulldown RIG-I increased when more m⁶A-deficient RNA (rhMPV-G1-14) was used in the competition assay.

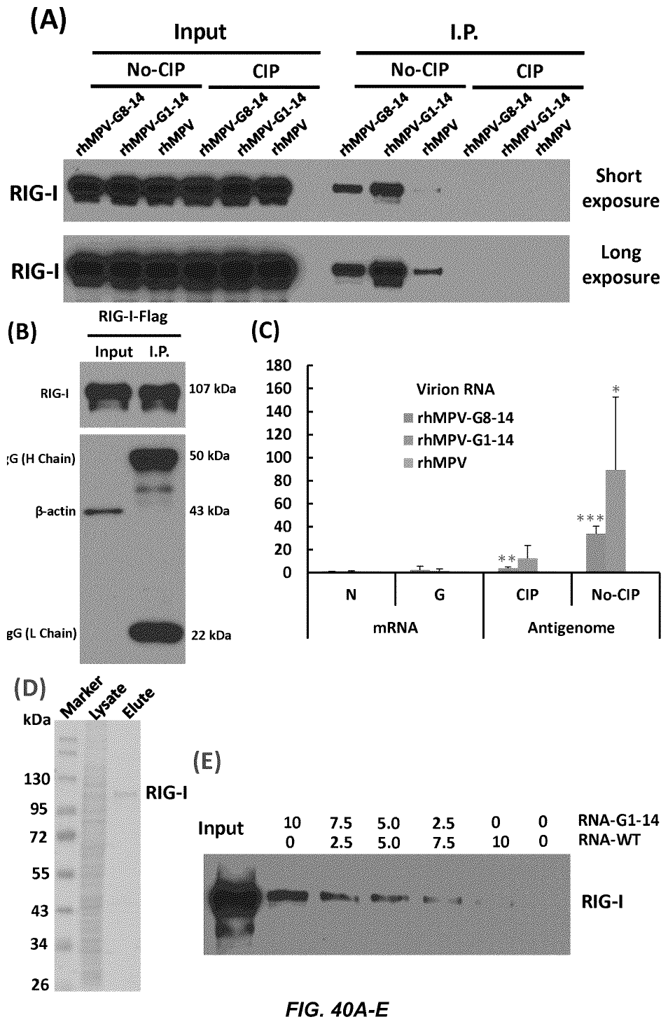

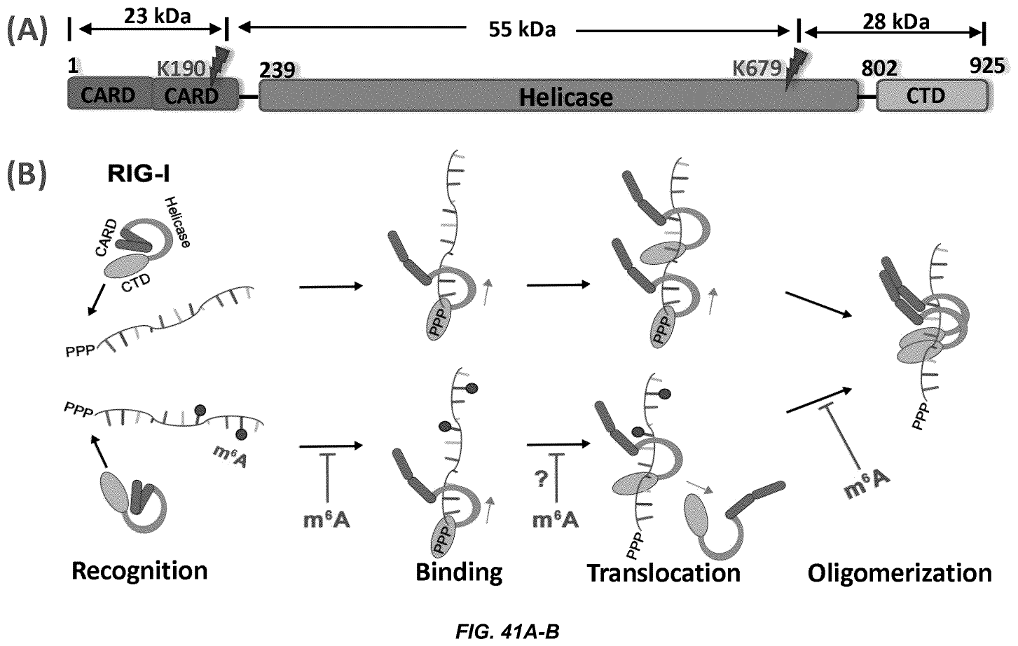

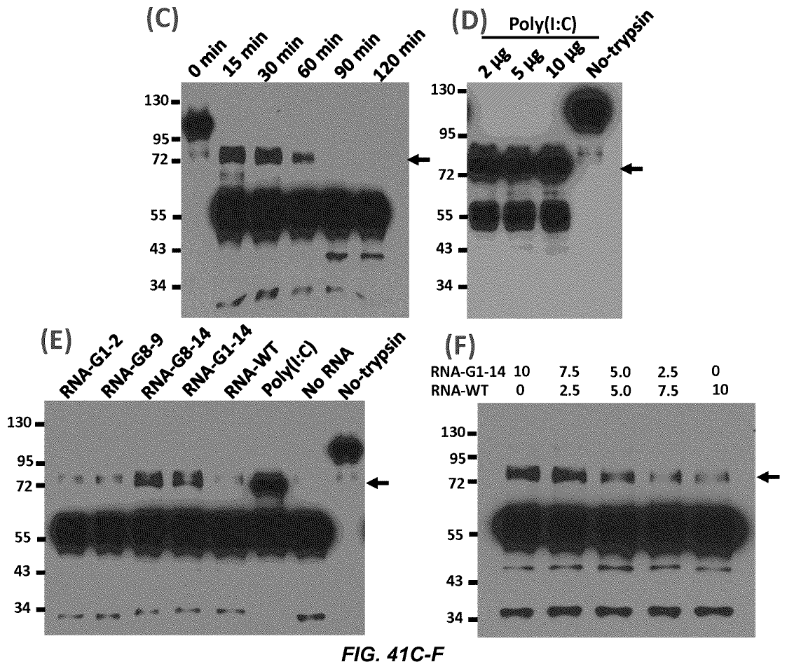

m⁶A-deficient virion RNA facilitates RIG-I conformational changes. To gain further mechanistic insight into how m⁶A-deficient RNA leads to RIG-I mediated IFN signaling, the inventors examined the conformational changes of RIG-I upon binding to RNA ligand. The RIG-I protein is comprised of three major domains, the caspase activation and recruitment domains (CARDs), a helicase domain (Helicase) that binds to ATP and interacts with RNA, and a C-terminal domain (CTD) which is a triphosphate recognition and RNA binding domain (58) (FIG. 41A). In the absence of ligand RNA, RIG-I is in an autorepressed conformation (FIG. 41B). Upon binding of the 5′ppp-RNA ligand to the RIG-I CTD, it enables subsequent ATP binding and hydrolysis and helicase:RNA interactions (59). This induces a RIG-I conformational change that releases the CARDs for K63-linked polyubiquitination and binding of unanchored K63-linked ubiquitin chains (59). These combined activities result in the interaction of CARDs with the adapter protein MAVS, causing MAVS oligomerization and subsequent downstream signaling (FIG. 41B).

The inventors hypothesized that m⁶A-deficient hMPV RNA facilitates the RIG-I conformational change for downstream signaling. To test this hypothesis, the inventors adopted limited proteolysis of RIG-I:RNA complexes using trypsin, which has been often used for examining the RIG-I conformational changes (60, 61). To do this, excess hMPV virion RNA (with or without m⁶A methylation) and AMP-PNP were added to highly purified RIG-I protein and the mixtures were incubated at 37° C. for 30 min to allow RIG-I:RNA complexes to form. Subsequently, trypsin was added to the mixture to partially digest the complexes. At different time points, the protease activity was stopped by adding SDS loading buffer. Tryptic protein fragments were separated by SDS-PAGE, and RIG-I-specific fragments were detected by Western blot using an anti-helicase domain antibody against RIG-I (Abcam). As shown in FIG. 41C, in the absence of ligand RNA, trypsin treatment of the full-length RIG-I protein (approximately 110 kDa) yielded a 55-kDa RIG-I fragment (helicase domain), representing the trypsin sensitivity of autorepressed RNA-free RIG-I conformation. As positive controls, trypsin treatment of poly (I:C) bound RIG-I yielded an 80-kDa fragment (CARD-Helicase domain) which represents the trypsin resistance of the RNA-bound RIG-I (FIG. 41D). Next, the inventors compared the trypsin sensitivity of RIG-I upon binding of virion RNA for each of the hMPV mutants. As shown in FIG. 41E, RNA of rhMPV-G1-14 and G8-14 yielded significantly more 80-kDa fragment than RNA of rhMPV-G1-2, G8-9, or rhMPV. In addition, the inventors performed a competition assay in which different ratios of RNAs rhMPV-G1-14 and rhMPV were mixed and incubated with RIG-I and AMP-PNP, followed by trypsin treatment. The RIG:RNA mixture containing more rhMPV-G1-14 RNA yielded significantly more of the trypsin-resistant 80-kDa RIG-I fragment (FIG. 41F). Collectively, these data suggest that the 5′ppp- on the genome is essential for RIG-I binding, but the m⁶A-deficient virion RNA either enhances RIG-I binding or facilitates the conformational change of RIG-I in the RIG-I:RNA complex which is known to enhanced downstream IFN induction.

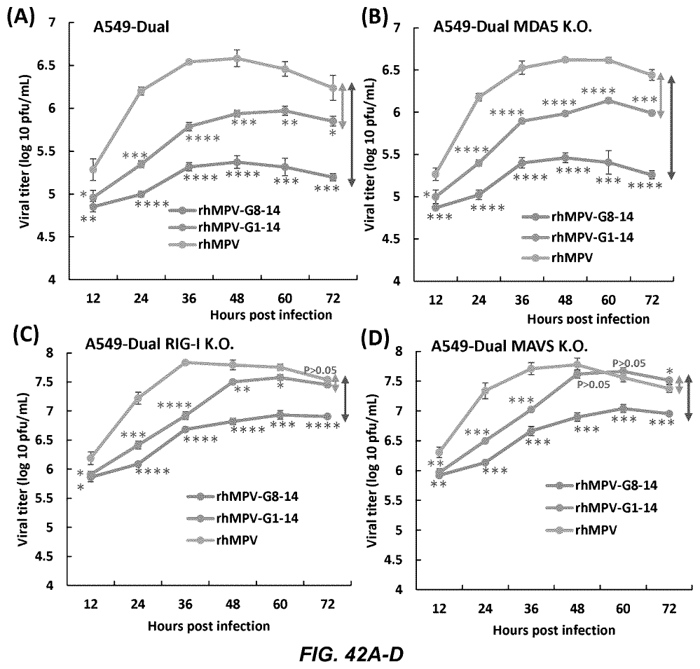

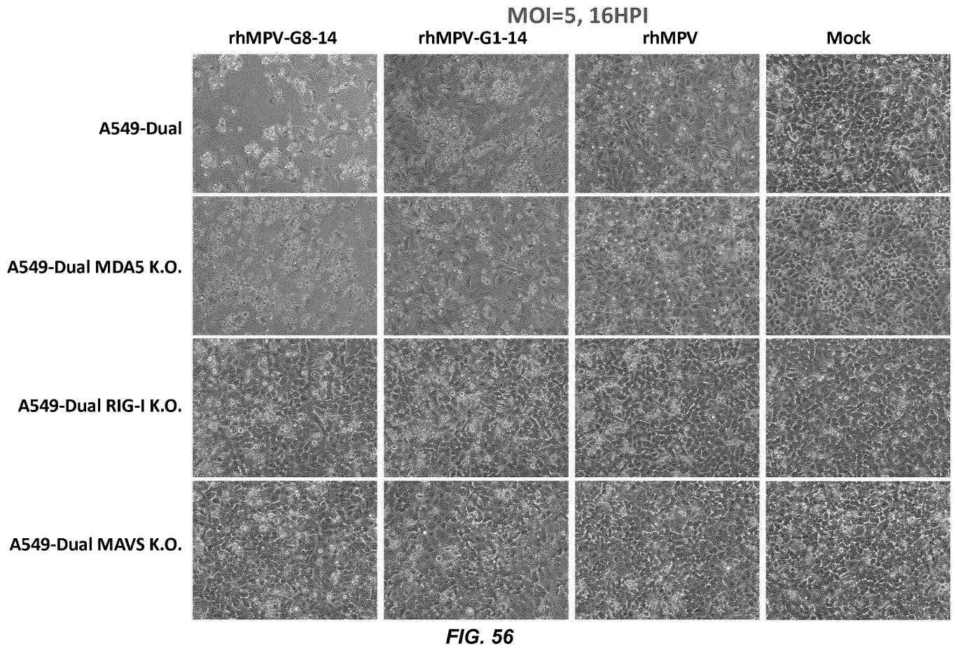

Restoration of the replication of m⁶A-deficient rhMPVs in RIG-I and MAVS knockout A549 cells. If RIG-I is indeed involved in recognition of nonself RNA, the growth of m⁶A-deficient rhMPVs should be restored when RIG-I and its adaptor MAVS proteins are depleted. Thus, the inventors performed a single step growth curve of m⁶A-deficient rhMPVs in wt, MDA5, RIG-I, and MAVS knockout A549 cells. Compared to rhMPV, both rhMPV-G1-14 and rhMPV-G8-14 had a significant defect in growth in wild type A549 cells (FIG. 42A). Interestingly, the growth of rhMPV-G1-14 was restored in RIG-I (FIG. 17C) and MAVs (FIG. 42D) knockout A549 cells although it had a delay in replication kinetics in early time points. Also, the growth of rhMPV-G8-14 was partially restored in RIG-I and MAVS knockout A549 cells. In contrast, the growth of neither rhMPV-G1-14 nor rhMPV-G8-14 was restored in MDA5 knockout cells (FIG. 42B), similar to the wt cell line. Interestingly, rhMPV-G1-14 and rhMPV-G8-14 displayed earlier cell death in wild type and MDA5 knockout A549 cells than in RIG-I and MAVs knockout A549 cells (FIG. 56). These results further support the notion that RIG-I but not MDA5 is involved in recognition of m⁶A-deficient antigenome RNA.

Contribution of other functions of m⁶A methylation to the attenuated phenotype of m⁶A-deficient rhMPVs. Although the peak titer of m⁶A-deficient hMPVs can be rescued in RIG-I and MAVS knockout cells, m⁶A-deficient hMPVs exhibited delayed replication kinetics at early time points. This suggests that innate sensing may not be the only factor which leads to the attenuation of m⁶A-deficient hMPVs. Previous studies have shown that m⁶A plays important roles in RNA stability and mRNA translation (29, 31). During the experiment, the inventors found that virion RNA extracted from m⁶A-deficient hMPVs was easily degraded when the RNA samples were stored in −80° C. These degraded RNA samples failed to trigger a higher IFN response, as fresh RNA did (data not shown), suggesting that viral m⁶A is important for RNA stability.

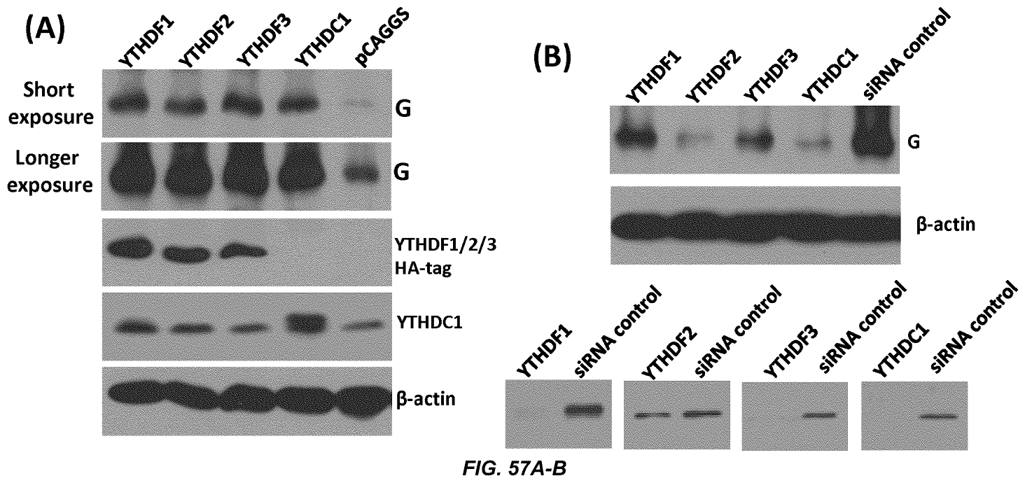

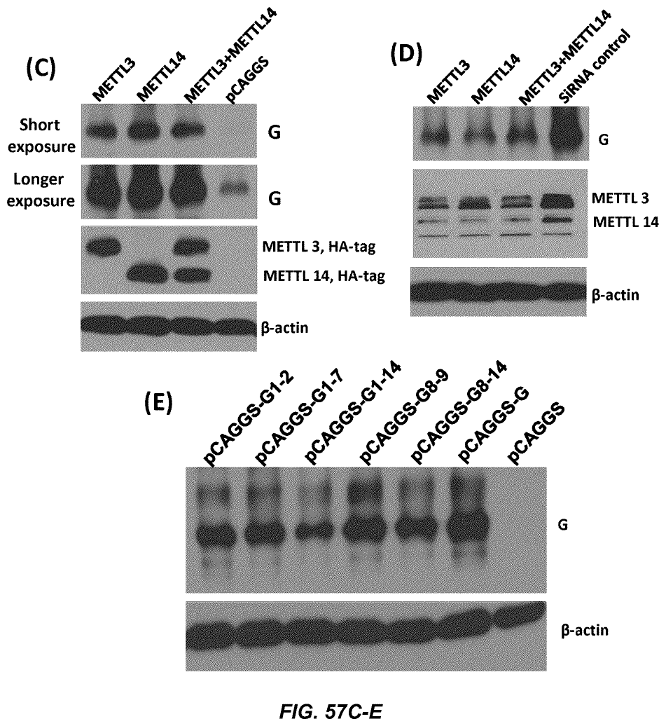

In hMPV-infected cells, the inventors found that G protein synthesis from m⁶A-deficient hMPVs decreased compared to rhMPV, suggesting that the m⁶A in G mRNA may be important for its stability or translation. To further test this hypothesis, the inventors first determined the impact of G protein expression by overexpression or knockdown of host m⁶A writer and reader proteins. To do this, pCAGGS expressing the hMPV G gene was constructed (pCAGGS-G). A549 cells were transfected with plasmids encoding YTHDF1-3, YTHDC1, METTL3, and METTL14, and 20 h later were transfected with pCAGGS-G. Interestingly, G protein expression dramatically increased in A549 cells that transiently overexpress m⁶A reader proteins (FIG. 57A) and writer proteins (FIG. 57C). Conversely, G protein expression was significantly reduced when individual, endogenous reader (YTHDF1-3 and YTHDC1) (FIG. 57B) and writer (METTL3 and METTL14) (FIG. 57D) were knocked down by siRNA. Thus, m^{6A} reader and writer proteins increase the expression of hMPV G protein from m⁶A-modified G mRNA. As a second approach, the inventors mutated m⁶A sites in the G mRNA and determined the G protein expression. These pCAGGS-G mutants were transfected into A549 cells and the effect of these mutations on G protein expression was analyzed by Western blot. Mutants pCAGGS-G1-14 and pCAGGS-G8-14 had a significant reduction in G protein expression compared to pCAGGS-G (FIG. 57E). Thus, abrogation of m⁶A site in G mRNA diminished G protein translation. Taken together, m⁶A in virion RNA plays a major role in the innate immune recognition and RNA stability whereas m⁶A in mRNA is important for protein translation.

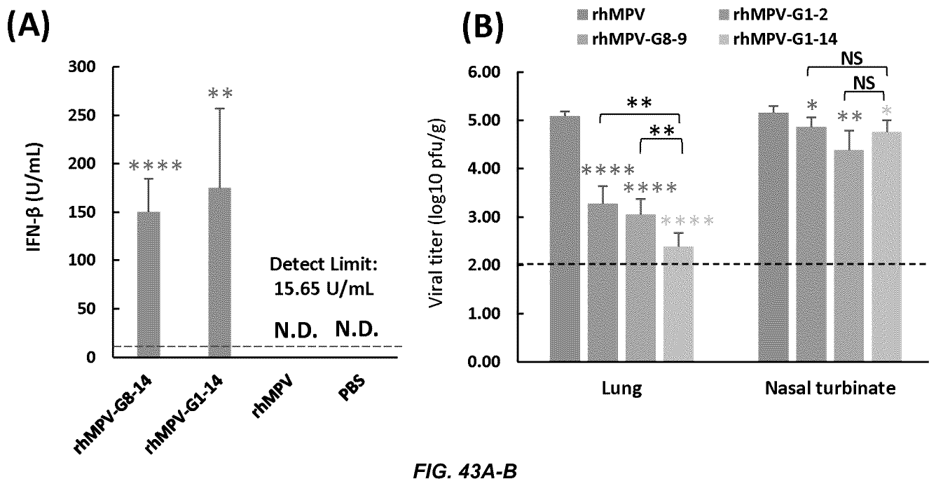

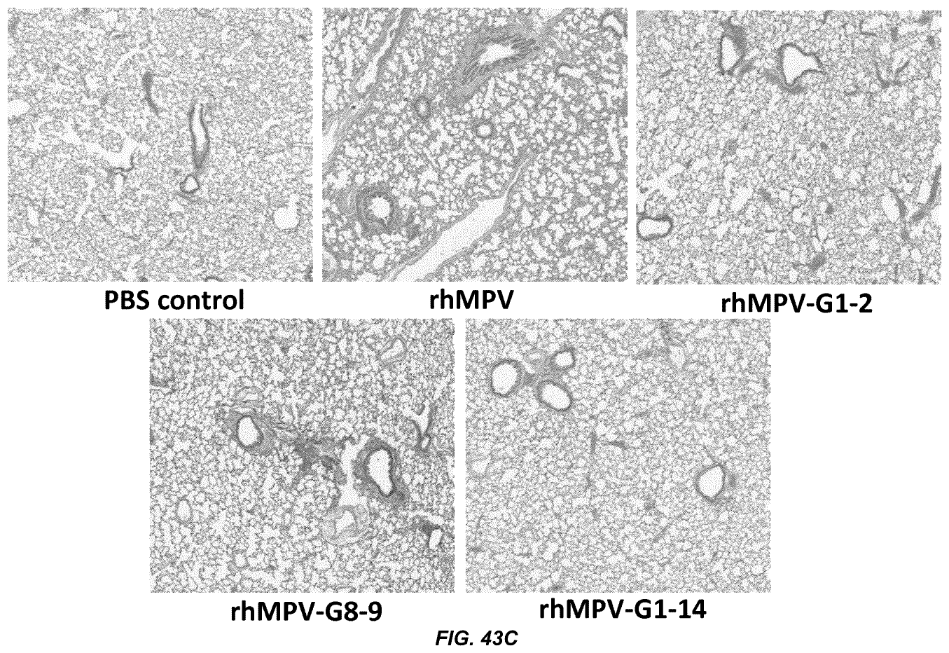

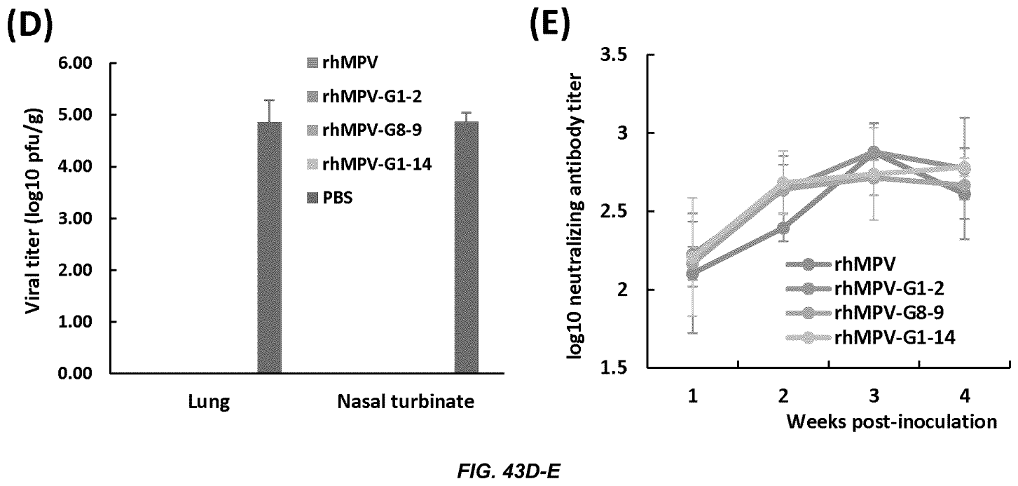

m⁶A-deficient rhMPVs induces higher type I interferon in vivo. The m⁶A-deficient rhMPVs induced significantly higher type I IFN in cell culture infections. The inventors next determined whether they also induce higher type I IFN in vivo using cotton rats, the best available small animal model for hMPV. Six-week-old SPF cotton rats were inoculated intranasally with 2.0×10⁵ PFU of rhMPV or m⁶A-deficient rhMPV mutants (rhMPV-G8-14 and rhMPV-G1-14) or PBS. At day 2 post-inoculation, cotton rats were terminated, bronchoalveolar lavage (BAL) was collected from the right lung and tested for IFN-β bioactivity. Briefly, CCRT cells were incubated with serially diluted BAL or human IFN-β, followed by infection with rVSV-GFP. The IFN-β level in BAL was calculated based on the inhibitory effect on rVSV-GFP infection using human IFN-β as the standard. Under these conditions, IFN-β bioactivity of BAL samples from rhMPV and PBS-inoculated cotton rats was below the detection limit (FIG. 43A). In contrast, IFN-β of BAL from rhMPV-G8-14 and rhMPV-G1-14 had average IFN-β bioactivity of 150.2 (P<0.0001) and 175.3 U/ml (P<0.01) respectively (FIG. 43A). This result demonstrated that m⁶A-deficient rhMPVs induced higher type I IFN responses in vivo compared to rhMPV.

m⁶A-deficient rhMPVs are significantly attenuated in replication in cotton rats. The m⁶A-deficient rhMPVs were significantly attenuated in cell culture. The inventors next determined whether they are attenuated in vivo using cotton rats as a model. Six-week-old SPF cotton rats were inoculated intranasally with 2.0×10⁵ PFU of rhMPV or m⁶A-deficient rhMPV mutant (rhMPV-G1-2, rhMPV-G8-9, and rhMPV-G1-14). At day 4 post-inoculation, cotton rats were terminated, and viral replication was determined. The average viral titers in lungs and nasal turbinates of rhMPV-inoculated cotton rats were 10^{5.08} and 10^{5.16} PFU/g tissue, respectively. Recombinant rhMPV-G1-2, rhMPV-G8-9, and rhMPV-G1-14 had 1.80, 2.03, and 2.7 log virus reductions in lungs compared to rhMPV, respectively (FIG. 43B). Particularly, rhMPV-G1-14 was only slightly above the detection limit for virus replication in the lungs. These m⁶A-deficient rhMPV mutants also had significant reductions (0.30-0.71 log, P<0.05) in viral replication in the nasal turbinates of cotton rats although the level of reduction was less than in the lungs (1.8-2.7 log) (FIG. 43B). No infectious virus was detected in either nasal turbinates or lungs in mock-infected cotton rats. These results demonstrated that m⁶A-deficient rhMPVs are more attenuated in replication in the lower respiratory tract than in the upper respiratory tract of cotton rats. The lungs of hMPV-infected cotton rats were also examined histologically (FIG. 43C). The parental hMPV caused moderate histologic lesions characterized by interstitial pneumonia, mononuclear cell infiltration, and edematous thickening of the bronchial submucosa. In contrast, fewer histological changes were found in the lungs of cotton rats infected with m⁶A-deficient rhMPVs. Thus, m⁶A-deficient rhMPVs were less pathogenic in cotton rats. Collectively, these results demonstrate that depletion of m⁶A sites in viral G gene significantly decreased hMPV replication and pathogenesis in vivo.

m⁶A-deficient rhMPVs provided complete protection against hMPV infection. Since m⁶A-deficient rhMPVs are significantly attenuated in vitro and in vivo, the inventors next determined whether they were immunogenic in cotton rats. Briefly, 6-week-old female cotton rats were intranasally immunized with 2.0×10⁵ PFU of rhMPV or m⁶A-deficient rhMPV mutants. After immunization, weekly serum was collected from each cotton rat. At week 4 post-immunization, cotton rats were challenged with 2.0×10⁵ PFU of rhMPV, terminated at day 4 after challenge, and viral titers in lungs and nasal turbinate were determined. For the unvaccinated challenged group, 10^{4.86} and 10^{4.87} PFU/g tissue were detected in the lungs and nasal turbinates, respectively (FIG. 43D). In contrast, cotton rats immunized with rhMPV, rhMPV-G1-2, rhMPV-G8-9, and rhMPV-G1-14 were completely protected from virus replication in both lungs and nasal turbinates (FIG. 43D). Also, rhMPV-G1-14 triggered significantly higher neutralizing antibody titers at weeks 1 and 2 than parental rhMPV (P<0.05) (FIG. 43E). At week 4, all m⁶A deficient rhMPVs had levels of antibody similar to rhMPV (FIG. 43E). Thus, m⁶A-deficient rhMPV retained wild type or even higher levels of immunogenicity and provided complete protection against hMPV infection.

### C. Discussion

The most prevalent epigenetic modification in all types of RNAs, rRNA, tRNA, snRNA, and mRNAs is m⁶A methylation. Viruses are obligatory intracellular parasites; their RNAs are also m⁶A methylated during replication in host cells. The presence of m⁶A in viral mRNA clearly enhances translation and mRNA stability (29, 31). However, the biological function of m⁶A in the viral genome and its replicative intermediate RNA has been mysterious. Using hMPV as a model, the inventors have demonstrated that m⁶A methylation of the antigenome and genome acts as a molecular signature for discriminating self from nonself RNA through the RNA sensor RIG-I. Several lines of evidence support this finding. First, the hMPV genome, antigenome, and mRNAs acquire m⁶A methylation during infection and hMPV infection enhances the expression of genes involved in innate immunity. Second, m⁶A methylation enhances hMPV replication and gene expression, and m⁶-deficient hMPVs are attenuated. Third, m⁶A-mutated rhMPVs, naturally m⁶A-deficient hMPV, and their antigenome and/or genome RNA triggered a higher type I IFN response. Fourth, m⁶A methylation protects the antigenome and genome from recognition by RIG-I thereby inhibiting RIG-I-dependent production of type I interferon in virus-infected cells and virion RNA-transfected cells. Fifth, m⁶A methylation of the viral antigenome and genome contributes to the evasion of the interferon-mediated restriction of viral replication. Finally, the deficiency of m⁶A methylation in the viral antigenome and genome RNA enhances the activation of the RIG-I pathway including RIG-I expression, RIG-I binding affinity, RIG-I conformational change, and IRF3 phosphorylation. The replication of m⁶A-deficient rhMPVs was restored when the RIG-I and MAVs signal pathways were knocked out. The data demonstrate that hMPV acquires m⁶A methylation in antigenome and genome as a means of mimicking host RNA to avoid the detection of innate immunity.

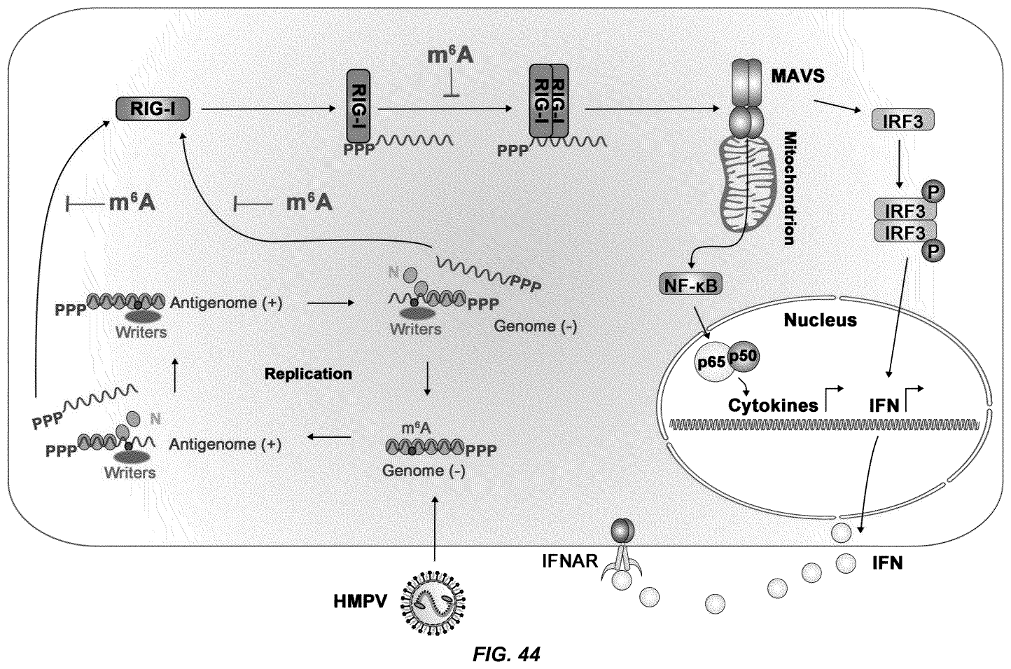

A model consistent with these findings is depicted in FIG. 44. Upon virus entry, the ribonucleoprotein (RNP) complex, composed of the genome wrapped in the nucleocapsid (N) protein, associated with the viral RNA-dependent RNA polymerase (RdRP), is delivered into the cytoplasm where viral transcription and replication occur. During transcription, the RdRP sequentially transcribes the 8 viral genes into 9 mRNAs which are m⁶A methylated and translated into 9 proteins, including the N protein. During replication, the RdRP initiates at the extreme 3′ end of the genome and synthesizes a full-length complementary antigenome, which is methylated by m⁶A writer proteins and subsequently encapsidated by soluble N protein in a helical nucleocapsid with 9 to 10 bases/rotation of the helix (62). This N-antigenomic RNA serves as template for synthesis of full-length progeny genomes, which are also m⁶A methylated and encapsidated by soluble N protein. RNA m⁶A methylation likely occurs prior to or concomitant with encapsidation, supported by the observation that N is partially co-localizes with METTL3 and strongly co-localizes with METTL14 in inclusion bodies where new RNP is assembled and active viral replication occurs. The antigenome and genome are 5′ triphosphorylated. Those that are not m⁶A methylated are recognized as a “nonself RNA” by RIG-I. The deficiency of m⁶A in virion RNA induces higher RIG-I expression, an enhanced RIG-I binding affinity and an enhanced ability to trigger the conformational change in RIG-I that corresponds to enhanced signaling to the downstream adaptor protein MAVS, activating IRF3 and NF-κB pathways, leading to higher production of type-I IFN and proinflammatory cytokines. In contrast, although the wild type hMPV virion RNA can be recognized by RIG-I due to the 5′ppp, the internal m⁶A modification appears to interfere with the high affinity binding of the RNA with the RIG-I helicase domain. Without this separate RNA interaction, the low binding affinity association with 5′ppp does not appear to efficiently induce the RIG-I mediated IFN signaling pathway. In addition, RIG-I is a 5′-triphosphate-dependent translocase, traveling from the 5′-ppp into the RNA chain to trigger oligomerization (63, 64). Recently, it was found that the translocation of RIG-I and the following RIG-I oligomerization is hindered by internal 2′-O methylation in dsRNA (65). Thus, it is likely that m⁶A methylation may also serve as a “brake” or “throttle” to prevent RIG-I translocation and oligomerization, leading to downstream signaling (FIG. 41B).

One aspect of this scenario would seem to be unlikely, that the m⁶A-modified genome or antigenome, tightly encapsidated by the N protein, would be accessible to RIG-I. And even if RIG-I could bind to the terminal 5′ppp, how would it be able to reach further into the RNA to find a non-methylated m⁶A site? A more likely scenario might be that all of the genomes and antigenomes that are synthesized are not encapsidated. Particularly early in the infectious cycle, when the concentration of the N protein is low, some of these full-length RNA genomes and antigenomes may not be encapsidated, enabling RIG-I access to both the 5′ppp and RNA downstream from it. Unencapsidated full-length genome or antigenome RNAs would likely be fragile, as they are susceptible to cytoplasmic RNases. However, only a 5′ fragment would be necessary to activate RIG-I in this scenario.

The m⁶A-seq analysis showed that all three species of viral RNA are m⁶A methylated and the strongest m⁶A peaks are located in the G gene mRNA and the region corresponding to the G gene in both genome and antigenome, leading the inventors to mutate these m⁶A sites. The inventors modified the positive-strand RNA, disrupting m⁶A sites of both the G mRNA (transcription product) and antigenome (replication intermediate). The inventors also mutated the m⁶A sites in the G gene in negative-sense genome RNA. By overexpressing m⁶A eraser protein, the inventors generated hMPV that is naturally defective in m⁶A methylation in its antigenome and genome. In all cases, these m⁶A-deficient rhMPVs and their virion RNAs induced significantly higher type I IFN responses. Both genome and antigenome ssRNAs contain 5′ triphosphate, a known ligand for RIG-I (4, 13). Removal of the 5′ triphosphate abrogated the RIG-I expression, RIG-I binding, IRF3 phosphorylation, and IFN response of both wild type antigenome and m⁶A-deficient antigenome, suggesting that 5′ triphosphate is absolutely required for RIG-I signaling. However, when m⁶A sites in the antigenome and genome were mutated or naturally removed by eraser proteins, the expression of RIG-I and the binding affinity of RIG-I for the m⁶A-deficient virion RNA was significantly enhanced compared to the wild type virion RNA, leading to a higher type I IFN response. Thus, marking antigenome and genome RNA with m⁶A methylation allows it to escape detection by RIG-I. The m⁶A sites in both genome and antigenome are involved in innate immune recognition.

Unlike genome and antigenome, hMPV mRNAs are capped and G-N-7 and ribose 2′-o are methylated at the 5′ end and the mRNA is polyadenylated at the 3′ end. Neither modification is recognized by RIG-I or MDA5. Previously, it was shown that viral mRNA lacking 2′-O methylation can be detected by MDA5 and the IFIT family, highlighting that 2′-o methylation also serves as a molecular marker for host innate immunity to discriminate self from nonself mRNA. Here the inventors found that m⁶A deficient G mRNA with G-N-7 and ribose 2′-O methylation is not recognized by RIG-I or MDA5, suggesting that m⁶A methylation in mRNA does not play a role in innate immunity. However, the data suggest that m⁶A methylation of viral mRNA plays an important role in enhancing mRNA translation. First, overexpression of m⁶A reader and writer proteins enhanced G protein expression whereas knockdown of these proteins inhibited G expression. Second, G protein expression was inhibited when m⁶A sites in G mRNA were mutated.

The inventors found that m⁶A-deficient hMPVs triggered significantly higher type I interferon responses compared to the parental hMPV, thereby contributing to the restriction of viral replication. In addition, both m⁶A-deficient rhMPV and isolated antigenome and/or genome RNA induced higher expression of RIG-I. However, IFN response was completely abrogated when RIG-I or MAVs but not MDA5 were knocked out from A549 cells. The binding affinity of RIG-I to m⁶A-deficient RNAs significantly increased compared to the m⁶A-sufficient RNAs. This suggests that RIG-I played a dominant role in recognizing m⁶A-deficient rhMPV and antigenome. This conclusion was further supported by the fact that the replication of m⁶A-deficient hMPVs was completely or partially restored in A549 cells when RIG-I or MAVs but not MDA5 were knocked out. In addition, the inventors found that m⁶A-deficient rhMPV and antigenome triggered a higher NF-κB driven SEAP activity. These results suggest that m⁶A-deficient RNA contributes to the enhanced activation of transcription factors belonging to the NF-κB and IRF families which lead to the enhanced expression of IFN.

Overall, the degrees of the defects in RNA m⁶A methylation are highly correlated with the levels of type I IFN responses and the levels of signaling molecules involved in the RIG-I mediated pathway. Antigenome of rhMPV-G1-14 contains more m⁶A site mutations than the antigenome of rhMPV-G1-2 and G8-9. Consistent with higher defects in m⁶A methylation, the antigenome of rhMPV-G1-14 induced significantly higher RIG-I expression, more RIG-I conformational changes, and more IFN production than the antigenome of rhMPV-G1-2 and G8-9 when their virion RNAs were transfected into A549 cells. Interestingly, in virus-infected cells, rhMPV-G1-2 and G8-9 induced more IFN than rhMPV-G1-14 under some conditions (e.g. MOI of 4.0 in A549 cells). The inventors interpret this discrepancy as being due to the complicated nature of IFN regulation during hMPV infection, involving viral RNA replication, protein synthesis, and alteration of host gene expression. In contrast, virion RNA transfection avoids these complicating factors, examining more directly the effects of m⁶A methylation of RNA on IFN production. Importantly, in addition to the RIG-I pathway, several other signaling pathways including MDA5 and TLR3/TLR4/TLR7 are triggered during hMPV infection (57, 72) and may play a role. Also, several viral proteins (G, M2-2, SH, and P) have been shown to inhibit these pathways (57, 72). Since the G gene region has the strongest m⁶A peaks in the hMPV genome, deletion of the G gene from the genome would result in a natural m⁶A-deficient virus. Thus, it is possible that m⁶A-deficient genome and antigenome produced by rhMPV-AG activated the RIG-I signaling pathway, rather than the loss of G protein expression suppressing RIG-I. Purified virion RNA from m⁶A deficient rhMPVs, which did not contain any viral proteins, directly triggered higher RIG-I expression and a more robust IFN response. In addition, compensation for the reduced G protein expression did not inhibit the IFN response of these m⁶A-deficient rhMPVs.

Viral RNA m⁶A methylation and its functions is an emerging field that has only been explored over the past two years. Detailed mechanisms by which m⁶A controls virus replication and gene expression are still poorly understood. The inventors demonstrated that the multiple biological functions of m⁶A methylation collectively contribute to enhanced hMPV replication and gene expression. First, during replication, the newly synthesized genome and replicative intermediate (antigenome) are m⁶A methylated by m⁶A writer proteins to prevent their detection by the innate immune system. Second, during transcription, viral mRNAs are also m⁶A methylated which enhances their translation which in turn may enhance virus spread. However, viral m⁶A appears to play an antiviral role in several flaviviruses such as HCV and Zika virus via an unknown mechanism(s) (40, 41). Resolving why m⁶A has a pro-viral function in some viruses whereas it has an antiviral function in other viruses may facilitate a strategy to develop m⁶A as an antiviral drug target.

One important application of this work is in the development of live attenuated vaccine candidates for hMPV by reducing m⁶A methylation in viral RNAs. Currently, hMPV is the second leading causative agent of acute respiratory disease in infants, children, and the elderly (76, 77), behind RSV. Despite major efforts, there is no FDA-approved vaccine for hMPV (77). Inactivated vaccines are not suitable for hMPV because they cause enhanced lung damage upon re-infection with the same virus (78). In contrast, enhanced lung damage has not been observed for live attenuated vaccine candidates (55, 79). Thus, a live attenuated vaccine is one of the most promising candidates for hMPV (80). However, it has been a challenge to identify a live attenuated vaccine strain that has an optimal balance between attenuation and immunogenicity. Since viral m⁶A acts in a pro-viral manner for hMPV, it should be feasible to generate an m⁶A-deficient rhMPV strain that is sufficiently attenuated yet retains high immunogenicity. In this study, the inventors showed that depletion of m⁶A sites in G mRNA resulted in a recombinant virus (rhMPV-G1-14) that is sufficiently attenuated in replication in the lungs but only had a mild defect in replication in nasal turbinate. Cotton rats immunized with this m⁶A-deficient hMPV expressed a high level of neutralizing antibody and were completely protected against challenge with parental rhMPV, highlighting the potential of utilizing an m⁶A-deficient hMPV mutant as a live vaccine candidate. This phenotype is similar to that of the cold-adapted attenuated viruses, which replicate in upper but not lower respiratory tracts. Cold-adapted (ca) temperature sensitive (ts) influenza virus vaccine has been licensed for use in humans since 1980 (81, 82).

A distinct advantage of targeting m⁶A sites for virus attenuation is that m⁶A-deficient hMPV mutants are capable of inducing a significantly higher type I IFN response compared to rhMPV. A higher IFN response will likely enhance adaptive immunity. Targeting different combinations of the many viral m⁶A sites could identify combinations with the optimal balance between attenuation and immunogenicity. A virus with mutations in multiple m⁶A sites would have enhanced genetic stability because reversion at any one site would have only a minor fitness gain. In fact, all m⁶A-deficient hMPV mutants were genetically stable; with no revertants or additional mutations detected after fifteen passages in A549 cells. In addition, m⁶A-deficient hMPV mutants grew to reasonably high titers in cell culture, especially in IFN-deficient cells, making vaccine production economically feasible. Thus, inhibition of viral m⁶A methylation is a novel approach to attenuating hMPV for the rational design of live attenuated vaccines.

In summary, the inventors discovered that the presence of m⁶A in virion RNA serves as a molecular signature for discrimination of self from non-self RNA by the cytoplasmic RNA sensor RIG-I. This work highlights that possibility of using m⁶A as a novel approach for the development of antiviral drugs and live attenuated vaccines for pneumoviruses.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All publications described herein are specifically incorporated by reference for all purposes.

### REFERENCES

The following references, to the extent that they provide exemplary procedural or other details supplementary to those set forth herein, are specifically incorporated herein by reference.

### Example 1 References

1. Yue, Y., J. Liu, and C. He, RNA N6-methyladenosine methylation in post-transcriptional gene expression regulation. Genes Dev, 2015. 29(13): p. 1343-55.
2. Roundtree, I. A., et al., Dynamic RNA Modifications in Gene Expression Regulation. Cell, 2017. 169(7): p. 1187-1200.
3. Desrosiers, R., K. Friderici, and F. Rottman, Identification of methylated nucleosides in messenger RNA from Novikoff hepatoma cells. Proc Natl Acad Sci USA, 1974. 71(10): p. 3971-5.
4. Lavi, S. and A. J. Shatkin, Methylated simian virus 40-specific RNA from nuclei and cytoplasm of infected BSC-1 cells. Proc Natl Acad Sci USA, 1975. 72(6): p. 2012-6.
5. Perry, R. P., et al., The methylated constituents of L cell messenger RNA: evidence for an unusual cluster at the 5′ terminus. Cell, 1975. 4(4): p. 387-94.
6. Wei, C. M., A. Gershowitz, and B. Moss, Methylated nucleotides block 5′ terminus of HeLa cell messenger RNA. Cell, 1975. 4(4): p. 379-86.
7. Jia, G., et al., N6-methyladenosine in nuclear RNA is a major substrate of the obesity-associated FTO. Nat Chem Biol, 2011. 7(12): p. 885-7.
8. Zheng, G., et al., ALKBH5 is a mammalian RNA demethylase that impacts RNA metabolism and mouse fertility. Mol Cell, 2013. 49(1): p. 18-29.
9. Meyer, K. D., et al., Comprehensive analysis of mRNA methylation reveals enrichment in Y UTRs and near stop codons. Cell, 2012. 149(7): p. 1635-46.
10. Linder, B., et al., Single-nucleotide-resolution mapping of m6A and m6Am throughout the transcriptome. Nat Methods, 2015. 12(8): p. 767-72.
11. Dominissini, D., et al., Transcriptome-wide mapping of N(6)-methyladenosine by m(6)A-seq based on immunocapturing and massively parallel sequencing. Nat Protoc, 2013. 8(1): p. 176-89.
12. Meyer, K. D. and S. R. Jaffrey, The dynamic epitranscriptome: N6-methyladenosine and gene expression control. Nat Rev Mol Cell Biol, 2014. 15(5): p. 313-26.
13. Vu, L. P., et al., The N6-methyladenosine (m6A)-forming enzyme METTL3 controls myeloid differentiation of normal hematopoietic and leukemia cells. Nat Med, 2017.
14. Jaffrey, S. R. and M. G. Kharas, Emerging links between m6A and misregulated mRNA methylation in cancer. Genome Med, 2017. 9(1): p. 2.
15. Wang, X., et al., N(6)-methyladenosine Modulates Messenger RNA Translation Efficiency. Cell, 2015. 161(6): p. 1388-99.
16. Zhao, B. S., et al., m6A-dependent maternal mRNA clearance facilitates zebrafish maternal-to-zygotic transition. Nature, 2017. 542(7642): p. 475-478.
17. Liu, J., et al., A METTL3-METTL14 complex mediates mammalian nuclear RNA N6-adenosine methylation. Nat Chem Biol, 2014. 10(2): p. 93-5.
18. Ping, X. L., et al., Mammalian WTAP is a regulatory subunit of the RNA N6-methyladenosine methyltransferase. Cell Res, 2014. 24(2): p. 177-89.
19. Dominissini, D., et al., Topology of the human and mouse m6A RNA methylomes revealed by m6A-seq. Nature, 2012. 485(7397): p. 201-6.
20. Wang, X., et al., N6-methyladenosine-dependent regulation of messenger RNA stability. Nature, 2014. 505(7481): p. 117-20.
21. Shi, H., et al., YTHDF3 facilitates translation and decay of N6-methyladenosine-modified RNA. Cell Res, 2017. 27(3): p. 315-328.
22. Li, A., et al., Cytoplasmic m6A reader YTHDF3 promotes mRNA translation. Cell Res, 2017. 27(3): p. 444-447.
23. Kane, S. E. and K. Beemon, Precise localization of m6A in Rous sarcoma virus RNA reveals clustering of methylation sites: implications for RNA processing. Mol Cell Biol, 1985. 5(9): p. 2298-306.
24. Canaani, D., et al., Identification and mapping of N6-methyladenosine containing sequences in simian virus 40 RNA. Nucleic Acids Res, 1979. 6(8): p. 2879-99.
25. Sommer, S., et al., The methylation of adenovirus-specific nuclear and cytoplasmic RNA. Nucleic Acids Res, 1976. 3(3): p. 749-65.
26. Moss, B., et al., 5′-Terminal and internal methylated nucleosides in herpes simplex virus type 1 mRNA. J Virol, 1977. 23(2): p. 234-9.
27. Furuichi, Y., et al., Blocked, methylated 5′-terminal sequence in avian sarcoma virus RNA. Nature, 1975. 257(5527): p. 618-20.
28. Krug, R. M., M. A. Morgan, and A. J. Shatkin, Influenza viral mRNA contains internal N6-methyladenosine and 5′-terminal 7-methylguanosine in cap structures. J Virol, 1976. 20(1): p. 45-53.
29. Kennedy, E. M., et al., Posttranscriptional m(6)A Editing of HIV-1 mRNAs Enhances Viral Gene Expression. Cell Host Microbe, 2016. 19(5): p. 675-85.
30. Lichinchi, G., et al., Dynamics of the human and viral m(6)A RNA methylomes during HIV-1 infection of T cells. Nat Microbiol, 2016. 1: p. 16011.
31. Tirumuru, N., et al., N(6)-methyladenosine of HIV-1 RNA regulates viral infection and HIV-1 Gag protein expression. Elife, 2016. 5.
32. Courtney, D. G., et al., Epitranscriptomic Enhancement of Influenza A Virus Gene Expression and Replication. Cell Host Microbe, 2017. 22(3): p. 377-386 e5.
33. Tsai, K., D. G. Courtney, and B. R. Cullen, Addition of m6A to SV40 late mRNAs enhances viral structural gene expression and replication. PLoS Pathog, 2018. 14(2): p. e1006919.
34. Hesser, C. R., et al., N6-methyladenosine modification and the YTHDF2 reader protein play cell type specific roles in lytic viral gene expression during Kaposi's sarcoma-associated herpesvirus infection. PLoS Pathog, 2018. 14(4): p. e1006995.
35. Ye, F., E. R. Chen, and T. W. Nilsen, Kaposi's Sarcoma-Associated Herpesvirus Utilizes and Manipulates RNA N(6)-Adenosine Methylation To Promote Lytic Replication. J Virol, 2017. 91(16).
36. Tan, B., et al., Viral and cellular N(6)-methyladenosine and N(6),2′-O-dimethyladenosine epitranscriptomes in the KSHV life cycle. Nat Microbiol, 2018. 3(1): p. 108-120.
37. Gokhale, N. S., et al., N6-Methyladenosine in Flaviviridae Viral RNA Genomes Regulates Infection. Cell Host Microbe, 2016. 20(5): p. 654-665.
38. Lichinchi, G., et al., Dynamics of Human and Viral RNA Methylation during Zika Virus Infection. Cell Host Microbe, 2016. 20(5): p. 666-673.
39. Cowton, V. M., D. R. McGivern, and R. Fearns, Unravelling the complexities of respiratory syncytial virus RNA synthesis. J Gen Virol, 2006. 87(Pt 7): p. 1805-21.
40. Fearns, R., M. E. Peeples, and P. L. Collins, Increased expression of the N protein of respiratory syncytial virus stimulates minigenome replication but does not alter the balance between the synthesis of mRNA and antigenome. Virology, 1997. 236(1): p. 188-201.
41. Ruigrok, R. W., T. Crepin, and D. Kolakofsky, Nucleoproteins and nucleocapsids of negative-strand RNA viruses. Curr Opin Microbiol, 2011. 14(4): p. 504-10.
42. Afonso, C. L., et al., Taxonomy of the order Mononegavirales: update 2016. Arch Virol, 2016. 161(8): p. 2351-60.
43. Collins, P. L. and B. S. Graham, Viral and host factors in human respiratory syncytial virus pathogenesis. J Virol, 2008. 82(5): p. 2040-55.
44. Nair, H., et al., Global burden of acute lower respiratory infections due to respiratory syncytial virus in young children: a systematic review and meta-analysis. Lancet, 2010. 375(9725): p. 1545-55.
45. van den Hoogen, B. G., et al., A newly discovered human pneumovirus isolated from young children with respiratory tract disease. Nat Med, 2001. 7(6): p. 719-24.
46. Edwards, K. M., et al., Burden of human metapneumovirus infection in young children. N Engl J Med, 2013. 368(7): p. 633-43.
47. Kolakofsky, D. and A. Bruschi, Antigenomes in Sendai virions and Sendai virus-infected cells. Virology, 1975. 66(1): p. 185-91.
48. Liu, L., et al., QNB: differential RNA methylation analysis for count-based small-sample sequencing data with a quad-negative binomial model. BMC Bioinformatics, 2017. 18(1): p. 387.
49. Kennedy, E. M., et al., Posttranscriptional m(6)A Editing of HIV-1 mRNAs Enhances Viral Gene Expression. Cell Host Microbe, 2017. 22(6): p. 830.
50. Whelan, S. P., J. N. Barr, and G. W. Wertz, Transcription and replication of nonsegmented negative-strand RNA viruses. Curr Top Microbiol Immunol, 2004. 283: p. 61-119.
51. Johnson, S. M., et al., Respiratory Syncytial Virus Uses CX3CR1 as a Receptor on Primary Human Airway Epithelial Cultures. PLoS Pathog, 2015. 11(12): p. e1005318.
52. de Clercq, E. and J. A. Montgomery, Broad-spectrum antiviral activity of the carbocyclic analog of 3-deazaadenosine. Antiviral Res, 1983. 3(1): p. 17-24.
53. Wyde, P. R., et al., Evaluation of the toxicity and antiviral activity of carbocyclic 3-deazaadenosine against respiratory syncytial and parainfluenza type 3 viruses in tissue culture and in cotton rats. Antiviral Res, 1990. 14(4-5): p. 215-25.
54. Boyoglu-Barnum, S., et al., Mutating the CX3C Motif in the G Protein Should Make a Live Respiratory Syncytial Virus Vaccine Safer and More Effective. J Virol, 2017. 91(10).
55. Teng, M. N. and P. L. Collins, The central conserved cystine noose of the attachment G protein of human respiratory syncytial virus is not required for efficient viral infection in vitro or in vivo. J Virol, 2002. 76(12): p. 6164-71.
56. Teng, M. N., S. S. Whitehead, and P. L. Collins, Contribution of the respiratory syncytial virus G glycoprotein and its secreted and membrane-bound forms to virus replication in vitro and in vivo. Virology, 2001. 289(2): p. 283-96.
57. Vanover, D., et al., RSV glycoprotein and genomic RNA dynamics reveal filament assembly prior to the plasma membrane. Nat Commun, 2017. 8(1): p. 667.
58. Durbin, A. F., et al., RNAs Containing Modified Nucleotides Fail To Trigger RIG-I Conformational Changes for Innate Immune Signaling. MBio, 2016. 7(5).
59. Mayers, D. L., et al., Anti-human immunodeficiency virus 1 (HIV-1) activities of 3-deazaadenosine analogs: increased potency against 3′-azido-3′-deoxythymidine-resistant HIV-1 strains. Proc Natl Acad Sci USA, 1995. 92(1): p. 215-9.
60. Kim, D., B. Langmead, and S. L. Salzberg, HISAT: a fast spliced aligner with low memory requirements. Nat Methods, 2015. 12(4): p. 357-60.
61. Ma, L., et al., Evolution of transcript modification by N6-methyladenosine in primates. Genome Res, 2017. 27(3): p. 385-392.
62. Cui, X., et al., Guitar: An R/Bioconductor Package for Gene Annotation Guided Transcriptomic Analysis of RNA-Related Genomic Features. Biomed Res Int, 2016. 2016: p. 8367534.
63. Collins, P. L., et al., Production of infectious human respiratory syncytial virus from cloned cDNA confirms an essential role for the transcription elongation factor from the 5′ proximal open reading frame of the M2 mRNA in gene expression and provides a capability for vaccine development. Proc Natl Acad Sci USA, 1995. 92(25): p. 11563-7.
64. Tripp, R. A., Jones, L. P., Haynes, L. M., Zheng, H., Murphy, P. M., and Anderson, L. J. CX3C chemokine mimicry by respiratory syncytial virus G glycoprotein. Nat Immunol, 2001. 2(8):732-8.

### Example 2 References

1. Takeuchi O, Akira S. 2009. Innate immunity to virus infection. Immunological Reviews 227:75-86.
2. Loo Y M, Gale M, Jr. 2007. Viral regulation and evasion of the host response. Current Topics in Microbiology and Immunology 316:295-313.
3. Chow K T, Gale M, Jr., Loo Y M. 2018. RIG-I and Other RNA Sensors in Antiviral Immunity. Annual Review of Immunology 36:667-694.
4. Hornung V, Ellegast J, Kim S, Brzozka K, Jung A, Kato H, Poeck H, Akira S, Conzelmann K K, Schlee M, Endres S, Hartmann G. 2006. 5′-Triphosphate RNA is the ligand for RIG-I. Science 314:994-997.
5. Pichlmair A, Schulz O, Tan C P, Naslund T I, Liljestrom P, Weber F, Reis e Sousa C. 2006. RIG-I-mediated antiviral responses to single-stranded RNA bearing 5′-phosphates. Science 314:997-1001.
6. Saito T, Owen D M, Jiang F, Marcotrigiano J, Gale M, Jr. 2008. Innate immunity induced by composition-dependent RIG-I recognition of hepatitis C virus RNA. Nature 454:523-527.
7. Uzri D, Gehrke L. 2009. Nucleotide sequences and modifications that determine RIG-I/RNA binding and signaling activities. Journal of Virology 83:4174-4184.
8. Schlee M, Roth A, Hornung V, Hagmann C A, Wimmenauer V, Barchet W, Coch C, Janke M, Mihailovic A, Wardle G, Juranek S, Kato H, Kawai T, Poeck H, Fitzgerald K A, Takeuchi O, Akira S, Tuschl T, Latz E, Ludwig J, Hartmann G. 2009. Recognition of 5′ triphosphate by RIG-I helicase requires short blunt double-stranded RNA as contained in panhandle of negative-strand virus. Immunity 31:25-34.
9. Kato H, Takeuchi O, Mikamo-Satoh E, Hirai R, Kawai T, Matsushita K, Hiiragi A, Dermody T S, Fujita T, Akira S. 2008. Length-dependent recognition of double-stranded ribonucleic acids by retinoic acid-inducible gene-I and melanoma differentiation-associated gene 5. The Journal of Experimental Medicine 205:1601-1610.
10. Runge S, Sparrer K M, Lassig C, Hembach K, Baum A, Garcia-Sastre A, Soding J, Conzelmann K K, Hopfner K P. 2014. In vivo ligands of MDA5 and RIG-I in measles virus-infected cells. PLoS Pathogens 10:e1004081.
11. Shuman S. 2002. What messenger RNA capping tells us about eukaryotic evolution. Nature reviews. Molecular Cell Biology 3:619-625.
12. Furuichi Y, LaFiandra A, Shatkin AJ. 1977. 5′-Terminal structure and mRNA stability. Nature 266:235-239.
13. Hyde J L, Diamond M S. 2015. Innate immune restriction and antagonism of viral RNA lacking 2-O methylation. Virology 479-480:66-74.
14. Li J, Wang J T, Whelan S P. 2006. A unique strategy for mRNA cap methylation used by vesicular stomatitis virus. Proceedings of the National Academy of Sciences of the United States of America 103:8493-8498.
15. Ray D, Shah A, Tilgner M, Guo Y, Zhao Y, Dong H, Deas T S, Zhou Y, Li H, Shi P Y. 2006. West Nile virus 5′-cap structure is formed by sequential guanine N-7 and ribose 2′-O methylations by nonstructural protein 5. Journal of Virology 80:8362-8370.
16. Chen Y, Su C, Ke M, Jin X, Xu L, Zhang Z, Wu A, Sun Y, Yang Z, Tien P, Ahola T, Liang Y, Liu X, Guo D. 2011. Biochemical and structural insights into the mechanisms of SARS coronavirus RNA ribose 2′-O-methylation by nsp16/nsp10 protein complex. PLoS Pathogens 7:e1002294.
17. Zust R, Cervantes-Barragan L, Habjan M, Maier R, Neuman B W, Ziebuhr J, Szretter K J, Baker S C, Barchet W, Diamond M S, Siddell S G, Ludewig B, Thiel V. 2011. Ribose 2′-O-methylation provides a molecular signature for the distinction of self and non-self mRNA dependent on the RNA sensor Mda5. Nature Immunology 12:137-143.
18. Daffis S, Szretter K J, Schriewer J, Li J, Youn S, Errett J, Lin T Y, Schneller S, Zust R, Dong H, Thiel V, Sen G C, Fensterl V, Klimstra W B, Pierson T C, Buller R M, Gale M, Jr., Shi P Y, Diamond M S. 2010. 2′-O methylation of the viral mRNA cap evades host restriction by IFIT family members. Nature 468:452-456.
19. Abbas Y M, Laudenbach B T, Martinez-Montero S, Cencic R, Habjan M, Pichlmair A, Damha M J, Pelletier J, Nagar B. 2017. Structure of human IFIT1 with capped RNA reveals adaptable mRNA binding and mechanisms for sensing N1 and N2 ribose 2′-O methylations. Proceedings of the National Academy of Sciences of the United States of America 114:E2106-E2115.
20. Yue Y, Liu J, He C. 2015. RNA N6-methyladenosine methylation in post-transcriptional gene expression regulation. Genes & Development 29:1343-1355.
21. Roundtree I A, Evans M E, Pan T, He C. 2017. Dynamic RNA Modifications in Gene Expression Regulation. Cell 169:1187-1200.
22. Desrosiers R, Friderici K, Rottman F. 1974. Identification of methylated nucleosides in messenger RNA from Novikoff hepatoma cells. Proceedings of the National Academy of Sciences of the United States of America 71:3971-3975.
23. Perry R P, Kelley D E, Friderici K, Rottman F. 1975. The methylated constituents of L cell messenger RNA: evidence for an unusual cluster at the 5′ terminus. Cell 4:387-394.
24. Wei C M, Gershowitz A, Moss B. 1975. Methylated nucleotides block 5′ terminus of HeLa cell messenger RNA. Cell 4:379-386.
25. Liu J, Yue Y, Han D, Wang X, Fu Y, Zhang L, Jia G, Yu M, Lu Z, Deng X, Dai Q, Chen W, He C. 2014. A METTL3-METTL14 complex mediates mammalian nuclear RNA N6-adenosine methylation. Nature Chemical Biology 10:93-95.
26. Ping X L, Sun B F, Wang L, Xiao W, Yang X, Wang W J, Adhikari S, Shi Y, Lv Y, Chen Y S, Zhao X, Li A, Yang Y, Dahal U, Lou X M, Liu X, Huang J, Yuan W P, Zhu X F, Cheng T, Zhao Y L, Wang X, Rendtlew Danielsen J M, Liu F, Yang Y G. 2014. Mammalian WTAP is a regulatory subunit of the RNA N6-methyladenosine methyltransferase. Cell Research 24:177-189.
27. Jia G, Fu Y, Zhao X, Dai Q, Zheng G, Yang Y, Yi C, Lindahl T, Pan T, Yang Y G, He C. 2011. N6-methyladenosine in nuclear RNA is a major substrate of the obesity-associated FTO. Nature Chemical Biology 7:885-887.
28. Zheng G, Dahl J A, Niu Y, Fedorcsak P, Huang C M, Li C J, Vagbo C B, Shi Y, Wang W L, Song S H, Lu Z, Bosmans R P, Dai Q, Hao Y J, Yang X, Zhao W M, Tong W M, Wang X J, Bogdan F, Furu K, Fu Y, Jia G, Zhao X, Liu J, Krokan H E, Klungland A, Yang Y G, He C. 2013. ALKBH5 is a mammalian RNA demethylase that impacts RNA metabolism and mouse fertility. Molecular Cell 49:18-29.
29. Wang X, Zhao B S, Roundtree I A, Lu Z, Han D, Ma H, Weng X, Chen K, Shi H, He C. 2015. N(6)-methyladenosine Modulates Messenger RNA Translation Efficiency. Cell 161:1388-1399.
30. Dominissini D, Moshitch-Moshkovitz S, Schwartz S, Salmon-Divon M, Ungar L, Osenberg S, Cesarkas K, Jacob-Hirsch J, Amariglio N, Kupiec M, Sorek R, Rechavi G. 2012. Topology of the human and mouse m6A RNA methylomes revealed by m6A-seq. Nature 485:201-206.
31. Wang X, Lu Z, Gomez A, Hon G C, Yue Y, Han D, Fu Y, Parisien M, Dai Q, Jia G, Ren B, Pan T, He C. 2014. N6-methyladenosine-dependent regulation of messenger RNA stability. Nature 505:117-120.
32. Frye M, Harada B T, Behm M, He C. 2018. RNA modifications modulate gene expression during development. Science 361:1346-1349.
33. Zhao B S, Wang X, Beadell A V, Lu Z, Shi H, Kuuspalu A, Ho R K, He C. 2017. m(6)A-dependent maternal mRNA clearance facilitates zebrafish maternal-to-zygotic transition. Nature 542:475-478.
34. Lavi S, Shatkin A J. 1975. Methylated simian virus 40-specific RNA from nuclei and cytoplasm of infected BSC-1 cells. Proceedings of the National Academy of Sciences of the United States of America 72:2012-2016.
35. Furuichi Y, Shatkin A J, Stavnezer E, Bishop J M. 1975. Blocked, methylated 5′-terminal sequence in avian sarcoma virus RNA. Nature 257:618-620.
36. Moss B, Gershowitz A, Stringer J R, Holland L E, Wagner E K. 1977. 5′-Terminal and internal methylated nucleosides in herpes simplex virus type 1 mRNA. Journal of Virology 23:234-239.
37. Sommer S, Salditt-Georgieff M, Bachenheimer S, Darnell J E, Furuichi Y, Morgan M, Shatkin A J. 1976. The methylation of adenovirus-specific nuclear and cytoplasmic RNA. Nucleic Acids Research 3:749-765.
38. Dominissini D, Moshitch-Moshkovitz S, Salmon-Divon M, Amariglio N, Rechavi G. 2013. Transcriptome-wide mapping of N(6)-methyladenosine by m(6)A-seq based on immunocapturing and massively parallel sequencing. Nature Protocols 8:176-189.
39. Linder B, Grozhik A V, Olarerin-George A O, Meydan C, Mason C E, Jaffrey S R. 2015. Single-nucleotide-resolution mapping of m6A and m6Am throughout the transcriptome. Nature Methods 12:767-772.
40. Gokhale N S, McIntyre A B R, McFadden M J, Roder A E, Kennedy E M, Gandara J A, Hopcraft S E, Quicke K M, Vazquez C, Willer J, Ilkayeva O R, Law B A, Holley C L, Garcia-Blanco M A, Evans M J, Suthar M S, Bradrick S S, Mason C E, Horner S M. 2016. N6-Methyladenosine in Flaviviridae Viral RNA Genomes Regulates Infection. Cell Host & Microbe 20:654-665.
41. Lichinchi G, Zhao B S, Wu Y, Lu Z, Qin Y, He C, Rana T M. 2016. Dynamics of Human and Viral RNA Methylation during Zika Virus Infection. Cell Host & Microbe 20:666-673.
42. Courtney D G, Kennedy E M, Dumm R E, Bogerd H P, Tsai K, Heaton N S, Cullen B R. 2017. Epitranscriptomic Enhancement of Influenza A Virus Gene Expression and Replication. Cell Host & Microbe 22:377-386 e375.
43. Tsai K, Courtney D G, Cullen B R. 2018. Addition of m6A to SV40 late mRNAs enhances viral structural gene expression and replication. PLoS Pathogens 14:e1006919.
44. Hesser C R, Karijolich J, Dominissini D, He C, Glaunsinger B A. 2018. N6-methyladenosine modification and the YTHDF2 reader protein play cell type specific roles in lytic viral gene expression during Kaposi's sarcoma-associated herpesvirus infection. PLoS Pathogens 14:e1006995.
45. Tan B, Liu H, Zhang S, da Silva S R, Zhang L, Meng J, Cui X, Yuan H, Sorel O, Zhang S W, Huang Y, Gao S J. 2018. Viral and cellular N(6)-methyladenosine and N(6),2′-O-dimethyladenosine epitranscriptomes in the KSHV life cycle. Nature Microbiology 3:108-120.
46. Ye F, Chen E R, Nilsen T W. 2017. Kaposi's Sarcoma-Associated Herpesvirus Utilizes and Manipulates RNA N(6)-Adenosine Methylation To Promote Lytic Replication. Journal of Virology 91.
47. Lichinchi G, Gao S, Saletore Y, Gonzalez G M, Bansal V, Wang Y, Mason C E, Rana T M. 2016. Dynamics of the human and viral m(6)A RNA methylomes during HIV-1 infection of T cells. Nature Microbiology 1:16011.
48. Kennedy E M, Bogerd H P, Kornepati A V, Kang D, Ghoshal D, Marshall J B, Poling B C, Tsai K, Gokhale N S, Horner S M, Cullen B R. 2016. Posttranscriptional m(6)A Editing of HIV-1 mRNAs Enhances Viral Gene Expression. Cell Host & Microbe 19:675-685.
49. Tirumuru N, Zhao B S, Lu W, Lu Z, He C, Wu L. 2016. N(6)-methyladenosine of HIV-1 RNA regulates viral infection and HIV-1 Gag protein expression. eLife 5.
50. Herfst S, de Graaf M, Schickli J H, Tang R S, Kaur J, Yang C F, Spaete R R, Haller A A, van den Hoogen B G, Osterhaus A D, Fouchier R A. 2004. Recovery of human metapneumovirus genetic lineages a and B from cloned cDNA. Journal of Virology 78:8264-8270.
51. Zhang Y, Wei Y, Li J. 2012. Development and optimization of a direct plaque assay for human and avian metapneumoviruses. J Virol Methods 185:61-68.
52. Kim D, Pertea G, Trapnell C, Pimentel H, Kelley R, Salzberg S L. 2013. TopHat2: accurate alignment of transcriptomes in the presence of insertions, deletions and gene fusions. Genome Biology 14:R36.
53. Tripathi S, Pohl M O, Zhou Y, Rodriguez-Frandsen A, Wang G, Stein D A, Moulton H M, DeJesus P, Che J, Mulder L C, Yanguez E, Andenmatten D, Pache L, Manicassamy B, Albrecht R A, Gonzalez M G, Nguyen Q, Brass A, Elledge S, White M, Shapira S, Hacohen N, Karlas A, Meyer T F, Shales M, Gatorano A, Johnson J R, Jang G, Johnson T, Verschueren E, Sanders D, Krogan N, Shaw M, Konig R, Stertz S, Garcia-Sastre A, Chanda S K. 2015. Meta- and Orthogonal Integration of Influenza “OMICs” Data Defines a Role for UBR4 in Virus Budding. Cell Host & Microbe 18:723-735.
54. Cai H, Zhang Y, Ma Y, Sun J, Liang X, Li J. 2015. Zinc binding activity of human metapneumovirus M2-1 protein is indispensable for viral replication and pathogenesis in vivo. Journal of Virology 89:6391-6405.
55. Zhang Y, Wei Y, Zhang X, Cai H, Niewiesk S, Li J. 2014. Rational design of human metapneumovirus live attenuated vaccine candidates by inhibiting viral mRNA cap methyltransferase. Journal of Virology 88:11411-11429.
56. Kolakofsky D, Bruschi A. 1975. Antigenomes in Sendai virions and Sendai virus-infected cells. Virology 66:185-191.
57. Kolli D, Bao X, Casola A. 2012. Human metapneumovirus antagonism of innate immune responses. Viruses 4:3551-3571.
58. Kolakofsky D, Kowalinski E, Cusack S. 2012. A structure-based model of RIG-I activation. RNA 18:2118-2127.
59. Zeng W, Sun L, Jiang X, Chen X, Hou F, Adhikari A, Xu M, Chen Z J. 2010. Reconstitution of the RIG-I pathway reveals a signaling role of unanchored polyubiquitin chains in innate immunity. Cell 141:315-330.
60. Durbin A F, Wang C, Marcotrigiano J, Gehrke L. 2016. RNAs Containing Modified Nucleotides Fail To Trigger RIG-I Conformational Changes for Innate Immune Signaling. mBio 7.
61. Jiang F, Ramanathan A, Miller M T, Tang G Q, Gale M, Jr., Patel S S, Marcotrigiano J. 2011. Structural basis of RNA recognition and activation by innate immune receptor RIG-I. Nature 479:423-427.
62. Green T J, Zhang X, Wertz G W, Luo M. 2006. Structure of the vesicular stomatitis virus nucleoprotein-RNA complex. Science 313:357-360.
63. Myong S, Cui S, Cornish P V, Kirchhofer A, Gack M U, Jung J U, Hopfner K P, Ha T. 2009. Cytosolic viral sensor RIG-I is a 5′-triphosphate-dependent translocase on double-stranded RNA. Science 323:1070-1074.
64. Zheng J, Wang C, Chang M R, Devarkar S C, Schweibenz B, Crynen G C, Garcia-Ordonez R D, Pascal B D, Novick S J, Patel S S, Marcotrigiano J, Griffin P R. 2018. HDX-MS reveals dysregulated checkpoints that compromise discrimination against self RNA during RIG-I mediated autoimmunity. Nature Communications 9:5366.
65. Devarkar S C, Schweibenz B, Wang C, Marcotrigiano J, Patel S S. 2018. RIG-I Uses an ATPase-Powered Translocation-Throttling Mechanism for Kinetic Proofreading of RNAs and Oligomerization. Molecular Cell 72:355-368 e354.
66. Kariko K, Buckstein M, Ni H, Weissman D. 2005. Suppression of RNA recognition by Toll-like receptors: the impact of nucleoside modification and the evolutionary origin of RNA. Immunity 23:165-175.
67. Sioud M, Furset G, Cekaite L. 2007. Suppression of immunostimulatory siRNA-driven innate immune activation by 2′-modified RNAs. Biochemical and Biophysical Research Communications 361:122-126.
68. Coots R A, Liu X M, Mao Y, Dong L, Zhou J, Wan J, Zhang X, Qian S B. 2017. m(6)A Facilitates eIF4F-Independent mRNA Translation. Molecular Cell 68:504-514 e507.
69. Rubio R M, Depledge D P, Bianco C, Thompson L, Mohr I. 2018. RNA m(6) A modification enzymes shape innate responses to DNA by regulating interferon beta. Genes & Development 32:1472-1484.
70. Zheng Q, Hou J, Zhou Y, Li Z, Cao X. 2017. The RNA helicase DDX46 inhibits innate immunity by entrapping m(6)A-demethylated antiviral transcripts in the nucleus. Nature Immunology 18:1094-1103.
71. Winkler R, Gillis E, Lasman L, Safra M, Geula S, Soyris C, Nachshon A, Tai-Schmiedel J, Friedman N, Le-Trilling V T K, Trilling M, Mandelboim M, Hanna J H, Schwartz S, Stern-Ginossar N. 2019. m(6)A modification controls the innate immune response to infection by targeting type I interferons. Nature Immunology 20:173-182.
72. Cespedes P F, Palavecino C E, Kalergis A M, Bueno S M. 2016. Modulation of Host Immunity by the Human Metapneumovirus. Clinical Microbiology Reviews 29:795-818.
73. Bao X, Liu T, Shan Y, Li K, Garofalo R P, Casola A. 2008. Human metapneumovirus glycoprotein G inhibits innate immune responses. PLoS Pathogens 4:e1000077.
74. Kolli D, Bao X, Liu T, Hong C, Wang T, Garofalo R P, Casola A. 2011. Human metapneumovirus glycoprotein G inhibits TLR4-dependent signaling in monocyte-derived dendritic cells. Journal of Immunology 187:47-54.
75. Biacchesi S, Skiadopoulos M H, Yang L, Lamirande E W, Tran K C, Murphy B R, Collins P L, Buchholz U J. 2004. Recombinant human Metapneumovirus lacking the small hydrophobic SH and/or attachment G glycoprotein: deletion of G yields a promising vaccine candidate. Journal of Virology 78:12877-12887.
76. van den Hoogen B G, de Jong J C, Groen J, Kuiken T, de Groot R, Fouchier R A, Osterhaus A D. 2001. A newly discovered human pneumovirus isolated from young children with respiratory tract disease. Nature Medicine 7:719-724.
77. Schildgen V, van den Hoogen B, Fouchier R, Tripp R A, Alvarez R, Manoha C, Williams J, Schildgen O. 2011. Human Metapneumovirus: lessons learned over the first decade. Clinical Microbiology Reviews 24:734-754.
78. Yim K C, Cragin R P, Boukhvalova M S, Blanco J C, Hamlin M E, Boivin G, Porter D D, Prince G A. 2007. Human metapneumovirus: enhanced pulmonary disease in cotton rats immunized with formalin-inactivated virus vaccine and challenged. Vaccine 25:5034-5040.
79. Buchholz U J, Nagashima K, Murphy B R, Collins P L. 2006. Live vaccines for human metapneumovirus designed by reverse genetics. Expert Review of Vaccines 5:695-706.
80. Wen S C, Williams J V. 2015. New Approaches for Immunization and Therapy against Human Metapneumovirus. Clinical and Vaccine Immunology: CVI 22:858-866.
81. Reeve P, Almond J W, Felsenreich V, Pibermann M, Maassab H F. 1980. Studies with a cold-recombinant A/Victoria/3/75 (H3N2) virus. I. biologic, genetic, and biochemical characterization. The Journal of Infectious Diseases 142:850-856.
82. Murphy B R, Holley H P, Jr., Berquist E J, Levine M M, Spring S B, Maassab H F, Kendal A P, Chanock R M. 1979. Cold-adapted variants of influenza A virus: evaluation in adult seronegative volunteers of A/Scotland/840/74 and A/Victoria/3/75 cold-adapted recombinants derived from the cold-adapted A/Ann Arbor/6/60 strain. Infection and Immunity 23:253-259.

1-46. (canceled)

47. A method for inhibiting a negative-sense single-stranded RNA virus of the family Pneumoviridae in a patient comprising administering to the patient an effective amount of a composition comprising an inhibitor of N6-methyladenosine (m6A) methylation.

48-53. (canceled)

54. A host cell comprising a heterologous nucleic acid encoding one or more N6-methyladenosine (m6A) reader or writer proteins.

55. The host cell of claim 54, wherein the writer protein comprises methyltransferase-like 3 (METTL3) and/or methyltransferase-like 14 (METTL14).

56. The host cell of claim 54, wherein the reader protein comprises YTH N6-methyladenosine RNA binding protein F1 (YTHDF1), YTH N6-methyladenosine RNA binding protein F2 (YTHDF2), YTH N6-methyladenosine RNA binding protein F3 (YTHDF3), and/or YTH N6-methyladenosine RNA binding protein C1 (YTHDC1).

57. The host cell of claim 54, further comprising a heterologous nucleic acid encoding an attenuated virus whose replication is positively affected by m6A modification.

58. The host cell of claim 57, wherein the attenuated virus is selected from the group consisting of Paramyxoviridae, Pneumoviridae, Rhabdoviridae, Filoviridae, and Bornaviridae.

59. The host cell of claim 57, wherein the attenuated virus is a human respiratory syncytial virus (RSV), a human metapneumovirus (hMPV), avian metapneumovirus (aMPV), pneumonia virus of mice (PVM), bovine RSV, poliovirus, measles virus, mumps virus, rubella virus, yellow fever virus, influenza virus, parainfluenza viruses, metapneumoviruses, Zika virus, dengue viruses, rhinoviruses, adenovirus, Sendai virus, vesicular stomatitis virus, parainfluenza viruses, measles virus, and/or Newcastle disease.

60. The host cell of claim 54, wherein the cell comprises a VERO, MRC-5, HEp-2, A549, HeLa, or human airway epithelial (HAE) cell.

61. The host cell of claim 54, wherein the heterologous nucleic acid is stably integrated into the genome of the host cell.

62. The host cell of claim 57, wherein the virus comprises one or more sequence alterations that reduce m6A modification of viral mRNA, genome, and/or antigenome.

63. The host cell of claim 62, wherein the sequence alterations change at least two nucleotides of at least one m6A consensus site.

64. The host cell of claim 62, wherein the sequence alterations in the viral genome or antigenome correspond to the G gene encoding one or more m6A consensus sites.

65. The host cell of claim 64, wherein the m6A consensus sites are in regions 392-467 nt, 567-660 nt, and/or 716-795 nt of the G gene in reference to SEQ ID NO: 1.

66. The host cell of claim 64, wherein the m6A consensus sites comprise one or more consensus sites selected from the following consensus sites in the MPV antigenome: site 1, 171-AAm⁶AC»TA-175; site 2, 187-GAm⁶A»GCA-191; site 3, 227-AAm⁶ACT»G-231; site 4, 246-AGm⁶AC»TA-250; site 5, 255-AGm⁶AC»TA-259; site 6, 341-AGm⁶ACA»G-345; site 7, 346-GAm⁶A»GCC-351; site 8, 422-GAm⁶ACA»G-426; site 9, 428-AGm⁶ACA»G-432; site 10, 453-AAm⁶AC»TA-457; site 11, 464-GGm⁶ACA»G-468; site 12, 476-GAm⁶ACA»G-480; site 13, 518-GAm⁶ACC»G-522; and site 14, 553-AGm⁶A»GCC-557 in reference to SEQ ID NO: 3.

67. A host cell for producing an attenuated virus comprising a heterologous nucleic acid encoding one or more m6A eraser proteins, one or more inhibitors of m6A writer proteins, one or more inhibitors of m6A reader proteins, or a combination thereof.

68. The host cell of claim 67, wherein the eraser proteins comprise fat mass and obesity-associated (FTO) and/or AlkB homolog 5 (ALKBH5); the writer protein comprises METTL3 and/or METTL14; and/or the reader protein comprises YTHDF1, YTHDF2, YTHDF3 and/or YTHDC1.

69. The host cell of claim 67, wherein the inhibitor of m6A writer and/or reader proteins comprises an siRNA, shRNA, morpholino, and/or antisense nucleic acids.

70. The host cell of claim 69, wherein the siRNA comprises the sequence:
```
  (SEQ ID NO: 24)
5'-CCGCGTCTAGTTGTTCATGAA-3';
  (SEQ ID NO: 25)
5'-AAGGACGTTCCCAATAGCCAA-3';
  (SEQ ID NO: 26)
5'-ATGGATTAAATCAGTATCTAA-3';
  (SEQ ID NO: 27)
5'-CTGCAAGTATGTTCACTATGA-3';
  (SEQ ID NO: 28)
5'-AAGGATGAGTTAATAGCTAAA-3';
  (SEQ ID NO: 29)
5'-AAACAAGTACTTCTTCGGCGA-3';
and/or
(SEQ ID NO: 30)
5'-AAATAGCCGCTGCTTGTGAGA-3'.
```

71. The host cell of claim 67, further comprising a virus selected from the group consisting of Paramyxoviridae, Pneumoviridae, Rhabdoviridae, Filoviridae, and Bornaviridae.

72. The host cell of claim 67, wherein the cell comprises a VERO, MRC-5, HEp-2, A549, HeLa, or HAE cell.
